SEQUENCE LISTING (207 sequences)

SEQ ID NO: 1 - protein, 345 aa, African swine fever virus
MFPSLQSFAKKVLARQQVSIEYHVILERCGLWWYKAPISLDCKHMLIKLPSFADGLDLNTALMLATKENNYQLIKLFTEWGADINYGLICANTPPVREFCWELGAKYRVDKKKIMHMFFKLIHPGTTSSNIILCLKLFNDNPFPAYVIIREIRSSIYWKLKRLVEDTDILSNMSDGDMLTIYCFIVALQDNLREAISYVYQHFKYLNTWWLICALCFNKLFDLHNLYEKEKIRMDVDEMMRMACTKDNNFLTIYYCFLLGANINSAMLACVRFYNMDNLFFCIDLGADAFEEAKALAEQRNYYLISHHLSLDIYSADSSLLTLKEADPNKIYHLLKNYKLKSMLA

SEQ ID NO: 2 - protein, 353 aa, African swine fever virus
MLPSLQSLTKKVLAGQCIPVDQYHVLKCCGLWWHNGPIMLHIRRNKLFIRSTCFSQGIELNIGLMKAVKENNHDLIKLFTEWGADINYGMICALTENTRDLCKELGAKEYLEREYILKIFFDTTRDKTSSNIIFCHEVFSNNPNLRIIDNLDLRGEIMWELRGLMEITFMLDHDDSFSTVLTKYWYAIAVDYDLKDAIRYFYQKYPRLHRWRLMCALFYNNVFDLHELYEIERVRMDIDEMMHIACIQDYSYSAIYYCFIMGANINQAMLVSIQNYNLGNLFFCIDLGADAFEEGKALAEQKENYLIAHALSLKHYNPVISLLSIVTDPEKINCMLKNYHSINMGIFLDYEQR

SEQ ID NO: 3 - protein, 350 aa, African swine fever virus
MLPSLQSLTKKVLAGQCVPTNQHYLLKCYDLWWHDAPITFDHNLRLIKSAGIKEGLNLNTALVKAVRENNYNLIKLFAEWGADINYGLVSVNTEHTWDLCRELGAKETLNEEEILQIFIDLKFHKTSSNIILCHEVFSNNPILQKVNNIKMRIEIFWELRELIVKTDLLNNEFSLSTLLLKYWYAIAIRYNLKEAIQYFYQKYTHLNTWRLTCALCFNNVFDLHEAYEKDKIHMDIEEMMRIACIKDHNLSTMYYCYVLGANINQAMLSSIQYYNIENMFFCIDLGADVFEEGTTALGEGYELIKNILSLKIYSPATTPLPKSTDPEIIDHALKNYVSKNMMIFLTYDLR

SEQ ID NO: 4 - protein, 353 aa, African swine fever virus
MSLPLSLQTLVKKTIASQCLSIDEHCILKYCGLWWHDAPLKLCMDRGRIQIKSGFLGEDIDLRVALIIAVKENNYSLIKLFTEWGANINYGLLSINTKHIRELCRQLGAKETLEDNDIFRIFTRIMHNKTSGSIILCHEIFMNNPILENKFVIQLRGLIYKRLWGLIEIKETDELNGLLVKYWYAKAVQYDCKDAICFLDEKYTDLNEWRLKCLLYYNKIYELHEMYHKENIQIDVHDMICLASTKDNNPLTIYYCYALGGNINQAMLTSVQYYNIGNIFFCIDLGGNAFEEGRAIAEQKGYNFLSHSLALDIYSSDASLPLNLKDPEEISSLLKDYKSKNLSIIWEYSHNIL

SEQ ID NO: 5 - protein, 357 aa, African swine fever virus
MLSLQTLAKKVVACNYLSSDYDYTLQRFGLWWDLGPIHLCNNCKQVFSYKHLQCFSEDDLCLEAALVKAVKSDNLELIRLFVDWGANPEYGLIRVPAVYLKRLCAELGGLTPVSEPRLLEILKEVARLKSCAGVLLGYDMFCHNPLLETVTRTTLDTVTYTCSNIPLTGDTAHHLLTKFWFALALRHNFTKAIHYFYKRHKNHLYWRVACSLYFNNIFDIHELCREKEICISPNLMMKFACLREKNYAAIYYCHRLGASLDYGMNLSIYNNNTLNMFFCIDLGAADFDRAQLIAHKAYMYNLSNIFLVKQLFSRDVTLVLDVTEPQEIYDMLKTYTSKNLKRAEEYLTAHPEIIVID

SEQ ID NO: 6 - protein, 531 aa, African swine fever virus
MFSLQNLCRKTLPNRKLPEFFDEYILQLLGLYWENHGTIQRAGNNCVLIQQHTLIPVNEALRTAASEENYEIVSLLLAWEGNLYYAIIGALEGNRHDLIRKYDDQIKDHHEILPFIDDPVIFHKCHIMRQCFFDCILYQAVKYSKFRVLLYFKHRLEDDLPFTHLLIEKACKDHNYEVIKWIYENLHIYNMIDTFECAIAHKDLHLYCLGYRFIYNRIVPDKYHHLDIRMLSSLQLLHKVAAKGYLDFILETLKYDHNKDNINIILTQAATYNHRKILIYFIPQSTHAQIEQCLLVAIKAKSSRKTLNLLLSHLNLSINLIKKISHYVATYNSTNIIGILSMRRKKKIYLDIILTKFVKKAIFNKFVVRCMDTFSINPERILKIAARINRMMLVKKISEHVWKNHAVRLKYLKHAVHTMKHKDGKNRLMNFIYDRCYYHMQGEEIFSLARFYAIHHAPKLFDVFYDCCILDTIRFKSLLLDCSHIIGKNAHDATNINIVNKYIGNLFVMGVLSKKEILQDYPSIYSKQYMP

SEQ ID NO: 7 - protein, 526 aa, African swine fever virus
MFSLQDLCRKHLFILPDVFGEHVLQRLGLYWRCHGSLQRIGDDHILIRRDLILSTNEALRMAGEEGNNEVVKLLLLWKGNLHYAVIGALQGDQYDLIHKYENQIGDFHFILPLIQDANTFEKCHALERFCGVSCLLKHATKYNMLPILQKYQEELSMRAYLHETLFELACLWQRYDVLKWIEQTIHVYDLKIMFNIAISKRDLTMYSLGYIFLFDRGNTEATLLTQHLKKTAAKGLLHFVLETLKYGGNIDTVLTQAVKYNHRKLLDYFLRQLPRKHIEKLLLLAVQEKASKKTLNLLLSHLNYSVKRIKKLPRYVIEYESTLVIKILLKKRVNLIDAMLEKMVRYFSATKVRTIMDELSISPERVIKMAIQKMRTDIVIHTSYVWEDDLERLTRLKNMVYTIKYEHGKKMLIKVMHGIYKNLLYGEREKVMFYLAKLYVAQNAATQFRDICKDCYKLDVARFKPRFKQLILDCLEIITKKSCYSILEILEKHIISLFTMKVMTEEEKNLCLEILYKVIHYKTIQC

SEQ ID NO: 8 - protein, 280 aa, African swine fever virus
MSSSLQELCRKKLPDCILPEFFDDYVLQLLGLHWQDHGSLQRIEKNQILVQQEPIHINEALKVAASEGNYEIVELLLSWEADPRYAVVGALESKYYDLVYKYYDQVKDCHDILPLIQNPETFERCHELNSTCSLKCLFKHAVINDMLPILQKYTDYLDRWEYCSQMLFELACSKKKYEMVVWIEGVLGVGKVTSLFTIAISNRDLQLYSLGYSIILENLYSCGQDPKFLLNHFLRDVSIKGLLPFVIKTIEYGGSKEIAITLAKKYQHKHILKYFETWES

SEQ ID NO: 9 - protein, 145 aa, African swine fever virus
MDHYLKKLQDIYTKLEGHPFLFSPSKTNEKEFITLLNQALASTQLYRSIQQLFLTMYKLDPIGFINYIKTSKQEYLCLLINPKLVTKFLKITSFKIYINFRLKTFYISPNKYNNFYTAPSEEKTNHLLKEEKTWAKIVEEGGEES

SEQ ID NO: 10 - protein, 125 aa, African swine fever virus
MAVYAKDLDNNKELNQKLINDQLKIIDTLLLAEKKNFLVYELPAPFDFSSGDPLASQRDIYYAIIKSLEERGFTVKICMKGDRALLFITWKKIQSIEINKKEEYLRMHFIQDEEKAFYCKFLESR

SEQ ID NO: 11 - DNA, 1071 bp, African swine fever virus
ttataacgatgtatcattgatgtcataattcaaataggccaacatactttttgatttatagttttttaatagacgatatattttgttaggatctgcttcttttaacgttaatagcgaggagtctggactataaatgtctaatgataaacgatgagatatcaaatagtaattctgttgctctgccaaggcttttgcctcttcaaaggcatcggcccccagatctatacaaaagaacaggttatccatattatagaatcgtatggaggcaatcatggccaaattaatattagctcctaagataaaacaataatatatagttaaaaaattgttatcttttgtgcaggctatccgcatcatttcatccatgtccatacggattttttctttttcgtacaaattatgtaggtcaaacagcttattatagcaaagagcacatgttaaccaccacgtattcagatacttaaaatgttggtaaacataagaaatggcctccctaagattatcctgcaatgccacgataaaacagtatatagttaacatatcaccatccgaaatattacttaatacgttggtatcttctgctaaattttttagcttccaatgtatacacgactttatttcccttataatgacataggctgaaaagggattgtcattaaaaaatttaagacataagataatattattactagtagtgttaggatgtattaatttaaagaatatgtgcataatcttctttttatccacttggtacttggctcctaattcccagcaaaattctcgaataggtggcgtattcgcgcaaattaacccatagttgatgtctgcgccccaatccgtaaacatttttattaattgatagttgttttcctttgtagccaacattagtgccgtattaaggtccaagccatctgcaaagtttggcagctttatcagcatatgtttgcaatcaagtgaaattggagccttataccaccatagcccgcagcgttctaagataacatggtaatcaatagatacatgctgactggctaatacctttttggcgaaggattgcaaggaaggaaccat

SEQ ID NO: 12 - DNA, 1038 bp, African swine fever virus
ctaggccaacatactttttaatttatagttttttaatagatgatatattttgctaggatctgcttcttttaacgttaatagcgaggagtctgcactataaatgtctaatgataaatgatgagatatcaaatagtaattccgttgctctgctagggcctttgcctcttcaaaggcgtcggctcccagatctatacaaaagaacaagttatccatattataaaatcgtacgcaggcaagcatagctgaattaatattagctcctaagagaaaacaataatatatggttaaaaaattgttatcttttgtgcaggccatccgcatcatttcatccacgtccatgcggatcttttccttttcatacaaattatgtaggtcaaacagcttattaaaacaaagagcacagattaaccaccacgtatttagatacttaaaatgttggtaaacataagaaatggcctccctaagattatcctgcaatgccactataaaacagtatatcgttaacatatcaccatccgacatattacttaatatgtcggtgtcttctactaaccttttcaacttccaatatatggatgaccttatttcccttataatgacataggctggaaagggattatcattaaaaagtttaagacataagataatattactgctagtagtgccagggtgtattaatttaaagaacatgtgcataatcttctttttatccacgcggtacttggctcctaattcccagcaaaattctcgaacaggcggcgtattggcgcaaattaacccatagttgatgtctgcgccccattctgtaaacagttttattaactgatagttgttttcctttgtagccaacattagtgccgtattaaggtccaagccgtctgcaaagcttggcagctttatcagcatatgtttgcaatcaagggaaattggggccttataccaccatagtccgcagcgttctaagataacatggtactcaatagatacttgctgtctggctagtacctttttggcgaaggattgtaaggaaggaaacat

SEQ ID NO: 13 - DNA, 1038 bp, African swine fever virus
ctaggccaacatactttttaatttatagttttttaatagatgatatattttgttaggatctgcttcttttaacgttaatagcgaggagtctgcactataaatgtctaatgataaatgatgagatatcaaatagtaattccgttgctctgctagggcctttgcctcttcaaaggcgtcggctcccagatctatacaaaagaacaagttatccatattataaaatcgtacgcaggcaagcatagctgaattaatattagctcctaagagaaaacaataatatatggttaaaaaattgttatcttttgtgcaggccatccgcatcatttcatccacgtccatgcggatcttttccttttcatacaaattatgtaggtcaaacagcttattaaaacaaagagcacagattaaccaccacgtatttagatacttaaaatgttggtaaacataagaaatggcctccctaagattatcctgcaatgccactataaaacagtatatcgttaacatatcaccatccgacatattacttaatatgtcggtgtcttctactaaccttttcaacttccaatatatggatgaccttatttcccttataatgacataggctggaaagggattatcattaaaaagtttaagacataagataatattactgctagtagtgccagggtgtattaatttaaagaacatgtgcataatcttctttttatccacgcggtacttggctcctaattcccagcaaaattctcgaacaggcggcgtattggcgcaaattaacccatagttgatgtctgcgccccattctgtaaacagttttattaactgatagttgttttcctttgtagccaacattagtgccgtattaaggtccaagccgtctgcaaagcttggcagctttatcagcatatgtttgcaatcaagggaaattggggccttataccaccatagtccgcagcgttctaagataacatggtactcaatagatacttgctgtctggctagtacctttttggcgaaggattgtaaggaaggaaacat

SEQ ID NO: 14 - DNA, 1071 bp, African swine fever virus
ttacgacgttgtatcattattgtgataatccaaataggccgacatactttttgatttataattgtttaatagatggtatatttttttagggttcgcttcttttagcgtgaatagagagggatctggatgatagatatctaatgacaaacgatgtgatattaaataatagtcccgctgctctgctagggcttttgcctcttcgaaggcattggcccccaagtctatacaaaagaacagattatctatattaaaaaactgtatggcagcaagcatcgccatattgatatcggcccctaaaataaaacaataatacatcgttaaaaagttgttatctttcgtgcaagctatgcgcatcatttcattaatgtctatgcgaattttttcctgctcatagagataatggaggtcaaacagtttattaaaacacagagcacagattaaccaccacgtattcaggtgcttaaaatgttggtaaaaatacgagacggcctccctaagaccattctgcaaggccaccataaaacaatataaagttagcatatcgccgtccgaaacatcgcgtagtaggtcttcgtcttctactaacctcctcagcctccaataaagagacgactttatttcccttataatcacataggtaggaaaaggattctcattaaaaagcttatgacataagataatatattgactcgttttgttctctataaatttaaaaaacatatgcatcaccttttttttatctaagcgatagttcgctcctaattcccaacaaaactctcgagtaggctgtgtattggcacaaatgtatccatagttgatgtttgcaccccactccgtgaacagttttattaattgatagttgttttcctttgcagctatcatcaatgccgtattgaggtccaggccatccgcaaagtatggcaaccttattaacatgtgtttacaatcaagtgaaattggagccttataccaccataacccgcagcgcttcaaaatatgatagtagtcttcaggcagcacatgctggctggctaatatctttttggcgaaagattgcagggaaggaaacat

SEQ ID NO: 15 - DNA, 1071 bp, African swine fever virus
ttacgacgttgtatcattgatgtgataatccaaataggccaacatactttttgatttatagttgtttaatagatggtatatttttttagggttcgcttcttttagcgtgaatagagagggatctggatgatagatatctaatgacaaacgttgtgatattaaataatagtcccgctgctctgctagggcttttgcctcttcgaaggcattggctcccaagtctatacaaaagaacagattatctatattaaagaactgtatggcagcaagcatggccatattgatatcggcccctaaaataaaacaataatacatcgttaaaaagttattatctttcgtgcaagctatgcgcatcatttcattaatgtctatgcgaatcttttcctgctcatagagataatggaggtcagacagtttattaaaatacagagcacagattaaccaccacgtattcatgtgcttaaaatgttcgtaaaaatacgagacggcctccctaagatcattctgcaaggccaccataaaacaatatatagttagcatatcgccgtccgaaacatcacgtagtaggtcttcatcttctactaacctcctcagcctccaataaagagacgactttatttcccttataatcacataggtaggaaaaggattctcattaaaaagcttatgacataagataatatattgacttgttttgttctctataaatttaaagaacatgtgtatcaccttttttttatccatgcgatagttcgctcctaattcccaacaaaactctcgggtaggctgtgtattggcacaaatgtatccatagttgatgtttgcaccccactccgtgaatagttttattaattgatagttgttttcctttgcagctatcatcaatgccgtattgagatccaggccatccgcaaagtatggcaaccttattaacatgtgtttgcaatcaagtgaaattggagccgtataccaccataacccgcagtatttcaaaatatgatagtagtcttcaggcagcacatgctgactggctaatatctttttggcgaaagactgtaaagaaggaaacat

SEQ ID NO: 16 - DNA, 1071 bp, African swine fever virus
ttacgacgttgtatcattaatgtgataatccaaataggccgacatactttttgatttatagttgtttaatagatggtatatttttttagggttcgcttcttttagcgtgaatagagagggatctggatgatagatatctaatgacaaacgatgtgatattaaataatagtcccgctgctctgctagggcttttgcctcttcgaaggcattggcccccaagtctatacaaaagaacagattatccatattaaagaactgtatggcagcaagcatggccatattgatatcggcccctaaaataaaacaataatacatcgttaaaaagttgttatctttcgtgcaagctatgcgcatcatttcattaatgtctatgcgaattttttcctgctcatagagataatggaggtcaaacagtttattaaaacacagagcacagattaaccaccacgtattcaggtgcttaaaatgttggtaaaaatacgagacggcctccctaagatcattctgcaaggccaccataaaacaatataaagttagcatatcgtcgtccgaaacatcgcgtagtaggtcttcgtcttctactaatctcctcagcctccaataaagagacgactttatttcccttataatcacataggtaggaaaaggattctcattaaaaagcttatgacataagataatatactgactcgttttgttctctataaatttaaaaaacatatgcatcaccttttttttatctaagcgatagtttgctcctaattcccaacaaaactctcgagtaggctgtgtattggcacaaatgtatccatagttgatgtttgcaccccactccgtgaacagttttattaattgatagttgttttcctttgcagctatcatcaatgccgtattgaggtccaggccatccgcaaagtatggcaaccttattaacatgtgtttacaatcaagtgaaattggagccttataccaccataacccgcagcgcttcaaaatatgatagtagtcttcaggcagcacatgctggctggctaatatctttttggcgaaagattgcagggaaggaaacat

SEQ ID NO: 17 - DNA, 1071 bp, African swine fever virus; identical to SEQ ID NO: 11

SEQ ID NO: 18 - DNA, 1068 bp, African swine fever virus
ttacactgtgtcatgcccatcatagtcaaaatataccatcatattttttgatttatagttttttaatagatgatatatttttttaggatctgcttcttttaacgttaatagcgaggagtctggactataaatatctattgataaacgatgatacatcaaaaagtaattccgctggtctgccagggcttttgcctcttcaaaggcatcggctcccaggtctatacaaaagaacaagttatccatattatagaattgtacgcaggcaagcatagcctgattaatattagctcctaagagaaaacagtaatatatggttaaaaagttgttatctttagtgcaggctatgcacatcatttcatccatgtccatgcggatcttttccttttcatacaaatcatgtaggtcaaacagcttattaaaacaaagagcacagattaaccaccacgtattcagatgcttaaaatgttggtaaaaataagaaatggcctccctaagattatcctgcaatgccaggataaaacagtatatagtcaacatatcatcatccgacatattacttaatatgtcagtgtcttctactaaccttctcagcttccaatatatagacgactttatttcccttataatgacataggttggaaaaggattattattaaaaagtttaagacataagataatattactactagtagtgccatgatgtattaatttaaaaaacatatgcataaccttatttttatccacttggtacttggctcctaattcccagcaaaattctcgaacaggcggcgtattagcgcaaattaatccatagttgatgtccgcgccccattccgtaaacagttttattaactgatagttgttttcctttgtagccaacattagtgccgtattaaggtccaagccatctgcaaagcttggcagctttattaacatatgtttgcaatcaagtgaaattggagccttataccaccatagtccgcaatgttttaagatataatgataatcaatggatacatgatgtctagctaatacctttttggcgaaggattgtaaagaaggaaacat

SEQ ID NO: 19 - DNA, 1071 bp, African swine fever virus
ttataacgatgtatcattgatgtcataattcaaataggccaacatactttttgatttatagttttttaatagacgatatattttgttaggatctgcttcttttaacgttaatagcgaggagtctggactataaatgtctaatgataaacgatgagatatcaaatagtaattctgttgctctgccaaggcctttgcctcttcaaaggcatcggcccccagatctatacaaaagaacaggttatccatattatagaatcgtatggaggcaatcatggccaaattaatattagctcctaagataaaacaataatatatagttaaaaaattgttatcttttgtgcaggctatccgcatcatttcatccatgtccatacggattttttctttttcgtacaaattatgtaggtcaaacagcttattatagcaaagagcacatgttaaccaccacgtattcaaatacttaaaatgttggtaaacataagaaatggcctccctaagattatcctgcaatgccacgataaaacagtatatagttaacatatcaccatccgaaatattacttaatacgttggtatcttctgctaaattttttagcttccaatgtatacacgactttatttcccttataatgacataggctgaaaagggattgtcattaaaaaatttaagacataagataatattactactagtagtgctaggatgtattaatttaaagaatatgtgcataatcttctttttatccacttggtgcttggctcctaattcccagcaaaattctcgaataggtggcgtattcgcgcaaattaacccatagttgatgtctgcgccccaatccgtaaacatttttattaattgatagttgttttcctttgtagccaacattagtgccgtattaaggtccaagccatctgcaaagtttggcagctttatcagcatatgtttgcaatcaagtgaaattggagccttataccaccatagcccgcagcgttctaagataacatggtgatcaatagatacatgctgactggctaatacctttttggcgaaggattgcaaggaaggaaacat

SEQ ID NO: 20 - DNA, 1071 bp, African swine fever virus
ttataacgttgtattattgacgtcataattcaaataggccaatatactttttgatttatagttttttaatagatgatatattttgttaggatccgcttcttttaacgttaataacgaggaatctggactataaatgtctaatgataaacaatgagatatcaaaaagtaattccgttgctctgccagggcttttgcctcctcaaaggcatcggcccccaggtctatacaaaagaataagttatctatgttatagaattgtatggaggcaatcatagctaaattaatattagctcctaagataaaacaataatatatggttaaaaaattgttatcttttgtgcaggctatccgcatcatttcatccatgtccatgcggatcttttccttttcatacaaatgatgtaggtcaaacaccttattatagcaaagaacacatgttaaccaccacgtattcagatacttaaaatgttggtaaacataagaaatggcctccctaagattatcctgcaatgccacgataaaacagtatatagttaacatatctccatccgaaatattacttaatacgttggtatcttctgctaacttttttagcttccaatgtatacacgactttatttcccttataatgacataggctgaaaagggattgtcattaaaaagtttaagacataagataatattactactagtagtgctaggatgtattaatttaaagaatatgtgcataatcttctttttatccacttggtacttggctcctaattcccagcaaaattctcgaacaggtggcgtattcgcgcaaattaacccatagttgatgtctgcgccccaatccgtaaacatttttattaattgatagttgttttcctttgtagccaacattagtgccgtattaaggtccaagccatctgcaaagtttggcagctttatcagcatatgtttgcaatcaagtgaaattggagccttataccaccatagcccgcagcgttctaagataatatggtgatcaatggatacatgctgtctggctagtacctttttggcgaaggattgtaaggaaggaaacat

SEQ ID NO: 21 - DNA, 1071 bp, African swine fever virus
ttataacaatgtatcattgatatcatcattcaaataggccaacatattttttgatttatagttttttaatagatgatttattttgttaggatctgtttcttttaacgttaatagcaaggagtctggcttataaatgtctaatgataaacgatgagatattaaatagtaattccgttgctctgccagggcttttgcctcttcaaaggcgtcggctcccagatctatacaaaagaacaagttatccatattataaaatcgtacgcaggcaagcatagctgaattaatattagctcctaagagaaaacaataatatatagttaaaaaattgttatcttttgtgcaggccatccgcatcatttcatccacgtccatgcggatcttttccttttcatacaaattatgtaggtcaaacagcttattaaaacaaagagcacagattaaccaccacgtatttagatacttaaaatgttggtaaacataagaaatggcctccctaagattatcctgcaatgccactataaaacagtatatcattaacatatcaccatccgacatattacttaatatgtcggtgtcttctactaaccttttcagcttccaatatatggatatccttatttcccttataatgacataggctggaaagggattatcattaaaaagtttaagacataagataatattactgctagtagtgccaggatgtattaatttaaagaacatgtgtataatcttctttttatccacgcggtgcttggctcctaattcccagcaaaattctcgaacagacggcgtattggcgcaaattaacccatagttgatgtctgcgccccattccgtaaacagttttattaactgatagttattttcctttgtagccaacattagtgccgtattaaggtccaagccgtctgcaaagcttggcagctttatcagcatatgtttgcagtcaagtgaaattggagccttataccaccatagtccgcaacgttctaagataacatggtactcaatagatacttgctgtctggctagtacctttttggcgaaggattgtaaggaaggaaacat

SEQ ID NO: 22 - DNA, 1062 bp, African swine fever virus
tcatattgtatcatcatggttaaaatatgccgtcatattttttgatttatagttttttaatagatgatatattttgttagggtccgcttcttttaacgttaatagcgaggagtctggactataaatgtctaatgataaacgatgagatatcaaaaagtaattccgttgctctgccagagcctttgcctcttcaaaggcgtcggcccccaggtctatacaaaagaacaggttatccatattatagaatcgtatggaggcaatcatggccaaattaatattagctcctaagataaaacaataatatatagttaaaaaattgttatcttttgtgcaggctatccgcatcatttcatccatgtccatgcggattttttccttttcgtacaaattatgtaggtcaaacagcttattaaaacaaagagcacagattagccaccacatattcagatacttaaaatgttggtaaacataagaaatggcctccctaagattatcctgcaatgccaccataaaacagtatatcgttaacatatcaccatctgaaatatcacttaatacgtcggtgtcttctactaactttctcagcttccaatatatggatgactttatttcccttataatgacataggctgaaaaggggttatcattaaaaagtttaagacataagataatattactgctagtagtgccaggatgtattaatttaaagaacatgtgcataatcttctttttatccacgcggtacttggctcctagttcccagcacaattctcgaacaggcggtgtattggcgcaaattaacccatagttgatgtctgcgccccattccgtaaacagttttattaactgatagttgttttcctttgtagccaacattagtgccgtattaaggtccaagccatctgcaaagcttggcagctttatcagcatatgtttgcaatcaagtgaaattggagccttataccaccatagtccgcagcgttctaagataacatggtaatcaatagatacatgctgtctggctagtacctttttggcgaaggattgtaaggaaggaaacat

SEQ ID NO: 23 - DNA, 1062 bp, African swine fever virus
tcatattgtatcatcatggttaaaatatgccgtcatattttttgatttatagttttttaatagatgatatattttgttagggtccgcttcttttaacgttaatagcgaggagtctggactataaatgtctaatgataaacgatgagatatcaaaaagtaattccgttgctctgccagggcttttgcctcttcaaaggcatcggcccccaagtctatacaaaagaacaggttatccatattataaaatcgtatggaggcaatcatggccaaattaatattagctcctaagataaaacaataatatatagttaaaaaattgttatcttttgtgcaggctatccgcatcatttcatccatgtccatgcggattttttccttttcgtacaaattatgtaggtcaaacagcttattaaaacaaagagcacagattagccaccacgtattcagatatttaaaatgttggtaaacataagaaatggcctccctaagattatcctgcaatgccaccataaaacagtatatcgttaacatatcaccatccgaaatattacttaatacgtcggtgtcttctactaactttttcagcttccaatatatggatgactttatttcccttataatgacataggctgaaaaggggttatcattaaaaagtttaagacataagataatattactgctagtagtgccaggatgtattaatttaaagaacatgtgcataatcttctttttatccacgcggtacttggctcctagttcccagcacaattctcgaacaggcggcgtattggcgcaaattaacccatagttgatgtctgcgccccattccgtaaacagttttattaactgatagttgttttcctttgtagccaacattagtgccgtattaaggtccaagccatctgcaaagcttggcagctttatcagcatatgtttgcaatcaagggaaattggagccttataccaccatagtccgcagcgttctaagataacatggtaatcaatagatacatgctgtctggctagtacctttttggcgaaggattgtaaggaaggaaacat

SEQ ID NO: 24 - DNA, 1062 bp, African swine fever virus
ttatctttgttcataatcaagaaaaatccccatatttattgagtgataattttttaacatgtaatttattttttcagggtccataacgttcgacaacagagaaatgactggattgtaatgctttaatgataaggcatgggctatcagataattttccttttgttctgccaaagctttgccctcctcaaaggcgttggcgcccaggtctatacaaaagaacaggtttccaagattatagttttgtatggaaaccagcatggcttgattgatgttggctcccatgataaaacagtagtaaatggccgaatagctataatcttggatgcaggctatgtgcatcatttcatcgatatccatgcggaccctttctatttcatacagctcgtgaaggtcgaacacgttgttgtaaaaaagggcgcacatgagccgccaccgatgtaggcgcgggtatttctggtaaaagtagcgaatagcatctttgaggtcatagtccaccgctatagcataccagtatttggttaaaacagtgctaaagctatcatcatggtctagcatgaaggttatctccatgagccctcttaactcccacatgatttcccccctcagatccagattatctataatccttaaattggggttattggaaaacacctcgtggcaaaagataatattgctactggttttatcgcgcgtcgtatcaaagaaaatttttaaaatatactctctttctaaatattctttggctcccagctctttgcacagatcacgggtattttccgtgagagcacaaatcattccatagttaatatctgcaccccattcagtaaacagctttatcaagtcatgattattctccttcacggctttcatcagtcctatgtttaactcgataccttgactaaagcaggttgaccttataaatagtttattacgtcgaatatgaagcataatggggccattatgccaccacaggccacaacacttcaggatatgataatgatccaccgacacgcactgcccggccagtactttcttcgtgagggattgcagggaaggcaacat

SEQ ID NO: 25 - DNA, 1062 bp, African swine fever virus
ttatctttgttcataatcaagaaaaatccccatatttattgagtgataattttttaacatgcaatttattttttcagggtccgtaacgatcgacaacagagaaataaccggattgtaatgctttaatgataaggcatgggctatcagataattttccttttgttctgccaaagctttgccctcctcaaaggcatcggcacccaggtctatacaaaagaacaggtttccaagattatagttttgtatggaaacaagcatggcttgattgatgttggctcccatgataaaacagtagtaaatggccgaatagctataatcttggatgcaggctatgtgcatcatttcatcaatatccatgcggaccctttctatttcgtacagctcgtgaaggtcgaacacgttgttgtaaaaaagggcgcacatgagccgccacctatgtagacgcgggtatttctggtaaaagtagcggatagcatctttgaggtcatagtccaccgctatcgcgtaccagtatttggttaaaacagtgctaaagctatcatcatggtccagcatgaaggttatctccatgagccctcttaactcccacatgatttcccccctcagatccagattatctataatccttaaattggggttattggaaaacacctcgtggcaaaagataatattgctactggttttatcgcgcgttgtatcaaagaaaatttttaaaatatactctctttctaaatattctttggctcccagctctttgcacagatcacgggtattttccgtgagagcacaaatcattccatagttaatatctgcaccccattcagtaaacagctttatcaagtcatgattattctccttcacggctttcatcagtcctatgtttaactcgataccttgactaaaacaggttgaccttataaataatttattgcgtcgaatatgaagcataatggggccattatgccaccacaggccacaacacttcaggacatgatattgatctaccggtatacactgcccggccagtactttcttcgtgagggattgcagggaaggcaacat

SEQ ID NO: 26 - DNA, 1062 bp, African swine fever virus; identical to SEQ ID NO: 25

SEQ ID NO: 27 - DNA, 1062 bp, African swine fever virus; identical to SEQ ID NO: 24

SEQ ID NO: 28 - DNA, 1062 bp, African swine fever virus
ttatctgcgttcataatcgagaaaaaccaccatattctttgagtgataattttttaacatagaatttatcttttctgggtcggtcacggccgacaacagggaaatcaccgggttataatgttttaatgataaggcatcggctatcaaataattttccttttgtattgctagggctttgccttcttcaaaggcgttggcacccaggtctatacaaaagaacatgttcccgagattatagttttgtatggaaacaaacatggcttgattgatgttggcccccatgataaaacagtagtaaatagctgaatagctataatcttggatgcaggctatgtgcatcatctcatcgatatccatgcggaccctttctatttcatacagctcgtgaaggtcgaacacattgttataaaaaagggcgcacataagccgccaccgatgtagatgggtatacttctgataaaaatagtggatggcctttttgaggccgtagtctaccgctatggcgtaccagtatttggttaacatattgctgaaggagttgtcatggtccaacatgtaggttatttccatgagtccccttagctcccacatgatttcctccctcagatccaaatcatccacatgtttcagattggcgttattggaaaacatttcgtggcataagataatattactgtcggttttgttgcgcgtcgtatcaaagaaaatttttaaaatatactctctatctaaacggtctttggctccaagttctctacatagattgcgggtgtgctccgaatgagcacaaaccagtccataattaatatcggcaccccactcaacaaacagccttatcaagtcatgattgttttccttcgcggctttcatcagtacggtgttcaattcaataccctcgctaaaacaggttgactttataaacattttgttacgatacgtataaaatgtaatagggccatcataccaccacaacccgcagcatttcaggatatgatattgatctaccggtatacactgtttggccagcactttcttggatagggattgcaaggaaggcaacat

SEQ ID NO: 29 - DNA, 1062 bp, African swine fever virus
ttatctgcgttcataatcgagaaaaaccaccatattctttgagtgataattttttaacatagaatttatcttttccgggtcggtcacggccgacaacagggaaatcaccgggttataatgttttaatgataaggcatcggctatcaaataattttccttttgtattgctagggctttgccttcttcaaaggcgttggcacccaggtctatacaaaagaacatgttcccgagattatagttttgtatggaaacaaacatggcttgattgatgttggcacccatgataaaacagtagtaaatagttgaatagctataatcttggatgcaggctatgtgcatcatctcatcgatatccatgcggatcctttctatttcatacagctcgtgaaggtcgaacacattgttataaaaaagggcgcacatgagccgccaccgatgtagatgggtatacttctgataaaaatagtggatggcctttttgaggccgtagtctaccgctatggcgtaccagtatttggttaacatgttgctgaaggagttgtcatggtccaacatgtaggttatctccatgagtccccttagctcccacatgatttcctccctcaggtccaaatcatccacatgtttcagattggggttattggaaaacatttcgtggcataagataatattactgtcggttttgttgcgcgtcgtatcaaagaaaatttttaaaatatactctttatctaaacggtctttggcccctagttctctgcatagatcgcgggtgtgctccgaatgagcacaaaccagtccataattaatatcggcaccccactcagtaaataattttattaggtcatgattgttttccttcgcggctttcatcagtacggtgttcaattcaataccctcgctaaaacaggttgactttataaacattttgttacgatacgtataaaatgtaatagggccatcataccaccacaacccgcaacttttcaggatatgatattgatctaccggtatacactgtttggccagcacttttttggatagggattgcaaggaaggcaacat

SEQ ID NO: 30 - DNA, 1062 bp, African swine fever virus
ttatctgcgttcataatcgagaaaaaccaccatattctttgagtgataattttttaacatagaatttatcttttctgggtcggtcacggccgacaacagggaaatcaccgggttataatgttttaatgataaggcatcggctatcaaataattttccttttgtattgctagggttttgccttcttcaaaggcgttggcacccaggtctatacaaaagaacatgttcccgagattatagttttgtatggaaacaaacatggcttgattgatgttggcccccatgataaaacagtagtagatagctgaatagctataatcttggatgcaggctatgtgcatcatctcatcgatatccatgcggaccctttctatttcatacagctcgtgaaggtcgaacacattgttataaaaaagggcgcacataagccgccaccgatgtagatgggtatacttctgataaaaatagtggatggcctttttgaggccgtagtctaccgctatggcgtaccagtatttggttaacatattgctgaaggagttgtcatggtccaacatgtaggttatctccatgagtccccttagctcccacatgatttcctccctcagatccaaatcatccacatgtttcagattggggttattggaaaacatttcgtggcataagataatattactgtcggttttgttgcgcgtcgtatcaaagaaaatttttaaaatatactctctatctaaacggtctttggctccaagttctctgcatagattgcgggtgtgctccgaatgagcacaaaccagtccataattaatatcggcaccccactcaacaaacagccttatcaagtcatgattgttttccttcgcggctttcatcagtacggtgttcaattcaataccctcgctaaaacaggttgactttataaacattttgttacgatacgtataaaatgtaatagggccatcataccaccacaacccgcagcatttcaggatatgatattgatctaccggtatacactgtttggccagcactttcttggatagggattgcaaggaaggcaacat

SEQ ID NO: 31 - DNA, 1062 bp, African swine fever virus
ttatcttcgttcataatcaagaaaaaccctcatatttattgagtgataattttttaacatgcaatttattttttcagggtccataatgaccgacaacagagaaatgaccggattgtaatgctttaatgataaggcatcggctatcagataattttccttttgttctgccaaagctttcccctcctcaaaggcgttggcccccagatctatacaaaagaacatgttcccaagattatagttttgtatagaaacaagcatggcttgattgatgttggcccccatgataaaacagtaataaatagccgcatagctataatcttggacgcaggctatgtgcatcatttcatcaatatccatgcggaccctttctattacatacagctcgtgaaggtcgaacacgttgttgtaaaaaagggcgcacataagccgccagtggtgtagatgggtatacctctgataaaaataatggatagcctttttgaggccatagtctaccgctatggcgtaccagtatttggttaacatattgctaaaggagttgtcatggtccaacatgtaggttatctccatgagtccccttagctcccacatgatttcctccctcagatccaaattatccacatttttcaggttggggttattggaaaacatttcgtggcataagataatattactgtcggttttgttgcgcgtcgtatcaaagaaaatttttaaaatataatctctatctaaacgatctttggctcctagttctctgcatagattacgggtgtgctccgtatgggcacaaatcagtccataattaatatcggcaccccactcggcaaacagccttatcaagtcatggttgttttccttcgcggctttcatcagtacggtgttcaattcaataccctcgctaaaacaggttgaccttataaacattttgtgacgataaacgtaaaatgtaatagggccatcataccaccacaacccgcagcactttaagacatgatattgatctaccggtacacactgtttggccagcactttcttagttaaagattgtaaggaaggcaacat

SEQ ID NO: 32 - DNA, 1062 bp, African swine fever virus
ttatctttgttcataatcaagaaaaatccccatatttattgagtgataattttttaacatgtaatttattttttcagggtccataacgatcgacaacagagaaatgaccggattgtaatgctttaatgataaggcatgggctatcagataatttcccttttgttctgccaaagctttgccctcctcaaaggcgttggcgcccaggtctatacaaaagaacaggtttccaagattatagttttgtatggaaaccagcatagcttgattgatgttggctcccatgataaaacagtagtaaatgaccgaatagctataatcttggatgcaggctatgtgcatcatttcatcaatatccatgcggaccctttctatttcatacagctcgtgaaggtcgaacacgttgttgtaaaaaagggcgcacatgagccgccacctatgtaggcgcgggtatttctggtaaaagtagcgaatagcatctttgaggtcatagtccaccgctatagcataccagtatttggttaaaacagtgctaaagctatcatcatggtctagcatgaaggttatctccatgagccctcttaactcccacatgatttctcccctcagatccagattatctataatccttaaattggggttattggaaaacacctcgtggcaaaagataatattgctactggttttatcgcgcgtcgtatcaaagaaaatttttaaaatatactctctttctaaatattctttggctcccagctctttgcacagatcacgggtattttccgtgagagcacaaatcattccatagttaatatctgcaccccattcagtaaacagctttatcaagtcatgattattctccttcacggctttcatcagtcctatgtttaactcgataccttgactaaagcaggttgaccttataaatagtttattacgtcgaatatgaagcataatggggccattatgccaccacaggccacaacacttcaggatatgataatgatccaccgacacgcactgcccggccagtactttcttcgtgagggattgcagggaaggcaacat

SEQ ID NO: 33 - DNA, 1062 bp, African swine fever virus
ttatctttgttcataatcaagaaaaatccccatatttattgagtgataattttttaacatgcgatttattttttcagggtccgtaaccaccgacaacagagaaatgaccggattgtaatgctttaatgataaggcattggctatcagataatttcccttttgttctgccaaagctttgccctcttcaaaggcgttggcacccaggtctatacaaaagaacaggttcccaagattatagttttgtatggaaaccagcatggcttgattgatgttggctcccatgataaaacagtagtaaatggctgaatagctataatcttggacgcaggctatgtgcatcatttcatcaatgtccatgcggaccctttctatttcatacagctcgtgaaggtcgaacacgttgttgtaaaaaagggcgcacatgagccgccacctatgtagccgcgggtatttctggtaaaagtagcggatagcatctttgaggtcatagtccaccgctatcgcgtaccagtatttggttaaaacagtgctaaagctatcatcatggtccagcatgaaggttatctccatgagccctcttaactcccacatgatttcccccctcagatccagattatctataatccttaaattggggttattggaaaacacctcgtggcaaaagataatattgttactggttttatcgcgcgttgtatcaaagaaaatttttaaaatatactctctttctaaatattctttggctcccagctctttgcacagatcacgggtattttccgtgagagcacaaatcattccatagttaatatctgcaccccattcagtaaacagctttatcaagtcatgattattctccttcacggctttcatcagtcctatgtttaactcgataccttgactaaagcaggttgaccttataaatattctattacgtcgaatatgaagcattatggggccattatgccaccacaggccacaacacttcaggatatgataatgatctaccgacacgcactgcccagccagtactttcttcgtgagggattgcagggaaggcaacat

SEQ ID NO: 34 - DNA, 1062 bp, African swine fever virus
ttatctttgttcataatcaagaaaaatccccatatttattgagtgataattttttaacatgcaatttatttttttagggtctgtaacgaccgacaacagagaaatgaccggattgtaatgctttaatgataaggcatcggctatcagataatttcccttttgttctgccaaagctttgccttcctcaaaggcgttggctcccaggtctatacaaaagaacaggttcccaagattatagttttgtatggaaaccagcatggcttgattgatgttggctcccatgataaaacagtagtaaatggccgaatagctataatcttggacgcaggctatgtgcatcatttcatcaatgtccatgcggaccctttctatttcgtacagctcgtgaaggtcgaacacgttgttgtaaaaaagggcgcacatgagccgccacctatgtagacgcgggtatttctggtaaaagtagcggatcgcatctttgaggtcatagtccaccgctatcgcgtaccagtatttggttaaaacagtgctaaagctatcatcatggtccagcatgaaggttatctccatgagccctcttaactcccacatgatttcccccctcagatccagattatctataatccttaaattggggttattggaaaacacctcgtggcaaaagataatattgttactggttttatcgcgcgtcgtatcaaagaaaatttttaaaatatactctctttctaaatattctttggcacccagctctttgcacagatcacgggtattttccgtgagagcacaaatcattccatagttaatatctgcaccccattcagtaaacagctttatcaagtcatgattattctccttcacggctttcatcagtcctatgtttaactcgataccttgactaaagcaggttgaccttataaatagtttattacgtcgaatctgaagcataatgggtccattatgccaccacaggccacaacacttcaggatatgataatgatccaccgacacgcactgcccggccagtactttcttcgtgagggattgcagggaaggcaacat

SEQ ID NO: 35 - DNA, 1062 bp, African swine fever virus
ttatctttgttcataatcaagaaaaatccccatatttattgagtgataattttttaacatgcgatttattttttcagggtccgtaaccaccgacaacagagaaatgaccggattgtaatgctttaatgataaggcattggctatcagataatttcccttttgttctgccaaagctttgccctcctcaaaggcgttggcacccaggtctatacaaaagaacaggttcccaagattatagttttgtatggaaaccagcatggcttgattgatgttggctcccatgataaaacagtagtagatggctgaatagctataatcttggacgcaggctatgtgcatcatttcatcaatgtccatgcggaccctttctatttcatacagctcgtgaaggtcgaacacgttgttgtaaaaaagggcgcacatgagccgccacctatgtagccgcgggtatttctggtaaaagtagcggatagcatctttgaggtcatagtccaccgctatcgcgtaccagtatttggttaaaacagtgctaaagctatcatcatggtccagcatgaaggttatctccatgagccctcttaactcccacatgatttcccccctcagatccagattatctataatccttaaattggggttattggaaaacacctcgtggcaaaagataatattgttactggttttatcgcgcgttgtatcaaagaaaatttttaaaatatactctctttctaaatattctttggctcccagctctttgcacagatcacgggtattttccgtgagagcacaaatcattccatagttaatatctgcaccccattcagtaaacagctttatcaagtcatgattattctccttcacggctttcatcagtcctatgtttaactcgataccttgactaaagcaggttgaccttataaatattctattacgtcgaatatgaagcattatggggccattatgccaccacaggccacaacacttcaggatatgataatgatccaccgacacgcactgcccggccagtactttcttcgtgagggattgcagggaaggcaacat

SEQ ID NO: 36 - DNA, 1062 bp, African swine fever virus
ttatctttgttcataatcaagaaaaatccccatatttattgagtgataattttttaacatgcgatttattttttcagggtccgtaaccaccgacaacagagaaatgaccggattgtaatgctttaatgataaggcattggctatcagataatttcccttttgttctgccaaagctttgccctcctcaaaggcgttggcccccaggtctatacaaaagaacaggttcccaagattatagttttgtatggaaaccagcatggcttgattgatgttggctcccatgataaaacagtagtaaatggctgaatagctataatcttggacgcaggctatgtgcatcatttcatcaatgtccatgcggaccctttctatttcatacagctcgtgaaggtcgaacacgttgttgtaaaaaagagcgcacatgagccgccacctatgtagccgcgggtatttctggtaaaagtagcggatagcatctttgaggtcatagtccaccgctatcgcgtaccagtatttggttaaaacagtactaaagctatcatcatggtccagcatgaaggttatctccatgagccctcttaactcccacatgatttctccccttagatccagattatctataatccttaaattggggttattggaaaacacctcgtggcaaaagataatattgttactggttttatcgcgcgttgtatcaaagaaaatttttaaaatatactctctttctaaatattctttggctcccagctctttgcacagatcacgggtattttccgtgagagcacaaatcattccatagttaatatctgcaccccattcagtaaacagctttatcaagtcatgattattctccttcacggctttcatcagtcctatgtttaactcgataccttgactaaagcaggttgaccttataaatattctattacgtcgaatatgaagcattatggggccattatgccaccacaggccacaacacttcaggatatgataatgatccaccgatacgcactgcccggccagtactttcttcgtgagggattgcagggaaggcaacat

SEQ ID NO: 37 - DNA, 1053 bp, African swine fever virus
tcatcttaaatcataggaaaggaagatcatcatattttttgaaaagtaattttttaacgcatgatctatgatttcagggtccgtgcttttaggcaacgggatggtggtcggactataaatctttagagataaaatgttttttataagctcatacccttcccctaaagctgtagtaccctcttcgaaaacatcagcccccagatccatacaaaagaacatgttttctatattatagtactgtattgaggtaagcatggcctgattgatgttggcgcccaacatatagcagtagtacatggttgaaaggttgtggtctttgatgcaggcaatccgcatcatctcttctatgtccatatggatcttgtccttttcatacgcctcatggaggtcaaacacattattaaaacaaagagcacatgttaaccgccacgtattcatgtgtgtatatttttggtaaaaatactgtatggcctctttcaggctatagcgtacggctatagcgtaccagtatttgagtagtaatgtactgagcaaaaactcattatttagcagatcggttttttctattaactcccttaactcccagaaaatttctatcctcaatttcagattatttactttttgtaatatcggattgttggaaaacacctcatggcataaaataatgttactactagttttatgaaactttagatctatgaaaatttgtaaaatttcctcttcattcaagatctccttggcacctaactcttgacagaggtcccgggtatgctccgtgttgacagataccagcccatagttgatgtccgccccccactctgtaaatagttttataaggctgtagttgttttcccttacagcctttactaacgccgtatttaggtctaagccctctttaatgcctgatgattttatgagccttaggttatgatcaaacgtgatcggagcattataccaccatagatcataatattttaaaagataatgttggttcgtgggcacgcattgtccagccaacacctttttggtcaaagattgcagggaaggcaacat

SEQ ID NO: 38 - DNA, 1053 bp, African swine fever virus
tcatcttaaatcataggtaaggaagatcatcatattttttgaaacgtaattttttaacgcatgatctatgatttcagggtccgtgcttttaggcaacggggtggtggccggactataaatctttagggataaaatgttctttataagctcatacccttcccctaaagctgtagtaccctcttcgaaaacatcagcccccagatctatacaaaagaacatgttttctatattatagtactgtattgagctaagcatggcttgattgatgttggcgcccaggacatagcagtagtacatggttgaaaggttgtggtctttgatgcaggcgatccgcatcatctcttctatgtccatatggatcttgtccttttcatacgcctcatgaaggtcaaacacattattaaaacaaagagcacatgttaaccgccacgtattcaggtgtgtatatttttggtaaaaatactgtatggcctctttcaggttatagcgtatggctatagcgtaccagtatttgagtagtaatgtactgagcgaaaactcattatttagcagatcggtttttactattaactcccttaactcccagaaaatttctatcctcatttttatattatttactttttgtaatatcggattgttggaaaacacctcatggcataaaataatgttactactagttttatgaaactttagatctataaaaatttgtaaaatttcttcttcattcaaggtttccttggcacctagctctcgacagaggtcccaggtgtgctccgtgttgacagataccagcccgtagttgatgtccgccccccactctgcaaacagttttataaggttgtagttgttttcccttacagccttcactaacgccgtatttaggtttaagccctctttaatacctgctgattttatgagccttaggttatgatcaaacgtgatcggagcatcatgccaccataggtcataacactttaaaagataatgttggttcgtgggcacgcattgtccagccaacacctttttggtcagagattgcagggaaggcaacat

SEQ ID NO: 39 - DNA, 1053 bp, African swine fever virus; identical to SEQ ID NO: 38

SEQ ID NO: 40 - DNA, 1056 bp, African swine fever virus
tcatcttagatcataagtaaggaagaccatcatattttttgaaaagtaattttttaacgcatgatctataatttcagggtccgtgcttttaggcaatggggtagtggccgggctataaatctttagggataaaatattccttataggctcatacccttcctgtcccactagagccataccctcttcaaaagcatcggcccccagatccatacaaaagaatatgttttctatattatagtactgtattgaggtaagcatggcctgattgatgttggcgcccagcatatagcagtagtatattgttgaaaggttgtggtctttgatgcaggctatccgcatcatctcttctaaatccatacagatcttgtccttttcatacgcctcatgaaggtcaaacacgttattaaaacaaagagcacatgttaaccgccacgtattcaggtgtgtatatttttggtaaaaatactgtatggcctctttcaagttgtagcgtacggctatagcgtaccagtatttgagtaatagtgtattgagcgaaaactcattatttagcagatcggttttttctattatctcccttaactcccaaaaaatttctatcctcattttcaggttatttaccctttttaaaagtaaattatcggaaaacacctcatgacataagataatgttactactggttttataaaattttaaatctatgaaaatttgtaaaatatccccttcatttaaggtcttcttggcgcctagctctcgacagaggtcccaggtgtgctccgtgttgacagatatcagcccgtagttaatgttcgccccccactccgtaaatagttttataagactgtagttgttttcctttacagccttcactaatgccatgtttaggtctaagccttcttgaatgcctgctaattttatcagccttaggttatgatcaaacgtgatcggagcattattccaccatagatcgtaacactttaaaagataatgttggtcctcgggcagacattgtctagccagcacctttttggtcaaagattgcagggaaggcaacat

SEQ ID NO: 41 - DNA, 1056 bp, African swine fever virus
tcatcttagatcataagtaaggaagaccatcatattttttgaaaagtaattttttaactcatgatctataatttcagggtccgtgcttttaggcaacggggtggtggccgggctataaatctttagggataaaatattccttataggctcatacccttcctgtcccactagagccataccctcttcaaaagcatcggcccccagatccatacaaaagaatatgttttctatattatagtactgtattgaggtaagcatggcctgattgatgttggcgcccagcatatagcagtagtatatcgttgaaaggctgtggtctttgatgcaggctatccgcatcatctcttctaaatccatacagatcttgtccttttcatacgcctcatgaaggtcaaacacgttattaaaacaaagagcacatgttaatcgccacgtattcaggtgcgtatatttttggtaaaaatactgtatggcctctttcaagttgtagcgtacggctatagcgtaccagtatttgagtaatagtgtattgagcgaaaactcattatttagcagatcggttttttctattatctcccttaactcccaaaaaatttctatcctcattttcaggttatttaccttttttaaaagtagattatcggaaaacacctcatgacataagataatgttactactggttttatgaaactttaaatctatgaaaatttgtaaaatatccccttcatttaaggtcttcttggcgcctagctctcgacagaggtcccaggtgtgctccgtgttgacagatatcagcccgtagttgatgttcgctccccactccgtaaatagttttataagactgtagttgttttcctttacagccttcactaatgccatgtttaggtctaagccttcttgaaggcctgctgattttatcagccttaggttatgatcaaacgtgatcggagcattattccaccatagatcgtaacactttaaaagataatgttggtcctcgggcagacattgtccagccagcacctttttggtcagagattgcagggaaggcaacat

SEQ ID NO: 42 - DNA, 1053 bp, African swine fever virus
tcatcttaaatcataggaaaggaagatcatcatattttttgaaaagtaattttttaacgcatgatctatgatttcagggtccgtgcttttaggcaacgggatggtggtcggactataaatctttagagataaaatgttttttataagctcatacccttcccctaaagctgtagtaccctcttcgaaaacatcagcccccagatccatacaaaagaacatgttttctatattatagtactgtattgaggtaagcatggcctgattgatgttggcgcccaacatatagcagtagtacatggttgaaaggttgtggtctttgatgcaggcaatccgcatcatctcttctatgtccatatggatcttgtccttttcatacgcctcatggaggtcaaacacattattaaaacaaagagcacatgttaaccgccacgtattcaggtgtgtatatttttggtaaaaatactgtatggcctctttcaggctatagcgtacggctatagcgtaccagtatttgagtagtaatgtactgagcaaaaactcattatttagcagatcggttttttctattaactcccttaactcccagaaaatttctatcctcaatttcagattatttactttttgtaatatcggattgttggaaaacacctcatggcataaaataatgttactactagttttatgaaactttagatctatgaaaatttgtaaaatttcctcttcattcaagatctccttggcacctaactcttgacagaggtcccgggtatgctccgtgttgacagataccagcccatagttgatgtccgccccccactctgtaaatagttttataaggctgtagttgttttcccttacagcctttactaacgccgtatttaggtctaagccctctttaatgcctgatgattttatgagccttaggttatgatcaaacgtgatcggagcattataccaccatagatcataatattttaaaagataatgttggttcgtgggcacgcattgtccagccaacacctttttggtcaaagattgcagggaaggcaacat

SEQ ID NO: 43 - DNA, 1050 bp, African swine fever virus
tcatcttaaatcgtaggaaaggaaggtcatcatattttttgatttataattttttaacacatgatctattatttcagggtccgtgtttttaggtaacggggcagtggacggactatagatctttagggataaaatgttccttataagctcatacccttcccctacaggcatattctcttcgaaaacatcagcccccagatccatacaaaagaacatgttttctatattatagtactgtattgaggcaagcatggcctgattaatgttggcgcccaggacatagcagtagtatatcgttgaaaggttgtggtctttgatgcaggctactcgcatcatctcttctaagtccatatagatcttgtccttttcatacgcctcatgaaggtcaaacacattattaaaacaaagagcacatgttaaccgccacgtattcaggtgtgtatatttttgataaaaatactgtatggcctctttcaggttatagcgtacggctatagcgtaccagtatttgagtaataatgtattgagtgaaaactcattatttaacagatcggttttttttattaactcccttaactcccagaaaatttctatcctcattttcagattatttactttttgtaataacggattgttggaaaacacctcatggcataaaataatgttactactagttttataaaactttagatcgatgaaaattcgtaaaatttcctcttcatttaaggtctccttggcgcctagctctctgcagaggtcccgggtgtgctccgtgttgacagataccagcccgtagttgatgtccgccccccactctgtaaaaagctttataaggttgtagttgttttcccttacagcctttactaacgccgtatttaggtctaagccatctttaatgcctgctgattttatcagccctaggttatgatcaaacgtgatcggagaatcataccaccataggtcgtaacactttaaaagataatattggtccgtcggcaggcattgtccagccagtacctttttggtcagagattgcagggaaggcaacat

SEQ ID NO: 44 - DNA, 1053 bp, African swine fever virus
tcatcttaaatcataggaaaggaagatcatcatattttttgaaaagtaattttttaacgcatgatctatgatttcagggtccgtgcttttaggcaacgggatggtggtcggactataaatctttagagataaaatgttttttataagctcatacccttcccctaaagctgtagtaccctcttcgaaaacatcagcccccagatccatacaaaagaacatgttttctatattatagtactgtattgaggtaagcatggcctgattgatgttggcgcccaacatatagcagtagtacatggttgaaaggttgtggtctttaatgcaggcgatccgcatcatctcttctatgtccatatagatcttgtccttttcatacgcctcatggaggtcaaacacattattaaaacaaagagcacatgttaaccgccacgtattcaggtgtgtatatttttggtaaaaatactgtatggcttctttcaggttatagcgtatggctatagcgtaccagtatttgagtagcaatgtactgagcgaaaactcattatttagcagatcggttttttctattaactcccttaactcccaaaaaatttctatcctcatttttatattatttactttttgtaatatcggattgttggaaaacacctcatggcataaaataatgttactactagttttatgaaactttagatctataaaaatttgtaaaatttctttttcattcaaggtctccttggcacctagctctcgacagaggtcccaggtgtgctccgagctgacagataccagcccatagttgatgtccgccccccactctgcaaacaattttataaggttgtagttgttttcctttacagccttcaccaacgccgtatttaggtctaagccctctttaatgcctgctgattttatgagccttaggttatgatcaaacgtgatcggagcatcatgccaccataggtcataatattttaaaagataatgttggttcgtgggcacgcattgtccggccaacacctttttggtcaaagattgcagggaaggcaacat

SEQ ID NO: 45 - DNA, 1053 bp, African swine fever virus
tcatcttaaatcgtaggtaaggaagatcatcatattttttgaaacgtaattttttaacgcatgatctataatttcagggtccgtgcttttaggcaacggggtggtggccggactataaatctttagggataaaatgttctttataagcttatacccttcccctaaagctatagtgccctcttcgaaagcatcagcccccagatccatacaaaagaacatgttttctatattatagtactgtattgaggtaagcatggcctgattgatgttgccccccaggacatagcagtagtacatggttgaaaggttgtggtctttgatgcaggcgatccgcatcatctcttctatgtccatatgaatcttgtccttttcatacgcctcatgaaggtcaaacacattattaaaacaaagagcacatgttaaccgccacgtattcaggtgtgtatatttttggtaaaaatactgtatggcctctttcaggttatagcgtatggctatagcgtaccagtatttaagtagtaatgtactgagcgaaaactcattatttagcagatcggttttttctattaactcccttaactcccagaaaatttctatcctcatttttatattatttactttttgtaatatcggattgttggaaaacacctcatggcataaaataatgttactactagttttatgaaactttagatctataaaaatttgtaaaatttcttcttcattcaaggtctccttggcacctagctctcgacagaggtcccaggtgtgctccgtgttgacagataccagcccatagttgatgttcgccccccactctgcaaacagttttataaggttgtagttgttttcccttacagccttcactaacgccgtatttaggtctaagccctctttaatgcctgctgattttatgagctttaggttatgatcaaacgtgatcggaccatcatgccaccataggtcgtaacactttaaaagataatgttggtttgtgggcacgcattgtccagccaacacctttttggtcagagattgcagggaaggcaacat

SEQ ID NO: 46 - DNA, 1053 bp, African swine fever virus
tcatcttaaatcataggtaaggaagatcatcatattttttgaaacgtaattttttaacgcatgatctatgatttcagggtccatgcttttaggcaacggggtggtggccggactataaatctttagggataaaatgttctttataagctcatacccttcccctaaagctgtagtaccctcttcgaaaacatcagcccccagatctatacaaaagaacatgttttctatattatagtactgtattgagctaagcatggcctgattgatgttggcgcccaggacatagcagtagtacatggttgaaaggttgtggtctttgatgcaggcgatccgcatcatctcttctatgtccatatggatcttgtccttttcatacgcctcatgaaggtcaaacacattattaaaacaaagagcacatgttaaccgccacgtattcaggtgtgtatatttttggtaaaaatactgtatggcctctttcaggttatagcgtatggctatagcgtaccagtatttgagtagtaatgtactgagcgaaaactcattatttagcagatcggttttttctattaactcccttaactcccagaaaatttctatcctcatttttatattatttactttttgtaatatcggattgttggaaaacacctcatggcataaaataatgttactactagttttatgaaactttagatctataaaaatttgtaaaatttcttcttcattcaaggtctccttggcacctagctctcgacagaggtcccaggtgtgctccgtgttgacagataccagcccgtagttgatgtccgccccccactctgcaaacagttttataaggttgtagttgttttcccttacagcctttactaacgccgtatttaggtttaagccctctttaatacctgctgattttatgagccttaggttatgatcaaacgtgatcggagcatcatgccaccataggtcataacactttaaaagataatgttggttcgtgggcacgcattgtccagccaacacctttttggtcagagattgcagggaaggcaacat

SEQ ID NO: 47 - DNA, 1053 bp, African swine fever virus
tcatcttaaatcgtaggtaaggaagatcatcatattttttgaaacgtaattttttaccgcatgatctatgatttcagggtccgtgcttttaggcaacggggtggtggccggactataaatctttagggataaaatgttctttataagcttatacccttcccctaaagctatagtaccctcttcgaaagcatcagcccccagatccatacaaaagaacatgttttctacattatagtactgtattgaggtaagcatggcctgattgatgttggcgcccagaacatagcagtagtacatggttgaaaggttgtggtctttgatgcaggcgatccgcatcatctcttctatgtccatatagatcttgtccttttcatacgcctcatgaaggtcaaacacattattaaaacaaagagcacatgttaaccgccatgtattcaggtgtgtatatttttggtaaaaatactgtatggcctctttcaggttatagcgtatggctatagcgtaccagtatttgagtagtaatgtactgagcgaaaactcattatttagcagatcggttttttctattaactcccttaactcccagaaaatttctatcctcatttttatattatttactttttgtaatatcggattgttggaaaacacctcatggcataaaataatgttactactagttttatgaaactttagatctataaaaatttgtaaaatttcttcttcattcaaggtctccttggcacctagctctcgacagaggtcccgggtgtgctccgtgttgacagataccagcccatagttgatgtccgccccccactctgcaaacagttttataaggttgtagttgttttcccttacagccttcactaacgccgtatttaggtctaagccctctttaatgcctgctgattttatgagctttaggttatgatcaaacgtgatcggaccatcatgccaccataggtcataacactttaaaagataatgttggttcgtgggcacgcattgtccagccaacacctttttggtcagagattgcagggaaggcaacat

SEQ ID NO: 48 - DNA, 1053 bp, African swine fever virus
tcatcttaaatcgtaggtaaggaagatcatcatattttttgaaacgtaattttttaacgcatgatctataatttcagggtccgtgcttttaggcaacggggtggtggccggactataaatctttagggataaaatgttctttataagcttatacccttcccctaaagctatagtgccctcttcgaaagcatcagcccccagatccatacaaaagaacatgttttctatattatagtactgtattgaggtaagcatggcctgattgatgttggcgcccaggacatagcagtagtacatggttgaaaggttgtggtctttgatgcaggcgatccgcatcatctcttctatgtccatatggatcttgtccttttcatacgcctcatgaaggtcaaacacattattaaaacaaagagcacatgttaaccgccacgtattcaggtgtgtatatttttggtaaaaatactgtatggcctctttcaggttatagcgtatggctatagcgtaccagtatttgagtagtaatgcactgagcgaaaactcattatttagcagatcggttttttctattaactcccttaactcccagaaaatttctatcctcatttttatattatttactttttgtaatatcggattgttggaaaacacctcatggcataaaataatgttactactagttttatgaaactttagatctataaaaatttgtaaaatttcttcttcattcaaggtctccttggcacctagctctcgacagaggtcccgggtgtgctccgtgttgacagataccagcccatagttgatgttcgccccccactctgcaaacagttttataaggttgtaattgttttcccttacagccttcactaacgccgtatttaggtctaagccctctttaatgcctgctgattttatgagctttaggttatgatcaaacgtgatcggaccatcatgccaccataggtcataacactttaaaagataatgttggttcgtgggcacgcattgtccagccaacacctttttggtcagagattgcagggaaggcaacat

SEQ ID NO: 49 - DNA, 1062 bp, African swine fever virus
ctataatatattatgagaatattcccaaatgatggataagttttttgatttataatcttttaataaactgcttattttttcggggtcctttaagtttaatggcaaggaagcatctgaactgtaaatatccaaagtcaagctatggcttaaaaaattataacctttttgttccgctatggcacgaccctcttcaaaggcattaccacccaaatctatacagaaaaatatattaccgacgttataatattgtactgaagtgagcatagcttggttgatgttgccccccagggcgtaacagtaatatattgttaatagattgttatccttggcacaagccagacatatcatgtcatggacgtctatttggacttttttcttgtggtacatctcatgaagctcatatattttgttataatacaggagacattttaatcgccactcgttaagatccgtatatttctcatctagaaagcaaatggcgttcttacaaacgtattgtactgctttggcataccaatacttcactagtaaatcatttaactcgtccgtttcttttatttctatgagcccccatagtcttttataaattaagccccttaattgtataacaaatttgttttctaacataggattattcataaaaatttcatggcacaaaataatactgccgctggttttattgtgcattatcctggtgaaaatacggaaaatatcgttgtcctctagagtttctttggcgcctagctgtctacacaactctcggatgtgcttcgtattgatagaaagcaaactatagttgatatttgcgccccactctgtaaagagctttatcagactatagttgttttccttaacagctattattaatgccacacgaaggtctatatcttctcctaaaaatcctgattttatttgtattcggccacgatccatacaaagcttgagaggagcatcatgccaccataggccacaatatttcaaaatgcagtgttcatctattgacaaacactggctggctaccgtctttttgacgagggtctgcagagagagcggcaacgacat

SEQ ID NO: 50 - DNA, 1062 bp, African swine fever virus
ctatagtatattatgagaatattcccaaatgatggataagttttttgatttataatcttttaataaactgcttatttcttcggggtcctttaagtttagtggcaaggaagcatctgagctgtaaatatccaaagccaaactatggctcagaaaattataacctttttgttccgctatggcacgaccctcttcaaaggcattaccacccaaatctatacagaaaaatatattaccgatgttataatattgtactgaagtaagcatagcttggttgatgttgccccccagcgcgtaacagtaatatattgttaatggattgttatccttggtagaagccagacatatcatgtcatggacgtctatttggatgttttccttgtggtacatctcatgaagctcatatattttgttataatacaggagacattttaatcgccattcattaagatccgtatatttctcatctagaaaacaaatggcgtccttacaatcgtattgtactgctttggcgtaccaatacttcactagtaaaccatttaactcgtccgtttcttttatttctatgagcccccatagtcttttataaattaagccccttaattgtataacaaatttgttttctaaaataggattattcataaaaatttcatggcacaaaataatactgccgctggttttattgtgcattatcctggtaaaaatacggaaaatatcgttgtcctctagagtttctttggcgcctagctgtctacacaactctcggatgtgcttcgtattgatagaaagcaaaccatagttgatatttgcgccccactctgtaaagagctttatcagactatagttgttttccttaacagctattattaatgccacacgaaggtctatatcttctcctaaaaatcctgattttatttgtattcggccacgatccatacaaagcttgagaggagcatcatgccaccataggccacaatatttcaaaatgcagtgttcatctattgacaaacactggctggctatcgtctttttgacgagggtctgcagagagagcggcaacgacat

SEQ ID NO: 51 - DNA, 1062 bp, African swine fever virus; identical to SEQ ID NO: 50

SEQ ID NO: 52 - DNA, 1062 bp, African swine fever virus
ttagagtattttatgagagtaatccagaatgatggataaattttttgatttataattttttaataaactgcttatttcttcgggatcctttaagtttaatggcaaggaagcatctgagctataaatattcaaaaccaaactatggcttaaaaaattataaccattttgtctcgctatggcactaccctcttcaaaggcattaccacccaaatctatacagaaatatatattaccgatgttataatattgtactgaagtaagcatagcttgattgatgttgccccccagggcataacaataatatattgttaatagattgttatccctgatacaagccagagataacatttcattgacgtctatttggattttttccttgtggtatatatcatgaagctcatatattttgttataacataggtaacattttaatcaccattcatcaagatccgtatatttttcatccagaaagcaaatggcatctttatggtcgtattgtactgcattggcgtaccaatatttcactagcaattcgcttaactcgtccgtttcttttatttctataagcccgcatagtcttttataaattaagcgccttaattgtgcagcaaagttgttttctaaattaggattatttataaatatctcatggcacaaaataatactgccgctagttttattatgcattatcttgttgaaaatacagaaaatatcgtagtcgtctagagtttctttggcgcctagctgtctacacaaatctcgggcgtgcttcgtattgatagaaagcagactatagttgatatatgcgccccactctgtaaataactttatcatactatagttgttttccttaacagctattattaatgccacacaaaggtctatatcttctcctaggaatcccgattttatgtatattcgcccacgatctatgtaaagcttgaggggagcatcatgccaccataagccacagtatttcaaaatgtgttcatctatcgacaaacatgatgtaccggctaccgtctttttgacgagggtctgcagagagagcggcgacgacat

SEQ ID NO: 53 - DNA, 1062 bp, African swine fever virus
ttagagtattttatgagagtaatccagaatgatggataaattttttgatttataattttttaataaactgcttatttcttcgggaacctttaagtttaatggcaaggaagcatctgagctataaatattcaaaattaaactatggcataaaaaattataaccattttgtctcgctatggcactaccctcttcaaaggcattaccacccaaatctatacagaaatatatattaccgatgttataatattgtactgaagtaagcatagcttggttgatgttgccccccagggcataacaataatatattgttaatagattgttatccctgatacaagccagagataacatttcattgacgtctatttggatttttttcttgtggtatatatcatgaagctcatatattttgttataacataggtaacattttaatcgccattcatcaagatccgtatatttttcatccagaaagcaaatggcatctttatgatcgtattgtactgcattggcgtaccaatatttcactagtaattcgcttaactcgtccgtttcttttatttctataagcccgcataatcttttataaattaagcgccttaattgtgcagcaaagttgttttctaatttaggattatttataaatatctcatggcacaaaataatactgccgctagttttattatgcattattttattgaaaatacagaaaatatcgtagtcgtctagagtttctttggcgcctagctgtctacacaaatctcgggcgtgcttcgtattgatagaaagcagactatagttgatatatgcgccccactctgtaaataactttatcagactatagttattttccttaacagctattattaatgccacacaaaggtctatatcttctcctaggaatcccgattttatgtatattcgtccacgatctatgtaaagcttgaggggagcatcatgccaccataagccacagtatttcaaaatgtgttcatctatcgacaaacatgatgtactggctaccgtctttttgacgagggtctgcagagagagcggcgacgacat

SEQ ID NO: 54 - DNA, 1062 bp, African swine fever virus
ctataatatattatgagaatattcccaaatgatggataagttttttgatttataatcttttaataaactgcttattttttcggggtcctttaagtttaatggcaaggaagcatctgaactgtaaatatccaaagtcaagctatggcttaaaaaattataacctttttgttccgctatggcacgaccctcttcaaaggcattaccacccaaatctatacagaaaaatatattaccgacgttataatattgtactgaagtgagcatagcttggttgatgttgccccccagggcgtaacagtaatatattgttaatagattgttatccttggcacaagccagacatatcatgtcatggacgtctatttggacttttttcttgtggtacatctcatgaagctcatatattttgttataatacaggagacattttaatcgccactcgttaagatccgtatatttctcatctagaaagcaaatggcgttcttacaaacgtattgtactgctttggcataccaatacttcactagtaaatcatttaactcgtccgtttcttttatttctatgagcccccatagtcttttataaattaagccccttaattgtataacaaatttgttttctaacataggattattcataaaaatttcatggcacaaaataatactgccgctggttttattgtgcattatcctggtgaaaatacggaaaatatcgttgtcctctagagtttctttggcgcctagctgtctacacaactctcggatgtgcttcgtattgatagaaagcaaactatagttgatatttgcgccccactctgtaaagagctttatcagactatagttgttttccttaacagctattattaatgccacacgaaggtctatatcttctcctaaaaatcctgattttatttgtattcggccacgatccatacaaagcttgagaggagcatcatgccaccataggccacagtatttcaaaatgcagtgttcatctattgacaaacactggctggctaccgtctttttgacgagggtctgcagagagagcggcaacgacat

SEQ ID NO: 55 - DNA, 1062 bp, African swine fever virus
ttagagtatattatgagagtaatccaaaatgatggataaattttttgatttataattttttaacaaactgcttatttcttcggggtcctttaagtttaatggcaaggaagcatccgagctataaatattcaaaaccaaactatggcttagaaaattataacctttttgtctcgctatggcactaccctcttcaaaggcattaccacccaaatctatacagaaatatatattaccgatgttataatattgcactgaagtaagcatagcttggttgatgttgccccccagggcataacagtaatatattgttaatggattgttatccctgatacaagccagggataacatttcattaacgtctatttggattttttccttgtggtatatatcatgaagctcatatattttgttataatataggtaacattttaatcgccattcattaagatccgtatatttctcatccagaaagcaaatggcatctttataatcgtattgtactgctttggcgtaccaatatttcactagtaattcacttaactcgtccgtttcttttatttctataagcccgcatagtcttttataaattaagcgccttaattgtgcagcaaatttgttttctaaattaggattatttataaatatctcatggcacaaaataacactgccgctggttttattatgcattatcttgttgaaaatacagaaaatatcgtagtcctccagagtttctttggcgcctagctgtctacacaaatctcggacgtgcttcgtattgatagaaagcagactatagttgatatttgcgcccaactctgtaaaaagctttattagattatagttgttttccttaacagctattattaatgccacacaaaggtctatatcttctcctaaaaatcccgattttatttgtattcggccacgatccatataaagcttgagaggggcatcatgccaccataagtcacagtatttcaaaatgtgttcatctatcgacaaacatgatgtactggctaccgtctttttgacgagggtttgtagagagagcggcgccgacat

SEQ ID NO: 56 - DNA, 1062 bp, African swine fever virus
ctataatatattatgagaatattcccaaatgatggataagttttttgatttataatcttttaataaactgcttatttcttcggggtcctttaagtttaatggcaaggaagcatctgaactgtaaatatccaaagtcaagctatggcttaaaaaattataacctttttgttccgctatggcacgaccctcttcaaaggcattaccccccaaatctatacagaaaaatatattaccgatgttataatattgtactgaagtgagcatagcttggttgatgttgctccccagggcgtaacagtaatatattgttaatagattgttatccttggcacaagccagacatatcatgtcatggacgtctatttggacttttttcttgtggtacatctcatgaagctcatatattttgttataatacaggagacattttaatcgccattcgttaagatccgtatatttctcatctagaaagcaaatggcgttcttacaaacgtattgtactgctttggcataccaatacttcactagtaaatcatttaacttgtccgtttcttttatttctatgagcccccatagtcttttataaattaagccccttaattgtataacaaatttgttttctaaaataggattattcataaaaatttcatggcacaaaataatactgccgctggttttattgtgcattatcctggtgaaaatacggaaaatatcgttgtcctctagagtttctttggcgcctagctgtctacacaactctcggatgtgcttcgtattgatagaaagcaaactatagttgatatttgcgccccactctgtaaagagctttatcagactatagttgttttccttaacagctattattaatgccacacgaaggtctatatcttctcctaaaaatcctgattttatttgtattcggccacgatccatacaaagcttgagaggagcatcatgccaccataggccacagtatttcaaaatgcagtgttcatctattgacaaacactggctggctaccgtctttttgacgagggtctgcagagagagcggcaacgacat

SEQ ID NO: 57 - DNA, 1062 bp, African swine fever virus
ctatagtatattatgagaatattcccaaattatggataagttttttgatttataatcctttaaaaaactgcttatttcttcggggtcctttaagtttagtggcaaggaagcatctgagctgaaaatatccaaagccaaactatggcttagaaaattataacctttttgttccgctatggcacgaccctcttcaaaggcattaccacccaaatctatacagaaaaatatattaccgatgttataatattgtattgaagtaagcatagcttggttgatgttgccccccagggcgtaacagtaatatattgttaatggattgttatccttggtagaagccagacatatcatgtcatggacgtctatttggactttttccttgtggtacatctcatgaagctcatatattttgttaaaatacaggagacattttaatcgccattcattaagatccgtatatttctcttctagaaagcaaatggcgtccttacactcgtattgtactgctttggcgtaccaatacttcactagtaaatcgtttaactcatccgtttcttttatttctatgagcccccatagtcttttacaaattaagccccttaattgtatagtaaatttgttttctatattaggattattcataaatatatcatggcacaaaataatacggccgctggttttattgtgcattatcttggtgaaaatacggaaaacatcgtcgtcttctagagtttctttggcgcctagctgtctacacaactctcggatgtgctccgtattaatagaaagcaaactatagttgatatgtgcgccccattctgtaaagagctttatcagactatagttgttttccttaacagctattattaatgctatatggaggtctatatcctctcctaaaaatcctgattttatttgtatttggccacgatccatacaaagcttgagggggacatcatgccaccataggccacagtgtttcagaatgcagtgttcatctatcgacaaacactggctggctaccgtctttttgacgagggtctgcagagagagcggcaacgacat

SEQ ID NO: 58 - DNA, 1062 bp, African swine fever virus
ctatagtatattatgagaatattcccaaatgatggataagttttttgatttataatcttttaataaactgcttatttcttcggggtcctttaagtttagtggcaaggaagcatctgagctgtaaatatccaaagccaaactatggctcagaaaattataacctttttgttccgctatggcacgaccctcttcaaaggcattaccacccaaatctatacagaaaaatatattaccgatgttataatattgtactgaagtaagcatagcttggttgatgttgccccccagcgcgtaacagtaatatattgttaatgggttgttatccttggtagaagccagacatatcatgtcatggacgtctatttggatgttttccttgtggtacatctcatgaagctcatatattttgttataatacaggagacattttaatcgccattcattaagatccgtatatttctcatctagaaagcaaatggcgtccttacactcgtattgtactgctttggcgtaccaatacttcactagtaaaccgtttaactcatccgtttcttttatttctatgagcccccatagtcttttataaattaagccccttaattgtatagtaaatttatcttctatattaggattattcataaatatatcatggcacaaaataatactgccgctggttttattgtgcattatcttggtgaaaatacggaaaatatcgtcgtcttctagagtttctttggcgcctagctgtctacacaactctcggatgtgcttcgtattgatagaaagcaaactatagttgatatttgcgccccactctgtaaagagctttatcagactatagttgttttccttaacagctattattaatgctatatggaggtctatatcttctcctaaaaatcctgattttatttgtatttggccacgatccatacaaagcttgagggggacatcatgccaccataggccacagtgtttcagaatgcagtgttcatctatcgacaaagactggttggctaccgtctttttgacgagggtctgcagagagagcggtaatgacat

SEQ ID NO: 59 - DNA, 1062 bp, African swine fever virus
ctatagtatattatgagaatattcccaaatgatggataagttttttgatttataaccctttaataaactgcttatttcttcggggtcctttaagtttagtggcaaggaagcatctgagctgtaaatatccaaagccaaactatggcttagaaaatgataacctttttgttccgctatggcacgaccctcttcaaaggcattaccacccaaatctatacagaaaaatatattaccgatgttataatattgtactgaagtaagcatagcttggttgatgttgccccccagggcgtaacagtaatatattgttaatggattgttatccttggtagaagccagacatatcatgtcatggacgtctatttggactttatccttgtggtacatctcatgaagctcatatattttgttataatacaggagacattttaatcgccattcattaagacccgtatatttctcatctagaaagcaaatggcgtccttacactcgtattgtactgctttggcgtaccaatacttcactagtaaatcgtttaactcatccgtttcttttatttctatgagcctccatagtcttttacaaattaagccccttaattgtatagtaaatttgttttctatattaggattattcataaatatctcatggcacaaaataatacggccgctggttttattgtgcattatcttggtgaaaatacggaaaatatcgttgtcctctagagtttttttggcacctagctgtctacacaactctcggatgtgttccgtattgatagaaagcaaactatagttgatatgtgcgccccattctgtaaagagctttatcagactatagttgttttccttaacagctattattaatgctacatggagatctatatcttctcctaaaaatcctgattttatttgtatttggccacgatccatacaaagcttgagggggacatcatgccaccataggccacagtgtttcagaatgcagtgttcatctgtcgacaaacactggctggctaccgtttttttgacgagggtctgcagagagagcggcaacgacat

SEQ ID NO: 60 - DNA, 1062 bp, African swine fever virus
ctatagtatattatgagaatattcccaaatgatggataagttttttgatttataatcctttaataaactgcttatttcttcggggtcctttaagtttagtggcaaggaagcatctgagctgtaaatatccaaagtcaaactatggcttagaaaatgataacctttttgttccgctatagcacgaccctcttcaaaggcattaccacccaaatctatacagaaaaatatattaccgatgttataatattgtactgaagtaagcatagcttggttgatgttgccccccagggcgtaacaataatatattgttaatggattgttatccttggtagaagccagacatatcatgtcatggacgtctatttggactttttccttgtggtacatctcatgaagctcatatattttgttataatacaggagacattttaatcgccattcattaagatccgtatatttctcatctagaaagcaaatggcgtccttacactcgtattgtactgctttggcgtaccaatacttcactagtaaatcgtttaactcatccgtttcttttatttctatgagcccccatagtcttttacaaattaagccccttaattgtatagtaaatttgttttctatattaggattattcataaatatctcatggcacaaaataatacggccgctggttttattgtgcattatcttggtgaaaatacggaaaatatcgttgtcctctagagtttctttggcacctagctgtctacacaactctcggatgtgctccgtattgatagaaagcaaactatagttgatatgtgcgccccattctgtaaagagctttatcagactatagttgttttccttaacagctattattaatgctatatggaggtctatatcttctcctaaaaatcctgattttatttgtatttggccacgatccatacaaagcttgagggggacatcatgccaccataggccacagtgtttcagaatgcagtgttcatctgtcgacaaacactggctggctaccgtctttttgacgagggtctgcagagagagcggcaacgacat

SEQ ID NO: 61 - DNA, 1074 bp, African swine fever virus
ttagtctataactataatttctggatgggctgtaagatactcttcggctcgtttcatattttttgaagtatatgtctttagcatatcatatatttcctggggttcggttacatctaataccaaggtcacatcacggctgaaaagctgctttactaagaaaatgttgctcaagttatacatataagctttgtgcgcaatgagttgtgccctatcaaaatcggcggcccccaaatcaatacagaaaaacatgtttaaagtattattgttatagatagaaagattcatgccataatcgagactagcccccaacctatgacagtaataaatggccgcgtaatttttttcccgcaagcaagcaaatttcatcatcagattagggctaatgcaaatctctttttcacgacacagctcgtgtatgtcaaaaatgttattaaaataaaggctacaagctacccgccaatagagctgatttttatgccttttataaaaatagtggatagcctttgtaaaattatgtcgtaatgccagggcaaaccaaaactttgttaataggaggtgcgccgtatcccccgtcaacggaatgtttgaacaggtgtacgtaactgtgtctaaagtggttctagttacggtttccaagagtggattatgacaaaacatgtcataacccagtagaactcctgcacaggattttaggttggccacttcttttaaaatttccagaagacggggttcggatacaggcgtcaagcctcccagttccgcacacagccgctttagatacacggcaggaacacgtataagcccatattcaggatttgcgccccaatccacaaataaacgtataagttcaagattatcgctcttcacggcctttactagcgccgcttcaagacaaagatcatcctcagaaaaacactgtaaatgtttatacgaaaaaacttgcttacaattgttacataggtgaataggacctaaatcccaccacaaaccaaaacgctgcaacgtataatcatagtcacttgaaagataattgcatgccacaacttttttggccaacgtttgtaaagacaacat

SEQ ID NO: 62 - DNA, 1074 bp, African swine fever virus
ttagtctataactataatttctggatgggctgtaagatactcttcggctcgtttcagattttttgaagtatatgtctttagcatatcatatatttcctggggttcggttacatctaataccaaggtcacatcacggctgaaaagctgctttactaagaaaatgttgctcaagttatacatataagctttgtgcgcaatgagttgtgccctatcaaaatcggcagcccccaaatcaatacagaaaaacatgtttaaagtattattgttatagatagaaagattcatgccataatcgagactagcccccaacctatgacagtaataaatggccgcgtaatttttttcccgcaagcaagcaaatttcatcatcagattagggctgatgcaaatctctttttcacgacacaactcgtgtatgtcaaaaatgttattaaaataaaggctacaagctacccgccaatagaggtgatttttatgccttttatagaaatagtgaatagcctttgtaaaattatgtcgtaatgccagggcaaaccaaaactttgttaataggtggtgcgccgtatcccccgtcaacggaatgtttgaacaggtgtacgtaactgtgtctaaagtggttctagttacggtttccaagagtggattatgacaaaacatgtcataacccagcagaactcctgcacaggattttagcctggccacttcttttaaaatttccagaagacggggttcggatacaggcgttaagcctcccagttccgcacacagccgctttagatacacggcaggaacacgtataagcccatattcaggatttgcgccccaatccacaaataaacgtataagttcaagattatcgctcttcacggcctttactagcgccgcttcgagacaaagatcatcctcagaaaaacactgtaaatgtttatacgaaaaaacttgcttacaattgttacataggtgaataggacctaaatcccaccacaaaccaaaacgctgcaacgtataatcatagtcacttgaaagataattgcatgccacaacttttttggccaacgtttgtaaagacaacat

SEQ ID NO: 63 - DNA, 1074 bp, African swine fever virus; identical to SEQ ID NO: 62

SEQ ID NO: 64 - DNA, 1074 bp, African swine fever virus
ttagtctataacgacaatttctggatgggctgtaaaatactcttcggctcgttttagattttttgacgtatatgtctttagcatatcatatattgcctggggttcggttatatttaataccaagctcgcatcacggctgaaaagctgttttactaaaaaaatgttgctcaagttatacatataaactctatgcgcaatgagtcgcgctctatcaaagttagtagcccccaaatcaatgcagaaaaataggtttaaagtattattgttatagatagatagattcatgccataatcgggactagcccccaacatatgacagtaataaatggctgcataattttcttcccgcaggcaagcaaatttcatcattagattagggctaatgcaaatctcttttttagaacacagctcatgcaagtcaaaaatgttattaaaataaaggctacaagttagccgccaatacagctgatttttatgtttttcgtagaaatagtgaatcgcttttgtaaaattatgccgtaatgccagggcaaaccaaaacttcgttaataggaggtgcgccgtatcccccgtcaacggaatttttgaacgggtatacataactgtgtttaaagtggttctagtcacggtttccataagtggattatgacaaaacatgtcataacccagtagaactcctgcacaggatttcagatcggccacttcttttaaaatttccagaagacgagattcggagacaggtgttaagcctcctagttccgtgcacagccgttttagatgcacggcaggaacacgtataagtccatattcaggatttgcgccccaatccacaaacaatcgtataagctcaagattatcgctcttcacggcctttactagcgccgcgtcaagacaaagatcatccacagaaaaacactgcaaatgttgatacgaaaaaatttgcttacatgtattacataagtgaataggacctaaatcccaccacaaaccaaaacgctgcagcgtataatcatagtcacttgaaagataattgcacgccacaacttttttggccaacgtttgtaaagacaacat

SEQ ID NO: 65 - DNA, 1074 bp, African swine fever virus
ttagtctataactacaatttctggatgggctgtaaaatactcttcggctcgttttagattttttgacgtatatgtctttagcatatcatatattgcctggggttcggttatatttaataccaagctcgcatcacggctgaaaagctgttttactaaaaaaatgttgctcaagttatacatataaactctatgcgcaatgagtcgcgctctatcaaagttagtagcccccaaatcaatacagaaaaataggtttaaagtattattgttatagatagatagattcatgccataatcgggactagcccccaacatatgacagtaataaatggctgcataattttcttcccgcaggcaagcaaatttcatcattagattagggctaatgcaaatctcttttttagaacacagctcatgcaagtcaaaaatattattaaaataaaggctacaagccagccgccaatagagctgatttttatgcttttcatagaaatagtgaatcgcttttgtaaaattatgtcgtaatgccagggcaaaccaaaacttcgttaataggaggtgcgccgtatcccccgtcaacggaatttttgaacgggtgtacataacggtgtttaaagtggttctagtcacggtttccaagagtggattatgacaaaacatgtcataacccagtagaactcctgcacaggatttcagatcggccacttcttttaaaatttccagaagacgagattcggagacaggtgttaagcctcctagttctgtgcacagccgttttagatccacggcaggaacacgtataagtccatattcaggatttgcgccccaatccacaaacaatcgtataagctcaagattatcgctcttcacggcctttactagcgccgcgtcaagacaaagatcatcctcagaaaaacactgcaaatgttgatacgaaaaaatttgcttacatgtattacataagtgaataggacctaaatcccaccacaaaccaaaacgctgcagcgtataatcatagtcacttgaaagataattgcatgccacaacctttttggccaacgtttgtaaagacaacat

SEQ ID NO: 66 - DNA, 1074 bp, African swine fever virus; identical to SEQ ID NO: 61

SEQ ID NO: 67 - DNA, 1074 bp, African swine fever virus
ttagtctataactacaatttctggatgggctgtaaaatactcttcggcttgttttagattttttgacgtatatgtatttagcatatcatatattgcctggggctcagttagatctaatatcaagctcacatcacggctgaaaagctgttttactaaaaaaaggttgctcaagttatacatataaactctacgcgcaatgagtcgcgccctatcaaagttagtagcccccaaatcaatgcagaaaaataggtttaaagtattattgttatagatagatagattcatgccataatcaagactagcccctaacatatgacagtaataaatggctgcataattttcttcccgcaagcaagcaaatttcatcatcagattagggctaatgcaaatctcttttttacaacacagctcatgcaagtcaaaaatgttgttaaaataaaggctacaagtcagtcgccaatagagctgatttttatgcttttcgtagaaatagtgaatagcttttgtaaaattatgtcgtaatgccagggcaaaccaaaactttgataataggaggtgcgccgtatcccccgtcaacggaatttttgaacgggtgtacataactgtgtctaaggtggttctagtcacggtttccaagagtggattatgacaaaacatgtcataacccagtagaactcctgcacaggatttcagatcggccacttcttttaaaatctccagaagacgagattcggatacaggtgttaagcctcctagttccatgcacagccgctttagatgcacggcaggaacacgtataagtccatattcaggatttgcgccccaatccacaaacaatcgtataagctcaagattatcgctcttcacggcctttactagcgccgcttcaagacaaagatcatcctcagaaaaacactgtaaatgtttatatgaaaaaatttgcttacatgtattacataagtgaataggacctaaatcccaccacaaaccaaaacgctgcagcgtataatcataatcacttgaaagataattgcatgccacaacttttttggccagcgtttgtaaagacaacat

SEQ ID NO: 68 - DNA, 1074 bp, African swine fever virus
ttagtctataactataatttctggatgggctgtaagatactcttcggcccgtttcatattttttgaagtatatgtctttagcatatcatatatttcctggggttcggttacatctaataccaaggtcacatcacggctgaaaagctgctttactaagaaaatgttgctcaagttatacatataagctttgtgcgcaatgagttgtgccctatcaaaatcggcagcccccaaatcaatacagaaaaacatgtttaaagtattattgttatagatagaaagattcatgccataatcgagactagcccccaacctatgacagtaataaatggccgcgtaatttttttcccgcaagcaagcaaatttcatcatcagattagggctaatgcaaatctctttttcacgacacagctcgtgtatgtcaaaaatgttattaaaataaaggctacaagctacccgccaatagagctgatttttatgccttttataaaaatagtggatagcctttgtaaaattatgtcgtaatgccagggcaaaccaaaactttgttaataggaggtgcgccgtatcccccgtcaacggaatgtttgaacaggtgtacgtaactgtgtctaaagtggttctagttacggtttccaagagtggattatgacaaaacatgtcataacccagtagaactcctgcacaggattttaggttggccacttcttttaaaatttccagaagacggggttcggatacaggcgttaagcctcccagttccgcacacagccgctttagatacacggcaggaacacgtataagcccatattcaggatttgcgccccaatccacaaataaacgtataagttcaagattatcgctcttcacggcctttactagcgccgcttcaagacaaagatcatcctcagaaaaacactgtaaatgtttatacgaaaaaacttgcttacaattgttacataggtgaataggacctaaatcccaccacaaaccaaaacgctgcaacgtataatcatagtcacttgaaagataattgcatgccacaacttttttggccaacgtttgtaaagacaacat

SEQ ID NO: 69 - DNA, 1074 bp, African swine fever virus
ttagtctataactataatttctggatgggctgtaagatactcttcggctcgtttcagattttttgaagtatatgactttagcctatcatatatttcctggggttcggttacatctaatgccaaggtcacatcacggctgaaaagctgctttactaagaaaatgttgctcaagttatacatataagctttgtgcgcaatgtgttgtgccctatcaaaatcagtagcccccaaatcgatgcagaaaaacatgtttaaagtattattgttatagatagaaagattcatgccataatcgagactagcccctaacctataacagtaataaatggccgcataattttttttctgcaagcaagcaaatttcatcatcagattagggctgatgcaaatctctttttcacgacacagctcgtgtatgtcaaaaatgttattaaaataaaggctacaagctacccgccaatagaggtgatttttatgccttttatagaaatagtgaatagcctttgtaaaattatgtcgtaatgccagggcaaaccaaaactttgttaataggaggtgcgccgtatcccccgtcaacggaatgtttgaacaggtgtacgtaactgtgtctaaagtggttctagttacggtttccaagagtggattatgacaaaacatgtcataacccagtagaactcctgcacaggattttagcttggccacttcttttaaaatttccagaagacggggttcggttacaggcgttaagcctcccagttccgtgcacagccgctttagatgcacggcaggaacacgtataagtccatattcaggatttgcgccccaatccacaaataaacgtataagctcaagattatcgctcttcacggcctttactagcgccgcttcaagacaaagatcatcctcagaaaaacactgtaaatgtttatacgaaaaaatttgcttacaattgttacacaggtgaataggacctaaatcccaccacaaaccaaaacgctgcaacgtataatcatagtcacttgaaagataattgcatgccacaacctttttggccaacgtttgtaaagacaacat

SEQ ID NO: 70 - DNA, 1074 bp, African swine fever virus
ttagtctataactataatttctggatgggctgtaagatactcttcagctcgtttcagattttttgaagtatatgactttagcatatcatatatttcctggggttcggttacatctaatgccaaggtcacatcacggctgaaaagctgctttactaagaaaatgttgctcaagttatacatataagctttgtgcgcaatgcgttgtgccctatcaaaatcagtagcccccaaatcgatgcagaaaaacatgtttaaagtattattgttatagatagaaagattcatgccataatcgagactagcccctaacctataacagtaataaatggccgcataattttttttctgcaagcaagcaaatttcatcatcagattagggctgatgcaaatctctttttcacgacacaactcgtgtatgtcaaaaatgttattaaaataaaggctacaagctacccgccaatagaggtgatttttatgccttttatagaaatagtgaatagcctttgtaaaattatgtcgtaatgccagggcaaaccaaaactttgttaataggtggtgcgccgtatcccccgtcaacggaatgtttgaacaggtgtacgtaactgtgtctaaagtggttctagttacggtttccaagagtggattatgacaaaacatgtcataacccagcagaactcctgcacaggattttagcctggccacttcttttaaaatttccagaagacgggattcggatacaggcgttaagccacccagttccgtgcacagccgctttagatgcacggcaggaacacgtataagtccatattcaggatttgcgccccaatccacaaataaacgtataagctcaagattatcgctcttcacggcctttactagcgccgcttcaagacaaagatcatcctcagaaaaacactgtacatgtttatacgaaaaaatttgcttacaattattacataggtgaataggacctaaatcccaccacaaaccaaaacgctgcaacgtataatcatagtcacttgaaagataattgcatgccacaacctttttggccaatgtttgtaaagacaacat

SEQ ID NO: 71 - DNA, 1074 bp, African swine fever virus
ttagtctataactataatttctggatgggctgtaagatactcttcggctcgtttcatattttttgaagtatatgcctttagcctatcatatatttcctggggttcggttacatctaatgccaaggtcacatcacggctgaaaagctgctttactaaaagaatgttgctcaagttatacatataagctttgtgcgcaatgcgttgtgccctatcaaaatcagtagcccccaaatcgatgcagaaaaacaggtttaaagaattattgttatagatagaaagattcatgccataatcgagactagctcccaacctataacagtaataaatggccgcgtaatttttttcccgcaagcaagcaaatttcatcatcagattagggctaatgcaaatctctttttcacgacacagctcgtgtatgtcaaaaatgttattaaaataaaggctacaagctacccgccaatagaggtgatttttatgccttttatagaaatagtgaatagcctttgtaaaattatgtcgtaatgccagcgcaaaccaaaactttgttaataggtggtgcgccgtatcccccatcaacggaatttttgaacagctgtacataactgtgtctaaagtggttctagttatggtttccaagagtgggttataacaaaacatgtcataacccagtagaactcctgcacaggattttaggttggccacttcttttaaaatttccagaagacggggttcggatacaggtgttaagcctcccagttctgtgcacagccgctttagatgcacggtaggaacacatataagtccatattcaggatttgcaccccaatccacaaataaacgtataagctcaagattatccctcttcacggcctttactagcgccgcttcaagacagagatcatcctcagaaaaacactgtaaatgtttatacgaaaaaatttgcttacaattgttacacaggtgaataggacctaaatcccaccacaaaccaaaacgctgcaacatataatcatagtcacttgaaagataattgcatgccacaacctttttggccaacgtttgtaaagacaacat

SEQ ID NO: 72 - DNA, 1074 bp, African swine fever virus
ttagtctataactataatttctggatgggctgtaagatactcttcggctcgtttcagattttttgaagtatatgactttagcatatcatatatttcctggggttcggttacatctaatgccaaggtcacatcacggctgaaaagctgctttactaaaaaaatgttgctcaagttatacatataagttttgtgcgcaatgcgttgtgccctatcaaaatcagtagcccccaaatcgatgcagaaaaacatgtttaaagtattattgttatagatagaaagattcatgccataatcgagactagcccctaacctataacagtaataaatggccgcataattttttttccgcaagcaagcaaatttcatcatcagattagggctgatacaaatctctttttcacgacacagctcgtgtatgtcaaaaatgttattaaaataaaggctacaagctacccgccaatagaggtgatttttatgccttttataaaaatagtgaatagcctttgtaaaattatgtcgtaatgccagggcaaaccaaaactttgttaataggtggtgcgccgtatcccccgtcaacggaatgtttgaacaggtgtacgtaactgtgtctaaagtggttctagttacggtttccaagagtggattatgacaaaacatgtcataacccagtagaactcctgcacaggattttagcttggccacttcttttaaaatttccagaagacggggttcggatacaggcgttaagcctcccagttccgtgcacagccgctttagatgcacggcaggaacacgtataagtccatattcaggatttgcgccccaatccacaaataaacgtataagctcaagattatcgctcttcacggcctttactagcgccgcttcaagacaaagatcatcctcagaaaaacactgtaaatgtttatacgaaaaaatttgcttacaattgttacataggtgaataggacctaaatcccaccacaaaccaaaacgctgcaacatataatcatagtcacttgaaagataattgcatgccacaacctttttggccaacgtttgtaaagacaacat

SEQ ID NO: 73 - DNA, 1596 bp, African swine fever virus
atgttctctctccagaacttatgtcgaaaaacattacctgactgtaaacttcctgaattttttgacgattatatattacaactgctgggattatactgggaaaaccatggaactattcaacgggcaggaaacaactgtgtgcttatacaacaacataccctcattcccgtaaatgaagccctaagaatagcagcatctgaagaaaattatgagatcgtgggccttttattagcgtgggaggggaacctttactatgctattataggggctctagagggtaaccgctacaacttaattcgtaaatatgatgatcaaatcaaggaccatcatgacattctgccattcattgatgatccaatcatatttcacaaatgccatatcatgcggcgatgcttttttgattgtattttatatcaagctgtaaaatatagtaagtttcgtgttcttctttattttaaatatacattagaggatgatttgcccctcgttcatttacttattgaaaaggcatgtgaagatcataattatgaagttattaaatggatatatgaaaacctacatgtctgccatataatagatacctttgactgtgctattgcccataaagatctacgtttatattgtttggggtatacatttatatacaacaggattgtaccctataagtatcatcatttagatattctcatactttcaagcctacaactcctacataaggtggcggccaaaggatacttagattttatcctagaaaccttaaagtatgatcataatatagataatttagatgttattctaacacaagctgcaacatataaccatagaaaaattttaacctattttattcctcaatcaacctacgcacaaatagaacaatgtttgttcgtggcgataaaaacaaaatcttccaagaaaaccttgaacttactactgtctcacctaaacctttccatcaaactcatccaaaaaatcagccaatatgttgccactttcaattcaacaaatataataggcattctgagtatgaagcggaaaaagaagatatatttggatatcatattgacaaaatttgtaaaaaatgctatttttaataaatttgtcgttcgatgtatggagagattttctataaacccggaaagaatcgtcaaaatggctgcgcgtataaataagatgatgttagtgaaaaaaatatctgaacatgtttggaaaaatcatgcggctagacttaaacaccttaaacatgcggtacacacgatgaagcataaagatgggaaaaatagactcatgaactttatctatgaacactgctattaccatatgcaaggggaagaaatttttagcctcgcaagattttatgcaatccatcatgcaccaaagttgttcgacgttttttataattgttgtatcctagatacgatacgattcaaaagccttcttttagattgttcacatatcataggtaaaaacgctcatgatgctactaatatcaacatcgtgaacaagtatattggcaacctgtttgctatgggagttcttagcaaaaaagaaatcttacaggactatccatccatctattctaaacattatatgccttag

SEQ ID NO: 74 - DNA, 1596 bp, African swine fever virus
atgttctctctccagaacttatgtcgaaaaacattacctaaccgtaaacttcctgaattttttgacgaatatatattacaactgctgggattatactgggaaaaccatggaactattcaacgagcaggaaacaactgtgtgcttatacagcaacataccctcattcccgtaaatgaagccctgagaacagcagcatctgaagaaaattatgagatcgtgagccttttattagcgtgggaggggaacctttactatgctattataggggctctagagggcaaccgccacgacttaattcgtaaatatgatgaccaaatcaaggaccatcatgaaattctgccattcattgacgatccagtcatatttcacaaatgccatatcatgcggcaatgcttttttgattgtattttatatcaagctgtaaaatatagtaagtttcgcgttcttctttactttaaacatagattagaggatgatttgcccttcactcatttacttattgaaaaggcatgtaaagatcataattatgaagttattaaatggatatatgaaaacctacatatctacaatatgatagatacctttgaatgtgctattgcccataaggatctacatctatattgtttggggtatagatttatatataacagaatcgtacccgataagtatcatcatttagatattcgcatgctttcaagcctacaactcctacataaggtggcagccaaaggatacttagattttatcctagaaaccttaaagtatgatcataataaagataatataaatattattctaacacaagctgcaacctataaccatagaaaaattttaatctatttcattcctcaatcaacccacgcacagatagaacaatgtttactagtggcgataaaagcaaaatcttccaggaaaaccttgaacttactactgtctcacctaaacctttccatcaacctcatcaaaaaaataagccattatgttgccacttacaattcaacaaatataataggcattctgagtatgcggcggaaaaagaagatatatttagatatcatattgacaaaatttgtaaaaaaagctatttttaataagtttgtcgttcgatgtatggatacattttctataaacccggaaagaatccttaaaatagccgcgcgaataaataggatgatgttagtgaaaaaaatatctgaacatgtttggaaaaatcatgcggttagacttaaataccttaaacatgcggtacacacgatgaagcataaagatgggaaaaatagactcatgaactttatctatgatcgctgttattaccatatgcaaggggaagaaatctttagcctcgcaagattttatgcaatccatcatgcaccaaagttgtttgacgttttttatgattgttgtatcctagatacgatacgattcaaaagccttcttttagattgttcacatatcataggtaaaaacgctcatgatgctaccaatatcaacatcgtgaacaagtatatcggcaacctgtttgttatgggagttcttagcaaaaaagaaatcttacaggactatccatccatttattctaaacaatacatgccttag

SEQ ID NO: 75 - DNA, 1596 bp, African swine fever virus; identical to SEQ ID NO: 74

SEQ ID NO: 76 - DNA, 1593 bp, African swine fever virus
atgttctctctccaaaacttatgtcgaaaaaccttacctgactgtaaacttcctgaattttttgacgattatgtattacaactgctgggattatactgggaaaaccatggaacgattcaacgagcaggaaacaactgtgtacttatccaacaacataacctcattcccgtaaatgaagccctaagaatagcagcatctgaagaaaattatgagatcgtgagccttttattagcgtgggaggggaacctttactatgctattataggggctctagagggtaaccgccacaacttaattcgtaaatatgatgaccaaatcaaggaccatcatgaaattctgccatttattgacgatccagttatatttcacaaatgtcatataatgcggcgatgcttttttaattgtatgttgtatcaagctgtaaaatatagtaagtttagcgttcttctatattttaaatatatattaaaggaaaatttgcccctcgtccactcactcattgaaaaagcgtataaatatcataattatgaggttattaaatggatctatgaaaatctacatatctatgatatcataaatacttttaaatatgctattgcccataaagatctacgtttatattgtttagggtatacatttgtatataataggatcgtaccctataagtattatcatttagatattcgcatccttttaaggctacaacttctacataaggtgacagccaaaggatacttggattttatcctagaaaccttaaagtatgatcataatacaaacaatatagatattatcctaacacaggctgcaacctataaccatagaaacattttaacctatttcattcctcaatcaacctacgcacaaatagaacaatgcttattcgtggcaataaaaacaaacgcttccaaaaaaaccttgaatttactattatctcacctaaacctttctatcaaactcgtcaaaaaattaagccaatatgttgtcgcttacaagtcaacaaatatcataagcattctgagtatgcagcaaaaaaagaagatatatttagatatcattttgacaaaggttgtaaaaaacgctgtttttattaaatttgtcattggatgtatggttacattttccataaacccagaaagaattgtcaaaatggccgcgcgaataaaaaagatgaagttagtaaaaaatatatctgaacatgtttggaaaaatcatgcggctaaacttaaacatcttaagcatgcggtacacacgatgaagcatcaagaaggaaaaaatagactcatgaactttatctatgatcactgctattaccacatgcaaggagaagagatctttagtcttgcaagattttatgcaatccatcatgcgccaaaattgttcgacgttttttatgattgttgtatcctagatacgatacgatttaaaagcctgcttttagattgttcacatattatagctaaaaacgctcatgatgctagtattaacatcgtgaataagtatattggcaatctatttgctatgggagttcttagcaaaaaagaaatcttacaagactatccatccatctattctaaatatgacatactttag

SEQ ID NO: 77 - DNA, 1593 bp, African swine fever virus
atgttctctctccagaacttatgtcgaaaaaccttacctgactgtaaacttcctgagttttttgacgattatgtattacaactgctgggcttatactgggaaaatcatggtacgattcaacgagcaggaaacaactgtgtacttatccaacaacataacctcattcccgtaaatgaagccctaagaatagcagcatctgaggaaaattatgagatcgtgagccttttattagcgtgggaggggaacctttactatgctattataggggctctagaggggaaccaccacaacttaattcgtaaatacgatgaccaaatcaaggaccatcatgaaattctgccatttattgacgatccagtcatatttcacaaatgtcatacaatgcggcgatgcttttttaattgtattttatatcaagctgtaaaatatagtaagttcagcgttcttctatactttaaatatatattaaaggagaatttgcccctcgtccactcacttattgaaaaggcgtataaatatcataattatgaggttattaaatggatctatgaaaacctacatatctatgatatcataaatacttttaaatgtgctattgcccataaagatctacgtttatattgtttagggtatacatttgtatataatagaatcgtaccctataagtattatcatttagatattcgcatccttttaaggctacaacttctacataaggtgacagccaaaggatacttggattttatcctagaaaccttaaagtatgatcataatacaaacaatatagatattatcctaacacaggctgcaacctataaccatagaaacattttaacctatttcattcctcaatcaacctacgcacaaatagaacaatgtttattcgtggcgataaaaacaaatgcttccaagaaaaccttgaatttactattatctcacctaaatctttctatcaaactcgtcaaaaaattaagccaatatgttgtcgcttacaagtcaacaaatatcataagcattctgagtaggcagcaaaaaaagaagatatatttagatatcattttgacaaaggttgtaaaaaatgctgtttttaataaatttgtcattggatgtatggttacattttccataaacccagaaagaattgtcaaaatggccgcgcgaataaaaaagatgaagttagtaaaaaatatatctgaacatgtttggaaaaatcatgcggttaaacttaaatatcttaagcatgcggtacacacgatgaagcatcaagaaggaaaaaatagattaatgaactttatctatgatcactgttattaccacatgcaagaagaagagatctttagcctcgcaagattttatgcaatccatcatgcgccaaaattgttcgacgttttttatgattgttgtatcctagatacgatacgatttaaaagcctgcttttagattgttcacatatcatagttaaaaacgctcatgatgctagtattaacatcgtgaacaagtatattggcaatctatttgctatgggagttcttagcaaaaaagaaatcttacaagactatccatctatctattctaaggattacatgctttag

SEQ ID NO: 78 - DNA, 1593 bp, African swine fever virus
atgttttctctccagaacttatgtcgaaaaaccttacctgaccgtaaacttcctgaattttttgacgattatgtattacaactgctgggattatactgggaaaaccatggaacgattcaacgagcaggaaacaactgtgtacttatccaacaacataacctcattcccgtaaatgaagccctaagaatagcagcatctgaagaaaattatgagatcgtgagccttttattagcttgggaggggaacctttactatgctattataggggctctagagggtaatcgccacaacttaattcgtaaatatgatgaccaaatcaaggaccatcatgaaattctgccatttattgacgatccagtcatatttcacaaatgtcatatgatgcggcgatgcttttttaattgtattttgtatcaagctgtaaaatatagtaagtttagcgttcttctatattttaaatatatattaaaggagaatttgcccctcgttcattcacttattgaaaaggcgtgtgaagatcataattatgaagttattaaatggatctatgaaaacctacatatctatgaaataatggatacctttaaatgtgctattgcccataaagatctacatttatatagtttaggatatacatttatatataataggatcgtaccctataagtatcatcatttagatattcgcatactttcaaggctacaacttctacataaggtgacagccaaaggatacttggactttatcctagaaaccttaaagtatgatcataataaagataatataaatattattctgacacaagctgcaacctataaccatagaaacattttaacctatttcattcctcaatcaacctacgcgcagatagaacaatgcttattcgtggcaataaaaacaaacgcttccaagaaaaccttgaatttactattatctcacctaaacctttctatcaaactcgtcaaaaaattaagccaatatgttgttgcttacaagtcaacaaatatcataagcattctgagtatgcagcaaaaaaagaagatatatttagatatcattttgacaaaggttgtaaaaaacgctatttttattaaatttgtcattggatgtatggttacattttccataaacccagaaagaattgtcaaaatggccgcgcgaataaaaaagatgaagttagtgaaaaatatatctgaacatgtttggaaaaatcatgcggctaaacttaaacaccttaagcatgcggtacacacgatgaagcatcaagaaggaaaaaatagattaatgaactttatctatgatcactgttattaccacatgcaaggagaagagatctttagtcttgcaagattttatgcaatccatcatgcgccaaaattgttcgacgttttttatgattgttgtatcctagatacgatacgatttaaaagcctgcttttagattgttcatatatcatagctaaaaacgctcatgatgctagtattaacatcgtgaataagtatattggcaatctatttgctatgggggttcttagcaaaaaagaaatcttacaagactatccatccatctattctaaggattacatgctttag

SEQ ID NO: 79 - DNA, 1596 bp, African swine fever virus; identical to SEQ ID NO: 73

SEQ ID NO: 80 - DNA, 1593 bp, African swine fever virus
atgttctctctccagaacttatgtcgaaaaacattacctgactgtaaacttcctgaattttttgacgaatatatattacaactgctgggattatactgggaaaaccatggaactattcaacgagcaggaaacaactgtgtgcttatccagcaacataaccttattcccgtaaatgaagccctaagaatagcagcatctgaagaaaattatgagatcgtgagccttttattagcgtgggagggaaacctttactatgctattataggggctctagagggcaaccgccccgacttaattcgtaaatatgatgaccaaatcaaggaccatcatgaaattttgccattcatcgacgatccaatcatatttcacaaatgtcacataatgcggcgatgcttttttaattgtattttatatcaagctgtaaaatatagtaagtttcgtgttcttctttattttaaacataggttaggggatgatttgcccctcactcatttacttattgaaaaggcatgtgaagatcataattatgaagttattaaatggatctatgaaaacctacatagctacaatataatggatacctttgaatgtgctattgcccataaggatctacgtctatattgtttggggtatacatttatatataataggatcgtaccctataagtatcatcatttagatatttgcatactttcaagcctacaactcctgcataaggtggcagccaaaggatacttggattttatcctagaaaccttaaagtatgatcataacataaataatatagatattattctaacacaagctgcaacctataaccatagaaaaattttaacctatttcattcctcaattaacctacgcacagatagaacaatgtttactagtggcgataaaaacaaaagcttccaagaaaaccttgaacttactactgtctcacctaaacctttccatcaaactcatcaaaaaaataagccaatatgttgtcacttacaattcaacaaatatcataagcattctgagtatgcggcggaaaaagaagatatatttagatatcattttgacagagtttgtaaaaaacgctatttttaataaatttgtcgttcgatgtatggatacattttccataaacccggaaagaattgtcaaaatggccgcacgaataaataggatgatgttagtgaaaaatatatctgaacgtgtttggaaaaatcatgcggttaaacttaaacaccttaagcatgcggtacatacgatgaagcatcaagaaggaaaaaatagactcatgaactttatctatgatcactgctattaccacatgcaaggggaagagatctttggcctcgcaagattttatgcaatccatcatgcacccaagttgtttgacgttttttatgattgctgcatgctagatgctacacgatttaaaagcctgcttttagattgtccacatatcataggtaaaaacgcttatgatgctggtatcaacctcgtgaacaaatatattggcaacctatttgctatgggggttcttagcaaaaaagaaatcttacaggactatccatctatctattctaaacatgatatgttttag

SEQ ID NO: 81 - DNA, 1596 bp, African swine fever virus
atgttctctctccagaacttatgtcgaaaaacattacctgattgtaaacttcctgaattttttgacgattatatattacaactgctgggattatactgggaaaaccatggaactattcaacgggcaggaaacaactgtgtgcttatacagcaacatacccttattcccataaatgaagccctgagaacagcagcatctgaagaaaattatgagatcgtgagccttttattagcatgggaggggaatctttactatgctattataggggctctagagggcaaccgccacgacttaattcgtaaatatgatgaccaaatcaaggaccatcatgaaattctgccattcattgacgatccagtaatatttcacaaatgccatatcatgcggcgatgcttttttgattgtattttatatcaagctgtaaaatatagtaagtttcgcgttcttctttattttaaatataggttagaggatgatttacccctcactcatttacttattgaaaaggcatgtgaaaatcataattatgaagttattagatggatatatgaaaacctacatatctacaatatgatagatacctttgaatgtgctattgcccataaggatctacgtctatattgtttggggtatacatttatatataacaggatcgtacccaataagtatcatcatatagatattctcattctttcaagcctacaactcctgcataaggtggcagccaaaggatacttagactttatcctagaaaccttaaagtatgatcataacaatgataatttagatattattctaacacaagctgcaacatataaccatagaaaaattttaacctattttattcctcaatcaacctacgcacaaatagaacaatgtttgatggtggcgataaaaacaaaatcttccaagaaaaccttgaatttactattgtcccacctaaacctttccatcaaactcatcaaaaaaataagccaatatgttgtcacttacaattcaacaaatataataggcattctgagtatgaagcgaaaaaagaagatatatttagatatcttgttgacaaaatttgtaaaaaatgctatttttaataaatttgtcgttcgatatatggatacgttttctataaacccggaaaaaatcgtcaaaatggccgcgcgaataaataagatgatgttagtgaaaaaaatatctgaacatatttggaaaaatcatgcggctagacttgaacaccttaaacatgcggtacacacgatgaagcataaagatgggaaaaatagactcatgaactttatctatgagtactgctattaccatatgcaaggggaagaaatttttagcctcgcaagattttatgcaatccatcatgcaccaaagttgttcgacgttttttataattgttgtatcctagatacgatacgattcaaaagccttcttttagattgttcacatatcataggtaaaaacgctcatgatgctactaatatcaacatcgtgaacaagtatattgacaacctgtttgctatgggagttcttagcaaaaaagaaatcttacaggactatccatccatttattctaaacattatatgccttag

SEQ ID NO: 82 - DNA, 1596 bp, African swine fever virus
atgttctctctccagaacttatgtcgaaaaacattacctgactgtaaacttcctgaattttttgacgaatatatattacaactgctgggattatactgggaaaaccatggaactattcaacgagcaggaaacaactgtgtgcttgtacaacaacataccctcattcccgtaaacgaagccctgagaatagcagcgtctgaagaaaattatgagatcgtgagccttttattagcgtgggagggaaacctttactatgctattataggagctctagagggcaaccgccacgacttaattcgtaaatatgatgaccaaatcaaggaccatcatgaaattctgccattcattgacaatccagtcatatttcacaaatgccatataatgcggcgatgcttttttgattgtattttatatcaagctgtaaaatatagtaagtttcgcgttcttctttattttaaatataggttagagaatgatttgcccctcgctcatttacttgttgaaaaggcatgtgaagatcataattatgaagttattaaatggttatatgaaaacctacatatctacaatataatggagacctttgaatgtgctattgcccataaggatctacgtctatatcgtttggggtatacatttatatataacaggatcgtaccctataagtatcattatttagatgttctcattctttcaggcctacatctcctgtataaggtggcagccaaaggatacttagattttatcctagaaaccttaaagtatgatcataacaatgataatttagatattattctaacacaagctgtaacatataaccatagaaaaattttaacctattacattcctcaattaacctatgcacaaatagaacaatgtttgttcatggcgattaaaaaaaaatcttccaagaaaaccttgaacttactactgtctcacctaaagctttccatcaaactcatcaaaaaaataagccaatatgttgccacttacaattcaacaaatataataggcattctgaatatgaagcggaaaaagaagatatatttagatatcatattgacaaaatttgtaaaatacgctatttttaataaatatgtcgttcgttgtatggatacattttccataaacccggaaagaatcatcaaaatggccgcgcgaataaataagatgttgttagtgaaaaaaatatctcaacatgcttggaaaaatcatgcggctagacttaaacaccttaagcatgcggtatacacgatgaaacataaagatgggaaaaatagactcatgaacttgatctatgatcactactattaccatatgcaaggggaagaaatctttagcctcgcaagattttatgcaatccatcatgcaccaaagttgtttgacgttttttatgattgttgtctcctagatactatacgatttaaaagccttcttttagattgttcacacatcataggtaaaaacgctcatgatgctactaatatcactatcgtgaacaagtatattggcaacctgtttgctatgggagttcttagcaaaaaagaaatcttacaggactatccatccatctattctaaacattacatgccttag

SEQ ID NO: 83 - DNA, 1596 bp, African swine fever virus
atgttctctcttcagaacctatgtcgaaaaacattacctgactgtaaacttcctgaattttttgacgaatatatattacaactgctgggattatactgggaaaaccatggaactattcaacgagcaggaaacaactgtgtgcttatacagcaacataacctcattcccgtaaatgaagccctaagaatagcagcatctgaagaaaattatgagatcgtgagtcttttattagcatgggaggggaacctttactatgctattataggggctctagagggcaaccgccacgacttaattcgtaagtatgatgaccaaatcaaggaccatcatgaaattctgccattcattgacgatccagtaatatttcacaaatgccatatcatgcggcaatgcttttttgattgtattttatatcaagctgtaaaatatagtaagtttcgcgttcttctttcttttaaacatagattaagggatgatttgcccttcactcatttacttattgaaaaggcatgtaaagatcataattatgaagtaattaaatggatatatgaaaacctacatatctacaatatgatagatacctttgaatgtgctattgcccataaggatctacgtctatattgtttggggtatagatttatatataacaggatcgtacccgataagtatcatcatttagatattcgcatgctttcaagcctacaactcctgcataaggtggcagccaaaggatacttagattttatcctagaaaccttaaagtatgatcataataaagataatataaatattattctaacacaagctgcaacctataaccatagaaaaattttaatctatttcattcctcaatcaacccacgcacagatagaacaatgtttactagtggcgataaaaacaaaatcttccaagaaaaccttgaacttactactgtctcacctaaacctttccatcaatctcatcaaaaaaataagccattatgttgccacttacaattcaacaaatataataggcattctgagtatgcggcggaaaaagaagatatatttagatatcatattgacaaaatttgtaaaaaaagctatttttaataaatttgtcgttcgatgtatggatacattttctataaacccggaaagaatccttaaaatagccgcgcgaataaataggacgatgttagtgaaaaaaatatctgaacatgtttggaaaaatcatgcggttagacttaaataccttaaacatgcggtacacacgatgaagcataaagatgggaaaaatagactcatgaactttatctatgatcgctgttattaccatatgcaaggggaagaaatctttagcctcgcaagattttatgcaatccatcatgcaccaaagttgtttgacgttttttatgattgttgtatcctagatacgatacgattcaaaagccttcttttagattgttcacatatcataggtaaaaacgctcatgatgctaccaatatcaacatcgtgaacaagtatatcggcaacctgtttgttatgggagttcttagcaaaaaagaaatcttacaggactatccatccatctattctaaacattacatgccttag

SEQ ID NO: 84 - DNA, 1596 bp, African swine fever virus
atgttctctctccagaacctatgtcgaaaaactttacctgactgtaaacttcctgaattttttgacgaatatatattacaactgctgggattatactgggaaaaccatggaactattcaacgagcaggaaacaactgtgtgcttatacagcaacgtaccctcattcccgtaaatgaagccctgagaatagcagcatctgaagaaaattatgagatcgtgggccttttattagcgtgggaggggaacctttactatgctattataggagctctagagggcaaccgccacgacttaattcgtaaatatgatgaccaaatcaaggaccatcatgaaattctgccattcattgacgatccagtcatatttcacaaatgccatataatccgacgatgcttttttaattgtattttatatcaagctgtaaaatatagtaagtttcgcgttcttctttactttaaacatagattagaggatgatttgcccctcactcatttacttattgaaaaggcatgtgaagatcataattatgaagttattaaatggatatatgaaaacctacatacctacgatataatggatacctttgaatgtgctattgcccataaggatctacgtctatattgtttggggtatacatttatatataacaggatcgtaccctatgagtatcatcatttagatattctcattctttcaagcctacaactcctgcataaggtggcagccaaaggatacttagattttatcctagaaaccttaaagtatgatcataacaatgataatttagatattattctaacacaagctgcaacatataaccatagaaaaattttaacctatttcattcctcaattaacctacgcacaaatagaacaatgtttgttcatggcgataaaaagaaaatcttccaagaaaaccttgaacttactactgtctcacctaaccctttccatcgaactcatcaaaaaaataagccaatatgttgtcacttacaattcaacaaatataataggcattctgagtatgaagcggaaaaagaagatatatttagatatcatgttgacaaaatatgtaaaatacgctatttttaataaatatgtcgttcgatgtatggatagattttccataaacccggaaagaatcatcaaaatggccgcgcgaataaataggatgatgttagtgaaaaaaatatctgaacatgtttggaaaaatcatgcggctagacttaaacaccttaagcatgcggtacacacgatgaagcataaagatgggaaaaatagactcatgaactttatctatgatcgctgctattaccatatgcaaggggaagaaatctttagcctcgcaagattttatgcaatccatcatgcaccaaagttgtttgacgttttttatgattgttgtatcctagatactatacaattcaaaagccttcttttagattgttcacatatcataggtaaaaacgctcatgatgctaccaatatcaacatcgtgaacaagtatattggcgacctgtttgctatgggagttcttagcaaaaaagaaatcttacaggactatccatccatctattctaaacattacatgctttag

SEQ ID NO: 85 - DNA, 1596 bp, African swine fever virus
atgttttctctccagaacttgtgtcgaaaaacattacctgactgtaaacttcctgaattttttgacgaatatatattacaactgctgggattatactgggaaaaccatggaactattcaacgagcaggaaacaactgtgtgcttatacagcaacataccctcattcccgtaaacgaagccctgagaatagcagcatctgaagaaaattatgagatcgtgagccttttattagcgtgggagggaaacctttactatgctattataggagctctagagggcaaccgccacgacttaattcgtaaatatgatgaccaaatcaaggaccatcatgaaattctgccattcattgacgatccagtcatatttcacaaatgccatatcatgcggcgatgcttttttgattgtattttatatcaagctgtaaaatatagtaagtttcgcgttcttctttattttaaatataggttagagaatgatttgcccctcgctcatttacttattaaaaaggcatgtgaagatcataattatgaagttattaaatggatatatgaaaacctacatatctacaatataatggatacctttggatgtgctattgcccataaggatctacgtctatatcgtttggggtatacatttatatataacaggatcgtaccctataagtatcattatttagatgttctcattctttcaggcctacatctcctgtataaggtggcagccaaaggatacttagattttatcctagaaaccttaaagtatgatcataacaatgataatttagatattattctaacacaagctgcaacatataaccatagaaaaattttaacctattacattcctcaattaacctatgcacaaatagaacaatgtttgttcatggcgattaaaaaaaaatcttccaagaaaaccttgaacttactactgtctcacctaaagctttccatcaaactcatcaaaaaaataagccaatatgttgccacttacaattcaacaaatataataggcattctgaatatgcggcggaaaaagaagatatatttagatatcatattgacaaaatttgtaaaaaaagctatttttaataaatttgtcgttcgatgtatggatacattttccataaacccggaaagaatcatcaaaatggccgcgcgaataaataagatgttgttagtgaaaaaaatatctgaacatgcttggaaaaatcatgcggctagacttaaacaccttaagcatgcggtatacacgatgaaacataaagatgggaaaaatagactcatgaacttgatctatgatcactactattaccatatgcaaggggaagaaatctttagcctcgcaagattttatgcaatccatcatgcaccaaagttgtttgacgttttttatgattgttgtctcctagatactatacgatttaaaaaccttcttttagattgttcacacatcataggtaaaaacgctcatgatgctactaatatcactatcgtgaacaagtatattggcaacctgtttgctatgggagttcttagcaaaaaagaaatcttacaggactatccatccatctattctaaacattacatgccttag

SEQ ID NO: 86 - DNA, 1581 bp, African swine fever virus
atgttttcccttcaagacctttgccgaaagcatctttttattcttcccgatgtttttggcgagcatgtactacaacgattaggactgtattggagatgtcacggctcccttcaacgcataggagacgaccacatactcatacgacgggatctcatcctttccaccaacgaggccttaagaatggcgggagaggaaggaaacaatgaagtagtaaagctcttgttactgtggaagggaaatcttcattacgccgtcataggagccctgcagggtgatcaatatgacctgatccataagtatgaaaaccaaatcggcgactttcattttatcttaccattgattcaagacgcgaatacgtttgaaaaatgccacgctttagaacgtttttgtggtgtttcatgtctgctaaaacatgctacaaaatacaacatgctccctattctccaaaaataccaagaagagctgtctatgagagcgtatcttcacgaaaccctatttgaactagcatgcctatggcagaggtatgatgtccttaaatggatagagcagaccatgcatgtttacgacctaaagattatgtttaatattgccatctccaagagggatctgactatgtactccttaggatatattttcctttttgatagagggaacaccgaagctacgttgctaacgcaacatctcgagaagacagcggccaaagggctcctccactttgtgctagaaacgttaaaatacggcggcaacatagataccgtcctgacccaagccgtaaagtacaaccatagaaaacttttagattattttctgcgtcaactacctcgtaaacatattgaaaagcttttgttgctggctgttcaggaaaaggcttctaaaaaaacattgaacttactgttgtcacatttaaactactccgtgaaacgcatcaaaaaactgctgcgctatgtgatagagtatgagtccaccttggtgataaagattttgttaaaaaaaagagtaaacctgatagatgccatgttggaaaagatggtaagatatttttctgcgacgaaagtgaggacgatcatggatgagctttcgattagtccggaaagagtcattaagatggctatacagaaaatgagaacggatatcgtaatccatacttcttatgtttgggaggatgatctagaacgtcttactcgtcttaaaaatatggtatacaccataaagtacgaacatgggaaaaaaatgttaattaaagtcatgcacggcatatacaaaaacttattatacggcgaaagggaaaaagtcatgtttcatttagccaagctctatgttgctcaaaatgcggccacccaattcagagacatttgtaaggactgttacaaactggatgtggcacggtttaaaccgcggtttaagcaactaatattagactgtttagaaattgttactaaaaaatcttgctatagtatcctggaaatcttagaaaaacatattatttccctatttactatgaaagttatgactgaagaagaaaaaaacctatgtttagaaatattatataaagtaattcattataaaacaatacaatgttaa

SEQ ID NO: 87 - DNA, 1581 bp, African swine fever virus
atgttttcccttcaagacctttgccgaaagcatctttttattcttcccgatgtttttggcgagcatgtactacaacgattaggactgtattggagatgtcacggctcccttcaacgcataggagacgaccacatactcatacgacgggatctcatcctttccaccaacgaggccttaagaatggcgggagaggaaggaaacaatgaagtagtaaagctcttgttactgtggaagggaaatcttcattacgccgtcataggagccttgcagggtgatcaatatgacctgatccataagtatgaaaaccaaatcggcgactttcattttatcttaccattgattcaagacgcgaatacgtttgaaaaatgccacgctttagaacgtttttgtggtgtttcatgtctgctaaaacatgctacaaaatacaacatgctccctattctccaaaaataccaagaagagctgtctatgagagcgtatcttcacgaaaccctatttgaactagcatgcctatggcagaggtatgatgtccttaaatggatagagcaaaccatacatgtttacgacctaaagattatgtttaatattgccatctccaagagggatctgactatgtactccttaggatatattttcctttttgatagagggaacaccgaagctacgttgctaacgcaacatctcaagaagacagcggccaaagggctcctccactttgtgctagaaacgttaaaatacggcggcaacatagataccgtcctgacccaagccgtaaagtacaatcatagaaaacttttagattattttctgcgtcaactacctcgtaaacatattgaaaaacttttgttgctggccgtgcaggaaaaggcttctaaaaaaacattgaacttactgttgtcacatttaaactactccgtgaaacgcatcaaaaaactaccgcgctatgtgatagagtacgagtccaccttggtgataaagattttattaaaaaaaagagtgaacctgatagatgccatgttggaaaagatggtaagatatttttctgcgacgaaagtgaggacgatcatggatgagctttcgattagtccggaaagagtcattaagatggctatacagaaaatgagaacggatatcgtaatccatacttcttatgtttgggaggatgatctagaacgtcttactcgtcttaaaaatatggtatacaccataaagtacgaacatgggaaaaaaatgttaattaaagtcatgcacggcatatacaaaaacttattatacggcgaaagggaaaaagtcatgttttatttagccaagctctatgttgctcaaaacgcggccacccaattcagagacatttgtaaggactgttacaaactggatgtggcacggtttaaaccgcggtttaagcaactaatattagactgtttagaaattattactaaaaaatcttgctatagtatcctggaaatcttagaaaaacatattatttccctgtttactatgaaagttatgactgaagaagaaaaaaacctatgtttagaaatattatataaagtaattcattataaaacaatacaatgttaa

SEQ ID NO: 88 - DNA, 1581 bp, African swine fever virus; identical to SEQ ID NO: 87

SEQ ID NO: 89 - DNA, 1581 bp, African swine fever virus
atgttttcccttcaagaccattgccgaaagcatctttttattcttcccgatgtttttggcgagcatgtactacaacggttaggactgtactggggacgtcacggctctcttcaacgaatcggggacgatcacatactcatacggcgggacctcatcctttccaccaacgaggccttaaaaatggcgggagaagaaggaaacaatgaagtagtaaagctcttgttactgtgggagggaaatcttcattatgctatcataggggctttacagggtgatcaatatgacctaatccataagtatgaaaaccaaatcgaagactatcatcatatcttaccattgattcaagatgcggaaacgtttgaaaaatgccacgccttagaacgtttttgtgatgttccatgtctgctagaacatgctacaaaacacaacatgctccctattctccaaaaatatcaagaagagttgtctataagagtgtatctacgcgaaaccctattcgaactagcatgcctatggcagaggtatgatattcttaaatggatagagcaaaccatgcatgtttacgatctaaaaattatatttaatattgccatctccaagagggatctaagcatgtactccttaggatatgttctcctttttgatagagggaacaccgaagctaccttgttaacgcaacacctcgagaagacagcggccaaggggctcctccactttgtgctagaaacgttaaaatacggcggtaacttaaatatcgtcctgttccaagccgtaaaatacaatcatagaaaacttttagattattttctgcgtcaactaccccgtaaaaatattgaaaaacttttgttgctggctgtgcaggaaaaagcttctaagaaaacattgaacttactgttgtcacatttaaactactccgtgaaacgcatcaaaaaactgctgcgctatgtgatagagtacgagtctaccttggtgataaagattttattaaaaaaaagagtaaacctaatagacgccgtgttggaaaagactgtaagatatttttctgagacgaaagtaaagactattatggatgagctttcgattaatccggaaaaagtcattaagatggccatacagaaaatgagaacggatattgtgatccaaacttcttatatttgggaggatgatctagaaagacttattcgtcttaaaaatatggtatacaccataaagtatgaacatgggaaaaaaatgttaatgaaagttattcacggcatatacaaaaacttattacacgatgaaaaagaaaaagtcatgtttcatttagccaagttctatattgctcaaaacgcggccacccaattcagagacatttgtaaagactgttgcaaactggatgtggcgcggtttaaaccgcggtttaaacaactaattttagactgtttagaaattgttactaaaaaatcttgctttagtattatagaaattttagaaaaccatattatttccctatttatgatgaaagttatcactgaagatgaaaaaaacctaggtttagaattattatataaagtaattagttacaaaatgatatcatattaa

SEQ ID NO: 90 - DNA, 1575 bp, African swine fever virus
atgttttcccttcaagacctttgccgaaagcatctttttattcttcccgatgtttttggcgagcatgtactacaacaattggggctgtactgggaaaaacacggctctcttcaacgaatagggaacgatcacatactcatacggcgggatctcatcctttctatcaacgaggccttaaaaatagcggcagaggaaggaaacaatgaagtagtaaagctcttgttactgtggaagggaaatcttcattatgccatcataggagccttgcagggtgaccaatatgacctcatccatacgtacgaaaaccaaatcgaagactatcatcatatcttgccattgattcaagatgcgaaaacgtttgaaaaatgccacgccttggaacgtttttgtgatgttccatgcctactagaacatgctacaaaacacaacatgctccctattctccaaaaataccaagaagagctgtttataagagtgtatctccgcgaaaccctatttgaactagcatgcctatggcagaggtatgatatccttaaatggatagagcaaaccatgcatgtttacgacctaaagattatgtttaatattgccatctccaagagggatctaagcatgtactctttagaatatattctcttttttaatagagggaacaccgatgttgcgttagcaacgttgctaacgcaacatctcgagaagacagcggccaaggggctcctccactttgtgctagaaacgttaaaatacggcggtaacataaacatcgtcctctcccaagccgtaaaatacaatcatagaaaacttttagattattttctgcgtcaactacctcgtaaaaatattgaaaaacttttgttgctggccgtacaggaaagggcttctaagaaaacattgaacttactgttgtcttatttaaactactccgtgaaacgcatcaaaaaactgctgcgctatgtgatagagcatgagtccacactggtgataaggattttattaaaaaaaagaataaacctaatagacgccgtgttggaaaagactgtaagatatttttctatgactaaagtgaggacgatcatggatgagctttcgattaatccggaaaaagtcattaaaatggccgtgcagaaaatgagaacggatatcgtgatccatacttcttatgtttgggaggatgatctagaaagacttattcgtcttaaaaatatgctatacactataaagtatgagcatggaaaaaaaatgctaattaaagtcattcacggcatatacaaaaacttatacggcgaaaaagaaaaagtcatgtttaatttggccaagttctatgctgctcaaaacgcggccacccaattcaaagacacttgtaaagactgttgcaaactggatgtggcgcggtttaaacaactaattttagactgtttagacattattactaaaaaaacgtgcctcagtatcatggaaatcttagaaaatcatattatttcgctatttgctatgaaaatgatgacggaggatgaaaaaaacttaggtttagaaatattatataaagtaattagttacaaaatgatatcatattaa

SEQ ID NO: 91 - DNA, 1575 bp, African swine fever virus; identical to SEQ ID NO: 90

SEQ ID NO: 92 - DNA, 1581 bp, African swine fever virus; identical to SEQ ID NO: 86

SEQ ID NO: 93 - DNA, 1581 bp, African swine fever virus
atgttttcccttcaagatctttgccgaaagcatctttttattcttcccgatgtttttggcgagcatgtactacaacagttaggactgtactggaaacgtcacggctctcttcaacgcataggagacgaccacatactcatacggcgggacctcatcctttccaccaacgaggccttaaaaatggcgggagaggaaggaaacaatgaagtagtaaagctcttgttactgtgggagggaaaccttcattatgccatcataggggccttacagggtgatcaatatgatctgatccataagtatgaaaaccaaatcgaagactatcatcatatcttgccattgattcaagatgcgaaaacgtttgaaaaatgccacgccttagaacgtttttgtgatgttccatgtctgctagaacatgctacaaaacacaacatgctccctattctccaaaaataccaagaagagctgtctataagagtgtatcttcgcgaaaccctattcgaactagcatgcctatggcagaggtatgatgttcttaaatggatagagcaaaccatgcatgtttacgacctaaagattatgtttaatattgccatctccaagagggatctaagcatgtactccttaggatatgttctcctttttgatagaggaaacatcgaagctacgttcctaacgcaacatctcgagaagacagcggccaaagggctcctccactttgtgctagaaacgttaaaatacggcggtaacttaaatatcgtcctgtcccaagccgtaaaatacaatcatagaaaacttttggattattttctgcgtcaactacctcgtaaaaatattgaaaaacttttgttgctggccgtgcaggaaaaggcttctaaaaaaacattgaacctactgttgtcacacttaaactactccgtgaaacacatcaaaaaactgctgcgctatgtgatagagtacgagtctaccttggtgataaaacttttattgaaaaaaagagtaaacctgatagacgccgtgttggaaaagaatgtaagatatttttctgcgattaaagtgaggactattatggatgagctttcgattagtccggaaagagtcattaagatggccatacagaaaatgagaacggatattgtgattcagacttcttatatttgggaggatgatctagaaagacttattcgtcttaaaaatatggtatacaccataaagtatgaacatgggaaaaaaatgttaattaaagttattcacggcatatacaaaaacttattatacggcgaaaaagaaaaagtcatgtttcatttagccaaactctatgttgctcaaaacgcggccacccaattcagggacatttgtaaggactgttgcaaactggatgtggcgcggtttaaaccgcggtttaaacaactaattttagactgtttagaaatggttactaaaaaatcttgctttagtattatagaaatcttagaaaactatattatttccctatttgtgatgaaagtcatcactgaagaagaaaaaaacctatgtttagaactattatataaagtaattagttacaaaacgatataatgttaa

SEQ ID NO: 94 - DNA, 1581 bp, African swine fever virus
atgttttcccttcaagacctttgccgaaagcatctttttattcttcccgatgtttttggcgagcatgtactacaacgattaggactgtattggagatgtcacggctcccttcaacgcataggagacgaccacatactcatacgacgggatctcatcctttccaccaacgaggccttaagaatggcgggagaggaaggaaacaatgaagtagtaaagctcttgttactgtggaagggaaatcttcattacgccgtcataggagccttgcagggtgatcaatatgacctgatccataagtatgaaaaccaaatcggcgactttcattttatcttaccattgattcaagacgcgaatacgtttgaaaaatgccacgctttagaacgtttttgtggtgtttcatgtctgctaaaacatgctacaaaatacaacatgctccctattctccaaaaataccaagaagagctgtctatgagagcgtatcttcacgaaaccctatttgaactagcatgcctatggcagaggtatgatgtccttaaatggatagagcagaccatgcatgtttacgacctaaagattatgtttaatattgccatctccaagagggatctgactatgtactccttaggatatattttcctttttgatagagggaacaccgaagctacgttgctaacgcaacatctcgagaagacagcggccaaagggctcctccactttgtgctagaaacgttaaaatacggcggcaacatagataccgtcctgacccaagccgtaaagtacaatcatagaaaacttttagattattttctgcgtcaactacctcgtaaacatattgaaaaacttttgttgctggctgttcaggaaaaggcttctaaaaaaacattgaacttactgttgtcacatttaaactactccgtgaaacgcatcaaaaaactgctgcgctatgtgatagagtatgagtccaccttggtgataaagattttgttaaaaaaaagagtaaacctgatagatgccatgttggaaaagatggtaagatatttttctgcgacaaaagtgaggacgatcatggatgagctttcgattagtccggaaagagtcattaagatggctatacagaaaatgagaacggatatcgtaatccatacttcttatgtttgggaggatgatctagaacgttttactcgtcttaaaaatatggtatacaccataaagtacgaacatgggaaaaaaatgttaattaaagtcatgcacggcatatacaaaaacttattatacggcgaaagggaaaaagtcatgtttcatttagccaagctctatgttgctcaaaatgcggccacccaattcagagacatttgtaaggactgttacaaactggatgtggcacggtttaaaccgcggtttaagcaactaatattagactgtttagaaattgttactaaaaaatcttgctatagtatcctggaaatcttagaaaaacatattatttccctatttactatgaaagttatgactgaagaagaaaaaaacctatgtttagaaatattatataaagtaattcattataaaacaatacaatgttaa

SEQ ID NO: 95 - DNA, 1554 bp, African swine fever virus
atgttttcccttcaagacctttgccgaaagcatctttttattcttcccgatgtttttggcgagcatgtactacaacgattaggactgtattggagatgtcacggctcccttcaacgcgtaggagacgaccacatcctcatacggcgggatctcatcctttccaccaacgaggccttaagaatggcgggagaagaaggaaacaatgaagtagtaaagctcttgttactgtggaagggaaatcttcattacgccatcataggagccttgcagggtgatcaatatgacctgatccataagtatgaaaaccaaatcggcgactttcatcttatcttaccattgattcaagatgcgaaaacgtttgaaaaatgccacgctttagaacgtttttgtggtgtttcatgtctgctagaacatgctacaaaatacaacatgctccctattctccaaacataccaagaagagctgtctatgagagcatatcttcgcgaaaccctatttgaactagcgtgcctgtggcagaggtatgatgtccttaaatggatagagcaaaccatgcatgtttacgacctaaagattatgtttaatattgccatctccaagagggatctgacgatgtattccttaggatatattcttctttttgatagagagaacaccgaagctacgttgttaacacaacatctcgagaagacagcggccaaagggctcctctactttgtgctagaaacgttaaaatacggcggtaacatagatatcgtcctgactcaagccgtaaagtacaatcatagaaaacttttagattattttctgcgtcaactacctcgtaaacatattgaaaagcttttgttgctggccgtgcaggaaaaggcttctaaaaaaacattgaacttactgttgtcccatctaaactactccgtgaaacgtatcaaaaaactgctgcgctatgtgatagagtacgagtccaccttggtgataaagattttattaaaaaaaagagtaaacctgatagatgccatgttggaaaagatggtaagatatttttctgcgacgaaagtgaggacgatcatggatgagcttttgattagtccggaaagagtcattaagatggctatacagaaaatgagaacggatatcgtaatccatacttcttatgtttgggaggatgatctagaacgacttactcgtcttaaaaatatggtatacaccataaagtacgaacatgggaaaaaaatgttaattaaagtcatgcacggcatatacaaaaacttattatacgacgaaagagaaaaggtcatgtttcatttagccaagctctatgttgctcaaaacgcggccacccaattcagagacatttgtaaggactgttacaaactggatgtggcacggtttaaaccgcggtttaagcaactaatgttagactgtttagaaattgttactaaaaaatcttgctatagtatcctggaaatcttagaaaaacatattatttccctatttactatgaaagttatgactgaagaggaaaaaaacctatgtttagaaatattatataagtaa

SEQ ID NO: 96 - DNA, 1554 bp, African swine fever virus
atgttttcccttcaagacctttgccgaaagcatctttttattcttcccgatgtttttggcgagcatgtactacaacgattaggactgtattggagatgtcacggctcccttcaacgcgtaggagacgaccacatactcatacggcgggatctcatcctttccaccaacgaggccttaagaatggcgggagaggaaggaaacaatgaagtagtaaagctcttgttactgtggaagggaaatcttcattacgccgtcataggagccttgcagggtgatcaatatgacctgatccataagtatgaaaaccaaatcggcgactttcatcttatcttaccattgattcaagacgcgaaaacgtttgaaaaatgccatgctttagaacgtttttgtggtgtttcatgtctgctaaaacatgctacaaaatacaacatgctccctgttctccaaaaataccaagaagagctgtccatgagagcatatctttgcgaaaccctatttgaactagcatgcctatggcagaggtatgatgtccttaaatggatagagcaaaccatgcatgtttacgacctaaagattatgtttaatattgccatctccaagagggatctgactatgtactccttaggatatattttcctttttgatagagggaacaccgaagctacgttgctaacgcaatatctcgagaagacagcggccaaagggctcctccactttgtgctagaaacgttaaaatacggcggtaacatagatattgtcctgacccaagccgtaaagtacaatcatagaaaacttttagattattttctgcgtcaactacctcgtaaacatattgaaaagcttttgttgctggccgtgcaggaaaaggcttctaaaaaaacattgaacttactgttgtcacatttaaactactccgtgaaacgcatcaaaaaactgctgcgctatgtgatagagtacgagtccaccttggtgataaagattttattaaaaaaaagagtaaacctgatagatgccatgttggaaaagatggtaagatatttttctgcgacgaaagtgaggacgatcatggatgagcttttgattagtccggaaagagtcattaagatggctatacagaaaatgagaacggatatcgtaatccatacttcttatgtatgggaggatgatctagaacgacttactcgtcttaaagatatggtatacaccgtaaagtacgaacatgggaaaaaaatgttaattaaagtcatacacggcatatacaaaaacttattatacggcgaaaaagaaaaggtcatgtttcatttagccaagctctatgttgctcaaaacgcggccacccaattcagagacatttgtaaggactgttacaaactggatgtggcacggtttaaaccgcggtttaagcaactaatgttagactgtttagaaattgttactaaaaaatcttgctatagtattctggaaatcttagaaaaacatatgatttccctatttactatgaaagttatgactgaagaagaaaaaaacctatgtttagaaatattatataagtaa

SEQ ID NO: 97 - DNA, 1569 bp, African swine fever virus
atgttttcccttcaagacctttgccgaaagcatctttttattcttcccgatgtttttggcgagcatgtattacaacgattaggactgtattggagatgtcacggctcccttcaacgcgtaggagacgaccacatactcatacggcgggatctcatcctttccaccaacgaggccttaagaatggcgggagaagaaggaaacaatgaagtagtaaagctcttgttactgtggaagggaaatcttcattacgccatcataggagccttgcagggtgatcaatatgacctaatccataagtatgaaaaccaaatcggcgactttcatcttatcttaccattgattcaagatgcgaaaacgtttgaaaaatgccacgctttagaacgtttttgtggtgtttcatgtctgctaaaacatgctacaaaatacaacatgctccctattctccaaacataccaagaagagctgtctatgagagtatatcttcgcgaaaccctatttgaactagcatgcctatggcagaggtatgatgtccttaaatggatagagcaaaccatgcatgtttacgacctaaaggttatgtttaatattgccatctccaagagagatctgactatgtattccttaggatatattcttctttttgatagagagaacaccgaagctacgttgctaacgcaacatctcgagaaaacagcggccaaggggctcctccactttgtgctagaaacgttaaaatacggcggtaacatagatatcgtcctgtcccaagccgtaaagtacaatcatagaaaacttttagattattttctgcgtcaactacctcgtaaaaatattgaaaaacttttgttgctggctgttcaggaaaaggcttctaaaaaaacattgaacttactgttgtcccatttaaactactccgtgaaacgcatcaaaaaactgctgcgctatgtgatagagtacgagtccaccttggtgataaagattttattaaaaaaaagagtaaacctgatagatgccatgttggaaaagatggtaagatatttttctgcgacgaaagtgaggacgatcatggatgagctttcgattagtccggaaagagtcattaagatggctatacagaaaatgagaacagatatcgtaatccatacttcttatgtttgggaggatgatctagaacgacttactcgtcttaaaaatatggtatacgccataaagtatgaacatgggaaaaaaatgttaatgaaagtcatgcacggcatatacaaaaacttattatacggcgaaagagaaaaggtcatgtttcatttagccaagctctatgttgctcaaaacgcggccacccaattcagagacatttgtaaggactgttacaaactggatgtggcacggtttaagcaactaacgttaaactgtttagaaattattactaaaaaatcttgctatagtatcctggaaatcctagaaaaacatattatttccctatttactatgaaagttatgactgaagaagaaaaaaacctatgtttagaaatattatataaagtaattcattataatacaatacaatgttaa

SEQ ID NO: 98 - DNA, 843 bp, African swine fever virus
atgtcctcttcccttcaggaactttgtcgaaaaaagctgcctgactgcatacttccagagttttttgacgactatgtattgcaactgttaggactgcactggcaagatcatggttcccttcagcgtatcgagaagaaccagatacttgttcaacaggaacccatccatatcaatgaagcactcaaagtagcagcatcggaagggaactatgaaatcgtagagctgttgttgtcatgggaggcagatccccgctacgccgtcgtaggagctctagaaagcaaatactatgacctggtttacaaatactatgacctggttaaagactgccatgatatcttgccgctgattcaaaatccggaaacatttgaaaaatgtcatgagttaaacaacccctgttctcttaaatgcttattcaagcatgctgtgatacatgacatgctgccgattcttcaaaaatatacatactttctggatgggtgggagtattgcaaccagatgctgttcgaactggcatgtagtaaaaaaaaatatgagatggttgtgtggatagagggagttctaggcatcggcaaagttacatctcttttcactattgcgattagcaacagagacctgcacctgtattccctgggccacttaatcattcttgagagaatgcagtcctgtggacaagaccccacgtttttactaaatcatttcctgcgagacgtttcaataaaagggcttctcccttttgtactgaaaaccatagaatatggtggaagcaaggagatagccataactctggctaaaaaatatcagcataaacatattttgaaatacttcgaaaccgggaaatgctaa

SEQ ID NO: 99 - DNA, 843 bp, African swine fever virus
atgtcctcttctcttcaggaactttgtcgaaaaaagctgcctgactgcatacttccagagttttttgacgactatgtattgcaactgttaggactgcactggcaagatcatggttcccttcagcgtatcgagaagaaccagatacttgttcaacaggaacccatccatatcaatgaagcactcaaagtagcagcatcggaagggaactatgaaatcgtagagctgttgttgtcatgggaggcagatccccgctacgccgtcgtaggagccctagaaagcaaatactatgacctggtttacaaatactatgaccaagttaaagactgccatgatatcttgccgctgattcaaaatccggaaacattcgaaagatgtcatgagttaaacagcacctgttcactgaaatgcttattcaagcatgctgtgataaatgacatgctgccgattcttcaaaaatatacagactatctggataggtgggagtattgcagccagatgctgttcgaactggcatgtagtaaaaaaaaatatgagatggttgtgtggatagagggagttctaggcgtcggcaaagttacatctcttttcaccattgcgattagcaacagagacctacagctgtattctctgggctactcaattatccttgagaatttgtactcctgtggacaggaccccaagtttttactaaatcatttcctgcgagacgtttcaataaaagggcttctaccctttgtaatcaaaaccatagaatatggtggaagcaaggagatagccataactctggctaaaaaatatcagcataaacatattttgaaatacttcgaaacctgggaaagctag

SEQ ID NO: 100 - DNA, 843 bp, African swine fever virus; identical to SEQ ID NO: 99

SEQ ID NO: 101 - DNA, 849 bp, African swine fever virus
atgtcctcttcccttcaggaactttgccgaaaaaacttacccgactacatacttccagagttctttgacgactatgtgttgcaactattaggactacactggcaagatcatggttctcttcaacgtaccggaaagaatcaggtacttgttcaacaggaacccattcatatcaatgaagcactaaaagtggcagcatcagagggaaactttgaaatcgtagaactgttgttgtcatggaaggcagacccccgctacgctgtcgtaggagcgctagaaagcaaatactatgacctcgtttacaagtattacaacctagttgaagaccgccatgatatgttgccgctgatccaaaattcagaaacgttcgaaagatgtcatgagttaaacaactgttctcttaaatgcttattcaagcatgctgtaatatatgacaagctgccgattctacaaaaatatgcagactatttggacgggtggccgtattgcaaccagatgctgttcgagttggcatgtaaaaaacaaaaatataacatggttgtgtggatagagggagtcctgggcgtcggcaacttcacaattcttttcacaattgcgattatcaaaagggacctacagctgtattccctgggctactccatcatccttgagagaatgtattcctgtggatacgaccccacgtttttactaaatcactatctgcgagtggtttcaacaaaagggcttctgccttttgtactgaaaaccatagaatatggtggaagcaaagagatagccttaactttggctaaaaaatatcagcatgaaactattttgagatacttcgaaaccaggaaatcccaggagtgctaa

SEQ ID NO: 102 - DNA, 849 bp, African swine fever virus
atgtcctcttcccttcaggaactttgccgaaaaaacttacccgaccacatacttccagagttctttgacgactatatattgcaactattaggactgcactggcaagatcatggttctcttcagcgtaccgagaagaatcaggtacttgttcaacaggaacccatccatatcaatgaagcactaaaagtggcagcatcagaaggaaactacgaaatagtagagctgttgttgtcatggaaggcagacccccgatacgctgtcgtaggagcgctagaaagcaaatactatgacctcgtttacaaatattacgaccaagttaaagactgccatgatatcttgccgctgatccaaaattcggaaacattcgaaagatgtcatgagttgaacaattgttctcttaaatgcttattcaagcatgctgtgatatatgacaagctgccgattcttcaaaaatatgcaagctatctggatgggtggccgtattgcaaccagatgctgttcgagttggcatgtaaaaaacaaaaatataacatggctgtatggatagagggagtcctgggcgtcggcaacttcacaattcttttcacgattgcgattatcaaaagagacctacagctgtattccctgggctactcaatgattcttgagaaaatgtactcctgtggatacgaccctacgtttttactaaatcattatctgcgaatcgtttcaacaaaggggcttctgccctttgtgctgaaaaccatagaatatggtggaagcaaagagatagccatcactttggctaaaaagtatcagcatgaaaatattttgagatacttcgaaaccaggaaaacccaagagtgctaa

SEQ ID NO: 103 - DNA, 849 bp, African swine fever virus
atgtcctcttcccttcaggaactttgccgaaaaaacttacccgacgacatacttccagagttctttgacgactatgtgttgcaactattaggactacactggcaagatcatggttctcttcaacgtaccggaaagaatcaggtacttgttcaacaggaacccattcatattaatgaagcactaaaagtggcagcatcagagggaaactttgaaatcgtagaactgttgttgtcatggaaggcagatccccgctacgctgtcgtaggagcgctagaaagcaaatactatgacctcgtttacaagtattacaacctaattgaagaccgccatgatatgttgccgctgatccaaaattcagaaacgttcgaaagatgtcatgagttaaacaactgttctcttaaatgcttattcaagcatgctgtgatatatgacaagctgccgattctacaaaaatatgcagactatttggacgggtggccgtattgcaaccagatgctgttcgagttggcatgtaaaaaacaaaaatataacatggttgtgtggatagagggagtcctgggcgtcggcaacttcacaattcttttcacgattgcgattatcaaaagggacctacagctgtattccctgggctactccatcatccttgagagaatgtactcctgtggatacgaccccacgtttttactaaatcactatctgcgagtggtttcaacaaaagggcttctgccttttgtactgaaaaccatagaatatggtggaagcaaagagatagccatcactttggctaaaaagtatcagcatgaaactattttgagatacttcgaaaccaggaaatcccaggagtgctaa

SEQ ID NO: 104 - DNA, 843 bp, African swine fever virus; identical to SEQ ID NO: 98

SEQ ID NO: 105 - DNA, 837 bp, African swine fever virus
atgtcccttcaggaactttgccgaaaaaacctgcccgactgcgaacttccagagttctttgacgactatgtattgcaactgttaggattgcactggcaagatcatggttctcttcagcgtaccgggaagaatcaggtacttgttcaacaggaacccatccatatcaatgaagcactaaaaagtgcggcatcagaagggaactatgaaatcgtagagctgctgttgtcatgggaggcagatccccgctacgctgtcgtaggagccctagaaagcaattactatgacctggttcacaaatattatgaccaagttaaagactgccatgatatgctaccactgatccaaaatccggaaatgttcgaaaaatgtcatgagttaaacaacacctgttctcttaaatgcttattcaaacatgctgtgatacatgacatgctgccgattcttcaaaaatattcagactatttggatgggtggcagtattgcaatcagatactgttcgaattggcatgtaaaagacaaaaatataatatggttgtgtggatagagggagttctaggcgtcggcaactttaaaattcttttcaccattgccattaacaacagagatctacagctgtattctctggggtacttaatcattcttgaaagattgtactcctgtggacaagaccccacgtttttactaaaccatttcctacgagacgtttcaatgaaggggcttctgccctttgtactgaaaaccatagaatttggtggaagtaaggagatagccataactctggctaaaaaatatcagcataaacatattttgaagtatttcgaaaccgaagaatgctaa

SEQ ID NO: 106 - DNA, 843 bp, African swine fever virus
atgtcctcttcccttcaggaactttgtcgaaaaaagctgcctgactgcatacttccagagttttttgacgactatgtattgcaactgttaggactgcactggcaagatcatggttcccttcagcgtatcgagaagaaccagatacttgttcaacaggaacccatccatatcaatgaagcactcaaagtagcagcatcggaagggaactatgaaatcgtagagctgttgttgtcatgggaggcagatccccgctacgccgtcgtaggagctctagaaagcaaatactatgacctggtttacaaatactatgaccaagttaaagactgccatgatatcttgccgctgattcaaaatccggaaacatttgaaaaatgtcatgagttaaacaacccctgttctcttaaatgcttattcaagcatgctgtgatacatgacatgctgccgattcttcaaaaatatacatactttctggatgggtgggagtattgcagccagatgctgttcgaattggcatgtagtaaaaaaaaatacgagatggttgtgtggatagagggagttctaggcatcggcaaagttacatctcttttcactattgcgattagcaacagagacctgcacctgtattccctgggccacttaatcattcttgagagaatgcagtcctgtggacaagaccccaagtttttactaaatcatttcttgcgagacgtttcaataaaagggcttctcccctttgtactgaaaaccatagaatatggtggaagcaaggagatagccataactctggctaaaaaatatcagcataaacatattttgaaatacttcgaaaccgggaaatgctaa

SEQ ID NO: 107 - DNA, 309 bp, African swine fever virus
atggttgtgtggatagagggagttctaggcatcggcaaagttacatctcttttcactattgcgattagcaacagagacctgcacctgtattccctgggccacttaatcattcttgagagaatgcagtcctgtggacaagaccccacgtttttactaaatcatttcctgcgagacgtttcaataaaagggcttctcccttttgtactgaaaaccatagaatatggtggaagcaaggagatagccataactctggctaaaaaatatcagcataaacatattttgaaatacttcgaaaccgggaaatgctaa

SEQ ID NO: 108 - DNA, 309 bp, African swine fever virus; identical to SEQ ID NO: 107

SEQ ID NO: 109 - DNA, 843 bp, African swine fever virus
atgtcttcttcccttcaggaactttgtcgaaaaaagctgcctgactgcatacttccagagttttttgacgactatgtattgcaactgttaggactgcactggcaagatcatggttctcttcagcgtatcgagaagaaccagatacttgttcaacaggaacccatccatatcaatgaagcactcaaagtagcagcatcggaagggaactatgaaatcgtagagctgttgttgtcatgggaggcagatccccgctacgccgtcgtaggagctctcgaaagcaaatactatgacctggttcacaaatactatggccaagttaaagactgccatgatatcttgccgctgattcaaaatccggaaacattcgaaaaatgtcatgagttaaacagcacctgttcactgaaatgcttattcaagcatgctgtgatgaatgacatgctgccgattcttgaaaaatatacagactatctggataggtgggagtattgcagccagatgctgttcgaattggcatgcagaaaaaaaaaatatgagatggttgtgtggatagagggagttctatgcgtcggcaaagttacgtctcttttcaccattgcgattagcaacagagacctacagctgtattctctgggctactcaattattcttgagaaaatgtactcctgtcgacaggaccccacgtttttactaaatcatttcctgcgagacgtttcgataaaggggcttctgccctttgtactgaaaaccatagaatatggtggaagcaaggagatagccataactctggctaaaaaatatcagcataaacatattttgaaatacttcgaaacctgggaaagctag

SEQ ID NO: 110 - DNA, 846 bp, African swine fever virus
atgtcctcttcctcccttcaggaactttgccgaaaaaagctgcctgactgcatccttccagagtttttcgacgactatgtattgcaactgttaggactgcactggcaagatcatggttctcttcagcgtatcgagaagaaccagatacttgttcaacaggaacccatccatatcaatgaagcactcaaagtagcagcatcggaagggaactatgaaatcgtagagctgttgttgtcatgggaggcagatccccgctacgccgtcgtaggagctctcgaaagcaaatactatgacctggttcacaaatactatgaccaagttaaagactgccatgatatcttgccgctgattcaaaatccggaaacattcgaaaagtgtcatgagttaaacagcacctgttcactgaaatgcttattcaagcatgctgtgataaatgacatgctgccgattcttcaaaaatatacagactatctggataggtgggagtattgtagccagatgctgttcgaattggcatgtagaaaaaaaaagtatgagatggttgtgtggatagagggagctctgggcgtcggcaaagttacatctcttttcaccattgcgattagcaacagagacctgcagctgtattctctgggctactcaatgattcttgagaaattgtactcctgtggacaggaccccacgtttttactaaatcatttcctgcgagacgtttcgataaaggggcttctgccctttgtactcaaaaccatagaatatggtggaagcaaggagatagccataactctggctaaaaaatatcagcataaacatattttgaaatacttcgaaacttgggaaagctag

SEQ ID NO: 111 - DNA, 843 bp, African swine fever virus
atgtcctcttcccttcaggaactttgtcgaaaaaagctgcctgactgcatacttccagagtttttcgacgactatgtattgcaactgttaggactgcactggcaagatcatggttctcttcagcgtatcgagaagaaccagatacttgttcaacaggaacccatccatatcaatgaagcactcaaagtagcagcatcggaagggaactatgaaatcgtagagctgttgttgtcatgggaggcagatccccgctacgccgtcgtaggagctctcgaaagcaaatactatgacctggttcacaaatactatggccaagttaaagactgccatgatatcttgccgctgattcaaaatccggaaacattcgaaaaatgtcatgagttaaacagcacctgttcactgaaatgcttattcaagcatgctgtgatgaatgacatgctgccgattcttgaaaaatatacagactatctggataggtgggagtattgcagccagatgctgttcgaattggcatgtagaaaaaaaaaatatgagatggttgtgtggatagagggagttctaggcatcggcaaagttacgcctcttttcaccattgcgattagcaacagagacctacagctgtattctctgggctactcaattattcttgagagaatgtattcctgtgaacaggatcccacgtttttactaaatcatttcctgcgagacgtttcgataaaggggcttctgccctttgtactgaaaaccatagaatatggtggaagcaaagagatagccataactctggctaaaaaatatcagcataaacatattttgaaatacttcgaaacatgggaaagctaa

SEQ ID NO: 112 - DNA, 843 bp, African swine fever virus
atgtcctcttcccttcaggaactttgccgaaaaaagctgcctgactgcatacttccagagtttttcgacgactatgtattgcaactgttaggattgcattggcaagatcatggttctcttcagcgtatcgagaagaaccagatacttgttcaacaggaacccatccatatcaatgaagcactcaaagtagcagcatcggaagggaactatgaaatcgtagagctgttgttgtcatgggaggcagatccccgctacgccgtcgtaggagctctcgaaagcaaatactatgacctggttcacaaatactatggccaagttaaagactgccatgatatcttgccgctgattcaaaatccggaaacatttgaaaagtgtcatgagttaaacagcacctgttcactgaaatgcttattcaagcatgctgtgatgaatgacatgctgccgattcttgaaaaatatacagactatctggataggtgggagtattgcagccagatgctgttcgaattggcatgtagaaaaaaaaaatatgagatggttgtgtggatagagggagttctaggcgtcggcaaagttacgtctcttttcaccattgcgattagcaacagagacctacagctgtattctctgggcttctcaattattcttgagaaattgtactcctgtggacaggaccccacgtttttactaaatcatttcctgcgagacgtttcgataaaggggcttctgccctttgtactcaaaaccatagaatatggtggaagcaaggagatagccataactctggctaaaaaatatcagcataaacatattttgaaatacttcgaaacctgggaaagctag

SEQ ID NO: 113 - protein, 350 aa, African swine fever virus
MLPSLQSLTKKVLAGQCVPTNQHYLLKYYDLWWYNAPITFDHNLRLIKSSGIKEGLDLNTALVKAVRENNYSLIKLFTEWGADINYGLVSVNTEHTRDLCQELGAKEILNEEEILQIFIDLKFHKTSSNIILCHEVFSNNPILQKVNNLKLRIEIFWELRELIEKTDLLNNEFLLSTLLLKYWYAIAVRYSLKEAIQYFYQKYTHMNTWRLTCALCFNNVFDLHEAYEKDKIHMDIEEMMRIACIKDHNLSTMYYCYMLGANINQAMLTSIQYYNIENMFFCMDLGADVFEEGTTALGEGYELIKNILSLKIYSPTTIPLPKSTDPEIIDHALKNYFSKNMMIFLSYDLR

SEQ ID NO: 114 - protein, 350 aa, African swine fever virus; identical to SEQ ID NO: 3

SEQ ID NO: 115 - protein, 351 aa, African swine fever virus
MLPSLQSLTKKVLARQCLPEDQHYLLKCYDLWWNNAPITFDHNLRLIKLAGIQEGLDLNMALVKAVKENNYSLIKLFTEWGANINYGLISVNTEHTWDLCRELGAKKTLNEGDILQIFIDLKFYKTSSNIILCHEVFSDNLLLKRVNNLKMRIEIFWELREIIEKTDLLNNEFSLNTLLLKYWYAIAVRYNLKEAIQYFYQKYTHLNTWRLTCALCFNNVFDLHEAYEKDKICMDLEEMMRIACIKDHNLSTIYYCYMLGANINQAMLTSIQYYNIENIFFCMDLGADAFEEGMALVGQEGYEPIRNILSLKIYSPATTPLPKSTDPEIIDHALKNYFSKNMMVFLTYDLR

SEQ ID NO: 116 - protein, 351 aa, African swine fever virus
MLPSLQSLTKKVLAGQCLPEDQHYLLKCYDLWWNNAPITFDHNLRLIKSAGLQEGLDLNMALVKAVKENNYSLIKLFTEWGANINYGLISVNTEHTWDLCRELGAKKTLNEGDILQIFIDLKFHKTSSNIILCHEVFSDNLLLKKVNNLKMRIEIFWELREIIEKTDLLNNEFSLNTLLLKYWYAIAVRYNLKEAIQYFYQKYTHLNTWRLTCALCFNNVFDLHEAYEKDKICMDLEEMMRIACIKDHSLSTIYYCYMLGANINQAMLTSIQYYNIENIFFCMDLGADAFEEGMALVGQEGYEPIRNILSLKIYSPATTPLPKSTDPEIIDHELKNYFSKNMMVFLTYDLR

SEQ ID NO: 117 - protein, 350 aa, African swine fever virus
MLPSLQSLTKKVLAGQCVPTNQHYLLKYYDLWWYNAPITFDHNLRLIKSSGIKEGLDLNTALVKAVRENNYSLIKLFTEWGADINYGLVSVNTEHTRDLCQELGAKEILNEEEILQIFIDLKFHKTSSNIILCHEVFSNNPILQKVNNLKLRIEIFWELRELIEKTDLLNNEFLLSTLLLKYWYAIAVRYSLKEAIQYFYQKYTHLNTWRLTCALCFNNVFDLHEAYEKDKIHMDIEEMMRIACIKDHNLSTMYYCYMLGANINQAMLTSIQYYNIENMFFCMDLGADVFEEGTTALGEGYELIKNILSLKIYSPTTIPLPKSTDPEIIDHALKNYFSKNMMIFLSYDLR

SEQ ID NO: 118 - protein, 349 aa, African swine fever virus
MLPSLQSLTKKVLAGQCLPTDQYYLLKCYDLWWYDSPITFDHNLGLIKSAGIKDGLDLNTALVKAVRENNYNLIKLFTEWGADINYGLVSVNTEHTRDLCRELGAKETLNEEEILRIFIDLKFYKTSSNIILCHEVFSNNPLLQKVNNLKMRIEIFWELRELIKKTDLLNNEFSLNTLLLKYWYAIAVRYNLKEAIQYFYQKYTHLNTWRLTCALCFNNVFDLHEAYEKDKIYMDLEEMMRVACIKDHNLSTIYYCYVLGANINQAMLASIQYYNIENMFFCMDLGADVFEENMPVGEGYELIRNILSLKIYSPSTAPLPKNTDPEIIDHVLKNYKSKNMMTFLSYDLR

SEQ ID NO: 119 - protein, 350 aa, African swine fever virus
MLPSLQSLTKKVLAGQCVPTNQHYLLKYYDLWWHDAPITFDHNLRLIKSAGIKEGLDLNTALVKAVKENNYNLIKLFAEWGADINYGLVSVSSEHTWDLCRELGAKETLNEKEILQIFIDLKFHKTSSNIILCHEVFSNNPILQKVNNIKMRIEIFWELRELIEKTDLLNNEFSLSTLLLKYWYAIAIRYNLKEAIQYFYQKYTHLNTWRLTCALCFNNVFDLHEAYEKDKIYMDIEEMMRIACIKDHNLSTMYYCYMLGANINQAMLTSIQYYNIENMFFCMDLGADVFEEGTTALGEGYELIKNILSLKIYSPTTIPLPKSTDPEIIDHALKNYFSKNMMIFLSYDLR

SEQ ID NO: 120 - protein, 350 aa, African swine fever virus
MLPSLQSLTKKVLAGQCVPTNQHYLLKCYDLWWHDGPITFDHNLKLIKSAGIKEGLDLNTALVKAVRENNYNLIKLFAEWGANINYGLVSVNTEHTWDLCRELGAKETLNEEEILQIFIDLKFHKTSSNIILCHEVFSNNPILQKVNNIKMRIEIFWELRELIEKTDLLNNEFSLSTLLLKYWYAIAIRYNLKEAIQYFYQKYTHLNTWRLTCALCFNNVFDLHEAYEKDKIHMDIEEMMRIACIKDHNLSTMYYCYVLGGNINQAMLTSIQYYNIENMFFCMDLGADAFEEGTIALGEGYKLIKNILSLKIYSPATTPLPKSTDPEIIDHALKNYVSKNMMIFLTYDLR

SEQ ID NO: 121 - protein, 350 aa, African swine fever virus
MLPSLQSLTKKVLAGQCVPTNQHYLLKCYDLWWHDAPITFDHNLRLIKSAGIKEGLNLNTALVKAVRENNYNLIKLFAEWGADINYGLVSVNTEHTWDLCRELGAKETLNEEEILQIFIDLKFHKTSSNIILCHEVFSNNPILQKVNNIKMRIEIFWELRELIEKTDLLNNEFSLSTLLLKYWYAIAIRYNLKEAIQYFYQKYTHLNTWRLTCALCFNNVFDLHEAYEKDKIHMDIEEMMRIACIKDHNLSTMYYCYVLGANINQAMLSSIQYYNIENMFFCIDLGADVFEEGTTALGEGYELIKNILSLKIYSPATTPLPKSMDPEIIDHALKNYVSKNMMIFLTYDLR

SEQ ID NO: 122 - protein, 350 aa, African swine fever virus
MLPSLQSLTKKVLAGQCVPTNQHYLLKCYDLWWHDGPITFDHNLKLIKSAGIKEGLDLNTALVKAVRENNYNLIKLFAEWGADINYGLVSVNTEHTRDLCRELGAKETLNEEEILQIFIDLKFHKTSSNIILCHEVFSNNPILQKVNNIKMRIEIFWELRELIEKTDLLNNEFSLSTLLLKYWYAIAIRYNLKEAIQYFYQKYTHLNTWRLTCALCFNNVFDLHEAYEKDKIYMDIEEMMRIACIKDHNLSTMYYCYVLGANINQAMLTSIQYYNVENMFFCMDLGADAFEEGTIALGEGYKLIKNILSLKIYSPATTPLPKSTDPEIIDHAVKNYVSKNMMIFLTYDLR

SEQ ID NO: 123 - protein, 350 aa, African swine fever virus
MLPSLQSLTKKVLAGQCVPTNQHYLLKCYDLWWHDGPITFDHNLKLIKSAGIKEGLDLNTALVKAVRENNYNLIKLFAEWGANINYGLVSVNTEHTRDLCRELGAKETLNEEEILQIFIDLKFHKTSSNIILCHEVFSNNPILQKVNNIKMRIEIFWELRELIEKTDLLNNEFSLSALLLKYWYAIAIRYNLKEAIQYFYQKYTHLNTWRLTCALCFNNVFDLHEAYEKDKIHMDIEEMMRIACIKDHNLSTMYYCYVLGANINQAMLTSIQYYNIENMFFCMDLGADAFEEGTIALGEGYKLIKNILSLKIYSPATTPLPKSTDPEIIDHALKNYVSKNMMIFLTYDLR

SEQ ID NO: 124 - protein, 353 aa, African swine fever virus
MSLPLSLQTLVKKTVASQCLSIDEHCILKYCGLWWHDAPLKLCMDRGRIQIKSGFLGEDIDLRVALIIAVKENNYSLIKLFTEWGANINYSLLSINTKHIRELCRQLGAKETLEDNDIFRIFTRIMHNKTSGSIILCHEIFMNNPMLENKFVIQLRGLIYKRLWGLIEIKETDELNDLLVKYWYAKAVQYVCKNAICFLDEKYTDLNEWRLKCLLYYNKIYELHEMYHKKKVQIDVHDMICLACAKDNNLLTIYYCYALGGNINQAMLTSVQYYNVGNIFFCIDLGGNAFEEGRAIAEQKGYNFLSHSLTLDIYSSDASLPLNLKDPEKISSLLKDYKSKNLSIIWEYSHNIL

SEQ ID NO: 125 - protein, 353 aa, African swine fever virus; identical to SEQ ID NO: 4

SEQ ID NO: 126 - protein, 352 aa, African swine fever virus
MSSPLSLQTLVKKTVAGTSCLSIDEHILKYCGLWWHDAPLKLYIDRGRIYIKSGFLGEDIDLCVALIIAVKENNYSMIKLFTEWGAYINYSLLSINTKHARDLCRQLGAKETLDDYDIFCIFNKIMHNKTSGSIILCHEIFINNPNLENNFAAQLRRLIYKRLCGLIEIKETDELSELLVKYWYANAVQYDHKDAICFLDEKYTDLDEWLKCYLCYNKIYELHDIYHKEKIQIDVNEMLSLACIRDNNLLTIYYCYALGGNINQAMLTSVQYYNIGNIYFCIDLGGNAFEEGSAIARQNGYNFLSHSLVLNIYSSDASLPLNLKDPEEISSLLKNYKSKNLSIILDYSHKIL

SEQ ID NO: 127 - protein, 353 aa, African swine fever virus
MSSPLSLQTLVKKTVASTSCLSIDEHILKYCGLWWHDAPLKLYIDRGRIYIKSGFLGEDIDLCVALIIAVKENNYSLIKLFTEWGAYINYSLLSINTKHARDLCRQLGAKETLDDYDIFCIFNKIMHNKTSGSIILCHEIFINNPKLENNFAAQLRRLIYKRLCGLIEIKETDELSELLVKYWYANAVQYDHKDAICFLDEKYTDLDEWRLKCYLCYNKIYELHDIYHKKKIQIDVNEMLSLACIRDNNLLTIYYCYALGGNINQAMLTSVQYYNIGNIYFCIDLGGNAFEEGSAIARQNGYNFLCHSLILNIYSSDASLPLNLKVPEEISSLLKNYKSKNLSIILDYSHKIL

SEQ ID NO: 128 - protein, 353 aa, African swine fever virus; identical to SEQ ID NO: 124

SEQ ID NO: 129 - protein, 353 aa, African swine fever virus
MSAPLSLQTLVKKTVASTSCLSIDEHILKYCDLWWHDAPLKLYMDRGRIQIKSGFLGEDIDLCVALIIAVKENNYNLIKLFTELGANINYSLLSINTKHVRDLCRQLGAKETLEDYDIFCIFNKIMHNKTSGSVILCHEIFINNPNLENKFAAQLRRLIYKRLCGLIEIKETDELSELLVKYWYAKAVQYDYKDAICFLDEKYTDLNEWRLKCYLYYNKIYELHDIYHKEKIQIDVNEMLSLACIRDNNPLTIYYCYALGGNINQAMLTSVQYYNIGNIYFCIDLGGNAFEEGSAIARQKGYNFLSHSLVLNIYSSDASLPLNLKDPEEISSLLKNYKSKNLSIILDYSHNIL

SEQ ID NO: 130 - protein, 353 aa, African swine fever virus
MSLPLSLQTLVKKTVASQCLSIDEHCILKYCGLWWHDAPLKLCMDRGRIQIKSGFLGEDIDLRVALIIAVKENNYSLIKLFTEWGANINYSLLSINTKHIRELCRQLGAKETLEDNDIFRIFTRIMHNKTSGSIILCHEIFMNNPILENKFVIQLRGLIYKRLWGLIEIKETDKLNDLLVKYWYAKAVQYVCKNAICFLDEKYTDLNEWRLKCLLYYNKIYELHEMYHKKKVQIDVHDMICLACAKDNNLLTIYYCYALGSNINQAMLTSVQYYNIGNIFFCIDLGGNAFEEGRAIAEQKGYNFLSHSLTLDIYSSDASLPLNLKDPEEISSLLKDYKSKNLSIIWEYSHNIL

SEQ ID NO: 131 - protein, 353 aa, African swine fever virus
MSLPLSLQTLVKKTVASQCLSIDEHCILKHCGLWWHDVPLKLCMDRGQIQIKSGFLGEDIDLHIALIIAVKENNYSLIKLFTEWGAHINYSLLSINTEHIRELCRQLGAKETLEDDDVFRIFTKIMHNKTSGRIILCHDIFMNNPNIENKFTIQLRGLICKRLWGLIEIKETDELNDLLVKYWYAKAVQYECKDAICFLEEKYTDLNEWRLKCLLYFNKIYELHEMYHKEKVQIDVHDMICLASTKDNNPLTIYYCYALGGNINQAMLTSIQYYNIGNIFFCIDLGGNAFEEGRAIAEQKGYNFLSHSLALDIFSSDASLPLNLKDPEEISSFLKDYKSKNLSIIWEYSHNIL

SEQ ID NO: 132 - protein, 353 aa, African swine fever virus
MSLPLSLQTLVKKTVANQSLSIDEHCILKHCGLWWHDVPLKLCMDRGQIQIKSGFLGEDIDLHIALIIAVKENNYSLIKLFTEWGANINYSLLSINTKHIRELCRQLGAKETLEDDDIFRIFTKIMHNKTSGSIILCHDIFMNNPNIEDKFTIQLRGLIYKRLWGLIEIKETDELNGLLVKYWYAKAVQYECKDAICFLDEKYTDLNEWRLKCLLYYNKIYELHEMYHKENIQIDVHDMICLASTKDNNPLTIYYCYALGGNINQAMLTSVQYYNIGNIFFCIDLGGNAFEEGRAIAEQKGYNFLSHSLALDIYSSDASLPLNLKDPEEISSLLKDYKSKNLSIIWEYSHNIL

SEQ ID NO: 133 - protein, 353 aa, African swine fever virus
MSLPLSLQTLVKKTVASQCLSTDEHCILKHCGLWWHDVPLKLCMDRGQIQIKSGFLGEDIDLHVALIIAVKENNYSLIKLFTEWGAHINYSLLSINTEHIRELCRQLGAKKTLEDNDIFRIFTKIMHNKTSGRIILCHEIFMNNPNIENKFTIQLRGLICKRLWRLIEIKETDELNDLLVKYWYAKAVQYECKDAICFLDEKYTGLNEWRLKCLLYYNKIYELHEMYHKDKVQIDVHDMICLASTKDNNPLTIYYCYALGGNINQAMLTSVQYYNIGNIFFCIDLGGNAFEEGRAIAEQKGYHFLSHSLALDIYSSDASLPLNLKDPEEISSLLKGYKSKNLSIIWEYSHNIL

SEQ ID NO: 134 - protein, 353 aa, African swine fever virus
MSLPLSLQTLVKKTVASQCLSTDEHCILKHCGLWWHDVPLKLCMDRGQIQIKSGFLGEDIDLHIALIIAVKENNYSLIKLFTEWGAHINYSLLSINTEHIRELCRQLGAKETLEDNDIFRIFTKIMHNKTSGRIILCHEIFMNNPNIENKFTIQLRGLICKRLWGLIEIKETDELNDLLVKYWYAKAVQYECKDAICFLDEKYTDLNEWRLKCLLYYNKIYELHEMYHKEKVQIDVHDMICLASTKDNNPLTIYYCYALGGNINQAMLTSVQYYNIGNIFFCIDLGGNAFEEGRAIAEQKGYHFLSHSLTLDIYSSDASLPLNLKDPEEISSLLKDYKSKNLSIIWEYSHNIL

SEQ ID NO: 135 - protein, 357 aa, African swine fever virus
MLSLQTLAKKVVACNYLSSDYDYTLQRFGLWWDLGPIHLCNNCKQVFSYKHLQCFSEDDLCLEAALVKAVKSDNLELIRLFVDWGANPEYGLIRVPAVYLKRLCAELGGLTPVSEPRLLEILKEVANLKSCAGVLLGYDMFCHNPLLETVTRTTLDTVTYTCSNIPLTGDTAHLLLTKFWFALALRHNFTKAIHYFYKRHKNQLYWRVACSLYFNNIFDIHELCREKEICISPNLMMKFACLREKNYAAIYYCHRLGASLDYGMNLSIYNNNTLNMFFCIDLGAADFDRAQLIAHKAYMYNLSNIFLVKQLFSRDVTLVLDVTEPQEIYDMLKTYTSKNMKRAEEYLTAHPEIIVID

SEQ ID NO: 136 - protein, 357 aa, African swine fever virus; identical to SEQ ID NO: 5

SEQ ID NO: 137 - protein, 357 aa, African swine fever virus
MLSLQTLAKKVVACNYLSSDYDYTLQRFGLWWDLGPIHLCNTCKQIFSYQHLQCFSVDDLCLDAALVKAVKSDNLELIRLFVDWGANPEYGLIRVPAVHLKRLCTELGGLTPVSESRLLEILKEVADLKSCAGVLLGYDMFCHNPLMETVTRTTLNTVMYTRSKIPLTGDTAHLLLTKFWFALALRHNFTKAIHYFYEKHKNQLYWRLTCSLYFNNIFDLHELCSKKEICISPNLMMKFACLREENYAAIYYCHMLGASPDYGMNLSIYNNNTLNLFFCIDLGATNFDRARLIAHRVYMYNLSNIFLVKQLFSRDASLVLNITEPQAIYDMLKTYTSKNLKRAEEYFTAHPEIVVID

SEQ ID NO: 138 - protein, 357 aa, African swine fever virus
MLSLQTLAKKVVACNYLSSDYDYTLQRFGLWWDLGPIHLCNTCKQIFSYQHLQCFSEDDLCLDAALVKAVKSDNLELIRLFVDWGANPEYGLIRVPAVDLKRLCTELGGLTPVSESRLLEILKEVADLKSCAGVLLGYDMFCHNPLLETVTRTTLNTVMYTRSKIPLTGDTAHLLLTKFWFALALRHNFTKAIHYFYEKHKNQLYWRLACSLYFNNIFDLHELCSKKEICISPNLMMKFACLREENYAAIYYCHMLGASPDYGMNLSIYNNNTLNLFFCIDLGATNFDRARLIAHRVYMYNLSNIFLVKQLFSRDASLVLNITEPQAIYDMLKTYTSKNLKRAEEYFTAHPEIVVID

SEQ ID NO: 139 - protein, 357 aa, African swine fever virus; identical to SEQ ID NO: 135

SEQ ID NO: 140 - protein, 357 aa, African swine fever virus
MLSLQTLAKKVVACNYLSSDYDYTLQRFGLWWDLGPIHLCNTCKQIFSYKHLQCFSEDDLCLEAALVKAVKSDNLELIRLFVDWGANPEYGLIRVPAVHLKRLCMELGGLTPVSESRLLEILKEVADLKSCAGVLLGYDMFCHNPLLETVTRTTLDTVMYTRSKIPLTGDTAHLLLSKFWFALALRHNFTKAIHYFYEKHKNQLYWRLTCSLYFNNIFDLHELCCKKEICISPNLMMKFACLREENYAAIYYCHMLGASLDYGMNLSIYNNNTLNLFFCIDLGATNFDRARLIARRVYMYNLSNLFLVKQLFSRDVSLILDLTEPQAIYDMLNTYTSKNLKQAEEYFTAHPEIVVID

SEQ ID NO: 141 - protein, 357 aa, African swine fever virus; identical to SEQ ID NO: 135

SEQ ID NO: 142 - protein, 357 aa, African swine fever virus
MLSLQTLAKKVVACNYLSSDYDYTLQRFGLWWDLGPIHLCNNCKQIFSYKHLQCFSEDDLCLEAALVKAVKSDNLELIRLFVDWGANPEYGLIRVPAVHLKRLCTELGGLTPVTEPRLLEILKEVAKLKSCAGVLLGYDMFCHNPLLETVTRTTLDTVTYTCSNIPLTGDTAHLLLTKFWFALALRHNFTKAIHYFYKRHKNHLYWRVACSLYFNNIFDIHELCREKEICISPNLMMKFACLQKKNYAAIYYCYRLGASLDYGMNLSIYNNNTLNMFFCIDLGATDFDRAQHIAHKAYMYNLSNIFLVKQLFSRDVTLALDVTEPQEIYDRLKSYTSKNLKRAEEYLTAHPEIIVID

SEQ ID NO: 143 - protein, 357 aa, African swine fever virus
MLSLQTLAKKVVACNYLSSDYDYTLQRFGLWWDLGPIHLCNNCKQIFSYKHVQCFSEDDLCLEAALVKAVKSDNLELIRLFVDWGANPEYGLIRVPAVHLKRLCTELGGLTPVSESRLLEILKEVARLKSCAGVLLGYDMFCHNPLLETVTRTTLDTVTYTCSNIPLTGDTAHHLLTKFWFALALRHNFTKAIHYFYKRHKNHLYWRVACSLYFNNIFDIHELCREKEICISPNLMMKFACLQKKNYAAIYYCYRLGASLDYGMNLSIYNNNTLNMFFCIDLGATDFDRAQRIAHKAYMYNLSNIFLVKQLFSRDVTLALDVTEPQEIYDMLKSYTSKNLKRAEEYLTAHPEIIVID

SEQ ID NO: 144 - protein, 357 aa, African swine fever virus
MLSLQTLAKKVVACNYLSSDYDYMLQRFGLWWDLGPIHLCNNCKQIFSYKHLQCFSEDDLCLEAALVKAVKRDNLELIRLFVDWGANPEYGLICVPTVHLKRLCTELGGLTPVSEPRLLEILKEVANLKSCAGVLLGYDMFCYNPLLETITRTTLDTVMYSCSKIPLMGDTAHHLLTKFWFALALRHNFTKAIHYFYKRHKNHLYWRVACSLYFNNIFDIHELCREKEICISPNLMMKFACLREKNYAAIYYCYRLGASLDYGMNLSIYNNNSLNLFFCIDLGATDFDRAQRIAHKAYMYNLSNILLVKQLFSRDVTLALDVTEPQEIYDRLKAYTSKNMKRAEEYLTAHPEIIVID

SEQ ID NO: 145 - protein, 357 aa, African swine fever virus
MLSLQTLAKKVVACNYLSSDYDYMLQRFGLWWDLGPIHLCNNCKQIFSYKHLQCFSEDDLCLEAALVKAVKSDNLELIRLFVDWGANPEYGLIRVPAVHLKRLCTELGGLTPVSEPRLLEILKEVAKLKSCAGVLLGYDMFCHNPLLETVTRTTLDTVTYTCSNIPLTGDTAHHLLTKFWFALALRHNFTKAIHYFYKRHKNHLYWRVACSLYFNNIFDIHELCREKEICISPNLMMKFACLRKKNYAAIYYCYRLGASLDYGMNLSIYNNNTLNMFFCIDLGATDFDRAQRIAHKTYMYNLSNIFLVKQLFSRDVTLALDVTEPQEIYDMLKSYTSKNLKRAEEYLTAHPEIIVID

SEQ ID NO: 146 - protein, 531 aa, African swine fever virus
MFSLQNLCRKTLPDCKLPEFFDDYILQLLGLYWENHGTIQRAGNNCVLIQQHTLIPVNEALRIAASEENYEIVGLLLAWEGNLYYAIIGALEGNRYNLIRKYDDQIKDHHDILPFIDDPIIFHKCHIMRRCFFDCILYQAVKYSKFRVLLYFKYTLEDDLPLVHLLIEKACEDHNYEVIKWIYENLHVCHIIDTFDCAIAHKDLRLYCLGYTFIYNRIVPYKYHHLDILILSSLQLLHKVAAKGYLDFILETLKYDHNIDNLDVILTQAATYNHRKILTYFIPQSTYAQIEQCLFVAIKTKSSKKTLNLLLSHLNLSIKLIQKISQYVATFNSTNIIGILSMKRKKKIYLDIILTKFVKNAIFNKFVVRCMERFSINPERIVKMAARINKMMLVKKISEHVWKNHAARLKHLKHAVHTMKHKDGKNRLMNFIYEHCYYHMQGEEIFSLARFYAIHHAPKLFDVFYNCCILDTIRFKSLLLDCSHIIGKNAHDATNINIVNKYIGNLFAMGVLSKKEILQDYPSIYSKHYMP

SEQ ID NO: 147 - protein, 531 aa, African swine fever virus; identical to SEQ ID NO: 6

SEQ ID NO: 148 - protein, 530 aa, African swine fever virus
MFSLQNLCRKTLPDCKLPEFFDDYVLQLLGLYWENHGTIQRAGNNCVLIQQHNLIPVNEALRIAASEENYEIVSLLLAWEGNLYYAIIGALEGNRHNLIRKYDDQIKDHHEILPFIDDPVIFHKCHIMRRCFFNCMLYQAVKYSKFSVLLYFKYILKENLPLVHSLIEKAYKYHNYEVIKWIYENLHIYDIINTFKYAIAHKDLRLYCLGYTFVYNRIVPYKYYHLDIRILLRLQLLHKVTAKGYLDFILETLKYDHNTNNIDIILTQAATYNHRNILTYFIPQSTYAQIEQCLFVAIKTNASKKTLNLLLSHLNLSIKLVKKLSQYVVAYKSTNIISILSMQQKKKIYLDIILTKVVKNAVFIKFVIGCMVTFSINPERIVKMAARIKKMKLVKNISEHVWKNHAAKLKHLKHAVHTMKHQEGKNRLMNFIYDHCYYHMQGEEIFSLARFYAIHHAPKLFDVFYDCCILDTIRFKSLLLDCSHIIAKNAHDASINIVNKYIGNLFAMGVLSKKEILQDYPSIYSKYDIL

SEQ ID NO: 149 - protein, 530 aa, African swine fever virus
MFSLQNLCRKTLPDCKLPEFFDDYVLQLLGLYWENHGTIQRAGNNCVLIQQHNLIPVNEALRIAASEENYEIVSLLLAWEGNLYYAIIGALEGNHHNLIRKYDDQIKDHHEILPFIDDPVIFHKCHTMRRCFFNCILYQAVKYSKFSVLLYFKYILKENLPLVHSLIEKAYKYHNYEVIKWIYENLHIYDIINTFKCAIAHKDLRLYCLGYTFVYNRIVPYKYYHLDIRILLRLQLLHKVTAKGYLDFILETLKYDHNTNNIDIILTQAATYNHRNILTYFIPQSTYAQIEQCLFVAIKTNASKKTLNLLLSHLNLSIKLVKKLSQYVVAYKSTNIISILSRQQKKKIYLDIILTKVVKNAVFNKFVIGCMVTFSINPERIVKMAARIKKMKLVKNISEHVWKNHAVKLKYLKHAVHTMKHQEGKNRLMNFIYDHCYYHMQEEEIFSLARFYAIHHAPKLFDVFYDCCILDTIRFKSLLLDCSHIIVKNAHDASINIVNKYIGNLFAMGVLSKKEILQDYPSIYSKDYML

SEQ ID NO: 150 - protein, 530 aa, African swine fever virus
MFSLQNLCRKTLPDRKLPEFFDDYVLQLLGLYWENHGTIQRAGNNCVLIQQHNLIPVNEALRIAASEENYEIVSLLLAWEGNLYYAIIGALEGNRHNLIRKYDDQIKDHHEILPFIDDPVIFHKCHMMRRCFFNCILYQAVKYSKFSVLLYFKYILKENLPLVHSLIEKACEDHNYEVIKWIYENLHIYEIMDTFKCAIAHKDLHLYSLGYTFIYNRIVPYKYHHLDIRILSRLQLLHKVTAKGYLDFILETLKYDHNKDNINIILTQAATYNHRNILTYFIPQSTYAQIEQCLFVAIKTNASKKTLNLLLSHLNLSIKLVKKLSQYVVAYKSTNIISILSMQQKKKIYLDIILTKVVKNAIFIKFVIGCMVTFSINPERIVKMAARIKKMKLVKNISEHVWKNHAAKLKHLKHAVHTMKHQEGKNRLMNFIYDHCYYHMQGEEIFSLARFYAIHHAPKLFDVFYDCCILDTIRFKSLLLDCSYIIAKNAHDASINIVNKYIGNLFAMGVLSKKEILQDYPSIYSKDYML

SEQ ID NO: 151 - protein, 531 aa, African swine fever virus; identical to SEQ ID NO: 146

SEQ ID NO: 152 - protein, 530 aa, African swine fever virus
MFSLQNLCRKTLPDCKLPEFFDEYILQLLGLYWENHGTIQRAGNNCVLIQQHNLIPVNEALRIAASEENYEIVSLLLAWEGNLYYAIIGALEGNRPDLIRKYDDQIKDHHEILPFIDDPIIFHKCHIMRRCFFNCILYQAVKYSKFRVLLYFKHRLGDDLPLTHLLIEKACEDHNYEVIKWIYENLHSYNIMDTFECAIAHKDLRLYCLGYTFIYNRIVPYKYHHLDICILSSLQLLHKVAAKGYLDFILETLKYDHNINNIDIILTQAATYNHRKILTYFIPQLTYAQIEQCLLVAIKTKASKKTLNLLLSHLNLSIKLIKKISQYVVTYNSTNIISILSMRRKKKIYLDIILTEFVKNAIFNKFVVRCMDTFSINPERIVKMAARINRMMLVKNISERVWKNHAVKLKHLKHAVHTMKHQEGKNRLMNFIYDHCYYHMQGEEIFGLARFYAIHHAPKLFDVFYDCCMLDATRFKSLLLDCPHIIGKNAYDAGINLVNKYIGNLFAMGVLSKKEILQDYPSIYSKHDMF

SEQ ID NO: 153 - protein, 531 aa, African swine fever virus
MFSLQNLCRKTLPDCKLPEFFDDYILQLLGLYWENHGTIQRAGNNCVLIQQHTLIPINEALRTAASEENYEIVSLLLAWEGNLYYAIIGALEGNRHDLIRKYDDQIKDHHEILPFIDDPVIFHKCHIMRRCFFDCILYQAVKYSKFRVLLYFKYRLEDDLPLTHLLIEKACENHNYEVIRWIYENLHIYNMIDTFECAIAHKDLRLYCLGYTFIYNRIVPNKYHHIDILILSSLQLLHKVAAKGYLDFILETLKYDHNNDNLDIILTQAATYNHRKILTYFIPQSTYAQIEQCLMVAIKTKSSKKTLNLLLSHLNLSIKLIKKISQYVVTYNSTNIIGILSMKRKKKIYLDILLTKFVKNAIFNKFVVRYMDTFSINPEKIVKMAARINKMMLVKKISEHIWKNHAARLEHLKHAVHTMKHKDGKNRLMNFIYEYCYYHMQGEEIFSLARFYAIHHAPKLFDVFYNCCILDTIRFKSLLLDCSHIIGKNAHDATNINIVNKYIDNLFAMGVLSKKEILQDYPSIYSKHYMP

SEQ ID NO: 154 - protein, 531 aa, African swine fever virus
MFSLQNLCRKTLPDCKLPEFFDEYILQLLGLYWENHGTIQRAGNNCVLVQQHTLIPVNEALRIAASEENYEIVSLLLAWEGNLYYAIIGALEGNRHDLIRKYDDQIKDHHEILPFIDNPVIFHKCHIMRRCFFDCILYQAVKYSKFRVLLYFKYRLENDLPLAHLLVEKACEDHNYEVIKWLYENLHIYNIMETFECAIAHKDLRLYRLGYTFIYNRIVPYKYHYLDVLILSGLHLLYKVAAKGYLDFILETLKYDHNNDNLDIILTQAVTYNHRKILTYYIPQLTYAQIEQCLFMAIKKKSSKKTLNLLLSHLKLSIKLIKKISQYVATYNSTNIIGILNMKRKKKIYLDIILTKFVKYAIFNKYVVRCMDTFSINPERIIKMAARINKMLLVKKISQHAWKNHAARLKHLKHAVYTMKHKDGKNRLMNLIYDHYYYHMQGEEIFSLARFYAIHHAPKLFDVFYDCCLLDTIRFKSLLLDCSHIIGKNAHDATNITIVNKYIGNLFAMGVLSKKEILQDYPSIYSKHYMP

SEQ ID NO: 155 - protein, 531 aa, African swine fever virus
MFSLQNLCRKTLPDCKLPEFFDEYILQLLGLYWENHGTIQRAGNNCVLIQQHNLIPVNEALRIAASEENYEIVSLLLAWEGNLYYAIIGALEGNRHDLIRKYDDQIKDHHEILPFIDDPVIFHKCHIMRQCFFDCILYQAVKYSKFRVLLSFKHRLRDDLPFTHLLIEKACKDHNYEVIKWIYENLHIYNMIDTFECAIAHKDLRLYCLGYRFIYNRIVPDKYHHLDIRMLSSLQLLHKVAAKGYLDFILETLKYDHNKDNINIILTQAATYNHRKILIYFIPQSTHAQIEQCLLVAIKTKSSKKTLNLLLSHLNLSINLIKKISHYVATYNSTNIIGILSMRRKKKIYLDIILTKFVKKAIFNKFVVRCMDTFSINPERILKIAARINRTMLVKKISEHVWKNHAVRLKYLKHAVHTMKHKDGKNRLMNFIYDRCYYHMQGEEIFSLARFYAIHHAPKLFDVFYDCCILDTIRFKSLLLDCSHIIGKNAHDATNINIVNKYIGNLFVMGVLSKKEILQDYPSIYSKHYMP

SEQ ID NO: 156 - protein, 531 aa, African swine fever virus
MFSLQNLCRKTLPDCKLPEFFDEYILQLLGLYWENHGTIQRAGNNCVLIQQRTLIPVNEALRIAASEENYEIVGLLLAWEGNLYYAIIGALEGNRHDLIRKYDDQIKDHHEILPFIDDPVIFHKCHIIRRCFFNCILYQAVKYSKFRVLLYFKHRLEDDLPLTHLLIEKACEDHNYEVIKWIYENLHTYDIMDTFECAIAHKDLRLYCLGYTFIYNRIVPYEYHHLDILILSSLQLLHKVAAKGYLDFILETLKYDHNNDNLDIILTQAATYNHRKILTYFIPQLTYAQIEQCLFMAIKRKSSKKTLNLLLSHLTLSIELIKKISQYVVTYNSTNIIGILSMKRKKKIYLDIMLTKYVKYAIFNKYVVRCMDRFSINPERIIKMAARINRMMLVKKISEHVWKNHAARLKHLKHAVHTMKHKDGKNRLMNFIYDRCYYHMQGEEIFSLARFYAIHHAPKLFDVFYDCCILDTIQFKSLLLDCSHIIGKNAHDATNINIVNKYIGDLFAMGVLSKKEILQDYPSIYSKHYML

SEQ ID NO: 157 - protein, 531 aa, African swine fever virus
MFSLQNLCRKTLPDCKLPEFFDEYILQLLGLYWENHGTIQRAGNNCVLIQQHTLIPVNEALRIAASEENYEIVSLLLAWEGNLYYAIIGALEGNRHDLIRKYDDQIKDHHEILPFIDDPVIFHKCHIMRRCFFDCILYQAVKYSKFRVLLYFKYRLENDLPLAHLLIKKACEDHNYEVIKWIYENLHIYNIMDTFGCAIAHKDLRLYRLGYTFIYNRIVPYKYHYLDVLILSGLHLLYKVAAKGYLDFILETLKYDHNNDNLDIILTQAATYNHRKILTYYIPQLTYAQIEQCLFMAIKKKSSKKTLNLLLSHLKLSIKLIKKISQYVATYNSTNIIGILNMRRKKKIYLDIILTKFVKKAIFNKFVVRCMDTFSINPERIIKMAARINKMLLVKKISEHAWKNHAARLKHLKHAVYTMKHKDGKNRLMNLIYDHYYYHMQGEEIFSLARFYAIHHAPKLFDVFYDCCLLDTIRFKNLLLDCSHIIGKNAHDATNITIVNKYIGNLFAMGVLSKKEILQDYPSIYSKHYMP

SEQ ID NO: 158 - DNA, 438 bp, African swine fever virus
atggatcattatcttaaaaaattacaagatatttatacgaagctcgagggccatccctttctttttagcccgtcgaaaaccaatgaaaaagagtttattactctgctaaaccaggccttggcctcaacgcagctttaccgcagcatacaacagctgtttttaacgatgtataagctagatcccattgggtttattaactatattaaaacgagtaaacaagagtatttatgcctgttgattaatcctaaactcgttactaagtttttaaaaataacgagctttaaaatttacattaatttcagactgaaaactttttatataagtcctaataagtataataatttttacactgctccctctgaagaaaagactaaccatctcctaaaagaagaaaaaacttgggcaaagattgttgaagaaggaggagaagaatcctaa

SEQ ID NO: 159 - DNA, 438 bp, African swine fever virus
atggatcattatcttaaaaaattacaagatatttatacgaagctcgagggtcatccctttctttttagcccgtcgaaaaccaatgaaaaagagtttattactctgctaaaccaggccttggcctcaacgcagctttaccgcagcatacaacagctgtttttaacgatgtataagctagatcccattgggtttattaactatattaaaacgagtaaacaagagtatttatgcctgttaattaatcctaaactcgttactaagtttttaaaaataacgagctttaaaatttacattaatttcaggctgaaaactttttatataagtcctaataagtataataatttttacaccgctccctctgaagaaaagactaaccatcttctaaaagaagaaaaaacttgggcaaagattgttgaagaaggaggagaagaatcctaa

SEQ ID NO: 160 - DNA, 438 bp, African swine fever virus; identical to SEQ ID NO: 159

SEQ ID NO: 161 - DNA, 438 bp, African swine fever virus
atggatcattatcttaaaaaattacaggatatttataagaagcttgagggtcacccctttctttttagtccgtcgaaaaccaatgaaaaagagtttattactctgctaaaccaagccttggcctcgacgcagctttaccgcagcatacaacagctgtttttaacgatgtataagctagatcccattgggtttgttaactatattaaagcgagtaaacaagagtatttatgtctgttgattaatcctaaactagtcactaagtttttaaaaataacgagctttaaaatttacattaattttaggctgaaaactttttatataagtcctaataagtataataatttttacatcgctccctctgaagaaaaggccaatcatctcctaaaagaagaaaaaacctgggcaaagattgttgaagaaggaggagaagaatcctaa

SEQ ID NO: 162 - DNA, 438 bp, African swine fever virus
atggatcattatcttaaaaaattacaggatatttataagaagcttgagggtcacccctttctttttagtccgtcgaaaaccaatgaaaaagagtttattactctgctaaaccaagccttggcctcgacgcagctttaccgcagcatacaacagctgtttttaacgatgtataagctagatcccattgggtttgttaactatattaaagcgagtaaacaagagtatttatgtctgttgattaatcctaaactagtcactaagtttttaaaaataacgagctttaaaatttacattaatttcaggctaaaaactttctatataagtcctaataagtataataatttttacatcgctccctctgaagaaaaggccaatcatctcctaaaagaagaaaaaacctgggcaaagattgttgaagaaggaggagaagaatcctaa

SEQ ID NO: 163 - DNA, 438 bp, African swine fever virus; identical to SEQ ID NO: 161

SEQ ID NO: 164 - DNA, 438 bp, African swine fever virus; identical to SEQ ID NO: 158

SEQ ID NO: 165 - DNA, 438 bp, African swine fever virus
atggatcattatcttaaaaaattagaggatatttataaaaagcttgagggtcatccctttctttttagtccgtcgaaaaccaatgaaaaagagtttattactctgttaaaccaagccttggcctcaacgcagctttatcgcagcatacaacagctgtttttaacgatgtataagctagatcccattgggtttattaactatattaaaacgagtaaacaagagtatttatgcctgttgatcaatcctaaactagttacgaagtttttaaaaataacgagttttaaaatttacattaatttcaggctgaaaactttttatataagtcctaataaatataataatttttacaccgctccctctgaagaaaaggccaatcatctcctaaaagaagaaaaaacctgggcaaagattgttgaagaaggaggagaagaatcctaa

SEQ ID NO: 166 - DNA, 441 bp, African swine fever virus
atggatcattatcttaaaaaattacaagatatttatacgaagctcgagggccatccctttctttttagcccgtcgaaaaccaatgaaaaagagtttattactctgctaaaccaggccttggcctcaacgcagctttaccgcagcatacaacagctgtttttaacgatgtataagctagatcctatagggtttattaactatattaaaacgagtaaacaagagtatttatgcctgttgattaatcctaaactcgttactaagtttttaaaaataacgagctttaaaatttacattaatttcagactgaaaactttttatataagtcctaataagtataataatttttacaccgctccctctgaagaaaagaccaaccatctcctaaaagaagaaaaaacttgggcaaagattgttgaagaaggaggaggagaagaatcctaa

SEQ ID NO: 167 - DNA, 438 bp, African swine fever virus
atggatcattatcttaaaaaattacaagaaatttatacgaagctcgagggccatccctttctttttagcccgtcgaaaaccaatgaaaaagagtttattactctgctaaaccaggccttggcctcaacgcagctttatcgcagcatacaacagctgtttttaacgatgtataagctagatcccattgggtttattaactatattaaaacgagtaaacaagagtatttatgcctgttgattaatcctaaactcgtcactaagtttttaaaaataacgagctttaaaatttacattaatttcagactgaaaactttttatataagtcctaataagtataataatttttacaccgctccctctgaagaaaagaccaaccatctcctaaaagaagaaaaaacttgggcaaagattgttgaagaaggaggagaagaatcctaa

SEQ ID NO: 168 - DNA, 438 bp, African swine fever virus
atggatcattatcttaaaaaattacaagatatttatacgaagctcgagggtcatccctttctttttagcccgtcgaaaaccaatgaaaaagagtttattactctgctaaaccaggccttggcctcaacgcagctttaccgcagcatacaacagctgtttttaacgatgtataagctagatcccattgggtttattaactatattaaaacgagtaaacaagagtatttatgcctgttgattaatcctaaactcgttactaagtttttaaaaataacgagctttaaaatttacattaatttcagactgaaaactttttatataagtcctaataagtataataatttttacaccgctccctctgaagaaaagactaaccatctcctaaaagaagaaaaaacttgggcaaagattgttgaagaaggaggagaagaatcctaa

SEQ ID NO: 169 - DNA, 441 bp, African swine fever virus; identical to SEQ ID NO: 166

SEQ ID NO: 170 - DNA, 438 bp, African swine fever virus
atggatcattatcttaaaaaattacaagatatttatacgaagctcgagggccatccctttctttttagcccgtcgaaaaccaatgaaaaagagtttattactctgctaaaccaggccttggcctcaacgcagctttaccgcagcatacaacagctgtttttaacgatgtataagctagatcccattgggtttattaactatattaaaacgagtaaacaagagtatttatgcctgttgattaatcctaaactcgtcactaagtttttaaaaataacgagctttaaaatttacattaatttcagactgaaaactttttatataagtcctaataagtataataatttttacaccgctccctctgaagaaaagaccaaccatctcctaaaagaagaaaaaacttgggcaaagattgttgaagaaggaggagaagaatcctaa

SEQ ID NO: 171 - protein, 145 aa, African swine fever virus; identical to SEQ ID NO: 9

SEQ ID NO: 172 - protein, 145 aa, African swine fever virus; identical to SEQ ID NO: 9

SEQ ID NO: 173 - protein, 145 aa, African swine fever virus
MDHYLKKLQDIYKKLEGHPFLFSPSKTNEKEFITLLNQALASTQLYRSIQQLFLTMYKLDPIGFVNYIKASKQEYLCLLINPKLVTKFLKITSFKIYINFRLKTFYISPNKYNNFYIAPSEEKANHLLKEEKTWAKIVEEGGEES

SEQ ID NO: 174 - protein, 145 aa, African swine fever virus; identical to SEQ ID NO: 173

SEQ ID NO: 175 - protein, 145 aa, African swine fever virus; identical to SEQ ID NO: 173

SEQ ID NO: 176 - protein, 145 aa, African swine fever virus; identical to SEQ ID NO: 9

SEQ ID NO: 177 - protein, 145 aa, African swine fever virus
MDHYLKKLEDIYKKLEGHPFLFSPSKTNEKEFITLLNQALASTQLYRSIQQLFLTMYKLDPIGFINYIKTSKQEYLCLLINPKLVTKFLKITSFKIYINFRLKTFYISPNKYNNFYTAPSEEKANHLLKEEKTWAKIVEEGGEES

SEQ ID NO: 178 - protein, 146 aa, African swine fever virus
MDHYLKKLQDIYTKLEGHPFLFSPSKTNEKEFITLLNQALASTQLYRSIQQLFLTMYKLDPIGFINYIKTSKQEYLCLLINPKLVTKFLKITSFKIYINFRLKTFYISPNKYNNFYTAPSEEKTNHLLKEEKTWAKIVEEGGGEES

SEQ ID NO: 179 - protein, 145 aa, African swine fever virus
MDHYLKKLQEIYTKLEGHPFLFSPSKTNEKEFITLLNQALASTQLYRSIQQLFLTMYKLDPIGFINYIKTSKQEYLCLLINPKLVTKFLKITSFKIYINFRLKTFYISPNKYNNFYTAPSEEKTNHLLKEEKTWAKIVEEGGEES

SEQ ID NO: 180 - protein, 145 aa, African swine fever virus; identical to SEQ ID NO: 9

SEQ ID NO: 181 - protein, 146 aa, African swine fever virus; identical to SEQ ID NO: 178

SEQ ID NO: 182 - protein, 145 aa, African swine fever virus; identical to SEQ ID NO: 9

SEQ ID NO: 183 - DNA, 378 bp, African swine fever virus
atggcggtttatgcgaaggatcttgataataacaaagagttaaaccaaaaattaattaacgatcagcttaaaattattgacacgctcttgctagcagaaaaaaaaaactttttggtgtatgaattgcctgccccttttgacttttcctccggcgaccctttggccagtcagcgcgacatatactatgccatcataaaaagcctcgaggagcgcgggtttactgtcaaaatatgtatgaaaggggatcgtgccctccttttcatcacttggaaaaaaatacaatccattgagataaacaaaaaagaagaatatctgcgcatgcacttcatacaagacgaagagaaagcattttattgtaaatttttagagtctagatga

SEQ ID NO: 184 - DNA, 378 bp, African swine fever virus
atggcggtttatgcgaaggatcttgataataacaaagagttaaaccaaaaattaattaacgatcagcttaaaattattgacacgctcttgctggcagaaaaaaaaaactttttggtgtatgaactacctgccccttttgacttttcctccggcgaccctttggccagtcagcgcgacatatactatgccatcataaaaagcctcgaggagcgcgggtttactgtcaaaatatgtatgaaaggggatcgtgccctccttttcatcacctggaaaaaaatacaatccattgagataaacaaaaaagaagaatatctgcgcatgcacttcatacaagacgaagagaaagcattttattgtaaatttttagagtctagatga

SEQ ID NO: 185 - DNA, 378 bp, African swine fever virus; identical to SEQ ID NO: 184

SEQ ID NO: 186 - DNA, 378 bp, African swine fever virus
atggcggtttatgcgaaggaccttgataataacaaagagttaaaccaaaaattgatcaacgatcagcttaaaatcattgacacgcttttgctagcagaaaaaaaaaactttttggtgtacgaactacctgcccattttgacttttcctccggcgaccctttgggcagtcagcgcgacatttactatgccatcataaaaagtctcgaggagcgcggatttactgtcaaaatatgtatgaagggagaccgcgcccttcttttcatcacctggaaaaaaatacaatccattgagatcaacaaaaaagaagaatatctgcgcatgcacttcatacaagacgaagagaaagcgttttattgtaaatttttagagtctagatga

SEQ ID NO: 187 - DNA, 378 bp, African swine fever virus
atggcggtttatgcgaaggaccttgataataacaaagagttaaaccaaaaattgatcaacgatcagcttaaaattattgacacgcttttgctggcagaaaaaaaaaactttttggtgtacgaactacctgcccattttgacttttcctccggcgaccctttgggcagtcagcgcgacatttactatgccatcataaaaagtctcgaggagcgcggatttactgtcaaaatatgtatgaagggagaccgcgcccttcttttcatcacctggaaaaaaatacaatccattgagatcaacaaaaaagaagaatatctgcgcatgcacttcatacaagacgaagagaaagcgttttattgtaaatttttagagtctagatga

SEQ ID NO: 188 - DNA, 378 bp, African swine fever virus; identical to SEQ ID NO: 186

SEQ ID NO: 189 - DNA, 378 bp, African swine fever virus; identical to SEQ ID NO: 183

SEQ ID NO: 190 - DNA, 378 bp, African swine fever virus
atggcagtttatgcgaaagatcttgataataacaaagagttaaaccaaaaattaattaacgatcaacttaaaattattgacacgctgttgctggcagaaaaaaaaaactttttggtgcatgaactacctgcccactatgacttttcctccggcgaccctttggccagtcagcgcgacatatactatgccatcataaaaagtctcgaggagcgcggatttactgtcaaaatttgtatgaaaggagatcgcgcccttcttttcatcacctggaaaaaaatacaatccattgagataaacaaaaaagaagaatatctgcgcatgcacttcatacaagatgaagagaaagcattttattgtaaatttttagagtctagatga

SEQ ID NO: 191 - DNA, 378 bp, African swine fever virus; identical to SEQ ID NO: 183

SEQ ID NO: 192 - DNA, 378 bp, African swine fever virus
atggcggtttatgcgaaggatcttgataataacaaagagttaaaccaaaaattaattaacgatcagcttaaaattattgacacgctcttgctggcagaaaaaaaaaactttttggtgtatgaactacctgccccttttgacttttcctccggcgaccctttggccagtcagcgcgacatatactatgccatcataaaaagtctcgaggagcgcgggtttactgtcaaaatatgtatgaaaggggatcgcgccctccttttcatcacctggaaaaaaatacaatccattgagataaacaaaaaagaagaatatctgcgcatgcacttcatacaagacgaagagaaagcattttattgtaaatttttagagtctagatga

SEQ ID NO: 193 - DNA, 378 bp, African swine fever virus
atggcggtgtatgcgaaggaccttgataataacaaagagttaaaccaaaaattaattaacgatcagcttaaaattattgacacgctcttgctggcagaaaaaaaaaactttttggtgtatgaactacctgccccttttgacttttcctccggcgaccctttggccagtcagcgtgacatatactatgccatcataaaaagtcttgaggagcgcgggtttactgtcaaaatatgtatgaaaggggatcgtgccctccttttcatcacctggaaaaaaatacaatccattgaaataaacaaaaaagaagaatatctgcgcatgcacttcatacaagacgaagagaaatcattttattgtaaatttttagagtctagatga

SEQ ID NO: 194 - DNA, 378 bp, African swine fever virus; identical to SEQ ID NO: 192

SEQ ID NO: 195 - DNA, 378 bp, African swine fever virus
atggcggtttatgcgaaggaccttgataataacaaagagttaaaccaaaaattaattaacgatcagcttaaaattattgacacgctcttgctggcagaaaaaaaaaactttttggtgtatgaactacctgccccttttgacttttcctccggcgaccctttggccagtcagcgcgacatatactatgccatcataaaaagtctcgaggagcgcgggtttactgtcaaaatatgtatgaaaggggatcgcgccctccttttcatcacctggaaaaaaatacaatccattgagataaacaaaaaagaagaatatctgcgcatgcacttcatacaagacgaagagaaagcattttattgtaaatttttagagtctagatga

SEQ ID NO: 196 - protein, 125 aa, African swine fever virus; identical to SEQ ID NO: 10

SEQ ID NO: 197 - protein, 125 aa, African swine fever virus; identical to SEQ ID NO: 10

SEQ ID NO: 198 - protein, 125 aa, African swine fever virus
MAVYAKDLDNNKELNQKLINDQLKIIDTLLLAEKKNFLVYELPAHFDFSSGDPLGSQRDIYYAIIKSLEERGFTVKICMKGDRALLFITWKKIQSIEINKKEEYLRMHFIQDEEKAFYCKFLESR

SEQ ID NO: 199 - protein, 125 aa, African swine fever virus; identical to SEQ ID NO: 198

SEQ ID NO: 200 - protein, 125 aa, African swine fever virus; identical to SEQ ID NO: 198

SEQ ID NO: 201 - protein, 125 aa, African swine fever virus; identical to SEQ ID NO: 10

SEQ ID NO: 202 - protein, 125 aa, African swine fever virus
MAVYAKDLDNNKELNQKLINDQLKIIDTLLLAEKKNFLVHELPAHYDFSSGDPLASQRDIYYAIIKSLEERGFTVKICMKGDRALLFITWKKIQSIEINKKEEYLRMHFIQDEEKAFYCKFLESR

SEQ ID NO: 203 - protein, 125 aa, African swine fever virus; identical to SEQ ID NO: 10

SEQ ID NO: 204 - protein, 125 aa, African swine fever virus; identical to SEQ ID NO: 10

SEQ ID NO: 205 - protein, 125 aa, African swine fever virus
MAVYAKDLDNNKELNQKLINDQLKIIDTLLLAEKKNFLVYELPAPFDFSSGDPLASQRDIYYAIIKSLEERGFTVKICMKGDRALLFITWKKIQSIEINKKEEYLRMHFIQDEEKSFYCKFLESR

SEQ ID NO: 206 - protein, 125 aa, African swine fever virus; identical to SEQ ID NO: 10

SEQ ID NO: 207 - protein, 125 aa, African swine fever virus; identical to SEQ ID NO: 10

(19) United States
(12) Patent Application Publication
(10) Pub. No.: US 2023/0124042 A1
(43) Pub. Date: Apr. 20, 2023

(54) VACCINE AGAINST AFRICAN SWINE FEVER VIRUS INFECTION

(71) Applicant: THE PIRBRIGHT INSTITUTE, Woking (GB)

(72) Inventors: Linda Dixon, Woking (GB); Ana Reis, Woking (GB); Samuel Connell, Woking (GB); Anusyah Rathakrishnan, Woking (GB)

(21) Appl. No.: 17/905,492

(22) PCT Filed: Mar. 5, 2021
(86) PCT No.: PCT/GB2021/050561

§ 371 Date: Sep. 1, 2022

(30) Foreign Application Priority Data
Mar. 6, 2020 (GB) 2003289.2
Mar. 6, 2020 (GB) 2003292.6
Apr. 22, 2020 (GB) 2005878.0
Apr. 22, 2020 (GB) 2005880.6
Aug. 28, 2020 (GB) 2013541.4

(51) Int. Cl. A61K 39/12; C12N 7/00; A61P 31/20

(52) U.S. Cl. CPC A61K 39/12; C12N 7/00; A61P 31/20; C12N 2710/12021; C12N 2710/12034; C12N 2710/12062; A61K 2039/5254

(57) ABSTRACT

The present invention relates to attenuated African Swine Fever Viruses. The attenuated viruses protect pigs against subsequent challenge with virulent virus. The present invention also relates to the use of such attenuated viruses to treat and/or prevent African Swine Fever.

### FIELD OF THE INVENTION

The present invention relates to attenuated African Swine Fever viruses. The attenuated viruses protect pigs against subsequent challenge with virulent virus. The present invention also relates to the use of such attenuated viruses to treat and/or prevent African Swine

Fever.

### BACKGROUND TO THE INVENTION

### African Swine Fever (ASF)

African swine fever is a devastating haemorrhagic disease of domestic pigs caused by a double-stranded DNA virus, African swine fever virus (ASFV). ASFV is the only member of the Asfarviridae family and replicates predominantly in the cytoplasm of cells. Virulent strains of ASFV can kill domestic pigs within about 5-14 days of infection with a mortality rate approaching 100%.

ASFV can infect and replicate in warthogs (Phacochoerus sp.), bushpigs (Potamocherus sp.) and soft ticks of the Ornithodoros species (which are thought to be a vector), but in these species few if any clinical signs are observed and long term persistent infections can be established. ASFV was first described after European settlers brought pigs into areas endemic with ASFV and, as such, is an example of an “emerging infection”. The disease is currently endemic in many sub-Saharan countries and in Europe in Sardinia. Following its introduction to Georgia in the Trans Caucasus region in 2007, ASFV has spread extensively through neighbouring countries including the Russian Federation. In 2012 the first outbreak was reported in Ukraine and in 2013 the first outbreaks in Belarus. In 2014 further outbreaks were reported in pigs in Ukraine and detection in wild boar in Lithuania and Poland.

There is currently no treatment for ASF. Prevention in countries outside Africa has been attempted on a national basis by restrictions on incoming pigs and pork products, compulsory boiling of waste animal products under license before feeding to pigs and the application of a slaughter policy when the disease is diagnosed. Prevention in Africa is based on measures to keep warthogs and materials contaminated by warthogs away from the herd.

There is thus a need for improved measures to control ASFV infection and prevent spread of the disease.

### African Swine Fever Virus (ASFV)

The complete genome sequences of ASFV isolate Benin 97/1 (a highly pathogenic virus from West Africa, Group1), isolate OURT88/3 (non-pathogenic, attenuated virus from Portugal, Group 1) and isolate BA71V (Vero cell tissue culture adapted non-pathogenic virus, Group 1) have been compared (Chapman et al. 2008 J. Gen Virol. 89: 397-408).

In the OURT88/3 genome, five MGF 360 genes (10L, 11L, 12L, 13L and 14L) and two MGF 505 genes (1R, 2R) are deleted, and the MGF 505R 3R gene is truncated. These genes are present in the genomes of all eight other pathogenic isolates of ASFV that have been sequenced. Chapman et al. (as above) does not determine any mutations which are causative of attenuation in OURT88/3. Further, although OURT88/3 has been shown to induce a protective immune response in certain animals, this effect does not appear to be universal. Immunisation with OURT88/3 appears to be ineffective in protecting some pigs from subsequent challenge. It is also associated with the induction of adverse immune responses, such as joint swelling, in some pigs.

In the attenuated tissue-culture adapted BA71V isolate, the genome contains the MGF 505 3R gene but lacks the other seven MGF genes and in addition the MGF 360 9L gene is truncated.

Previously six MGF 360 genes (9L, 10L, 11L, 12L, 13L and 14L) and two MGF 505 genes (1R and 2R) were deleted from the highly pathogenic South African isolate Pr4. This deletion markedly reduced viral growth in primary macrophage cell cultures (Zsak et al. 2001, as above) and led to attenuation of the virus (cited as unpublished results, Afonso et al. 2004 J. Virol 78:1858-1864). However no experiments were carried out to challenge the recovered pigs to determine if they were protected. In fact, at an African swine fever virus workshop at the Biosecurity Research Institute, Manhattan Kansas in May 2011 it was mentioned that the Pr4 deletion mutant was not protective and induced a chronic form of the disease.

It has also been shown that deletion of three MGF 360 genes (12L, 13L and 14L) and four MGF 505 genes (1R, 2R, 3R and 4R truncation) from the pathogenic virus MalawiΔDP71 reduces virus replication in pigs and in addition attenuated the virus (Neilan et al. (2002) J. Virol. 76:3095-3104). However again, no experiments were reported to determine if the recovered pigs were protected against challenge.

Deleting MGF505-1R, -2R and -3R, and MGF360-12L, -13L and -14L produced an attenuated virus that conferred protection against parental virus (O'Donnell et al. 2015 J. Virol. 89:6048-6056). However, deletion of MGF360-13L and -14L only did not result in viral attenuation (Borca et al. 2017 Sci Rep. 7: 46747).

### Differentiation of Infected and Vaccinated Animals (DIVA) Vaccines

A DIVA vaccine allows differentiation of animals that have been infected with a wild type pathogen from animals that have been immunised with the vaccine. DIVA vaccines lack at least one immunogenic antigen (a DIVA marker) which is present in the wild type pathogen. Animals infected with the wild type pathogen produce antibodies against the DIVA marker, whereas vaccinated animals do not. Antibodies to the DIVA marker may be detected using a serological assay. Infected animals (which have antibodies to the DIVA marker) may thus be differentiated from vaccinated animals (which do not have antibodies to the DIVA marker), despite both groups of animals having antibodies to other immunogens of the pathogen.

A DIVA marker should be immunogenic, but deletion of the gene should not affect the vaccine's protective capacity.

### SUMMARY OF THE INVENTION

In one aspect, the present invention relates to an attenuated African Swine Fever virus in which expression and/or activity of particular multigene-family (MGF) genes is disrupted, whilst expression and/or activity of other particular MGF genes is not disrupted.

The invention also relates to the finding that the K145R and B125R genes of African Swine Fever virus are useful as DIVA markers.

The invention provides an attenuated African Swine Fever (ASF) virus in which the expression and/or activity of the following genes is disrupted:
multigene-family (MGF) 360 12L, 13L and 14L, and
MGF 505 1R,

and which comprises a functional version of the following genes:
(i) MGF 360 10L and/or 11L, and
(ii) MGF 505 2R and/or 3R.

The invention also provides an attenuated ASF virus which lacks a functional version of the K145R gene and/or the B125R gene.

The invention also provides a vaccine comprising an attenuated ASF virus according to the invention.

The invention further provides a vaccine of the invention for use in treating and/or preventing African Swine Fever in a subject.

The invention further provides a method for treating and/or preventing African Swine Fever in a subject which comprises the step of administering to the subject an effective amount of a vaccine according to the invention.

The invention yet further provides a method of attenuating an ASF virus which comprises the step of disrupting the activity and/or expression of the following genes:
MGF 360 12L, 13L and 14L, and
MGF 505 1R,

whilst retaining the function of the following genes:
(i) MGF 360 10L and/or 11L, and
(ii) MGF 505 2R and/or 3R.

### DESCRIPTION OF THE FIGURES

FIG. 1 shows luciferase expression in HEK293T cells transfected with a luciferase reporter linked to an IRF3-activatable (A), NF-κB-activatable (B) or GAL4-activatable (C, D) promoter and expressing the indicated MGF gene. Expression from the promoters is activated by stimulation with Sendai virus (A), NF-κB p65 transfection (B) or transfection with a IRF3-GAL4DBD chimera (C) or a p65-GAL4DBD chimera (D). MGF genes inhibit luciferase expression (i.e. IRF3 and NF-κB activity) (A, B) and MGF360-12L in particular inhibits specifically the ability of IRF3 and NF-κB to activate transcription (C, D).

FIG. 2 is a schematic depiction of the MGFA and MGFB deletions. Below each gene name, information on the inhibition of IRF3- and NF-κB-dependent luciferase reporters is summarised and any identified host targets listed.

FIG. 3 depicts the experimental protocol used to immunise, boost and challenge pigs with GeorgiaΔK145RΔMGFA (Group C), GeorgiaΔK145RΔMGFB (Group D) and GeorgiaΔB125RΔMGFA (Group BA) viruses.

FIG. 4 shows survival of pigs in Groups D, C and BA after immunisation and challenge.

FIG. 5 shows temperatures of pigs in Group C (A), Group BA (B), Group D (C) and control Group F (D) following immunisation and challenge.

FIG. 6 shows clinical scores of pigs in Group C (A), Group BA (B), Group D (C) and control Group F (D) following immunisation and challenge.

FIG. 7 shows levels of virus genome (viremia) in whole blood were measured by quantitative PCR in pigs in Group C (A), Group BA (B), Group D (C) and control Group F (D) following immunisation and challenge.

FIG. 8 shows levels of infectious virus in whole blood after challenge.

FIG. 9 shows T cell responses in pigs from Group C (A, B) and Group BA (C, D) pre-immunisation, pre-boost and pre-challenge. Peripheral blood mononuclear cells (PBMCs) were collected pre-immunisation, pre-boost and pre-challenge and stimulated with live ASFV of either the homologous virulent Georgia 2007/1 strain (A, C) or heterologous genotype I virulent strain Benin 97/1 (B, D). Numbers of interferon gamma producing cells were measured by Elispot assays.

FIG. 10 shows antibody response to the major ASFV capsid protein p72/B646L measured post-immunisation using a commercial competitive ELISA (Ingenasa) in pigs from Group C (A) and Group BA (B).

FIG. 11 shows Vero cells transfected with a plasmid expressing the B125R gene fused to an HA-epitope tag. Cells were fixed and permeabilised then stained with an anti-HA antibody followed by appropriate secondary antibody. The B125R protein shown in green localises to the cell surface. Nuclei are shown in blue.

### DETAILED DESCRIPTION

### African Swine Fever Virus (ASFV)

African swine fever virus (ASFV) is the causative agent of African swine fever (ASF). The genome structure of ASFV is known in the art, as detailed in Chapman et al. 2008 J. Gen. Virol. 89:397-408. ASFV is a large, icosahedral, double-stranded DNA virus with a linear genome containing at least 150 genes. The number of genes differs slightly between different isolates of the virus. ASFV has similarities to the other large DNA viruses, e.g., poxvirus, iridovirus and mimivirus. In common with other viral haemorrhagic fevers, the main target cells for replication are those of monocyte, macrophage lineage.

Based on sequence variation in the C-terminal region of the B646L gene encoding the major capsid protein p72, 22 ASFV genotypes (I-XXII) have been identified. All ASFV p72 genotypes have been circulating in eastern and southern Africa. Genotype I has been circulating in Europe, South America, the Caribbean and western Africa. Genotype VIII is confined to four East African countries.

Examples of strains from some of the genotypes are given below:

Genotype I: OURT88/3; Brazil/79; Lisbon/60; BA715; Pret; Benin 97/1; IC/1/96; IC/576; CAM/82; Madrid/62; Malta/78; ZAR85; Katange63; Togo; Dakar59; Ourt88/1; BEN/1/97; Dom_Rep; VAL/76; IC/2/96; Awoshie/99; NIG/1/99; NIG/1/98; ANG/70; BEL/85; SPEC120; Lisbon/57; ASFV-Warm; GHA/1/00; GAM/1/00; Ghana; HOL/86; NAM/1/80; NUR/90/1; CAM/4/85; ASFV-Teng; Tegani; ASFV-E75.

Genotype II: Georgia 2007/1; POL/2015/Podlaskie (Polish strain); Belgium/Etalle/wb/2018; ASFV/Kyiv/2016/131; China/2018/AnhuiXCGQ

Genotype III: BOT 1/99

Genotype IV: ASFV-War; RSA/1/99/W

Genotype VI: MOZ 94/1

Genotype VII: VICT/90/1; ASFV-Mku; RSA/1/98

Genotype VIII: NDA/1/90; KAL88/1; ZAM/2/84; JON89/13; KAV89/1; DEZda; AFSV-Mal; Malawi LIL 20/1

Genotype IX: UGA/1/95

Genotype X: BUR/1/84; BUR/2/84; BUR/90/1; UGA/3/95; TAN/Kwh12; HindeII; ASFV-Ken; Virulent Uganda 65.

### ASFV Isolates

The attenuated ASF virus of the present invention may be derivable from a wild-type ASF virus isolate, but includes mutations in its genome such that the expression and/or activity of the following genes is disrupted: MGF 360 genes 12L, 13L and 14L and MGF 505 gene 1R.

The attenuated ASF virus of the present invention may be derivable from a wild-type ASF virus isolate, but lacks a functional version of the K145R gene or the B125R gene.

The term “wild-type” indicates that the virus existed (at some point) in the field, and was isolated from a natural host, such as a domestic pig, tick or warthog. ASFV isolates described to date are summarised in Table 1 below, together with their Genbank Accession numbers.

TABLE 1

| Isolate | Country | Host | Year | Virulence | GenBank accession no. |
|---|---|---|---|---|---|
| Georgia 2007/1 | Georgia | Pig | 2007 | High | FR682468 |
| BA71qqV | Spain | Pig | 1971 | Tissue culture adapted | U18466 |
| Benin 97/1 | Spain | Pig | 1997 | High | AM712239 |
| OURT88/3 | Portugal | Tick | 1988 | Low | AM712240 |
| Kenya | Kenya | Pig | 1950 | High | AY261360 |
| Malawi Lil20/1 | Malawi | Tick | 1983 | High | AY261361 |
| Mkuzi | Zululand | Tick | 1978 | Unknown | AY261362 |
| Pretorisuskop/96/4 | South Africa | Tick | 1996 | High | AY261363 |
| Tangani 62 | Malawi | Pig | 1962 | High | AY261364 |
| Warmbaths | South Africa | Tick | 1987 | Unknown | AY261365 |
| Warthog | Namibia | Warthog | 1980 | Unknown | AY261366 |
| Pol16_20186_07 | Poland | Pig | 2018 | High | MG939583 |
| Pig/HLJ/2018 | China | Pig | 2018 | Virulent | MK333180 |
| DB/LN/2018 | China | Dried blood pig feed | 2018 | Unknown | MK333181 |
| Belgium 2018/1 | Belgium | Wild boar | 2018 | Virulent | LR536725 |
| China/2018/AnhuiXCGQ | China | Pig | 2018 | Virulent | MK128995 |
| Ken05/TK1, Ken06 Bus | Kenya | | 2005/06 | Unknown | NC_044945 KM111295 |
| R35, R25, R7, R8, N10 | Uganda | | 2018 | Unknown | MH025920, MH025918, MH025917, |
| | | | | | MH025916, MH025919 |
| Pol_17_03029_C201 | Poland | Pig | 2017 | Unknown | MG939587 |
| 26544/OG10 | Sardinia/Italy | Pig | 2017 | Unknown | KM102979 |
| 47/Ss?2008 | Sardinia/Italy | Pig | 2008 | Unknown | KX354450 |
| Belgium/Etalle/wb/2018 | Belgium | Wild boar | 2018 | Virulent | MK543947 |
| ASFV/Kyiv/2016/131 | Ukraine | Pig | 2016 | Unknown | MN194591 |
| ASFV-SY18 | China | Pig | 2018 | Unknown | MH766894 |
| ASFV_HU_2018 | Hungary | Wild boar | 2019 | Unknown | MN715134 |
| ASFV-wbBS01 | China | Wild boar | 2019 | Unknown | MK645909 |
| ASFV Georgia 2007/1 (new version) | Georgia | Pig | 2007 | Virulent | LR743116 |
| ASFV/pig/China/CAS19-01/2019 | China | Pig | 2019 | Unknown | MN172368 |
| ASFV/LT14/1490 | Latvia | Wild boar | 2019 | Unknown | MK628478 |
| Odintsovo_02/14 | Russia | Pig | 2014 | | NC_044948 |
| ASFV CzechRepublic 2017/1 | Czech Republic | Wild boar | 2017 | Unknown | LR722600 |
| ASFV Moldova 2017/1 | Moldova | Pig | 2019 | Unknown | LR722599 |
| RSA_2_2008 | South Africa | Pig | 2008 | Unknown | MN336500 |
| LIV_5_40 | Zambia | Tick | | Unknown | MN318203 |
| ASFV/POL/2015/Podlaskie | Poland | Wild boar | 2015 | Virulent | MH681419 |

The genome of the attenuated ASFV of the invention may correspond to any ASFV genotype. The genome of the attenuated ASFV of the invention may essentially correspond to any ASFV genotype.

The term “corresponds to” means that the remainder of the genome of the attenuated ASFV of the invention is the same as the wild type strain. “The remainder of the genome” refers to all genes other than those to which the invention relates: MGF 360 10L, 11L, 12L, 13L and 14L, and MGF 505 1R, 2R and 3R, and B125R and K145R. Genes to which the invention relates may also correspond to the wild type strain. In an embodiment MGF 360 10L, 11L, 12L, 13L and 14L, and MGF 505 1R, 2R and 3R, and B125R and K145R correspond to the wild type strain. Where MGF 360 12L, 13L and 14L and MGF 505 1R correspond to the wild type strain, their expression and activity may be disrupted by a mutation in an intergenic region such as a promoter. In an embodiment MGF 360 12L, 13L and 14L, and MGF 505 2R and 3R, and B125R and K145R correspond to the wild type strain. In other words, the genes of the attenuated ASFV of the invention may be the same as the genes of the wild type strain. In an embodiment the genes of the attenuated ASFV of the invention are the same as the genes of the wild type strain, except for MGF 360 12L, 13L and 14L, and MGF 505 1R. In an embodiment the genes of the attenuated ASFV of the invention are the same as the genes of the wild type strain, except for MGF 360 12L, 13L and 14L, and MGF 505 1R, and B125R and/or K145R.

The term “essentially corresponds to” means the same as “corresponds to” with the additional exception that the remainder of the genome may comprise one or more mutations. The one or more mutations may be in other genes (i.e. not in genes to which the invention relates).

The genome of the attenuated ASFV may correspond or essentially correspond to genotype I. The genome of the attenuated ASFV may correspond or essentially correspond to genotype II. The genome of the attenuated ASFV may correspond or essentially correspond to genotype III. The genome of the attenuated ASFV may correspond or essentially correspond to genotype IV. The genome of the attenuated ASFV may correspond or essentially correspond to genotype V. The genome of the attenuated ASFV may correspond or essentially correspond to genotype VI. The genome of the attenuated ASFV may correspond or essentially correspond to genotype VII. The genome of the attenuated ASFV may correspond or essentially correspond to genotype VIII. The genome of the attenuated ASFV may correspond or essentially correspond to genotype IX. The genome of the attenuated ASFV may correspond or essentially correspond to genotype X.

The genome of the attenuated ASFV of the invention may correspond or essentially correspond to that of a virulent ASFV strain. Known virulent ASF virus strains include: Georgia 2007/1, Benin 97/1, Kenyan, Malawi Lil20/1, Pretorisuskop/96/4 and Tengani 62. The genome of the attenuated ASFV may correspond or essentially correspond to that of the Georgia 2007/1 strain. The genome of the attenuated ASFV may correspond or essentially correspond to that of the Benin 97/1 strain.

The genome of the attenuated ASFV of the invention may correspond or essentially correspond to that of an ASFV strain whose virulence is currently unknown, for example: Mkuzi, Warmbaths and Warthog.

In an embodiment the genome of the attenuated ASFV of the invention does not correspond to that of OURT88/3. In an embodiment the attenuated ASFV of the invention is not OURT88/3.

### Multigene Families (MGFs)

ASFV contains five multi-gene families which are present in the left and right variable regions of the genome. The MGFs are named after the average number of codons present in each gene: MGF100, 110, 300, 360 and 505/530. The N-terminal regions of members of MGFs 300, 360 and 505/530 share significant similarity with each other. It has been shown the MGF 360 and 505 families encode genes essential for host range function that involves promotion of infected-cell survival and suppression of type I interferon response.

The attenuated ASFV according to the present invention has disrupted expression and/or activity of the following genes:
multigene-family (MGF) 360 12L, 13L and 14L, and
MGF 505 1R,

and comprises a functional version of the following genes:
(i) MGF 360 10L and/or 11L, and
(ii) MGF 505 2R and/or 3R.

Alternatively expressed, the attenuated ASFV according to the present invention lacks a functional version of the following genes:
MGF 360 12L, 13L and 14L, and
MGF 505 1R,

and comprises a functional version of the following genes:
(i) MGF 360 10L and/or 11L, and
(ii) MGF 505 2R and/or 3R.

The location of these genes in the genomes of a variety of ASFV strains is provided below in Table 2. The sequence identity of each gene to the corresponding Benin 97/1 gene is also provided.

TABLE 2

| | | | |
|---|---|---|---|
| | Start | Stop | |
| | Nucleotide | Nucleotide | % Nucleotide |
| Strain | Number | number | Identity |

a - MGF 360-10L

| | | | |
|---|---|---|---|
| Benin 97/1 | 20677 | 19607 | 100 |
| Georgia 2007/1 | 26438 | 25368 | 93 |
| BA71V | Deleted | | |
| OURT88/3 | Deleted | | |
| Kenya 1950 | 30628 | 29563 | 82 |
| Malawi-Lil/20 | 24802 | 23763 | 91 |
| Mkuzi 1979 | 28139 | 27069 | 99 |
| Pretorisuskop/96/4 | 27186 | 26116 | 96 |
| Tengani 62 | 21600 | 20530 | 92 |
| Warmbaths | 26361 | 25322 | 94 |
| Warthog | 23130 | 22091 | 95 |
| E75 | 19981 | 18911 | 100 |

b - MGF 360-11L

| | | | |
|---|---|---|---|
| Benin 97/1 | 21764 | 20703 | 100 |
| Georgia 2007/1 | 27526 | 26465 | 97 |
| BA71V | Deleted | | |
| OURT88/3 | Deleted | | |
| Kenya 1950 | 31716 | 30655 | 84 |
| Malawi-Lil/20 | 25892 | 24831 | 86 |
| Mkuzi 1979 | 29225 | 28164 | 99 |
| Pretorisuskop/96/4 | 28275 | 27214 | 97 |
| Tengani 62 | 22688 | 21627 | 97 |
| Warmbaths | 27449 | 26388 | 97 |
| Warthog | 24218 | 23157 | 97 |
| E75 | 21068 | 20007 | 100 |

c - MGF 360-12L

| | | | |
|---|---|---|---|
| Benin 97/1 | 24668 | 23616 | 100 |
| Georgia 2007/1 | 30434 | 29382 | 96 |
| BA71V | Deleted | | |
| OURT88/3 | Deleted | | |
| Kenya 1950 | 34566 | 33549 | 90 |
| Malawi-Lil/20 | 28731 | 27682 | 92 |
| Mkuzi 1979 | 32125 | 31073 | 97 |
| Pretorisuskop/96/4 | 31150 | 30098 | 95 |
| Tengani 62 | 25592 | 24540 | 96 |
| Warmbaths | 30346 | 29294 | 96 |
| Warthog | 27088 | 26036 | 96 |
| E75 | 23971 | 22920 | 99 |

d - MGF 360-13L

| | | | |
|---|---|---|---|
| Benin 97/1 | 25901 | 24840 | 100 |
| Georgia 2007/1 | 31656 | 30595 | 97 |
| BA71V | Deleted | | |
| OURT88/3 | Deleted | | |
| Kenya 1950 | 35812 | 34757 | 90 |
| Malawi-Lil/20 | 29980 | 28925 | 91 |
| Mkuzi 1979 | 33347 | 32286 | 99 |
| Pretorisuskop/96/4 | 32368 | 31307 | 95 |
| Tengani 62 | 26814 | 25753 | 95 |
| Warmbaths | 31559 | 30498 | 95 |
| Warthog | 28338 | 27277 | 95 |
| E75 | 25204 | 24143 | 99 |

e - MGF 360-14L

| | | | |
|---|---|---|---|
| Benin 97/1 | 27146 | 26073 | 100 |
| Georgia 2007/1 | 32913 | 31840 | 99 |
| BA71V | Deleted | | |
| OURT88/3 | Deleted | | |
| Kenya 1950 | 37194 | 36121 | 92 |
| Malawi-Lil/20 | 31266 | 30193 | 92 |
| Mkuzi 1979 | 34620 | 33547 | 99 |
| Pretorisuskop/96/4 | 33598 | 32525 | 97 |
| Tengani 62 | 28056 | 26983 | 97 |
| Warmbaths | 32820 | 31747 | 96 |
| Warthog | 29568 | 28495 | 97 |
| E75 | 26449 | 25376 | 100 |

f - MGF 505-1R

| | | | |
|---|---|---|---|
| Benin 97/1 | 21971 | 23566 | 100 |
| Georgia 2007/1 | 27734 | 29329 | 95 |
| BA71V | Deleted | | |
| OURT88/3 | Deleted | | |
| Kenya 1950 | 31904 | 33496 | 91 |
| Malawi-Lil/20 | 26041 | 27627 | 93 |
| Mkuzi 1979 | 29425 | 31020 | 96 |
| Pretorisuskop/96/4 | 28449 | 30044 | 94 |
| Tengani 62 | 22891 | 24486 | 95 |
| Warmbaths | 27651 | 29246 | 95 |
| Warthog | 24387 | 25982 | 95 |
| E75 | 21275 | 22870 | 100 |

g - MGF 505-2R

| | | | |
|---|---|---|---|
| Benin 97/1 | 27352 | 28932 | 100 |
| Georgia 2007/1 | 33119 | 34699 | 99 |
| BA71V | 17725 | 19304 | 99 |
| OURT88/3 | 29532 | 29981 | 76 |
| Kenya 1950 | 37419 | 38985 | 90 |
| Malawi-Lil/20 | 31541 | 33121 | 93 |
| Mkuzi 1979 | 34826 | 36406 | 99 |
| Pretorisuskop/96/4 | 33795 | 35374 | 97 |
| Tengani 62 | 28261 | 29830 | 98 |
| Warmbaths | 33029 | 34597 | 96 |
| Warthog | 29773 | 31352 | 97 |
| E75 | 26655 | 28236 | 99 |

h - MGF 505-3R

| | | | |
|---|---|---|---|
| Benin 97/1 | 29019 | 29861 | 100 |
| Georgia 2007/1 | 34786 | 35625 | 96 |
| BA71V | 20398 | 21915 | 100 |
| OURT88/3 | 20850 | 22367 | 100 |
| Kenya 1950 | 40295 | 41815 | 89 |
| Malawi-Lil/20 | 34322 | 35842 | 89 |
| Mkuzi 1979 | 37500 | 39017 | 99 |
| Pretorisuskop/96/4 | 36480 | 37985 | 89 |
| Tengani 62 | 30926 | 32443 | 90 |
| Warmbaths | 35712 | 37232 | 93 |
| Warthog | 32449 | 33954 | 90 |
| E75 | 29330 | 30847 | 100 |

The gene (i.e. nucleotide) sequences of MGF genes from different strains are given below.

Benin 97/1 MGF 360 10L
(NC_044956.1:19607-20677)
SEQ ID No. 11
TTATAACGATGTATCATTGATGTCATAATTCAAATAGGCCAACAT
ACTTTTTGATTTATAGTTTTTTAATAGACGATATATTTTGTTAGG
ATCTGCTTCTTTTAACGTTAATAGCGAGGAGTCTGGACTATAAAT
GTCTAATGATAAACGATGAGATATCAAATAGTAATTCTGTTGCTC
TGCCAAGGCTTTTGCCTCTTCAAAGGCATCGGCCCCCAGATCTAT
ACAAAAGAACAGGTTATCCATATTATAGAATCGTATGGAGGCAAT
CATGGCCAAATTAATATTAGCTCCTAAGATAAAACAATAATATAT
AGTTAAAAAATTGTTATCTTTTGTGCAGGCTATCCGCATCATTTC
ATCCATGTCCATACGGATTTTTTCTTTTTCGTACAAATTATGTAG
GTCAAACAGCTTATTATAGCAAAGAGCACATGTTAACCACCACGT
ATTCAGATACTTAAAATGTTGGTAAACATAAGAAATGGCCTCCCT
AAGATTATCCTGCAATGCCACGATAAAACAGTATATAGTTAACAT
ATCACCATCCGAAATATTACTTAATACGTTGGTATCTTCTGCTAA
ATTTTTTAGCTTCCAATGTATACACGACTTTATTTCCCTTATAAT
GACATAGGCTGAAAAGGGATTGTCATTAAAAAATTTAAGACATAA
GATAATATTATTACTAGTAGTGTTAGGATGTATTAATTTAAAGAA
TATGTGCATAATCTTCTTTTTATCCACTTGGTACTTGGCTCCTAA
TTCCCAGCAAAATTCTCGAATAGGTGGCGTATTCGCGCAAATTAA
CCCATAGTTGATGTCTGCGCCCCAATCCGTAAACATTTTTATTAA
TTGATAGTTGTTTTCCTTTGTAGCCAACATTAGTGCCGTATTAAG
GTCCAAGCCATCTGCAAAGTTTGGCAGCTTTATCAGCATATGTTT
GCAATCAAGTGAAATTGGAGCCTTATACCACCATAGCCCGCAGCG
TTCTAAGATAACATGGTAATCAATAGATACATGCTGACTGGCTAA
TACCTTTTTGGCGAAGGATTGCAAGGAAGGAACCAT
China/2018/AnhuiXCGQ MGF 360 10L
(MK128995.1:25402-26439)
SEQ ID No. 12
CTAGGCCAACATACTTTTTAATTTATAGTTTTTTAATAGATGATA
TATTTTGCTAGGATCTGCTTCTTTTAACGTTAATAGCGAGGAGTC
TGCACTATAAATGTCTAATGATAAATGATGAGATATCAAATAGTA
ATTCCGTTGCTCTGCTAGGGCCTTTGCCTCTTCAAAGGCGTCGGC
TCCCAGATCTATACAAAAGAACAAGTTATCCATATTATAAAATCG
TACGCAGGCAAGCATAGCTGAATTAATATTAGCTCCTAAGAGAAA
ACAATAATATATGGTTAAAAAATTGTTATCTTTTGTGCAGGCCAT
CCGCATCATTTCATCCACGTCCATGCGGATCTTTTCCTTTTCATA
CAAATTATGTAGGTCAAACAGCTTATTAAAACAAAGAGCACAGAT
TAACCACCACGTATTTAGATACTTAAAATGTTGGTAAACATAAGA
AATGGCCTCCCTAAGATTATCCTGCAATGCCACTATAAAACAGTA
TATCGTTAACATATCACCATCCGACATATTACTTAATATGTCGGT
GTCTTCTACTAACCTTTTCAACTTCCAATATATGGATGACCTTAT
TTCCCTTATAATGACATAGGCTGGAAAGGGATTATCATTAAAAAG
TTTAAGACATAAGATAATATTACTGCTAGTAGTGCCAGGGTGTAT
TAATTTAAAGAACATGTGCATAATCTTCTTTTTATCCACGCGGTA
CTTGGCTCCTAATTCCCAGCAAAATTCTCGAACAGGCGGCGTATT
GGCGCAAATTAACCCATAGTTGATGTCTGCGCCCCATTCTGTAAA
CAGTTTTATTAACTGATAGTTGTTTTCCTTTGTAGCCAACATTAG
TGCCGTATTAAGGTCCAAGCCGTCTGCAAAGCTTGGCAGCTTTAT
CAGCATATGTTTGCAATCAAGGGAAATTGGGGCCTTATACCACCA
TAGTCCGCAGCGTTCTAAGATAACATGGTACTCAATAGATACTTG
CTGTCTGGCTAGTACCTTTTTGGCGAAGGATTGTAAGGAAGGAAA
CAT
Georgia 2007/1 MGF 360 10L
(LR743116.1:26373-27410)
SEQ ID No. 13
CTAGGCCAACATACTTTTTAATTTATAGTTTTTTAATAGATGATA
TATTTTGTTAGGATCTGCTTCTTTTAACGTTAATAGCGAGGAGTC
TGCACTATAAATGTCTAATGATAAATGATGAGATATCAAATAGTA
ATTCCGTTGCTCTGCTAGGGCCTTTGCCTCTTCAAAGGCGTCGGC
TCCCAGATCTATACAAAAGAACAAGTTATCCATATTATAAAATCG
TACGCAGGCAAGCATAGCTGAATTAATATTAGCTCCTAAGAGAAA
ACAATAATATATGGTTAAAAAATTGTTATCTTTTGTGCAGGCCAT
CCGCATCATTTCATCCACGTCCATGCGGATCTTTTCCTTTTCATA
CAAATTATGTAGGTCAAACAGCTTATTAAAACAAAGAGCACAGAT
TAACCACCACGTATTTAGATACTTAAAATGTTGGTAAACATAAGA
AATGGCCTCCCTAAGATTATCCTGCAATGCCACTATAAAACAGTA
TATCGTTAACATATCACCATCCGACATATTACTTAATATGTCGGT
GTCTTCTACTAACCTTTTCAACTTCCAATATATGGATGACCTTAT
TTCCCTTATAATGACATAGGCTGGAAAGGGATTATCATTAAAAAG
TTTAAGACATAAGATAATATTACTGCTAGTAGTGCCAGGGTGTAT
TAATTTAAAGAACATGTGCATAATCTTCTTTTTATCCACGCGGTA
CTTGGCTCCTAATTCCCAGCAAAATTCTCGAACAGGCGGCGTATT
GGCGCAAATTAACCCATAGTTGATGTCTGCGCCCCATTCTGTAAA
CAGTTTTATTAACTGATAGTTGTTTTCCTTTGTAGCCAACATTAG
TGCCGTATTAAGGTCCAAGCCGTCTGCAAAGCTTGGCAGCTTTAT
CAGCATATGTTTGCAATCAAGGGAAATTGGGGCCTTATACCACCA
TAGTCCGCAGCGTTCTAAGATAACATGGTACTCAATAGATACTTG
CTGTCTGGCTAGTACCTTTTTGGCGAAGGATTGTAAGGAAGGAAA
CAT
Ken05/Tk1 MGF 360 10L (NC_044945.1:27310-28380)
SEQ ID No. 14
TTACGACGTTGTATCATTATTGTGATAATCCAAATAGGCCGACAT
ACTTTTTGATTTATAATTGTTTAATAGATGGTATATTTTTTTAGG
GTTCGCTTCTTTTAGCGTGAATAGAGAGGGATCTGGATGATAGAT
ATCTAATGACAAACGATGTGATATTAAATAATAGTCCCGCTGCTC
TGCTAGGGCTTTTGCCTCTTCGAAGGCATTGGCCCCCAAGTCTAT
ACAAAAGAACAGATTATCTATATTAAAAAACTGTATGGCAGCAAG
CATCGCCATATTGATATCGGCCCCTAAAATAAAACAATAATACAT
CGTTAAAAAGTTGTTATCTTTCGTGCAAGCTATGCGCATCATTTC
ATTAATGTCTATGCGAATTTTTTCCTGCTCATAGAGATAATGGAG
GTCAAACAGTTTATTAAAACACAGAGCACAGATTAACCACCACGT
ATTCAGGTGCTTAAAATGTTGGTAAAAATACGAGACGGCCTCCCT
AAGACCATTCTGCAAGGCCACCATAAAACAATATAAAGTTAGCAT
ATCGCCGTCCGAAACATCGCGTAGTAGGTCTTCGTCTTCTACTAA
CCTCCTCAGCCTCCAATAAAGAGACGACTTTATTTCCCTTATAAT
CACATAGGTAGGAAAAGGATTCTCATTAAAAAGCTTATGACATAA
GATAATATATTGACTCGTTTTGTTCTCTATAAATTTAAAAAACAT
ATGCATCACCTTTTTTTTATCTAAGCGATAGTTCGCTCCTAATTC
CCAACAAAACTCTCGAGTAGGCTGTGTATTGGCACAAATGTATCC
ATAGTTGATGTTTGCACCCCACTCCGTGAACAGTTTTATTAATTG
ATAGTTGTTTTCCTTTGCAGCTATCATCAATGCCGTATTGAGGTC
CAGGCCATCCGCAAAGTATGGCAACCTTATTAACATGTGTTTACA
ATCAAGTGAAATTGGAGCCTTATACCACCATAACCCGCAGCGCTT
CAAAATATGATAGTAGTCTTCAGGCAGCACATGCTGGCTGGCTAA
TATCTTTTTGGCGAAAGATTGCAGGGAAGGAAACAT
Ken06.Bus MGF 360 10L
(NC_044946.1:23437-24507)
SEQ ID No. 15
TTAGGAGGTTGTATCATTGATGTGATAATCCAAATAGGCCAACAT
ACTTTTTGATTTATAGTTGTTTAATAGATGGTATATTTTTTTAGG
GTTCGCTTCTTTTAGCGTGAATAGAGAGGGATCTGGATGATAGAT
ATCTAATGACAAACGTTGTGATATTAAATAATAGTCCCGCTGCTC
TGCTAGGGCTTTTGCCTCTTCGAAGGCATTGGCTCCCAAGTCTAT
ACAAAAGAACAGATTATCTATATTAAAGAACTGTATGGCAGCAAG
CATGGCCATATTGATATCGGCCCCTAAAATAAAACAATAATACAT
CGTTAAAAAGTTATTATCTTTCGTGCAAGCTATGCGCATCATTTC
ATTAATGTCTATGCGAATCTTTTCCTGCTCATAGAGATAATGGAG
GTCAGACAGTTTATTAAAATACAGAGCACAGATTAACCACCACGT
ATTCATGTGCTTAAAATGTTCGTAAAAATACGAGACGGCCTCCCT
AAGATCATTCTGCAAGGCCACCATAAAACAATATATAGTTAGCAT
ATCGCCGTCCGAAACATCACGTAGTAGGTCTTCATCTTCTACTAA
CCTCCTCAGCCTCCAATAAAGAGACGACTTTATTTCCCTTATAAT
CACATAGGTAGGAAAAGGATTCTCATTAAAAAGCTTATGACATAA
GATAATATATTGACTTGTTTTGTTCTCTATAAATTTAAAGAACAT
GTGTATCACCTTTTTTTTATCCATGCGATAGTTCGCTCCTAATTC
CCAACAAAACTCTCGGGTAGGCTGTGTATTGGCACAAATGTATCC
ATAGTTGATGTTTGCACCCCACTCCGTGAATAGTTTTATTAATTG
ATAGTTGTTTTCCTTTGCAGCTATCATCAATGCCGTATTGAGATC
CAGGCCATCCGCAAAGTATGGCAACCTTATTAACATGTGTTTGCA
ATCAAGTGAAATTGGAGCCGTATACCACCATAACCCGCAGTATTT
CAAAATATGATAGTAGTCTTCAGGCAGCACATGCTGACTGGCTAA
TATCTTTTTGGCGAAAGACTGTAAAGAAGGAAACAT
Kenya 1950 MGF 360 10L
(AY261360.1:29558-30628)
SEQ ID No. 16
TTACGACGTTGTATCATTAATGTGATAATCCAAATAGGCCGACAT
ACTTTTTGATTTATAGTTGTTTAATAGATGGTATATTTTTTTAGG
GTTCGCTTCTTTTAGCGTGAATAGAGAGGGATCTGGATGATAGAT
ATCTAATGACAAACGATGTGATATTAAATAATAGTCCCGCTGCTC
TGCTAGGGCTTTTGCCTCTTCGAAGGCATTGGCCCCCAAGTCTAT
ACAAAAGAACAGATTATCCATATTAAAGAACTGTATGGCAGCAAG
CATGGCCATATTGATATCGGCCCCTAAAATAAAACAATAATACAT
CGTTAAAAAGTTGTTATCTTTCGTGCAAGCTATGCGCATCATTTC
ATTAATGTCTATGCGAATTTTTTCCTGCTCATAGAGATAATGGAG
GTCAAACAGTTTATTAAAACACAGAGCACAGATTAACCACCACGT
ATTCAGGTGCTTAAAATGTTGGTAAAAATACGAGACGGCCTCCCT
AAGATCATTCTGCAAGGCCACCATAAAACAATATAAAGTTAGCAT
ATCGTCGTCCGAAACATCGCGTAGTAGGTCTTCGTCTTCTACTAA
TCTCCTCAGCCTCCAATAAAGAGACGACTTTATTTCCCTTATAAT
CACATAGGTAGGAAAAGGATTCTCATTAAAAAGCTTATGACATAA
GATAATATACTGACTCGTTTTGTTCTCTATAAATTTAAAAAACAT
ATGCATCACCTTTTTTTTATCTAAGCGATAGTTTGCTCCTAATTC
CCAACAAAACTCTCGAGTAGGCTGTGTATTGGCACAAATGTATCC
ATAGTTGATGTTTGCACCCCACTCCGTGAACAGTTTTATTAATTG
ATAGTTGTTTTCCTTTGCAGCTATCATCAATGCCGTATTGAGGTC
CAGGCCATCCGCAAAGTATGGCAACCTTATTAACATGTGTTTACA
ATCAAGTGAAATTGGAGCCTTATACCACCATAACCCGCAGCGCTT
CAAAATATGATAGTAGTCTTCAGGCAGCACATGCTGGCTGGCTAA
TATCTTTTTGGCGAAAGATTGCAGGGAAGGAAACAT
L60 MGF 360 10L (NC_044941.1:19956-21026)
SEQ ID No. 17
TTATAACGATGTATCATTGATGTCATAATTCAAATAGGCCAACAT
ACTTTTTGATTTATAGTTTTTTAATAGACGATATATTTTGTTAGG
ATCTGCTTCTTTTAACGTTAATAGCGAGGAGTCTGGACTATAAAT
GTCTAATGATAAACGATGAGATATCAAATAGTAATTCTGTTGCTC
TGCCAAGGCTTTTGCCTCTTCAAAGGCATCGGCCCCCAGATCTAT
ACAAAAGAACAGGTTATCCATATTATAGAATCGTATGGAGGCAAT
CATGGCCAAATTAATATTAGCTCCTAAGATAAAACAATAATATAT
AGTTAAAAAATTGTTATCTTTTGTGCAGGCTATCCGCATCATTTC
ATCCATGTCCATACGGATTTTTTCTTTTTCGTACAAATTATGTAG
GTCAAACAGCTTATTATAGCAAAGAGCACATGTTAACCACCACGT
ATTCAGATACTTAAAATGTTGGTAAACATAAGAAATGGCCTCCCT
AAGATTATCCTGCAATGCCACGATAAAACAGTATATAGTTAACAT
ATCACCATCCGAAATATTACTTAATACGTTGGTATCTTCTGCTAA
ATTTTTTAGCTTCCAATGTATACACGACTTTATTTCCCTTATAAT
GACATAGGCTGAAAAGGGATTGTCATTAAAAAATTTAAGACATAA
GATAATATTATTACTAGTAGTGTTAGGATGTATTAATTTAAAGAA
TATGTGCATAATCTTCTTTTTATCCACTTGGTACTTGGCTCCTAA
TTCCCAGCAAAATTCTCGAATAGGTGGCGTATTCGCGCAAATTAA
CCCATAGTTGATGTCTGCGCCCCAATCCGTAAACATTTTTATTAA
TTGATAGTTGTTTTCCTTTGTAGCCAACATTAGTGCCGTATTAAG
GTCCAAGCCATCTGCAAAGTTTGGCAGCTTTATCAGCATATGTTT
GCAATCAAGTGAAATTGGAGCCTTATACCACCATAGCCCGCAGCG
TTCTAAGATAACATGGTAATCAATAGATACATGCTGACTGGCTAA
TACCTTTTTGGCGAAGGATTGCAAGGAAGGAACCAT
Malawi Lil-20/1 (1983) MGF 360 10L
(AY261361.1:23735-24802)
SEQ ID No. 18
TTACACTGTGTCATGCCCATCATAGTCAAAATATACCATCATATT
TTTTGATTTATAGTTTTTTAATAGATGATATATTTTTTTAGGATC
TGCTTCTTTTAACGTTAATAGCGAGGAGTCTGGACTATAAATATC
TATTGATAAACGATGATACATCAAAAAGTAATTCCGCTGGTCTGC
CAGGGCTTTTGCCTCTTCAAAGGCATCGGCTCCCAGGTCTATACA
AAAGAACAAGTTATCCATATTATAGAATTGTACGCAGGCAAGCAT
AGCCTGATTAATATTAGCTCCTAAGAGAAAACAGTAATATATGGT
TAAAAAGTTGTTATCTTTAGTGCAGGCTATGCACATCATTTCATC
CATGTCCATGCGGATCTTTTCCTTTTCATACAAATCATGTAGGTC
AAACAGCTTATTAAAACAAAGAGCACAGATTAACCACCACGTATT
CAGATGCTTAAAATGTTGGTAAAAATAAGAAATGGCCTCCCTAAG
ATTATCCTGCAATGCCAGGATAAAACAGTATATAGTCAACATATC
ATCATCCGACATATTACTTAATATGTCAGTGTCTTCTACTAACCT
TCTCAGCTTCCAATATATAGACGACTTTATTTCCCTTATAATGAC
ATAGGTTGGAAAAGGATTATTATTAAAAAGTTTAAGACATAAGAT
AATATTACTACTAGTAGTGCCATGATGTATTAATTTAAAAAACAT
ATGCATAACCTTATTTTTATCCACTTGGTACTTGGCTCCTAATTC
CCAGCAAAATTCTCGAACAGGCGGCGTATTAGCGCAAATTAATCC
ATAGTTGATGTCCGCGCCCCATTCCGTAAACAGTTTTATTAACTG
ATAGTTGTTTTCCTTTGTAGCCAACATTAGTGCCGTATTAAGGTC
CAAGCCATCTGCAAAGCTTGGCAGCTTTATTAACATATGTTTGCA
ATCAAGTGAAATTGGAGCCTTATACCACCATAGTCCGCAATGTTT
TAAGATATAATGATAATCAATGGATACATGATGTCTAGCTAATAC
CTTTTTGGCGAAGGATTGTAAAGAAGGAAACAT
Mkuzi 1979 MGF 360 10L
(AY261362.1:27069-28139)
SEQ ID No. 19
TTATAACGATGTATCATTGATGTCATAATTCAAATAGGCCAACAT
ACTTTTTGATTTATAGTTTTTTAATAGACGATATATTTTGTTAGG
ATCTGCTTCTTTTAACGTTAATAGCGAGGAGTCTGGACTATAAAT
GTCTAATGATAAACGATGAGATATCAAATAGTAATTCTGTTGCTC
TGCCAAGGCCTTTGCCTCTTCAAAGGCATCGGCCCCCAGATCTAT
ACAAAAGAACAGGTTATCCATATTATAGAATCGTATGGAGGCAAT
CATGGCCAAATTAATATTAGCTCCTAAGATAAAACAATAATATAT
AGTTAAAAAATTGTTATCTTTTGTGCAGGCTATCCGCATCATTTC
ATCCATGTCCATACGGATTTTTTCTTTTTCGTACAAATTATGTAG
GTCAAACAGCTTATTATAGCAAAGAGCACATGTTAACCACCACGT
ATTCAAATACTTAAAATGTTGGTAAACATAAGAAATGGCCTCCCT
AAGATTATCCTGCAATGCCACGATAAAACAGTATATAGTTAACAT
ATCACCATCCGAAATATTACTTAATACGTTGGTATCTTCTGCTAA
ATTTTTTAGCTTCCAATGTATACACGACTTTATTTCCCTTATAAT
GACATAGGCTGAAAAGGGATTGTCATTAAAAAATTTAAGACATAA
GATAATATTACTACTAGTAGTGCTAGGATGTATTAATTTAAAGAA
TATGTGCATAATCTTCTTTTTATCCACTTGGTGCTTGGCTCCTAA
TTCCCAGCAAAATTCTCGAATAGGTGGCGTATTCGCGCAAATTAA
CCCATAGTTGATGTCTGCGCCCCAATCCGTAAACATTTTTATTAA
TTGATAGTTGTTTTCCTTTGTAGCCAACATTAGTGCCGTATTAAG
GTCCAAGCCATCTGCAAAGTTTGGCAGCTTTATCAGCATATGTTT
GCAATCAAGTGAAATTGGAGCCTTATACCACCATAGCCCGCAGCG
TTCTAAGATAACATGGTGATCAATAGATACATGCTGACTGGCTAA
TACCTTTTTGGCGAAGGATTGCAAGGAAGGAAACAT
Pretorisuskop/96/4 MGF 360 10L
(AY261363.1:26116-27186)
SEQ ID No. 20
TTATAACGTTGTATTATTGACGTCATAATTCAAATAGGCCAATAT
ACTTTTTGATTTATAGTTTTTTAATAGATGATATATTTTGTTAGG
ATCCGCTTCTTTTAACGTTAATAACGAGGAATCTGGACTATAAAT
GTCTAATGATAAACAATGAGATATCAAAAAGTAATTCCGTTGCTC
TGCCAGGGCTTTTGCCTCCTCAAAGGCATCGGCCCCCAGGTCTAT
ACAAAAGAATAAGTTATCTATGTTATAGAATTGTATGGAGGCAAT
CATAGCTAAATTAATATTAGCTCCTAAGATAAAACAATAATATAT
GGTTAAAAAATTGTTATCTTTTGTGCAGGCTATCCGCATCATTTC
ATCCATGTCCATGCGGATCTTTTCCTTTTCATACAAATGATGTAG
GTCAAACACCTTATTATAGCAAAGAACACATGTTAACCACCACGT
ATTCAGATACTTAAAATGTTGGTAAACATAAGAAATGGCCTCCCT
AAGATTATCCTGCAATGCCACGATAAAACAGTATATAGTTAACAT
ATCTCCATCCGAAATATTACTTAATACGTTGGTATCTTCTGCTAA
CTTTTTTAGCTTCCAATGTATACACGACTTTATTTCCCTTATAAT
GACATAGGCTGAAAAGGGATTGTCATTAAAAAGTTTAAGACATAA
GATAATATTACTACTAGTAGTGCTAGGATGTATTAATTTAAAGAA
TATGTGCATAATCTTCTTTTTATCCACTTGGTACTTGGCTCCTAA
TTCCCAGCAAAATTCTCGAACAGGTGGCGTATTCGCGCAAATTAA
CCCATAGTTGATGTCTGCGCCCCAATCCGTAAACATTTTTATTAA
TTGATAGTTGTTTTCCTTTGTAGCCAACATTAGTGCCGTATTAAG
GTCCAAGCCATCTGCAAAGTTTGGCAGCTTTATCAGCATATGTTT
GCAATCAAGTGAAATTGGAGCCTTATACCACCATAGCCCGCAGCG
TTCTAAGATAATATGGTGATCAATGGATACATGCTGTCTGGCTAG
TACCTTTTTGGCGAAGGATTGTAAGGAAGGAAACAT
Tengani 62 MGF 360 10L
(AY261364.1:20530-21600)
SEQ ID No. 21
TTATAACAATGTATCATTGATATCATCATTCAAATAGGCCAACAT
ATTTTTTGATTTATAGTTTTTTAATAGATGATTTATTTTGTTAGG
ATCTGTTTCTTTTAACGTTAATAGCAAGGAGTCTGGCTTATAAAT
GTCTAATGATAAACGATGAGATATTAAATAGTAATTCCGTTGCTC
TGCCAGGGCTTTTGCCTCTTCAAAGGCGTCGGCTCCCAGATCTAT
ACAAAAGAACAAGTTATCCATATTATAAAATCGTACGCAGGCAAG
CATAGCTGAATTAATATTAGCTCCTAAGAGAAAACAATAATATAT
AGTTAAAAAATTGTTATCTTTTGTGCAGGCCATCCGCATCATTTC
ATCCACGTCCATGCGGATCTTTTCCTTTTCATACAAATTATGTAG
GTCAAACAGCTTATTAAAACAAAGAGCACAGATTAACCACCACGT
ATTTAGATACTTAAAATGTTGGTAAACATAAGAAATGGCCTCCCT
AAGATTATCCTGCAATGCCACTATAAAACAGTATATCATTAACAT
ATCACCATCCGACATATTACTTAATATGTCGGTGTCTTCTACTAA
CCTTTTCAGCTTCCAATATATGGATATCCTTATTTCCCTTATAAT
GACATAGGCTGGAAAGGGATTATCATTAAAAAGTTTAAGACATAA
GATAATATTACTGCTAGTAGTGCCAGGATGTATTAATTTAAAGAA
CATGTGTATAATCTTCTTTTTATCCACGCGGTGCTTGGCTCCTAA
TTCCCAGCAAAATTCTCGAACAGACGGCGTATTGGCGCAAATTAA
CCCATAGTTGATGTCTGCGCCCCATTCCGTAAACAGTTTTATTAA
CTGATAGTTATTTTCCTTTGTAGCCAACATTAGTGCCGTATTAAG
GTCCAAGCCGTCTGCAAAGCTTGGCAGCTTTATCAGCATATGTTT
GCAGTCAAGTGAAATTGGAGCCTTATACCACCATAGTCCGCAACG
TTCTAAGATAACATGGTACTCAATAGATACTTGCTGTCTGGCTAG
TACCTTTTTGGCGAAGGATTGTAAGGAAGGAAACAT
Warmbaths MGF 360 10L
(AY261365.1:25300-26361)
SEQ ID No. 22
TCATATTGTATCATCATGGTTAAAATATGCCGTCATATTTTTTGA
TTTATAGTTTTTTAATAGATGATATATTTTGTTAGGGTCCGCTTC
TTTTAACGTTAATAGCGAGGAGTCTGGACTATAAATGTCTAATGA
TAAACGATGAGATATCAAAAAGTAATTCCGTTGCTCTGCCAGAGC
CTTTGCCTCTTCAAAGGCGTCGGCCCCCAGGTCTATACAAAAGAA
CAGGTTATCCATATTATAGAATCGTATGGAGGCAATCATGGCCAA
ATTAATATTAGCTCCTAAGATAAAACAATAATATATAGTTAAAAA
ATTGTTATCTTTTGTGCAGGCTATCCGCATCATTTCATCCATGTC
CATGCGGATTTTTTCCTTTTCGTACAAATTATGTAGGTCAAACAG
CTTATTAAAACAAAGAGCACAGATTAGCCACCACATATTCAGATA
CTTAAAATGTTGGTAAACATAAGAAATGGCCTCCCTAAGATTATC
CTGCAATGCCACCATAAAACAGTATATCGTTAACATATCACCATC
TGAAATATCACTTAATACGTCGGTGTCTTCTACTAACTTTCTCAG
CTTCCAATATATGGATGACTTTATTTCCCTTATAATGACATAGGC
TGAAAAGGGGTTATCATTAAAAAGTTTAAGACATAAGATAATATT
ACTGCTAGTAGTGCCAGGATGTATTAATTTAAAGAACATGTGCAT
AATCTTCTTTTTATCCACGCGGTACTTGGCTCCTAGTTCCCAGCA
CAATTCTCGAACAGGCGGTGTATTGGCGCAAATTAACCCATAGTT
GATGTCTGCGCCCCATTCCGTAAACAGTTTTATTAACTGATAGTT
GTTTTCCTTTGTAGCCAACATTAGTGCCGTATTAAGGTCCAAGCC
ATCTGCAAAGCTTGGCAGCTTTATCAGCATATGTTTGCAATCAAG
TGAAATTGGAGCCTTATACCACCATAGTCCGCAGCGTTCTAAGAT
AACATGGTAATCAATAGATACATGCTGTCTGGCTAGTACCTTTTT
GGCGAAGGATTGTAAGGAAGGAAACAT
Warthog MGF 360 10L
(AY261366.1:22069-23130)
SEQ ID No. 23
TCATATTGTATCATCATGGTTAAAATATGCCGTCATATTTTTTGA
TTTATAGTTTTTTAATAGATGATATATTTTGTTAGGGTCCGCTTC
TTTTAACGTTAATAGCGAGGAGTCTGGACTATAAATGTCTAATGA
TAAACGATGAGATATCAAAAAGTAATTCCGTTGCTCTGCCAGGGC
TTTTGCCTCTTCAAAGGCATCGGCCCCCAAGTCTATACAAAAGAA
CAGGTTATCCATATTATAAAATCGTATGGAGGCAATCATGGCCAA
ATTAATATTAGCTCCTAAGATAAAACAATAATATATAGTTAAAAA
ATTGTTATCTTTTGTGCAGGCTATCCGCATCATTTCATCCATGTC
CATGCGGATTTTTTCCTTTTCGTACAAATTATGTAGGTCAAACAG
CTTATTAAAACAAAGAGCACAGATTAGCCACCACGTATTCAGATA
TTTAAAATGTTGGTAAACATAAGAAATGGCCTCCCTAAGATTATC
CTGCAATGCCACCATAAAACAGTATATCGTTAACATATCACCATC
CGAAATATTACTTAATACGTCGGTGTCTTCTACTAACTTTTTCAG
CTTCCAATATATGGATGACTTTATTTCCCTTATAATGACATAGGC
TGAAAAGGGGTTATCATTAAAAAGTTTAAGACATAAGATAATATT
ACTGCTAGTAGTGCCAGGATGTATTAATTTAAAGAACATGTGCAT
AATCTTCTTTTTATCCACGCGGTACTTGGCTCCTAGTTCCCAGCA
CAATTCTCGAACAGGCGGCGTATTGGCGCAAATTAACCCATAGTT
GATGTCTGCGCCCCATTCCGTAAACAGTTTTATTAACTGATAGTT
GTTTTCCTTTGTAGCCAACATTAGTGCCGTATTAAGGTCCAAGCC
ATCTGCAAAGCTTGGCAGCTTTATCAGCATATGTTTGCAATCAAG
GGAAATTGGAGCCTTATACCACCATAGTCCGCAGCGTTCTAAGAT
AACATGGTAATCAATAGATACATGCTGTCTGGCTAGTACCTTTTT
GGCGAAGGATTGTAAGGAAGGAAACAT

In an embodiment the attenuated ASFV of the invention comprises a functional version of MGF 360 10L. Suitably the functional version of MGF 360 10L comprises the sequence of SEQ ID No. 11, 12, 13, 14, 15, 16, 17, 18, 19, 20, 21, 22 or 23. Suitably the functional version of MGF 360 10L comprises a sequence having at least 70%, at least 80%, at least 90% or at least 95% identity with SEQ ID No. 11, 12, 13, 14, 15, 16, 17, 18, 19, 20, 21, 22 or 23. Suitably the functional version of MGF 360 10L consists of the sequence of SEQ ID No. 11, 12, 13, 14, 15, 16, 17, 18, 19, 20, 21, 22 or 23.

MGF 360 11L gene sequences
SEQ ID No. 24-Benin 97/1 MGF 360 11L (NC_044956.1:20703-21764)
TTATCTTTGTTCATAATCAAGAAAAATCCCCATATTTATTGAGTGATAATTTTTTAACATGTAATTTATTTTTTC
AGGGTCCATAACGTTCGACAACAGAGAAATGACTGGATTGTAATGCTTTAATGATAAGGCATGGGCTATCAGATA
ATTTTCCTTTTGTTCTGCCAAAGCTTTGCCCTCCTCAAAGGCGTTGGCGCCCAGGTCTATACAAAAGAACAGGTT
TCCAAGATTATAGTTTTGTATGGAAACCAGCATGGCTTGATTGATGTTGGCTCCCATGATAAAACAGTAGTAAAT
GGCCGAATAGCTATAATCTTGGATGCAGGCTATGTGCATCATTTCATCGATATCCATGCGGACCCTTTCTATTTC
ATACAGCTCGTGAAGGTCGAACACGTTGTTGTAAAAAAGGGCGCACATGAGCCGCCACCGATGTAGGCGCGGGTA
TTTCTGGTAAAAGTAGCGAATAGCATCTTTGAGGTCATAGTCCACCGCTATAGCATACCAGTATTTGGTTAAAAC
AGTGCTAAAGCTATCATCATGGTCTAGCATGAAGGTTATCTCCATGAGCCCTCTTAACTCCCACATGATTTCCCC
CCTCAGATCCAGATTATCTATAATCCTTAAATTGGGGTTATTGGAAAACACCTCGTGGCAAAAGATAATATTGCT
ACTGGTTTTATCGCGCGTCGTATCAAAGAAAATTTTTAAAATATACTCTCTTTCTAAATATTCTTTGGCTCCCAG
CTCTTTGCACAGATCACGGGTATTTTCCGTGAGAGCACAAATCATTCCATAGTTAATATCTGCACCCCATTCAGT
AAACAGCTTTATCAAGTCATGATTATTCTCCTTCACGGCTTTCATCAGTCCTATGTTTAACTCGATACCTTGACT
AAAGCAGGTTGACCTTATAAATAGTTTATTACGTCGAATATGAAGCATAATGGGGCCATTATGCCACCACAGGCC
ACAACACTTCAGGATATGATAATGATCCACCGACACGCACTGCCCGGCCAGTACTTTCTTCGTGAGGGATTGCAG
GGAAGGCAACAT
SEQ ID No. 25-China/2018/AnhuiXCGQ MGF 360 11L (MK128995.1:26467-27528)
TTATCTTTGTTCATAATCAAGAAAAATCCCCATATTTATTGAGTGATAATTTTTTAACATGCAATTTATTTTTTC
AGGGTCCGTAACGATCGACAACAGAGAAATAACCGGATTGTAATGCTTTAATGATAAGGCATGGGCTATCAGATA
ATTTTCCTTTTGTTCTGCCAAAGCTTTGCCCTCCTCAAAGGCATCGGCACCCAGGTCTATACAAAAGAACAGGTT
TCCAAGATTATAGTTTTGTATGGAAACAAGCATGGCTTGATTGATGTTGGCTCCCATGATAAAACAGTAGTAAAT
GGCCGAATAGCTATAATCTTGGATGCAGGCTATGTGCATCATTTCATCAATATCCATGCGGACCCTTTCTATTTC
GTACAGCTCGTGAAGGTCGAACACGTTGTTGTAAAAAAGGGCGCACATGAGCCGCCACCTATGTAGACGCGGGTA
TTTCTGGTAAAAGTAGCGGATAGCATCTTTGAGGTCATAGTCCACCGCTATCGCGTACCAGTATTTGGTTAAAAC
AGTGCTAAAGCTATCATCATGGTCCAGCATGAAGGTTATCTCCATGAGCCCTCTTAACTCCCACATGATTTCCCC
CCTCAGATCCAGATTATCTATAATCCTTAAATTGGGGTTATTGGAAAACACCTCGTGGCAAAAGATAATATTGCT
ACTGGTTTTATCGCGCGTTGTATCAAAGAAAATTTTTAAAATATACTCTCTTTCTAAATATTCTTTGGCTCCCAG
CTCTTTGCACAGATCACGGGTATTTTCCGTGAGAGCACAAATCATTCCATAGTTAATATCTGCACCCCATTCAGT
AAACAGCTTTATCAAGTCATGATTATTCTCCTTCACGGCTTTCATCAGTCCTATGTTTAACTCGATACCTTGACT
AAAACAGGTTGACCTTATAAATAATTTATTGCGTCGAATATGAAGCATAATGGGGCCATTATGCCACCACAGGCC
ACAACACTTCAGGACATGATATTGATCTACCGGTATACACTGCCCGGCCAGTACTTTCTTCGTGAGGGATTGCAG
GGAAGGCAACAT
SEQ ID No. 26-Georgia 2007/1 MGF 360 11L (NC_044959.1:26465-27526)
TTATCTTTGTTCATAATCAAGAAAAATCCCCATATTTATTGAGTGATAATTTTTTAACATGCAATTTATTTTTTC
AGGGTCCGTAACGATCGACAACAGAGAAATAACCGGATTGTAATGCTTTAATGATAAGGCATGGGCTATCAGATA
ATTTTCCTTTTGTTCTGCCAAAGCTTTGCCCTCCTCAAAGGCATCGGCACCCAGGTCTATACAAAAGAACAGGTT
TCCAAGATTATAGTTTTGTATGGAAACAAGCATGGCTTGATTGATGTTGGCTCCCATGATAAAACAGTAGTAAAT
GGCCGAATAGCTATAATCTTGGATGCAGGCTATGTGCATCATTTCATCAATATCCATGCGGACCCTTTCTATTTC
GTACAGCTCGTGAAGGTCGAACACGTTGTTGTAAAAAAGGGCGCACATGAGCCGCCACCTATGTAGACGCGGGTA
TTTCTGGTAAAAGTAGCGGATAGCATCTTTGAGGTCATAGTCCACCGCTATCGCGTACCAGTATTTGGTTAAAAC
AGTGCTAAAGCTATCATCATGGTCCAGCATGAAGGTTATCTCCATGAGCCCTCTTAACTCCCACATGATTTCCCC
CCTCAGATCCAGATTATCTATAATCCTTAAATTGGGGTTATTGGAAAACACCTCGTGGCAAAAGATAATATTGCT
ACTGGTTTTATCGCGCGTTGTATCAAAGAAAATTTTTAAAATATACTCTCTTTCTAAATATTCTTTGGCTCCCAG
CTCTTTGCACAGATCACGGGTATTTTCCGTGAGAGCACAAATCATTCCATAGTTAATATCTGCACCCCATTCAGT
AAACAGCTTTATCAAGTCATGATTATTCTCCTTCACGGCTTTCATCAGTCCTATGTTTAACTCGATACCTTGACT
AAAACAGGTTGACCTTATAAATAATTTATTGCGTCGAATATGAAGCATAATGGGGCCATTATGCCACCACAGGCC
ACAACACTTCAGGACATGATATTGATCTACCGGTATACACTGCCCGGCCAGTACTTTCTTCGTGAGGGATTGCAG
GGAAGGCAACAT
SEQ ID No. 27-L60 MGF 360 11L (NC_044941.1:21052-22113)
TTATCTTTGTTCATAATCAAGAAAAATCCCCATATTTATTGAGTGATAATTTTTTAACATGTAATTTATTTTTTC
AGGGTCCATAACGTTCGACAACAGAGAAATGACTGGATTGTAATGCTTTAATGATAAGGCATGGGCTATCAGATA
ATTTTCCTTTTGTTCTGCCAAAGCTTTGCCCTCCTCAAAGGCGTTGGCGCCCAGGTCTATACAAAAGAACAGGTT
TCCAAGATTATAGTTTTGTATGGAAACCAGCATGGCTTGATTGATGTTGGCTCCCATGATAAAACAGTAGTAAAT
GGCCGAATAGCTATAATCTTGGATGCAGGCTATGTGCATCATTTCATCGATATCCATGCGGACCCTTTCTATTTC
ATACAGCTCGTGAAGGTCGAACACGTTGTTGTAAAAAAGGGCGCACATGAGCCGCCACCGATGTAGGCGCGGGTA
TTTCTGGTAAAAGTAGCGAATAGCATCTTTGAGGTCATAGTCCACCGCTATAGCATACCAGTATTTGGTTAAAAC
AGTGCTAAAGCTATCATCATGGTCTAGCATGAAGGTTATCTCCATGAGCCCTCTTAACTCCCACATGATTTCCCC
CCTCAGATCCAGATTATCTATAATCCTTAAATTGGGGTTATTGGAAAACACCTCGTGGCAAAAGATAATATTGCT
ACTGGTTTTATCGCGCGTCGTATCAAAGAAAATTTTTAAAATATACTCTCTTTCTAAATATTCTTTGGCTCCCAG
CTCTTTGCACAGATCACGGGTATTTTCCGTGAGAGCACAAATCATTCCATAGTTAATATCTGCACCCCATTCAGT
AAACAGCTTTATCAAGTCATGATTATTCTCCTTCACGGCTTTCATCAGTCCTATGTTTAACTCGATACCTTGACT
AAAGCAGGTTGACCTTATAAATAGTTTATTACGTCGAATATGAAGCATAATGGGGCCATTATGCCACCACAGGCC
ACAACACTTCAGGATATGATAATGATCCACCGACACGCACTGCCCGGCCAGTACTTTCTTCGTGAGGGATTGCAG
GGAAGGCAACAT
SEQ ID No. 28-Ken05/Tk1 MGF 360 11L (NC_044945.1:28407-29468)
TTATCTGCGTTCATAATCGAGAAAAACCACCATATTCTTTGAGTGATAATTTTTTAACATAGAATTTATCTTTTC
TGGGTCGGTCACGGCCGACAACAGGGAAATCACCGGGTTATAATGTTTTAATGATAAGGCATCGGCTATCAAATA
ATTTTCCTTTTGTATTGCTAGGGCTTTGCCTTCTTCAAAGGCGTTGGCACCCAGGTCTATACAAAAGAACATGTT
CCCGAGATTATAGTTTTGTATGGAAACAAACATGGCTTGATTGATGTTGGCCCCCATGATAAAACAGTAGTAAAT
AGCTGAATAGCTATAATCTTGGATGCAGGCTATGTGCATCATCTCATCGATATCCATGCGGACCCTTTCTATTTC
ATACAGCTCGTGAAGGTCGAACACATTGTTATAAAAAAGGGCGCACATAAGCCGCCACCGATGTAGATGGGTATA
CTTCTGATAAAAATAGTGGATGGCCTTTTTGAGGCCGTAGTCTACCGCTATGGCGTACCAGTATTTGGTTAACAT
ATTGCTGAAGGAGTTGTCATGGTCCAACATGTAGGTTATTTCCATGAGTCCCCTTAGCTCCCACATGATTTCCTC
CCTCAGATCCAAATCATCCACATGTTTCAGATTGGCGTTATTGGAAAACATTTCGTGGCATAAGATAATATTACT
GTCGGTTTTGTTGCGCGTCGTATCAAAGAAAATTTTTAAAATATACTCTCTATCTAAACGGTCTTTGGCTCCAAG
TTCTCTACATAGATTGCGGGTGTGCTCCGAATGAGCACAAACCAGTCCATAATTAATATCGGCACCCCACTCAAC
AAACAGCCTTATCAAGTCATGATTGTTTTCCTTCGCGGCTTTCATCAGTACGGTGTTCAATTCAATACCCTCGCT
AAAACAGGTTGACTTTATAAACATTTTGTTACGATACGTATAAAATGTAATAGGGCCATCATACCACCACAACCC
GCAGCATTTCAGGATATGATATTGATCTACCGGTATACACTGTTTGGCCAGCACTTTCTTGGATAGGGATTGCAA
GGAAGGCAACAT
SEQ ID No. 29- KenO6.Bus MGF 360 11L (NC_044946.1:24534-25595)
TTATCTGCGTTCATAATCGAGAAAAACCACCATATTCTTTGAGTGATAATTTTTTAACATAGAATTTATCTTTTC
CGGGTCGGTCACGGCCGACAACAGGGAAATCACCGGGTTATAATGTTTTAATGATAAGGCATCGGCTATCAAATA
ATTTTCCTTTTGTATTGCTAGGGCTTTGCCTTCTTCAAAGGCGTTGGCACCCAGGTCTATACAAAAGAACATGTT
CCCGAGATTATAGTTTTGTATGGAAACAAACATGGCTTGATTGATGTTGGCACCCATGATAAAACAGTAGTAAAT
AGTTGAATAGCTATAATCTTGGATGCAGGCTATGTGCATCATCTCATCGATATCCATGCGGATCCTTTCTATTTC
ATACAGCTCGTGAAGGTCGAACACATTGTTATAAAAAAGGGCGCACATGAGCCGCCACCGATGTAGATGGGTATA
CTTCTGATAAAAATAGTGGATGGCCTTTTTGAGGCCGTAGTCTACCGCTATGGCGTACCAGTATTTGGTTAACAT
GTTGCTGAAGGAGTTGTCATGGTCCAACATGTAGGTTATCTCCATGAGTCCCCTTAGCTCCCACATGATTTCCTC
CCTCAGGTCCAAATCATCCACATGTTTCAGATTGGGGTTATTGGAAAACATTTCGTGGCATAAGATAATATTACT
GTCGGTTTTGTTGCGCGTCGTATCAAAGAAAATTTTTAAAATATACTCTTTATCTAAACGGTCTTTGGCCCCTAG
TTCTCTGCATAGATCGCGGGTGTGCTCCGAATGAGCACAAACCAGTCCATAATTAATATCGGCACCCCACTCAGT
AAATAATTTTATTAGGTCATGATTGTTTTCCTTCGCGGCTTTCATCAGTACGGTGTTCAATTCAATACCCTCGCT
AAAACAGGTTGACTTTATAAACATTTTGTTACGATACGTATAAAATGTAATAGGGCCATCATACCACCACAACCC
GCAACTTTTCAGGATATGATATTGATCTACCGGTATACACTGTTTGGCCAGCACTTTTTTGGATAGGGATTGCAA
GGAAGGCAACAT
SEQ ID No. 30- Kenya 1950 MGF 360 11L (AY261360.1:30655-31716)
TTATCTGCGTTCATAATCGAGAAAAACCACCATATTCTTTGAGTGATAATTTTTTAACATAGAATTTATCTTTTC
TGGGTCGGTCACGGCCGACAACAGGGAAATCACCGGGTTATAATGTTTTAATGATAAGGCATCGGCTATCAAATA
ATTTTCCTTTTGTATTGCTAGGGTTTTGCCTTCTTCAAAGGCGTTGGCACCCAGGTCTATACAAAAGAACATGTT
CCCGAGATTATAGTTTTGTATGGAAACAAACATGGCTTGATTGATGTTGGCCCCCATGATAAAACAGTAGTAGAT
AGCTGAATAGCTATAATCTTGGATGCAGGCTATGTGCATCATCTCATCGATATCCATGCGGACCCTTTCTATTTC
ATACAGCTCGTGAAGGTCGAACACATTGTTATAAAAAAGGGCGCACATAAGCCGCCACCGATGTAGATGGGTATA
CTTCTGATAAAAATAGTGGATGGCCTTTTTGAGGCCGTAGTCTACCGCTATGGCGTACCAGTATTTGGTTAACAT
ATTGCTGAAGGAGTTGTCATGGTCCAACATGTAGGTTATCTCCATGAGTCCCCTTAGCTCCCACATGATTTCCTC
CCTCAGATCCAAATCATCCACATGTTTCAGATTGGGGTTATTGGAAAACATTTCGTGGCATAAGATAATATTACT
GTCGGTTTTGTTGCGCGTCGTATCAAAGAAAATTTTTAAAATATACTCTCTATCTAAACGGTCTTTGGCTCCAAG
TTCTCTGCATAGATTGCGGGTGTGCTCCGAATGAGCACAAACCAGTCCATAATTAATATCGGCACCCCACTCAAC
AAACAGCCTTATCAAGTCATGATTGTTTTCCTTCGCGGCTTTCATCAGTACGGTGTTCAATTCAATACCCTCGCT
AAAACAGGTTGACTTTATAAACATTTTGTTACGATACGTATAAAATGTAATAGGGCCATCATACCACCACAACCC
GCAGCATTTCAGGATATGATATTGATCTACCGGTATACACTGTTTGGCCAGCACTTTCTTGGATAGGGATTGCAA
GGAAGGCAACAT
SEQ ID No. 31-Malawi Lil-20/1 (1983) MGF 360 11L (AY261361.1:24831-25892)
TTATCTTCGTTCATAATCAAGAAAAACCCTCATATTTATTGAGTGATAATTTTTTAACATGCAATTTATTTTTTC
AGGGTCCATAATGACCGACAACAGAGAAATGACCGGATTGTAATGCTTTAATGATAAGGCATCGGCTATCAGATA
ATTTTCCTTTTGTTCTGCCAAAGCTTTCCCCTCCTCAAAGGCGTTGGCCCCCAGATCTATACAAAAGAACATGTT
CCCAAGATTATAGTTTTGTATAGAAACAAGCATGGCTTGATTGATGTTGGCCCCCATGATAAAACAGTAATAAAT
AGCCGCATAGCTATAATCTTGGACGCAGGCTATGTGCATCATTTCATCAATATCCATGCGGACCCTTTCTATTAC
ATACAGCTCGTGAAGGTCGAACACGTTGTTGTAAAAAAGGGCGCACATAAGCCGCCAGTGGTGTAGATGGGTATA
CCTCTGATAAAAATAATGGATAGCCTTTTTGAGGCCATAGTCTACCGCTATGGCGTACCAGTATTTGGTTAACAT
ATTGCTAAAGGAGTTGTCATGGTCCAACATGTAGGTTATCTCCATGAGTCCCCTTAGCTCCCACATGATTTCCTC
CCTCAGATCCAAATTATCCACATTTTTCAGGTTGGGGTTATTGGAAAACATTTCGTGGCATAAGATAATATTACT
GTCGGTTTTGTTGCGCGTCGTATCAAAGAAAATTTTTAAAATATAATCTCTATCTAAACGATCTTTGGCTCCTAG
TTCTCTGCATAGATTACGGGTGTGCTCCGTATGGGCACAAATCAGTCCATAATTAATATCGGCACCCCACTCGGC
AAACAGCCTTATCAAGTCATGGTTGTTTTCCTTCGCGGCTTTCATCAGTACGGTGTTCAATTCAATACCCTCGCT
AAAACAGGTTGACCTTATAAACATTTTGTGACGATAAACGTAAAATGTAATAGGGCCATCATACCACCACAACCC
GCAGCACTTTAAGACATGATATTGATCTACCGGTACACACTGTTTGGCCAGCACTTTCTTAGTTAAAGATTGTAA
GGAAGGCAACAT
SEQ ID No. 32-Mkuzi 1979 MGF 360 11L (AY261362.1:28164-29225)
TTATCTTTGTTCATAATCAAGAAAAATCCCCATATTTATTGAGTGATAATTTTTTAACATGTAATTTATTTTTTC
AGGGTCCATAACGATCGACAACAGAGAAATGACCGGATTGTAATGCTTTAATGATAAGGCATGGGCTATCAGATA
ATTTCCCTTTTGTTCTGCCAAAGCTTTGCCCTCCTCAAAGGCGTTGGCGCCCAGGTCTATACAAAAGAACAGGTT
TCCAAGATTATAGTTTTGTATGGAAACCAGCATAGCTTGATTGATGTTGGCTCCCATGATAAAACAGTAGTAAAT
GACCGAATAGCTATAATCTTGGATGCAGGCTATGTGCATCATTTCATCAATATCCATGCGGACCCTTTCTATTTC
ATACAGCTCGTGAAGGTCGAACACGTTGTTGTAAAAAAGGGCGCACATGAGCCGCCACCTATGTAGGCGCGGGTA
TTTCTGGTAAAAGTAGCGAATAGCATCTTTGAGGTCATAGTCCACCGCTATAGCATACCAGTATTTGGTTAAAAC
AGTGCTAAAGCTATCATCATGGTCTAGCATGAAGGTTATCTCCATGAGCCCTCTTAACTCCCACATGATTTCTCC
CCTCAGATCCAGATTATCTATAATCCTTAAATTGGGGTTATTGGAAAACACCTCGTGGCAAAAGATAATATTGCT
ACTGGTTTTATCGCGCGTCGTATCAAAGAAAATTTTTAAAATATACTCTCTTTCTAAATATTCTTTGGCTCCCAG
CTCTTTGCACAGATCACGGGTATTTTCCGTGAGAGCACAAATCATTCCATAGTTAATATCTGCACCCCATTCAGT
AAACAGCTTTATCAAGTCATGATTATTCTCCTTCACGGCTTTCATCAGTCCTATGTTTAACTCGATACCTTGACT
AAAGCAGGTTGACCTTATAAATAGTTTATTACGTCGAATATGAAGCATAATGGGGCCATTATGCCACCACAGGCC
ACAACACTTCAGGATATGATAATGATCCACCGACACGCACTGCCCGGCCAGTACTTTCTTCGTGAGGGATTGCAG
GGAAGGCAACAT
SEQ ID No. 33-Pretorisuskop/96/4 MGF 360 11L (AY261363.1:27214-28275)
TTATCTTTGTTCATAATCAAGAAAAATCCCCATATTTATTGAGTGATAATTTTTTAACATGCGATTTATTTTTTC
AGGGTCCGTAACCACCGACAACAGAGAAATGACCGGATTGTAATGCTTTAATGATAAGGCATTGGCTATCAGATA
ATTTCCCTTTTGTTCTGCCAAAGCTTTGCCCTCTTCAAAGGCGTTGGCACCCAGGTCTATACAAAAGAACAGGTT
CCCAAGATTATAGTTTTGTATGGAAACCAGCATGGCTTGATTGATGTTGGCTCCCATGATAAAACAGTAGTAAAT
GGCTGAATAGCTATAATCTTGGACGCAGGCTATGTGCATCATTTCATCAATGTCCATGCGGACCCTTTCTATTTC
ATACAGCTCGTGAAGGTCGAACACGTTGTTGTAAAAAAGGGCGCACATGAGCCGCCACCTATGTAGCCGCGGGTA
TTTCTGGTAAAAGTAGCGGATAGCATCTTTGAGGTCATAGTCCACCGCTATCGCGTACCAGTATTTGGTTAAAAC
AGTGCTAAAGCTATCATCATGGTCCAGCATGAAGGTTATCTCCATGAGCCCTCTTAACTCCCACATGATTTCCCC
CCTCAGATCCAGATTATCTATAATCCTTAAATTGGGGTTATTGGAAAACACCTCGTGGCAAAAGATAATATTGTT
ACTGGTTTTATCGCGCGTTGTATCAAAGAAAATTTTTAAAATATACTCTCTTTCTAAATATTCTTTGGCTCCCAG
CTCTTTGCACAGATCACGGGTATTTTCCGTGAGAGCACAAATCATTCCATAGTTAATATCTGCACCCCATTCAGT
AAACAGCTTTATCAAGTCATGATTATTCTCCTTCACGGCTTTCATCAGTCCTATGTTTAACTCGATACCTTGACT
AAAGCAGGTTGACCTTATAAATATTCTATTACGTCGAATATGAAGCATTATGGGGCCATTATGCCACCACAGGCC
ACAACACTTCAGGATATGATAATGATCTACCGACACGCACTGCCCAGCCAGTACTTTCTTCGTGAGGGATTGCAG
GGAAGGCAACAT
SEQ ID No. 34-Tengani 62 MGF 360 11L (AY261364.1:21627-22688)
TTATCTTTGTTCATAATCAAGAAAAATCCCCATATTTATTGAGTGATAATTTTTTAACATGCAATTTATTTTTTT
AGGGTCTGTAACGACCGACAACAGAGAAATGACCGGATTGTAATGCTTTAATGATAAGGCATCGGCTATCAGATA
ATTTCCCTTTTGTTCTGCCAAAGCTTTGCCTTCCTCAAAGGCGTTGGCTCCCAGGTCTATACAAAAGAACAGGTT
CCCAAGATTATAGTTTTGTATGGAAACCAGCATGGCTTGATTGATGTTGGCTCCCATGATAAAACAGTAGTAAAT
GGCCGAATAGCTATAATCTTGGACGCAGGCTATGTGCATCATTTCATCAATGTCCATGCGGACCCTTTCTATTTC
GTACAGCTCGTGAAGGTCGAACACGTTGTTGTAAAAAAGGGCGCACATGAGCCGCCACCTATGTAGACGCGGGTA
TTTCTGGTAAAAGTAGCGGATCGCATCTTTGAGGTCATAGTCCACCGCTATCGCGTACCAGTATTTGGTTAAAAC
AGTGCTAAAGCTATCATCATGGTCCAGCATGAAGGTTATCTCCATGAGCCCTCTTAACTCCCACATGATTTCCCC
CCTCAGATCCAGATTATCTATAATCCTTAAATTGGGGTTATTGGAAAACACCTCGTGGCAAAAGATAATATTGTT
ACTGGTTTTATCGCGCGTCGTATCAAAGAAAATTTTTAAAATATACTCTCTTTCTAAATATTCTTTGGCACCCAG
CTCTTTGCACAGATCACGGGTATTTTCCGTGAGAGCACAAATCATTCCATAGTTAATATCTGCACCCCATTCAGT
AAACAGCTTTATCAAGTCATGATTATTCTCCTTCACGGCTTTCATCAGTCCTATGTTTAACTCGATACCTTGACT
AAAGCAGGTTGACCTTATAAATAGTTTATTACGTCGAATCTGAAGCATAATGGGTCCATTATGCCACCACAGGCC
ACAACACTTCAGGATATGATAATGATCCACCGACACGCACTGCCCGGCCAGTACTTTCTTCGTGAGGGATTGCAG
GGAAGGCAACAT
SEQ ID No. 35-Warmbaths MGF 360 11L (AY261365.1:26388-27449)
TTATCTTTGTTCATAATCAAGAAAAATCCCCATATTTATTGAGTGATAATTTTTTAACATGCGATTTATTTTTTC
AGGGTCCGTAACCACCGACAACAGAGAAATGACCGGATTGTAATGCTTTAATGATAAGGCATTGGCTATCAGATA
ATTTCCCTTTTGTTCTGCCAAAGCTTTGCCCTCCTCAAAGGCGTTGGCACCCAGGTCTATACAAAAGAACAGGTT
CCCAAGATTATAGTTTTGTATGGAAACCAGCATGGCTTGATTGATGTTGGCTCCCATGATAAAACAGTAGTAGAT
GGCTGAATAGCTATAATCTTGGACGCAGGCTATGTGCATCATTTCATCAATGTCCATGCGGACCCTTTCTATTTC
ATACAGCTCGTGAAGGTCGAACACGTTGTTGTAAAAAAGGGCGCACATGAGCCGCCACCTATGTAGCCGCGGGTA
TTTCTGGTAAAAGTAGCGGATAGCATCTTTGAGGTCATAGTCCACCGCTATCGCGTACCAGTATTTGGTTAAAAC
AGTGCTAAAGCTATCATCATGGTCCAGCATGAAGGTTATCTCCATGAGCCCTCTTAACTCCCACATGATTTCCCC
CCTCAGATCCAGATTATCTATAATCCTTAAATTGGGGTTATTGGAAAACACCTCGTGGCAAAAGATAATATTGTT
ACTGGTTTTATCGCGCGTTGTATCAAAGAAAATTTTTAAAATATACTCTCTTTCTAAATATTCTTTGGCTCCCAG
CTCTTTGCACAGATCACGGGTATTTTCCGTGAGAGCACAAATCATTCCATAGTTAATATCTGCACCCCATTCAGT
AAACAGCTTTATCAAGTCATGATTATTCTCCTTCACGGCTTTCATCAGTCCTATGTTTAACTCGATACCTTGACT
AAAGCAGGTTGACCTTATAAATATTCTATTACGTCGAATATGAAGCATTATGGGGCCATTATGCCACCACAGGCC
ACAACACTTCAGGATATGATAATGATCCACCGACACGCACTGCCCGGCCAGTACTTTCTTCGTGAGGGATTGCAG
GGAAGGCAACAT
SEQ ID No. 36-Warthog MGF 360 11L (AY261366.1:23157-24218)
TTATCTTTGTTCATAATCAAGAAAAATCCCCATATTTATTGAGTGATAATTTTTTAACATGCGATTTATTTTTTC
AGGGTCCGTAACCACCGACAACAGAGAAATGACCGGATTGTAATGCTTTAATGATAAGGCATTGGCTATCAGATA
ATTTCCCTTTTGTTCTGCCAAAGCTTTGCCCTCCTCAAAGGCGTTGGCCCCCAGGTCTATACAAAAGAACAGGTT
CCCAAGATTATAGTTTTGTATGGAAACCAGCATGGCTTGATTGATGTTGGCTCCCATGATAAAACAGTAGTAAAT
GGCTGAATAGCTATAATCTTGGACGCAGGCTATGTGCATCATTTCATCAATGTCCATGCGGACCCTTTCTATTTC
ATACAGCTCGTGAAGGTCGAACACGTTGTTGTAAAAAAGAGCGCACATGAGCCGCCACCTATGTAGCCGCGGGTA
TTTCTGGTAAAAGTAGCGGATAGCATCTTTGAGGTCATAGTCCACCGCTATCGCGTACCAGTATTTGGTTAAAAC
AGTACTAAAGCTATCATCATGGTCCAGCATGAAGGTTATCTCCATGAGCCCTCTTAACTCCCACATGATTTCTCC
CCTTAGATCCAGATTATCTATAATCCTTAAATTGGGGTTATTGGAAAACACCTCGTGGCAAAAGATAATATTGTT

ACTGGTTTTATCGCGCGTTGTATCAAAGAAAATTTTTAAAATATACTCTCTTTCTAAATATTCTTTGGCTCCCAG
CTCTTTGCACAGATCACGGGTATTTTCCGTGAGAGCACAAATCATTCCATAGTTAATATCTGCACCCCATTCAGT
AAACAGCTTTATCAAGTCATGATTATTCTCCTTCACGGCTTTCATCAGTCCTATGTTTAACTCGATACCTTGACT
AAAGCAGGTTGACCTTATAAATATTCTATTACGTCGAATATGAAGCATTATGGGGCCATTATGCCACCACAGGCC
ACAACACTTCAGGATATGATAATGATCCACCGATACGCACTGCCCGGCCAGTACTTTCTTCGTGAGGGATTGCAG
GGAAGGCAACAT

In an embodiment the attenuated ASFV of the invention comprises a functional version of MGF 360 11L. Suitably the functional version of MGF 360 11L comprises the sequence of SEQ ID No. 24, 25, 26, 27, 28, 29, 30, 31, 32, 33, 34, 35 or 36. Suitably the functional 10 version of MGF 360 11L comprises a sequence having at least 70%, at least 80%, at least 90% or at least 95% identity with SEQ ID No. 24, 25, 26, 27, 28, 29, 30, 31, 32, 33, 34, 35 or 36. Suitably the functional version of MGF 360 11L consists of the sequence of SEQ ID No. 24, 25, 26, 27, 28, 29, 30, 31, 32, 33, 34, 35 or 36.

MGF 360 12L gene sequences
SEQ ID No. 37-Benin 97/1 MGF 360 12L (NC_044956.1:23616-24668)
TCATCTTAAATCATAGGAAAGGAAGATCATCATATTTTTTGAAAAGTAATTTTTTAACGCATGATCTATGATTTC
AGGGTCCGTGCTTTTAGGCAACGGGATGGTGGTCGGACTATAAATCTTTAGAGATAAAATGTTTTTTATAAGCTC
ATACCCTTCCCCTAAAGCTGTAGTACCCTCTTCGAAAACATCAGCCCCCAGATCCATACAAAAGAACATGTTTTC
TATATTATAGTACTGTATTGAGGTAAGCATGGCCTGATTGATGTTGGCGCCCAACATATAGCAGTAGTACATGGT
TGAAAGGTTGTGGTCTTTGATGCAGGCAATCCGCATCATCTCTTCTATGTCCATATGGATCTTGTCCTTTTCATA
CGCCTCATGGAGGTCAAACACATTATTAAAACAAAGAGCACATGTTAACCGCCACGTATTCATGTGTGTATATTT
TTGGTAAAAATACTGTATGGCCTCTTTCAGGCTATAGCGTACGGCTATAGCGTACCAGTATTTGAGTAGTAATGT
ACTGAGCAAAAACTCATTATTTAGCAGATCGGTTTTTTCTATTAACTCCCTTAACTCCCAGAAAATTTCTATCCT
CAATTTCAGATTATTTACTTTTTGTAATATCGGATTGTTGGAAAACACCTCATGGCATAAAATAATGTTACTACT
AGTTTTATGAAACTTTAGATCTATGAAAATTTGTAAAATTTCCTCTTCATTCAAGATCTCCTTGGCACCTAACTC
TTGACAGAGGTCCCGGGTATGCTCCGTGTTGACAGATACCAGCCCATAGTTGATGTCCGCCCCCCACTCTGTAAA
TAGTTTTATAAGGCTGTAGTTGTTTTCCCTTACAGCCTTTACTAACGCCGTATTTAGGTCTAAGCCCTCTTTAAT
GCCTGATGATTTTATGAGCCTTAGGTTATGATCAAACGTGATCGGAGCATTATACCACCATAGATCATAATATTT
TAAAAGATAATGTTGGTTCGTGGGCACGCATTGTCCAGCCAACACCTTTTTGGTCAAAGATTGCAGGGAAGGCAA
CAT
SEQ ID No. 38-China/2018/AnhuiXCGQ MGF 360 12L (MK128995.1:29384-30436)
TCATCTTAAATCATAGGTAAGGAAGATCATCATATTTTTTGAAACGTAATTTTTTAACGCATGATCTATGATTTC
AGGGTCCGTGCTTTTAGGCAACGGGGTGGTGGCCGGACTATAAATCTTTAGGGATAAAATGTTCTTTATAAGCTC
ATACCCTTCCCCTAAAGCTGTAGTACCCTCTTCGAAAACATCAGCCCCCAGATCTATACAAAAGAACATGTTTTC
TATATTATAGTACTGTATTGAGCTAAGCATGGCTTGATTGATGTTGGCGCCCAGGACATAGCAGTAGTACATGGT
TGAAAGGTTGTGGTCTTTGATGCAGGCGATCCGCATCATCTCTTCTATGTCCATATGGATCTTGTCCTTTTCATA
CGCCTCATGAAGGTCAAACACATTATTAAAACAAAGAGCACATGTTAACCGCCACGTATTCAGGTGTGTATATTT
TTGGTAAAAATACTGTATGGCCTCTTTCAGGTTATAGCGTATGGCTATAGCGTACCAGTATTTGAGTAGTAATGT
ACTGAGCGAAAACTCATTATTTAGCAGATCGGTTTTTACTATTAACTCCCTTAACTCCCAGAAAATTTCTATCCT
CATTTTTATATTATTTACTTTTTGTAATATCGGATTGTTGGAAAACACCTCATGGCATAAAATAATGTTACTACT
AGTTTTATGAAACTTTAGATCTATAAAAATTTGTAAAATTTCTTCTTCATTCAAGGTTTCCTTGGCACCTAGCTC
TCGACAGAGGTCCCAGGTGTGCTCCGTGTTGACAGATACCAGCCCGTAGTTGATGTCCGCCCCCCACTCTGCAAA
CAGTTTTATAAGGTTGTAGTTGTTTTCCCTTACAGCCTTCACTAACGCCGTATTTAGGTTTAAGCCCTCTTTAAT
ACCTGCTGATTTTATGAGCCTTAGGTTATGATCAAACGTGATCGGAGCATCATGCCACCATAGGTCATAACACTT
TAAAAGATAATGTTGGTTCGTGGGCACGCATTGTCCAGCCAACACCTTTTTGGTCAGAGATTGCAGGGAAGGCAA
CAT
SEQ ID No. 39-Georgia 2007/1 MGF 360 12L (LR743116.1:30355-31407)
TCATCTTAAATCATAGGTAAGGAAGATCATCATATTTTTTGAAACGTAATTTTTTAACGCATGATCTATGATTTC
AGGGTCCGTGCTTTTAGGCAACGGGGTGGTGGCCGGACTATAAATCTTTAGGGATAAAATGTTCTTTATAAGCTC
ATACCCTTCCCCTAAAGCTGTAGTACCCTCTTCGAAAACATCAGCCCCCAGATCTATACAAAAGAACATGTTTTC
TATATTATAGTACTGTATTGAGCTAAGCATGGCTTGATTGATGTTGGCGCCCAGGACATAGCAGTAGTACATGGT
TGAAAGGTTGTGGTCTTTGATGCAGGCGATCCGCATCATCTCTTCTATGTCCATATGGATCTTGTCCTTTTCATA
CGCCTCATGAAGGTCAAACACATTATTAAAACAAAGAGCACATGTTAACCGCCACGTATTCAGGTGTGTATATTT
TTGGTAAAAATACTGTATGGCCTCTTTCAGGTTATAGCGTATGGCTATAGCGTACCAGTATTTGAGTAGTAATGT
ACTGAGCGAAAACTCATTATTTAGCAGATCGGTTTTTACTATTAACTCCCTTAACTCCCAGAAAATTTCTATCCT
CATTTTTATATTATTTACTTTTTGTAATATCGGATTGTTGGAAAACACCTCATGGCATAAAATAATGTTACTACT
AGTTTTATGAAACTTTAGATCTATAAAAATTTGTAAAATTTCTTCTTCATTCAAGGTTTCCTTGGCACCTAGCTC
TCGACAGAGGTCCCAGGTGTGCTCCGTGTTGACAGATACCAGCCCGTAGTTGATGTCCGCCCCCCACTCTGCAAA
CAGTTTTATAAGGTTGTAGTTGTTTTCCCTTACAGCCTTCACTAACGCCGTATTTAGGTTTAAGCCCTCTTTAAT
ACCTGCTGATTTTATGAGCCTTAGGTTATGATCAAACGTGATCGGAGCATCATGCCACCATAGGTCATAACACTT
TAAAAGATAATGTTGGTTCGTGGGCACGCATTGTCCAGCCAACACCTTTTTGGTCAGAGATTGCAGGGAAGGCAA
CAT
SEQ ID No. 40-Ken06.Bus MGF 360 12L (NC_044946.1:27428-28483)
TCATCTTAGATCATAAGTAAGGAAGACCATCATATTTTTTGAAAAGTAATTTTTTAACGCATGATCTATAATTTC
AGGGTCCGTGCTTTTAGGCAATGGGGTAGTGGCCGGGCTATAAATCTTTAGGGATAAAATATTCCTTATAGGCTC
ATACCCTTCCTGTCCCACTAGAGCCATACCCTCTTCAAAAGCATCGGCCCCCAGATCCATACAAAAGAATATGTT
TTCTATATTATAGTACTGTATTGAGGTAAGCATGGCCTGATTGATGTTGGCGCCCAGCATATAGCAGTAGTATAT
TGTTGAAAGGTTGTGGTCTTTGATGCAGGCTATCCGCATCATCTCTTCTAAATCCATACAGATCTTGTCCTTTTC
ATACGCCTCATGAAGGTCAAACACGTTATTAAAACAAAGAGCACATGTTAACCGCCACGTATTCAGGTGTGTATA
TTTTTGGTAAAAATACTGTATGGCCTCTTTCAAGTTGTAGCGTACGGCTATAGCGTACCAGTATTTGAGTAATAG
TGTATTGAGCGAAAACTCATTATTTAGCAGATCGGTTTTTTCTATTATCTCCCTTAACTCCCAAAAAATTTCTAT
CCTCATTTTCAGGTTATTTACCCTTTTTAAAAGTAAATTATCGGAAAACACCTCATGACATAAGATAATGTTACT
ACTGGTTTTATAAAATTTTAAATCTATGAAAATTTGTAAAATATCCCCTTCATTTAAGGTCTTCTTGGCGCCTAG
CTCTCGACAGAGGTCCCAGGTGTGCTCCGTGTTGACAGATATCAGCCCGTAGTTAATGTTCGCCCCCCACTCCGT
AAATAGTTTTATAAGACTGTAGTTGTTTTCCTTTACAGCCTTCACTAATGCCATGTTTAGGTCTAAGCCTTCTTG
AATGCCTGCTAATTTTATCAGCCTTAGGTTATGATCAAACGTGATCGGAGCATTATTCCACCATAGATCGTAACA
CTTTAAAAGATAATGTTGGTCCTCGGGCAGACATTGTCTAGCCAGCACCTTTTTGGTCAAAGATTGCAGGGAAGG
CAACAT
SEQ ID No. 41-Kenya 1950 MGF 360 12L (AY261360.1:33549-34604)
TCATCTTAGATCATAAGTAAGGAAGACCATCATATTTTTTGAAAAGTAATTTTTTAACTCATGATCTATAATTTC
AGGGTCCGTGCTTTTAGGCAACGGGGTGGTGGCCGGGCTATAAATCTTTAGGGATAAAATATTCCTTATAGGCTC
ATACCCTTCCTGTCCCACTAGAGCCATACCCTCTTCAAAAGCATCGGCCCCCAGATCCATACAAAAGAATATGTT
TTCTATATTATAGTACTGTATTGAGGTAAGCATGGCCTGATTGATGTTGGCGCCCAGCATATAGCAGTAGTATAT
CGTTGAAAGGCTGTGGTCTTTGATGCAGGCTATCCGCATCATCTCTTCTAAATCCATACAGATCTTGTCCTTTTC
ATACGCCTCATGAAGGTCAAACACGTTATTAAAACAAAGAGCACATGTTAATCGCCACGTATTCAGGTGCGTATA
TTTTTGGTAAAAATACTGTATGGCCTCTTTCAAGTTGTAGCGTACGGCTATAGCGTACCAGTATTTGAGTAATAG
TGTATTGAGCGAAAACTCATTATTTAGCAGATCGGTTTTTTCTATTATCTCCCTTAACTCCCAAAAAATTTCTAT
CCTCATTTTCAGGTTATTTACCTTTTTTAAAAGTAGATTATCGGAAAACACCTCATGACATAAGATAATGTTACT
ACTGGTTTTATGAAACTTTAAATCTATGAAAATTTGTAAAATATCCCCTTCATTTAAGGTCTTCTTGGCGCCTAG
CTCTCGACAGAGGTCCCAGGTGTGCTCCGTGTTGACAGATATCAGCCCGTAGTTGATGTTCGCTCCCCACTCCGT
AAATAGTTTTATAAGACTGTAGTTGTTTTCCTTTACAGCCTTCACTAATGCCATGTTTAGGTCTAAGCCTTCTTG
AAGGCCTGCTGATTTTATCAGCCTTAGGTTATGATCAAACGTGATCGGAGCATTATTCCACCATAGATCGTAACA
CTTTAAAAGATAATGTTGGTCCTCGGGCAGACATTGTCCAGCCAGCACCTTTTTGGTCAGAGATTGCAGGGAAGG
CAACAT
SEQ ID No. 42-L60 MGF 360 12L (NC_044941.1:23965-25017)
TCATCTTAAATCATAGGAAAGGAAGATCATCATATTTTTTGAAAAGTAATTTTTTAACGCATGATCTATGATTTC
AGGGTCCGTGCTTTTAGGCAACGGGATGGTGGTCGGACTATAAATCTTTAGAGATAAAATGTTTTTTATAAGCTC
ATACCCTTCCCCTAAAGCTGTAGTACCCTCTTCGAAAACATCAGCCCCCAGATCCATACAAAAGAACATGTTTTC
TATATTATAGTACTGTATTGAGGTAAGCATGGCCTGATTGATGTTGGCGCCCAACATATAGCAGTAGTACATGGT
TGAAAGGTTGTGGTCTTTGATGCAGGCAATCCGCATCATCTCTTCTATGTCCATATGGATCTTGTCCTTTTCATA
CGCCTCATGGAGGTCAAACACATTATTAAAACAAAGAGCACATGTTAACCGCCACGTATTCAGGTGTGTATATTT
TTGGTAAAAATACTGTATGGCCTCTTTCAGGCTATAGCGTACGGCTATAGCGTACCAGTATTTGAGTAGTAATGT
ACTGAGCAAAAACTCATTATTTAGCAGATCGGTTTTTTCTATTAACTCCCTTAACTCCCAGAAAATTTCTATCCT
CAATTTCAGATTATTTACTTTTTGTAATATCGGATTGTTGGAAAACACCTCATGGCATAAAATAATGTTACTACT
AGTTTTATGAAACTTTAGATCTATGAAAATTTGTAAAATTTCCTCTTCATTCAAGATCTCCTTGGCACCTAACTC
TTGACAGAGGTCCCGGGTATGCTCCGTGTTGACAGATACCAGCCCATAGTTGATGTCCGCCCCCCACTCTGTAAA
TAGTTTTATAAGGCTGTAGTTGTTTTCCCTTACAGCCTTTACTAACGCCGTATTTAGGTCTAAGCCCTCTTTAAT
GCCTGATGATTTTATGAGCCTTAGGTTATGATCAAACGTGATCGGAGCATTATACCACCATAGATCATAATATTT
TAAAAGATAATGTTGGTTCGTGGGCACGCATTGTCCAGCCAACACCTTTTTGGTCAAAGATTGCAGGGAAGGCAA
CAT
SEQ ID No. 43-Malawi Lil-20/1 (1983) MGF 360 12L (AY261361.1:27682-28731)
TCATCTTAAATCGTAGGAAAGGAAGGTCATCATATTTTTTGATTTATAATTTTTTAACACATGATCTATTATTTC
AGGGTCCGTGTTTTTAGGTAACGGGGCAGTGGACGGACTATAGATCTTTAGGGATAAAATGTTCCTTATAAGCTC
ATACCCTTCCCCTACAGGCATATTCTCTTCGAAAACATCAGCCCCCAGATCCATACAAAAGAACATGTTTTCTAT
ATTATAGTACTGTATTGAGGCAAGCATGGCCTGATTAATGTTGGCGCCCAGGACATAGCAGTAGTATATCGTTGA
AAGGTTGTGGTCTTTGATGCAGGCTACTCGCATCATCTCTTCTAAGTCCATATAGATCTTGTCCTTTTCATACGC
CTCATGAAGGTCAAACACATTATTAAAACAAAGAGCACATGTTAACCGCCACGTATTCAGGTGTGTATATTTTTG
ATAAAAATACTGTATGGCCTCTTTCAGGTTATAGCGTACGGCTATAGCGTACCAGTATTTGAGTAATAATGTATT
GAGTGAAAACTCATTATTTAACAGATCGGTTTTTTTTATTAACTCCCTTAACTCCCAGAAAATTTCTATCCTCAT
TTTCAGATTATTTACTTTTTGTAATAACGGATTGTTGGAAAACACCTCATGGCATAAAATAATGTTACTACTAGT
TTTATAAAACTTTAGATCGATGAAAATTCGTAAAATTTCCTCTTCATTTAAGGTCTCCTTGGCGCCTAGCTCTCT
GCAGAGGTCCCGGGTGTGCTCCGTGTTGACAGATACCAGCCCGTAGTTGATGTCCGCCCCCCACTCTGTAAAAAG
CTTTATAAGGTTGTAGTTGTTTTCCCTTACAGCCTTTACTAACGCCGTATTTAGGTCTAAGCCATCTTTAATGCC
TGCTGATTTTATCAGCCCTAGGTTATGATCAAACGTGATCGGAGAATCATACCACCATAGGTCGTAACACTTTAA
AAGATAATATTGGTCCGTCGGCAGGCATTGTCCAGCCAGTACCTTTTTGGTCAGAGATTGCAGGGAAGGCAACAT
SEQ ID No. 44-Mkuzi 1979 MGF 360 12L (AY261362.1:31073-32125)
TCATCTTAAATCATAGGAAAGGAAGATCATCATATTTTTTGAAAAGTAATTTTTTAACGCATGATCTATGATTTC
AGGGTCCGTGCTTTTAGGCAACGGGATGGTGGTCGGACTATAAATCTTTAGAGATAAAATGTTTTTTATAAGCTC
ATACCCTTCCCCTAAAGCTGTAGTACCCTCTTCGAAAACATCAGCCCCCAGATCCATACAAAAGAACATGTTTTC
TATATTATAGTACTGTATTGAGGTAAGCATGGCCTGATTGATGTTGGCGCCCAACATATAGCAGTAGTACATGGT
TGAAAGGTTGTGGTCTTTAATGCAGGCGATCCGCATCATCTCTTCTATGTCCATATAGATCTTGTCCTTTTCATA
CGCCTCATGGAGGTCAAACACATTATTAAAACAAAGAGCACATGTTAACCGCCACGTATTCAGGTGTGTATATTT
TTGGTAAAAATACTGTATGGCTTCTTTCAGGTTATAGCGTATGGCTATAGCGTACCAGTATTTGAGTAGCAATGT
ACTGAGCGAAAACTCATTATTTAGCAGATCGGTTTTTTCTATTAACTCCCTTAACTCCCAAAAAATTTCTATCCT
CATTTTTATATTATTTACTTTTTGTAATATCGGATTGTTGGAAAACACCTCATGGCATAAAATAATGTTACTACT
AGTTTTATGAAACTTTAGATCTATAAAAATTTGTAAAATTTCTTTTTCATTCAAGGTCTCCTTGGCACCTAGCTC
TCGACAGAGGTCCCAGGTGTGCTCCGAGCTGACAGATACCAGCCCATAGTTGATGTCCGCCCCCCACTCTGCAAA
CAATTTTATAAGGTTGTAGTTGTTTTCCTTTACAGCCTTCACCAACGCCGTATTTAGGTCTAAGCCCTCTTTAAT
GCCTGCTGATTTTATGAGCCTTAGGTTATGATCAAACGTGATCGGAGCATCATGCCACCATAGGTCATAATATTT
TAAAAGATAATGTTGGTTCGTGGGCACGCATTGTCCGGCCAACACCTTTTTGGTCAAAGATTGCAGGGAAGGCAA
CAT
SEQ ID No. 45-Pretorisuskop/96/4 MGF 360 12L (AY261363.1:30098-31150)
TCATCTTAAATCGTAGGTAAGGAAGATCATCATATTTTTTGAAACGTAATTTTTTAACGCATGATCTATAATTTC
AGGGTCCGTGCTTTTAGGCAACGGGGTGGTGGCCGGACTATAAATCTTTAGGGATAAAATGTTCTTTATAAGCTT
ATACCCTTCCCCTAAAGCTATAGTGCCCTCTTCGAAAGCATCAGCCCCCAGATCCATACAAAAGAACATGTTTTC
TATATTATAGTACTGTATTGAGGTAAGCATGGCCTGATTGATGTTGCCCCCCAGGACATAGCAGTAGTACATGGT
TGAAAGGTTGTGGTCTTTGATGCAGGCGATCCGCATCATCTCTTCTATGTCCATATGAATCTTGTCCTTTTCATA
CGCCTCATGAAGGTCAAACACATTATTAAAACAAAGAGCACATGTTAACCGCCACGTATTCAGGTGTGTATATTT
TTGGTAAAAATACTGTATGGCCTCTTTCAGGTTATAGCGTATGGCTATAGCGTACCAGTATTTAAGTAGTAATGT
ACTGAGCGAAAACTCATTATTTAGCAGATCGGTTTTTTCTATTAACTCCCTTAACTCCCAGAAAATTTCTATCCT
CATTTTTATATTATTTACTTTTTGTAATATCGGATTGTTGGAAAACACCTCATGGCATAAAATAATGTTACTACT
AGTTTTATGAAACTTTAGATCTATAAAAATTTGTAAAATTTCTTCTTCATTCAAGGTCTCCTTGGCACCTAGCTC
TCGACAGAGGTCCCAGGTGTGCTCCGTGTTGACAGATACCAGCCCATAGTTGATGTTCGCCCCCCACTCTGCAAA
CAGTTTTATAAGGTTGTAGTTGTTTTCCCTTACAGCCTTCACTAACGCCGTATTTAGGTCTAAGCCCTCTTTAAT
GCCTGCTGATTTTATGAGCTTTAGGTTATGATCAAACGTGATCGGACCATCATGCCACCATAGGTCGTAACACTT
TAAAAGATAATGTTGGTTTGTGGGCACGCATTGTCCAGCCAACACCTTTTTGGTCAGAGATTGCAGGGAAGGCAA
CAT
SEQ ID No. 46-Tengani 62 MGF 360 12L (AY261364.1:24540-25592)
TCATCTTAAATCATAGGTAAGGAAGATCATCATATTTTTTGAAACGTAATTTTTTAACGCATGATCTATGATTTC
AGGGTCCATGCTTTTAGGCAACGGGGTGGTGGCCGGACTATAAATCTTTAGGGATAAAATGTTCTTTATAAGCTC
ATACCCTTCCCCTAAAGCTGTAGTACCCTCTTCGAAAACATCAGCCCCCAGATCTATACAAAAGAACATGTTTTC
TATATTATAGTACTGTATTGAGCTAAGCATGGCCTGATTGATGTTGGCGCCCAGGACATAGCAGTAGTACATGGT
TGAAAGGTTGTGGTCTTTGATGCAGGCGATCCGCATCATCTCTTCTATGTCCATATGGATCTTGTCCTTTTCATA
CGCCTCATGAAGGTCAAACACATTATTAAAACAAAGAGCACATGTTAACCGCCACGTATTCAGGTGTGTATATTT
TTGGTAAAAATACTGTATGGCCTCTTTCAGGTTATAGCGTATGGCTATAGCGTACCAGTATTTGAGTAGTAATGT
ACTGAGCGAAAACTCATTATTTAGCAGATCGGTTTTTTCTATTAACTCCCTTAACTCCCAGAAAATTTCTATCCT
CATTTTTATATTATTTACTTTTTGTAATATCGGATTGTTGGAAAACACCTCATGGCATAAAATAATGTTACTACT
AGTTTTATGAAACTTTAGATCTATAAAAATTTGTAAAATTTCTTCTTCATTCAAGGTCTCCTTGGCACCTAGCTC
TCGACAGAGGTCCCAGGTGTGCTCCGTGTTGACAGATACCAGCCCGTAGTTGATGTCCGCCCCCCACTCTGCAAA
CAGTTTTATAAGGTTGTAGTTGTTTTCCCTTACAGCCTTTACTAACGCCGTATTTAGGTTTAAGCCCTCTTTAAT
ACCTGCTGATTTTATGAGCCTTAGGTTATGATCAAACGTGATCGGAGCATCATGCCACCATAGGTCATAACACTT
TAAAAGATAATGTTGGTTCGTGGGCACGCATTGTCCAGCCAACACCTTTTTGGTCAGAGATTGCAGGGAAGGCAA
CAT
SEQ ID No. 47-Warmbaths MGF 360 12L (AY261365.1:29294-30346)
TCATCTTAAATCGTAGGTAAGGAAGATCATCATATTTTTTGAAACGTAATTTTTTACCGCATGATCTATGATTTC
AGGGTCCGTGCTTTTAGGCAACGGGGTGGTGGCCGGACTATAAATCTTTAGGGATAAAATGTTCTTTATAAGCTT
ATACCCTTCCCCTAAAGCTATAGTACCCTCTTCGAAAGCATCAGCCCCCAGATCCATACAAAAGAACATGTTTTC
TACATTATAGTACTGTATTGAGGTAAGCATGGCCTGATTGATGTTGGCGCCCAGAACATAGCAGTAGTACATGGT
TGAAAGGTTGTGGTCTTTGATGCAGGCGATCCGCATCATCTCTTCTATGTCCATATAGATCTTGTCCTTTTCATA
CGCCTCATGAAGGTCAAACACATTATTAAAACAAAGAGCACATGTTAACCGCCATGTATTCAGGTGTGTATATTT
TTGGTAAAAATACTGTATGGCCTCTTTCAGGTTATAGCGTATGGCTATAGCGTACCAGTATTTGAGTAGTAATGT
ACTGAGCGAAAACTCATTATTTAGCAGATCGGTTTTTTCTATTAACTCCCTTAACTCCCAGAAAATTTCTATCCT
CATTTTTATATTATTTACTTTTTGTAATATCGGATTGTTGGAAAACACCTCATGGCATAAAATAATGTTACTACT
AGTTTTATGAAACTTTAGATCTATAAAAATTTGTAAAATTTCTTCTTCATTCAAGGTCTCCTTGGCACCTAGCTC
TCGACAGAGGTCCCGGGTGTGCTCCGTGTTGACAGATACCAGCCCATAGTTGATGTCCGCCCCCCACTCTGCAAA
CAGTTTTATAAGGTTGTAGTTGTTTTCCCTTACAGCCTTCACTAACGCCGTATTTAGGTCTAAGCCCTCTTTAAT
GCCTGCTGATTTTATGAGCTTTAGGTTATGATCAAACGTGATCGGACCATCATGCCACCATAGGTCATAACACTT
TAAAAGATAATGTTGGTTCGTGGGCACGCATTGTCCAGCCAACACCTTTTTGGTCAGAGATTGCAGGGAAGGCAA
CAT
SEQ ID No. 48-Warthog MGF 360 12L (AY261366.1:26036-27088)
TCATCTTAAATCGTAGGTAAGGAAGATCATCATATTTTTTGAAACGTAATTTTTTAACGCATGATCTATAATTTC
AGGGTCCGTGCTTTTAGGCAACGGGGTGGTGGCCGGACTATAAATCTTTAGGGATAAAATGTTCTTTATAAGCTT
ATACCCTTCCCCTAAAGCTATAGTGCCCTCTTCGAAAGCATCAGCCCCCAGATCCATACAAAAGAACATGTTTTC
TATATTATAGTACTGTATTGAGGTAAGCATGGCCTGATTGATGTTGGCGCCCAGGACATAGCAGTAGTACATGGT
TGAAAGGTTGTGGTCTTTGATGCAGGCGATCCGCATCATCTCTTCTATGTCCATATGGATCTTGTCCTTTTCATA
CGCCTCATGAAGGTCAAACACATTATTAAAACAAAGAGCACATGTTAACCGCCACGTATTCAGGTGTGTATATTT
TTGGTAAAAATACTGTATGGCCTCTTTCAGGTTATAGCGTATGGCTATAGCGTACCAGTATTTGAGTAGTAATGC
ACTGAGCGAAAACTCATTATTTAGCAGATCGGTTTTTTCTATTAACTCCCTTAACTCCCAGAAAATTTCTATCCT
CATTTTTATATTATTTACTTTTTGTAATATCGGATTGTTGGAAAACACCTCATGGCATAAAATAATGTTACTACT
AGTTTTATGAAACTTTAGATCTATAAAAATTTGTAAAATTTCTTCTTCATTCAAGGTCTCCTTGGCACCTAGCTC
TCGACAGAGGTCCCGGGTGTGCTCCGTGTTGACAGATACCAGCCCATAGTTGATGTTCGCCCCCCACTCTGCAAA
CAGTTTTATAAGGTTGTAATTGTTTTCCCTTACAGCCTTCACTAACGCCGTATTTAGGTCTAAGCCCTCTTTAAT
GCCTGCTGATTTTATGAGCTTTAGGTTATGATCAAACGTGATCGGACCATCATGCCACCATAGGTCATAACACTT
TAAAAGATAATGTTGGTTCGTGGGCACGCATTGTCCAGCCAACACCTTTTTGGTCAGAGATTGCAGGGAAGGCAA
CAT

In an embodiment of the attenuated ASFV of the invention, the expression and/or activity of the MGF 360 12L gene is disrupted. Suitably the MGF 360 12L gene comprises the sequence of SEQ ID No. 37, 38, 39, 40, 41, 42, 43, 44, 45, 46, 47 or 48. Suitably the MGF 360 12L gene comprises a sequence having at least 70%, at least 80%, at least 90% or at least 95% identity with SEQ ID No. 37, 38, 39, 40, 41, 42, 43, 44, 45, 46, 47 or 48. Suitably the MGF 360 12L gene consists of the sequence of SEQ ID No. 37, 38, 39, 40, 41, 42, 43, 44, 45, 46, 47 or 48.

MGF 360 13L gene sequences
SEQ ID No. 49-Benin 97/1 MGF 360 13L (NC_044956.1:24840-25901)
CTATAATATATTATGAGAATATTCCCAAATGATGGATAAGTTTTTTGATTTATAATCTTTTAATAAACTGCTTAT
TTTTTCGGGGTCCTTTAAGTTTAATGGCAAGGAAGCATCTGAACTGTAAATATCCAAAGTCAAGCTATGGCTTAA
AAAATTATAACCTTTTTGTTCCGCTATGGCACGACCCTCTTCAAAGGCATTACCACCCAAATCTATACAGAAAAA
TATATTACCGACGTTATAATATTGTACTGAAGTGAGCATAGCTTGGTTGATGTTGCCCCCCAGGGCGTAACAGTA
ATATATTGTTAATAGATTGTTATCCTTGGCACAAGCCAGACATATCATGTCATGGACGTCTATTTGGACTTTTTT
CTTGTGGTACATCTCATGAAGCTCATATATTTTGTTATAATACAGGAGACATTTTAATCGCCACTCGTTAAGATC
CGTATATTTCTCATCTAGAAAGCAAATGGCGTTCTTACAAACGTATTGTACTGCTTTGGCATACCAATACTTCAC
TAGTAAATCATTTAACTCGTCCGTTTCTTTTATTTCTATGAGCCCCCATAGTCTTTTATAAATTAAGCCCCTTAA
TTGTATAACAAATTTGTTTTCTAACATAGGATTATTCATAAAAATTTCATGGCACAAAATAATACTGCCGCTGGT
TTTATTGTGCATTATCCTGGTGAAAATACGGAAAATATCGTTGTCCTCTAGAGTTTCTTTGGCGCCTAGCTGTCT
ACACAACTCTCGGATGTGCTTCGTATTGATAGAAAGCAAACTATAGTTGATATTTGCGCCCCACTCTGTAAAGAG
CTTTATCAGACTATAGTTGTTTTCCTTAACAGCTATTATTAATGCCACACGAAGGTCTATATCTTCTCCTAAAAA
TCCTGATTTTATTTGTATTCGGCCACGATCCATACAAAGCTTGAGAGGAGCATCATGCCACCATAGGCCACAATA
TTTCAAAATGCAGTGTTCATCTATTGACAAACACTGGCTGGCTACCGTCTTTTTGACGAGGGTCTGCAGAGAGAG
CGGCAACGACAT
SEQ ID No. 50-China/2018/AnhuiXCGQ MGF 360 13L (MK128995.1:30597-31658)
CTATAGTATATTATGAGAATATTCCCAAATGATGGATAAGTTTTTTGATTTATAATCTTTTAATAAACTGCTTAT
TTCTTCGGGGTCCTTTAAGTTTAGTGGCAAGGAAGCATCTGAGCTGTAAATATCCAAAGCCAAACTATGGCTCAG
AAAATTATAACCTTTTTGTTCCGCTATGGCACGACCCTCTTCAAAGGCATTACCACCCAAATCTATACAGAAAAA
TATATTACCGATGTTATAATATTGTACTGAAGTAAGCATAGCTTGGTTGATGTTGCCCCCCAGCGCGTAACAGTA
ATATATTGTTAATGGATTGTTATCCTTGGTAGAAGCCAGACATATCATGTCATGGACGTCTATTTGGATGTTTTC
CTTGTGGTACATCTCATGAAGCTCATATATTTTGTTATAATACAGGAGACATTTTAATCGCCATTCATTAAGATC
CGTATATTTCTCATCTAGAAAACAAATGGCGTCCTTACAATCGTATTGTACTGCTTTGGCGTACCAATACTTCAC
TAGTAAACCATTTAACTCGTCCGTTTCTTTTATTTCTATGAGCCCCCATAGTCTTTTATAAATTAAGCCCCTTAA
TTGTATAACAAATTTGTTTTCTAAAATAGGATTATTCATAAAAATTTCATGGCACAAAATAATACTGCCGCTGGT
TTTATTGTGCATTATCCTGGTAAAAATACGGAAAATATCGTTGTCCTCTAGAGTTTCTTTGGCGCCTAGCTGTCT
ACACAACTCTCGGATGTGCTTCGTATTGATAGAAAGCAAACCATAGTTGATATTTGCGCCCCACTCTGTAAAGAG
CTTTATCAGACTATAGTTGTTTTCCTTAACAGCTATTATTAATGCCACACGAAGGTCTATATCTTCTCCTAAAAA
TCCTGATTTTATTTGTATTCGGCCACGATCCATACAAAGCTTGAGAGGAGCATCATGCCACCATAGGCCACAATA
TTTCAAAATGCAGTGTTCATCTATTGACAAACACTGGCTGGCTATCGTCTTTTTGACGAGGGTCTGCAGAGAGAG
CGGCAACGACAT
SEQ ID No. 51-Georgia 2007/1 MGF 360 13L (LR743116.1:31568-32629)
CTATAGTATATTATGAGAATATTCCCAAATGATGGATAAGTTTTTTGATTTATAATCTTTTAATAAACTGCTTAT
TTCTTCGGGGTCCTTTAAGTTTAGTGGCAAGGAAGCATCTGAGCTGTAAATATCCAAAGCCAAACTATGGCTCAG
AAAATTATAACCTTTTTGTTCCGCTATGGCACGACCCTCTTCAAAGGCATTACCACCCAAATCTATACAGAAAAA
TATATTACCGATGTTATAATATTGTACTGAAGTAAGCATAGCTTGGTTGATGTTGCCCCCCAGCGCGTAACAGTA
ATATATTGTTAATGGATTGTTATCCTTGGTAGAAGCCAGACATATCATGTCATGGACGTCTATTTGGATGTTTTC
CTTGTGGTACATCTCATGAAGCTCATATATTTTGTTATAATACAGGAGACATTTTAATCGCCATTCATTAAGATC
CGTATATTTCTCATCTAGAAAACAAATGGCGTCCTTACAATCGTATTGTACTGCTTTGGCGTACCAATACTTCAC
TAGTAAACCATTTAACTCGTCCGTTTCTTTTATTTCTATGAGCCCCCATAGTCTTTTATAAATTAAGCCCCTTAA
TTGTATAACAAATTTGTTTTCTAAAATAGGATTATTCATAAAAATTTCATGGCACAAAATAATACTGCCGCTGGT
TTTATTGTGCATTATCCTGGTAAAAATACGGAAAATATCGTTGTCCTCTAGAGTTTCTTTGGCGCCTAGCTGTCT
ACACAACTCTCGGATGTGCTTCGTATTGATAGAAAGCAAACCATAGTTGATATTTGCGCCCCACTCTGTAAAGAG
CTTTATCAGACTATAGTTGTTTTCCTTAACAGCTATTATTAATGCCACACGAAGGTCTATATCTTCTCCTAAAAA
TCCTGATTTTATTTGTATTCGGCCACGATCCATACAAAGCTTGAGAGGAGCATCATGCCACCATAGGCCACAATA
TTTCAAAATGCAGTGTTCATCTATTGACAAACACTGGCTGGCTATCGTCTTTTTGACGAGGGTCTGCAGAGAGAG
CGGCAACGACAT
SEQ ID No. 52-Ken06.Bus MGF 360 13L (NC_044946.1:28644-29704)
TTAGAGTATTTTATGAGAGTAATCCAGAATGATGGATAAATTTTTTGATTTATAATTTTTTAATAAACTGCTTAT
TTCTTCGGGATCCTTTAAGTTTAATGGCAAGGAAGCATCTGAGCTATAAATATTCAAAACCAAACTATGGCTTAA
AAAATTATAACCATTTTGTCTCGCTATGGCACTACCCTCTTCAAAGGCATTACCACCCAAATCTATACAGAAATA
TATATTACCGATGTTATAATATTGTACTGAAGTAAGCATAGCTTGATTGATGTTGCCCCCCAGGGCATAACAATA
ATATATTGTTAATAGATTGTTATCCCTGATACAAGCCAGAGATAACATTTCATTGACGTCTATTTGGATTTTTTC
CTTGTGGTATATATCATGAAGCTCATATATTTTGTTATAACATAGGTAACATTTTAATCACCATTCATCAAGATC
CGTATATTTTTCATCCAGAAAGCAAATGGCATCTTTATGGTCGTATTGTACTGCATTGGCGTACCAATATTTCAC
TAGCAATTCGCTTAACTCGTCCGTTTCTTTTATTTCTATAAGCCCGCATAGTCTTTTATAAATTAAGCGCCTTAA
TTGTGCAGCAAAGTTGTTTTCTAAATTAGGATTATTTATAAATATCTCATGGCACAAAATAATACTGCCGCTAGT
TTTATTATGCATTATCTTGTTGAAAATACAGAAAATATCGTAGTCGTCTAGAGTTTCTTTGGCGCCTAGCTGTCT
ACACAAATCTCGGGCGTGCTTCGTATTGATAGAAAGCAGACTATAGTTGATATATGCGCCCCACTCTGTAAATAA
CTTTATCATACTATAGTTGTTTTCCTTAACAGCTATTATTAATGCCACACAAAGGTCTATATCTTCTCCTAGGAA
TCCCGATTTTATGTATATTCGCCCACGATCTATGTAAAGCTTGAGGGGAGCATCATGCCACCATAAGCCACAGTA
TTTCAAAATGTGTTCATCTATCGACAAACATGATGTACCGGCTACCGTCTTTTTGACGAGGGTCTGCAGAGAGAG
CGGCGACGACAT
SEQ ID No. 53-Kenya 1950 MGF 360 13L (AY261360.1:34752-35812)
TTAGAGTATTTTATGAGAGTAATCCAGAATGATGGATAAATTTTTTGATTTATAATTTTTTAATAAACTGCTTAT
TTCTTCGGGAACCTTTAAGTTTAATGGCAAGGAAGCATCTGAGCTATAAATATTCAAAATTAAACTATGGCATAA
AAAATTATAACCATTTTGTCTCGCTATGGCACTACCCTCTTCAAAGGCATTACCACCCAAATCTATACAGAAATA
TATATTACCGATGTTATAATATTGTACTGAAGTAAGCATAGCTTGGTTGATGTTGCCCCCCAGGGCATAACAATA
ATATATTGTTAATAGATTGTTATCCCTGATACAAGCCAGAGATAACATTTCATTGACGTCTATTTGGATTTTTTT
CTTGTGGTATATATCATGAAGCTCATATATTTTGTTATAACATAGGTAACATTTTAATCGCCATTCATCAAGATC
CGTATATTTTTCATCCAGAAAGCAAATGGCATCTTTATGATCGTATTGTACTGCATTGGCGTACCAATATTTCAC
TAGTAATTCGCTTAACTCGTCCGTTTCTTTTATTTCTATAAGCCCGCATAATCTTTTATAAATTAAGCGCCTTAA
TTGTGCAGCAAAGTTGTTTTCTAATTTAGGATTATTTATAAATATCTCATGGCACAAAATAATACTGCCGCTAGT
TTTATTATGCATTATTTTATTGAAAATACAGAAAATATCGTAGTCGTCTAGAGTTTCTTTGGCGCCTAGCTGTCT
ACACAAATCTCGGGCGTGCTTCGTATTGATAGAAAGCAGACTATAGTTGATATATGCGCCCCACTCTGTAAATAA
CTTTATCAGACTATAGTTATTTTCCTTAACAGCTATTATTAATGCCACACAAAGGTCTATATCTTCTCCTAGGAA
TCCCGATTTTATGTATATTCGTCCACGATCTATGTAAAGCTTGAGGGGAGCATCATGCCACCATAAGCCACAGTA
TTTCAAAATGTGTTCATCTATCGACAAACATGATGTACTGGCTACCGTCTTTTTGACGAGGGTCTGCAGAGAGAG
CGGCGACGACAT
SEQ ID No. 54-L60 MGF 360 13L (NC_044941.1:25189-26250)
CTATAATATATTATGAGAATATTCCCAAATGATGGATAAGTTTTTTGATTTATAATCTTTTAATAAACTGCTTAT
TTTTTCGGGGTCCTTTAAGTTTAATGGCAAGGAAGCATCTGAACTGTAAATATCCAAAGTCAAGCTATGGCTTAA
AAAATTATAACCTTTTTGTTCCGCTATGGCACGACCCTCTTCAAAGGCATTACCACCCAAATCTATACAGAAAAA
TATATTACCGACGTTATAATATTGTACTGAAGTGAGCATAGCTTGGTTGATGTTGCCCCCCAGGGCGTAACAGTA
ATATATTGTTAATAGATTGTTATCCTTGGCACAAGCCAGACATATCATGTCATGGACGTCTATTTGGACTTTTTT
CTTGTGGTACATCTCATGAAGCTCATATATTTTGTTATAATACAGGAGACATTTTAATCGCCACTCGTTAAGATC
CGTATATTTCTCATCTAGAAAGCAAATGGCGTTCTTACAAACGTATTGTACTGCTTTGGCATACCAATACTTCAC
TAGTAAATCATTTAACTCGTCCGTTTCTTTTATTTCTATGAGCCCCCATAGTCTTTTATAAATTAAGCCCCTTAA
TTGTATAACAAATTTGTTTTCTAACATAGGATTATTCATAAAAATTTCATGGCACAAAATAATACTGCCGCTGGT
TTTATTGTGCATTATCCTGGTGAAAATACGGAAAATATCGTTGTCCTCTAGAGTTTCTTTGGCGCCTAGCTGTCT
ACACAACTCTCGGATGTGCTTCGTATTGATAGAAAGCAAACTATAGTTGATATTTGCGCCCCACTCTGTAAAGAG
CTTTATCAGACTATAGTTGTTTTCCTTAACAGCTATTATTAATGCCACACGAAGGTCTATATCTTCTCCTAAAAA
TCCTGATTTTATTTGTATTCGGCCACGATCCATACAAAGCTTGAGAGGAGCATCATGCCACCATAGGCCACAGTA
TTTCAAAATGCAGTGTTCATCTATTGACAAACACTGGCTGGCTACCGTCTTTTTGACGAGGGTCTGCAGAGAGAG
CGGCAACGACAT
SEQ ID No. 55-Malawi Lil-20/1 (1983) MGF 360 13L (AY261361.1:28920-29980)
TTAGAGTATATTATGAGAGTAATCCAAAATGATGGATAAATTTTTTGATTTATAATTTTTTAACAAACTGCTTAT
TTCTTCGGGGTCCTTTAAGTTTAATGGCAAGGAAGCATCCGAGCTATAAATATTCAAAACCAAACTATGGCTTAG
AAAATTATAACCTTTTTGTCTCGCTATGGCACTACCCTCTTCAAAGGCATTACCACCCAAATCTATACAGAAATA
TATATTACCGATGTTATAATATTGCACTGAAGTAAGCATAGCTTGGTTGATGTTGCCCCCCAGGGCATAACAGTA
ATATATTGTTAATGGATTGTTATCCCTGATACAAGCCAGGGATAACATTTCATTAACGTCTATTTGGATTTTTTC
CTTGTGGTATATATCATGAAGCTCATATATTTTGTTATAATATAGGTAACATTTTAATCGCCATTCATTAAGATC
CGTATATTTCTCATCCAGAAAGCAAATGGCATCTTTATAATCGTATTGTACTGCTTTGGCGTACCAATATTTCAC
TAGTAATTCACTTAACTCGTCCGTTTCTTTTATTTCTATAAGCCCGCATAGTCTTTTATAAATTAAGCGCCTTAA
TTGTGCAGCAAATTTGTTTTCTAAATTAGGATTATTTATAAATATCTCATGGCACAAAATAACACTGCCGCTGGT
TTTATTATGCATTATCTTGTTGAAAATACAGAAAATATCGTAGTCCTCCAGAGTTTCTTTGGCGCCTAGCTGTCT
ACACAAATCTCGGACGTGCTTCGTATTGATAGAAAGCAGACTATAGTTGATATTTGCGCCCAACTCTGTAAAAAG
CTTTATTAGATTATAGTTGTTTTCCTTAACAGCTATTATTAATGCCACACAAAGGTCTATATCTTCTCCTAAAAA
TCCCGATTTTATTTGTATTCGGCCACGATCCATATAAAGCTTGAGAGGGGCATCATGCCACCATAAGTCACAGTA
TTTCAAAATGTGTTCATCTATCGACAAACATGATGTACTGGCTACCGTCTTTTTGACGAGGGTTTGTAGAGAGAG
CGGCGCCGACAT
SEQ ID No. 56-Mkuzi 1979 MGF 360 13L (AY261362.1:32286-33347)
CTATAATATATTATGAGAATATTCCCAAATGATGGATAAGTTTTTTGATTTATAATCTTTTAATAAACTGCTTAT
TTCTTCGGGGTCCTTTAAGTTTAATGGCAAGGAAGCATCTGAACTGTAAATATCCAAAGTCAAGCTATGGCTTAA
AAAATTATAACCTTTTTGTTCCGCTATGGCACGACCCTCTTCAAAGGCATTACCCCCCAAATCTATACAGAAAAA
TATATTACCGATGTTATAATATTGTACTGAAGTGAGCATAGCTTGGTTGATGTTGCTCCCCAGGGCGTAACAGTA
ATATATTGTTAATAGATTGTTATCCTTGGCACAAGCCAGACATATCATGTCATGGACGTCTATTTGGACTTTTTT
CTTGTGGTACATCTCATGAAGCTCATATATTTTGTTATAATACAGGAGACATTTTAATCGCCATTCGTTAAGATC
CGTATATTTCTCATCTAGAAAGCAAATGGCGTTCTTACAAACGTATTGTACTGCTTTGGCATACCAATACTTCAC
TAGTAAATCATTTAACTTGTCCGTTTCTTTTATTTCTATGAGCCCCCATAGTCTTTTATAAATTAAGCCCCTTAA
TTGTATAACAAATTTGTTTTCTAAAATAGGATTATTCATAAAAATTTCATGGCACAAAATAATACTGCCGCTGGT
TTTATTGTGCATTATCCTGGTGAAAATACGGAAAATATCGTTGTCCTCTAGAGTTTCTTTGGCGCCTAGCTGTCT
ACACAACTCTCGGATGTGCTTCGTATTGATAGAAAGCAAACTATAGTTGATATTTGCGCCCCACTCTGTAAAGAG
CTTTATCAGACTATAGTTGTTTTCCTTAACAGCTATTATTAATGCCACACGAAGGTCTATATCTTCTCCTAAAAA
TCCTGATTTTATTTGTATTCGGCCACGATCCATACAAAGCTTGAGAGGAGCATCATGCCACCATAGGCCACAGTA
TTTCAAAATGCAGTGTTCATCTATTGACAAACACTGGCTGGCTACCGTCTTTTTGACGAGGGTCTGCAGAGAGAG
CGGCAACGACAT
SEQ ID No. 57-Pretorisuskop/96/4 MGF 360 13L (AY261363.1:31307-32368)
CTATAGTATATTATGAGAATATTCCCAAATTATGGATAAGTTTTTTGATTTATAATCCTTTAAAAAACTGCTTAT
TTCTTCGGGGTCCTTTAAGTTTAGTGGCAAGGAAGCATCTGAGCTGAAAATATCCAAAGCCAAACTATGGCTTAG
AAAATTATAACCTTTTTGTTCCGCTATGGCACGACCCTCTTCAAAGGCATTACCACCCAAATCTATACAGAAAAA
TATATTACCGATGTTATAATATTGTATTGAAGTAAGCATAGCTTGGTTGATGTTGCCCCCCAGGGCGTAACAGTA
ATATATTGTTAATGGATTGTTATCCTTGGTAGAAGCCAGACATATCATGTCATGGACGTCTATTTGGACTTTTTC
CTTGTGGTACATCTCATGAAGCTCATATATTTTGTTAAAATACAGGAGACATTTTAATCGCCATTCATTAAGATC
CGTATATTTCTCTTCTAGAAAGCAAATGGCGTCCTTACACTCGTATTGTACTGCTTTGGCGTACCAATACTTCAC
TAGTAAATCGTTTAACTCATCCGTTTCTTTTATTTCTATGAGCCCCCATAGTCTTTTACAAATTAAGCCCCTTAA
TTGTATAGTAAATTTGTTTTCTATATTAGGATTATTCATAAATATATCATGGCACAAAATAATACGGCCGCTGGT
TTTATTGTGCATTATCTTGGTGAAAATACGGAAAACATCGTCGTCTTCTAGAGTTTCTTTGGCGCCTAGCTGTCT
ACACAACTCTCGGATGTGCTCCGTATTAATAGAAAGCAAACTATAGTTGATATGTGCGCCCCATTCTGTAAAGAG
CTTTATCAGACTATAGTTGTTTTCCTTAACAGCTATTATTAATGCTATATGGAGGTCTATATCCTCTCCTAAAAA
TCCTGATTTTATTTGTATTTGGCCACGATCCATACAAAGCTTGAGGGGGACATCATGCCACCATAGGCCACAGTG
TTTCAGAATGCAGTGTTCATCTATCGACAAACACTGGCTGGCTACCGTCTTTTTGACGAGGGTCTGCAGAGAGAG
CGGCAACGACAT
SEQ ID No. 58-Tengani 62 MGF 360 13L (AY261364.1:25753-26814)
CTATAGTATATTATGAGAATATTCCCAAATGATGGATAAGTTTTTTGATTTATAATCTTTTAATAAACTGCTTAT
TTCTTCGGGGTCCTTTAAGTTTAGTGGCAAGGAAGCATCTGAGCTGTAAATATCCAAAGCCAAACTATGGCTCAG
AAAATTATAACCTTTTTGTTCCGCTATGGCACGACCCTCTTCAAAGGCATTACCACCCAAATCTATACAGAAAAA
TATATTACCGATGTTATAATATTGTACTGAAGTAAGCATAGCTTGGTTGATGTTGCCCCCCAGCGCGTAACAGTA
ATATATTGTTAATGGGTTGTTATCCTTGGTAGAAGCCAGACATATCATGTCATGGACGTCTATTTGGATGTTTTC
CTTGTGGTACATCTCATGAAGCTCATATATTTTGTTATAATACAGGAGACATTTTAATCGCCATTCATTAAGATC
CGTATATTTCTCATCTAGAAAGCAAATGGCGTCCTTACACTCGTATTGTACTGCTTTGGCGTACCAATACTTCAC
TAGTAAACCGTTTAACTCATCCGTTTCTTTTATTTCTATGAGCCCCCATAGTCTTTTATAAATTAAGCCCCTTAA
TTGTATAGTAAATTTATCTTCTATATTAGGATTATTCATAAATATATCATGGCACAAAATAATACTGCCGCTGGT
TTTATTGTGCATTATCTTGGTGAAAATACGGAAAATATCGTCGTCTTCTAGAGTTTCTTTGGCGCCTAGCTGTCT
ACACAACTCTCGGATGTGCTTCGTATTGATAGAAAGCAAACTATAGTTGATATTTGCGCCCCACTCTGTAAAGAG
CTTTATCAGACTATAGTTGTTTTCCTTAACAGCTATTATTAATGCTATATGGAGGTCTATATCTTCTCCTAAAAA
TCCTGATTTTATTTGTATTTGGCCACGATCCATACAAAGCTTGAGGGGGACATCATGCCACCATAGGCCACAGTG
TTTCAGAATGCAGTGTTCATCTATCGACAAAGACTGGTTGGCTACCGTCTTTTTGACGAGGGTCTGCAGAGAGAG
CGGTAATGACAT
SEQ ID No. 59-Warmbaths MGF 360 13L (AY261365.1:30498-31559)
CTATAGTATATTATGAGAATATTCCCAAATGATGGATAAGTTTTTTGATTTATAACCCTTTAATAAACTGCTTAT
TTCTTCGGGGTCCTTTAAGTTTAGTGGCAAGGAAGCATCTGAGCTGTAAATATCCAAAGCCAAACTATGGCTTAG
AAAATGATAACCTTTTTGTTCCGCTATGGCACGACCCTCTTCAAAGGCATTACCACCCAAATCTATACAGAAAAA
TATATTACCGATGTTATAATATTGTACTGAAGTAAGCATAGCTTGGTTGATGTTGCCCCCCAGGGCGTAACAGTA
ATATATTGTTAATGGATTGTTATCCTTGGTAGAAGCCAGACATATCATGTCATGGACGTCTATTTGGACTTTATC
CTTGTGGTACATCTCATGAAGCTCATATATTTTGTTATAATACAGGAGACATTTTAATCGCCATTCATTAAGACC
CGTATATTTCTCATCTAGAAAGCAAATGGCGTCCTTACACTCGTATTGTACTGCTTTGGCGTACCAATACTTCAC
TAGTAAATCGTTTAACTCATCCGTTTCTTTTATTTCTATGAGCCTCCATAGTCTTTTACAAATTAAGCCCCTTAA
TTGTATAGTAAATTTGTTTTCTATATTAGGATTATTCATAAATATCTCATGGCACAAAATAATACGGCCGCTGGT
TTTATTGTGCATTATCTTGGTGAAAATACGGAAAATATCGTTGTCCTCTAGAGTTTTTTTGGCACCTAGCTGTCT
ACACAACTCTCGGATGTGTTCCGTATTGATAGAAAGCAAACTATAGTTGATATGTGCGCCCCATTCTGTAAAGAG
CTTTATCAGACTATAGTTGTTTTCCTTAACAGCTATTATTAATGCTACATGGAGATCTATATCTTCTCCTAAAAA
TCCTGATTTTATTTGTATTTGGCCACGATCCATACAAAGCTTGAGGGGGACATCATGCCACCATAGGCCACAGTG
TTTCAGAATGCAGTGTTCATCTGTCGACAAACACTGGCTGGCTACCGTTTTTTTGACGAGGGTCTGCAGAGAGAG
CGGCAACGACAT
SEQ ID No. 60-Warthog MGF 360 13L (AY261366.1:27277-28338)
CTATAGTATATTATGAGAATATTCCCAAATGATGGATAAGTTTTTTGATTTATAATCCTTTAATAAACTGCTTAT
TTCTTCGGGGTCCTTTAAGTTTAGTGGCAAGGAAGCATCTGAGCTGTAAATATCCAAAGTCAAACTATGGCTTAG
AAAATGATAACCTTTTTGTTCCGCTATAGCACGACCCTCTTCAAAGGCATTACCACCCAAATCTATACAGAAAAA
TATATTACCGATGTTATAATATTGTACTGAAGTAAGCATAGCTTGGTTGATGTTGCCCCCCAGGGCGTAACAATA
ATATATTGTTAATGGATTGTTATCCTTGGTAGAAGCCAGACATATCATGTCATGGACGTCTATTTGGACTTTTTC
CTTGTGGTACATCTCATGAAGCTCATATATTTTGTTATAATACAGGAGACATTTTAATCGCCATTCATTAAGATC
CGTATATTTCTCATCTAGAAAGCAAATGGCGTCCTTACACTCGTATTGTACTGCTTTGGCGTACCAATACTTCAC
TAGTAAATCGTTTAACTCATCCGTTTCTTTTATTTCTATGAGCCCCCATAGTCTTTTACAAATTAAGCCCCTTAA
TTGTATAGTAAATTTGTTTTCTATATTAGGATTATTCATAAATATCTCATGGCACAAAATAATACGGCCGCTGGT
TTTATTGTGCATTATCTTGGTGAAAATACGGAAAATATCGTTGTCCTCTAGAGTTTCTTTGGCACCTAGCTGTCT
ACACAACTCTCGGATGTGCTCCGTATTGATAGAAAGCAAACTATAGTTGATATGTGCGCCCCATTCTGTAAAGAG
CTTTATCAGACTATAGTTGTTTTCCTTAACAGCTATTATTAATGCTATATGGAGGTCTATATCTTCTCCTAAAAA
TCCTGATTTTATTTGTATTTGGCCACGATCCATACAAAGCTTGAGGGGGACATCATGCCACCATAGGCCACAGTG
TTTCAGAATGCAGTGTTCATCTGTCGACAAACACTGGCTGGCTACCGTCTTTTTGACGAGGGTCTGCAGAGAGAG
CGGCAACGACAT

In an embodiment of the attenuated ASFV of the invention, the expression and/or activity of 50 the MGF 360 13L gene is disrupted. Suitably the MGF 360 13L gene comprises the sequence of SEQ ID No. 49, 50, 51, 52, 53, 54, 55, 56, 57, 58, 59 or 60. Suitably the MGF 360 13L gene comprises a sequence having at least 70%, at least 80%, at least 90% or at least 95% identity with SEQ ID No. 49, 50, 51, 52, 53, 54, 55, 56, 57, 58, 59 or 60. Suitably the MGF 360 13L gene consists of the sequence of SEQ ID No. 49, 50, 51, 52, 53, 54, 55, 56, 57, 58, 59 or 60.

MGF 360 14L gene sequences
SEQ ID No. 61-Benin 97/1 MGF 360 14L (NC_044956.1:26073-27146)
TTAGTCTATAACTATAATTTCTGGATGGGCTGTAAGATACTCTTCGGCTCGTTTCATATTTTTTGAAGTATATGT
CTTTAGCATATCATATATTTCCTGGGGTTCGGTTACATCTAATACCAAGGTCACATCACGGCTGAAAAGCTGCTT
TACTAAGAAAATGTTGCTCAAGTTATACATATAAGCTTTGTGCGCAATGAGTTGTGCCCTATCAAAATCGGCGGC
CCCCAAATCAATACAGAAAAACATGTTTAAAGTATTATTGTTATAGATAGAAAGATTCATGCCATAATCGAGACT
AGCCCCCAACCTATGACAGTAATAAATGGCCGCGTAATTTTTTTCCCGCAAGCAAGCAAATTTCATCATCAGATT
AGGGCTAATGCAAATCTCTTTTTCACGACACAGCTCGTGTATGTCAAAAATGTTATTAAAATAAAGGCTACAAGC
TACCCGCCAATAGAGCTGATTTTTATGCCTTTTATAAAAATAGTGGATAGCCTTTGTAAAATTATGTCGTAATGC
CAGGGCAAACCAAAACTTTGTTAATAGGAGGTGCGCCGTATCCCCCGTCAACGGAATGTTTGAACAGGTGTACGT
AACTGTGTCTAAAGTGGTTCTAGTTACGGTTTCCAAGAGTGGATTATGACAAAACATGTCATAACCCAGTAGAAC
TCCTGCACAGGATTTTAGGTTGGCCACTTCTTTTAAAATTTCCAGAAGACGGGGTTCGGATACAGGCGTCAAGCC
TCCCAGTTCCGCACACAGCCGCTTTAGATACACGGCAGGAACACGTATAAGCCCATATTCAGGATTTGCGCCCCA
ATCCACAAATAAACGTATAAGTTCAAGATTATCGCTCTTCACGGCCTTTACTAGCGCCGCTTCAAGACAAAGATC
ATCCTCAGAAAAACACTGTAAATGTTTATACGAAAAAACTTGCTTACAATTGTTACATAGGTGAATAGGACCTAA
ATCCCACCACAAACCAAAACGCTGCAACGTATAATCATAGTCACTTGAAAGATAATTGCATGCCACAACTTTTTT
GGCCAACGTTTGTAAAGACAACAT
SEQ ID No. 62-China/2018/AnhuiXCGQ MGF 360 14L (MK128995.1:31843-32916)
TTAGTCTATAACTATAATTTCTGGATGGGCTGTAAGATACTCTTCGGCTCGTTTCAGATTTTTTGAAGTATATGT
CTTTAGCATATCATATATTTCCTGGGGTTCGGTTACATCTAATACCAAGGTCACATCACGGCTGAAAAGCTGCTT
TACTAAGAAAATGTTGCTCAAGTTATACATATAAGCTTTGTGCGCAATGAGTTGTGCCCTATCAAAATCGGCAGC
CCCCAAATCAATACAGAAAAACATGTTTAAAGTATTATTGTTATAGATAGAAAGATTCATGCCATAATCGAGACT
AGCCCCCAACCTATGACAGTAATAAATGGCCGCGTAATTTTTTTCCCGCAAGCAAGCAAATTTCATCATCAGATT
AGGGCTGATGCAAATCTCTTTTTCACGACACAACTCGTGTATGTCAAAAATGTTATTAAAATAAAGGCTACAAGC
TACCCGCCAATAGAGGTGATTTTTATGCCTTTTATAGAAATAGTGAATAGCCTTTGTAAAATTATGTCGTAATGC
CAGGGCAAACCAAAACTTTGTTAATAGGTGGTGCGCCGTATCCCCCGTCAACGGAATGTTTGAACAGGTGTACGT
AACTGTGTCTAAAGTGGTTCTAGTTACGGTTTCCAAGAGTGGATTATGACAAAACATGTCATAACCCAGCAGAAC
TCCTGCACAGGATTTTAGCCTGGCCACTTCTTTTAAAATTTCCAGAAGACGGGGTTCGGATACAGGCGTTAAGCC
TCCCAGTTCCGCACACAGCCGCTTTAGATACACGGCAGGAACACGTATAAGCCCATATTCAGGATTTGCGCCCCA
ATCCACAAATAAACGTATAAGTTCAAGATTATCGCTCTTCACGGCCTTTACTAGCGCCGCTTCGAGACAAAGATC
ATCCTCAGAAAAACACTGTAAATGTTTATACGAAAAAACTTGCTTACAATTGTTACATAGGTGAATAGGACCTAA
ATCCCACCACAAACCAAAACGCTGCAACGTATAATCATAGTCACTTGAAAGATAATTGCATGCCACAACTTTTTT
GGCCAACGTTTGTAAAGACAACAT
SEQ ID No. 63-Georgia 2007/1 MGF 360 14L (LR743116.1:32814-33887)
TTAGTCTATAACTATAATTTCTGGATGGGCTGTAAGATACTCTTCGGCTCGTTTCAGATTTTTTGAAGTATATGT
CTTTAGCATATCATATATTTCCTGGGGTTCGGTTACATCTAATACCAAGGTCACATCACGGCTGAAAAGCTGCTT
TACTAAGAAAATGTTGCTCAAGTTATACATATAAGCTTTGTGCGCAATGAGTTGTGCCCTATCAAAATCGGCAGC
CCCCAAATCAATACAGAAAAACATGTTTAAAGTATTATTGTTATAGATAGAAAGATTCATGCCATAATCGAGACT
AGCCCCCAACCTATGACAGTAATAAATGGCCGCGTAATTTTTTTCCCGCAAGCAAGCAAATTTCATCATCAGATT
AGGGCTGATGCAAATCTCTTTTTCACGACACAACTCGTGTATGTCAAAAATGTTATTAAAATAAAGGCTACAAGC
TACCCGCCAATAGAGGTGATTTTTATGCCTTTTATAGAAATAGTGAATAGCCTTTGTAAAATTATGTCGTAATGC
CAGGGCAAACCAAAACTTTGTTAATAGGTGGTGCGCCGTATCCCCCGTCAACGGAATGTTTGAACAGGTGTACGT
AACTGTGTCTAAAGTGGTTCTAGTTACGGTTTCCAAGAGTGGATTATGACAAAACATGTCATAACCCAGCAGAAC
TCCTGCACAGGATTTTAGCCTGGCCACTTCTTTTAAAATTTCCAGAAGACGGGGTTCGGATACAGGCGTTAAGCC
TCCCAGTTCCGCACACAGCCGCTTTAGATACACGGCAGGAACACGTATAAGCCCATATTCAGGATTTGCGCCCCA
ATCCACAAATAAACGTATAAGTTCAAGATTATCGCTCTTCACGGCCTTTACTAGCGCCGCTTCGAGACAAAGATC
ATCCTCAGAAAAACACTGTAAATGTTTATACGAAAAAACTTGCTTACAATTGTTACATAGGTGAATAGGACCTAA
ATCCCACCACAAACCAAAACGCTGCAACGTATAATCATAGTCACTTGAAAGATAATTGCATGCCACAACTTTTTT
GGCCAACGTTTGTAAAGACAACAT
SEQ ID No. 64- Ken06.Bus MGF 360 14L (NC_044946.1:29917-30990)
TTAGTCTATAACGACAATTTCTGGATGGGCTGTAAAATACTCTTCGGCTCGTTTTAGATTTTTTGACGTATATGT
CTTTAGCATATCATATATTGCCTGGGGTTCGGTTATATTTAATACCAAGCTCGCATCACGGCTGAAAAGCTGTTT
TACTAAAAAAATGTTGCTCAAGTTATACATATAAACTCTATGCGCAATGAGTCGCGCTCTATCAAAGTTAGTAGC
CCCCAAATCAATGCAGAAAAATAGGTTTAAAGTATTATTGTTATAGATAGATAGATTCATGCCATAATCGGGACT
AGCCCCCAACATATGACAGTAATAAATGGCTGCATAATTTTCTTCCCGCAGGCAAGCAAATTTCATCATTAGATT
AGGGCTAATGCAAATCTCTTTTTTAGAACACAGCTCATGCAAGTCAAAAATGTTATTAAAATAAAGGCTACAAGT
TAGCCGCCAATACAGCTGATTTTTATGTTTTTCGTAGAAATAGTGAATCGCTTTTGTAAAATTATGCCGTAATGC
CAGGGCAAACCAAAACTTCGTTAATAGGAGGTGCGCCGTATCCCCCGTCAACGGAATTTTTGAACGGGTATACAT
AACTGTGTTTAAAGTGGTTCTAGTCACGGTTTCCATAAGTGGATTATGACAAAACATGTCATAACCCAGTAGAAC
TCCTGCACAGGATTTCAGATCGGCCACTTCTTTTAAAATTTCCAGAAGACGAGATTCGGAGACAGGTGTTAAGCC
TCCTAGTTCCGTGCACAGCCGTTTTAGATGCACGGCAGGAACACGTATAAGTCCATATTCAGGATTTGCGCCCCA
ATCCACAAACAATCGTATAAGCTCAAGATTATCGCTCTTCACGGCCTTTACTAGCGCCGCGTCAAGACAAAGATC
ATCCACAGAAAAACACTGCAAATGTTGATACGAAAAAATTTGCTTACATGTATTACATAAGTGAATAGGACCTAA
ATCCCACCACAAACCAAAACGCTGCAGCGTATAATCATAGTCACTTGAAAGATAATTGCACGCCACAACTTTTTT
GGCCAACGTTTGTAAAGACAACAT
SEQ ID No. 65-Kenya 1950 MGF 360 14L (AY261360.1:36121-37194)
TTAGTCTATAACTACAATTTCTGGATGGGCTGTAAAATACTCTTCGGCTCGTTTTAGATTTTTTGACGTATATGT
CTTTAGCATATCATATATTGCCTGGGGTTCGGTTATATTTAATACCAAGCTCGCATCACGGCTGAAAAGCTGTTT
TACTAAAAAAATGTTGCTCAAGTTATACATATAAACTCTATGCGCAATGAGTCGCGCTCTATCAAAGTTAGTAGC
CCCCAAATCAATACAGAAAAATAGGTTTAAAGTATTATTGTTATAGATAGATAGATTCATGCCATAATCGGGACT
AGCCCCCAACATATGACAGTAATAAATGGCTGCATAATTTTCTTCCCGCAGGCAAGCAAATTTCATCATTAGATT
AGGGCTAATGCAAATCTCTTTTTTAGAACACAGCTCATGCAAGTCAAAAATATTATTAAAATAAAGGCTACAAGC
CAGCCGCCAATAGAGCTGATTTTTATGCTTTTCATAGAAATAGTGAATCGCTTTTGTAAAATTATGTCGTAATGC
CAGGGCAAACCAAAACTTCGTTAATAGGAGGTGCGCCGTATCCCCCGTCAACGGAATTTTTGAACGGGTGTACAT
AACGGTGTTTAAAGTGGTTCTAGTCACGGTTTCCAAGAGTGGATTATGACAAAACATGTCATAACCCAGTAGAAC
TCCTGCACAGGATTTCAGATCGGCCACTTCTTTTAAAATTTCCAGAAGACGAGATTCGGAGACAGGTGTTAAGCC
TCCTAGTTCTGTGCACAGCCGTTTTAGATCCACGGCAGGAACACGTATAAGTCCATATTCAGGATTTGCGCCCCA
ATCCACAAACAATCGTATAAGCTCAAGATTATCGCTCTTCACGGCCTTTACTAGCGCCGCGTCAAGACAAAGATC
ATCCTCAGAAAAACACTGCAAATGTTGATACGAAAAAATTTGCTTACATGTATTACATAAGTGAATAGGACCTAA
ATCCCACCACAAACCAAAACGCTGCAGCGTATAATCATAGTCACTTGAAAGATAATTGCATGCCACAACCTTTTT
GGCCAACGTTTGTAAAGACAACAT
SEQ ID No. 66-L60 MGF 360 14L (NC_044941.1:26422-27495)
TTAGTCTATAACTATAATTTCTGGATGGGCTGTAAGATACTCTTCGGCTCGTTTCATATTTTTTGAAGTATATGT
CTTTAGCATATCATATATTTCCTGGGGTTCGGTTACATCTAATACCAAGGTCACATCACGGCTGAAAAGCTGCTT
TACTAAGAAAATGTTGCTCAAGTTATACATATAAGCTTTGTGCGCAATGAGTTGTGCCCTATCAAAATCGGCGGC
CCCCAAATCAATACAGAAAAACATGTTTAAAGTATTATTGTTATAGATAGAAAGATTCATGCCATAATCGAGACT
AGCCCCCAACCTATGACAGTAATAAATGGCCGCGTAATTTTTTTCCCGCAAGCAAGCAAATTTCATCATCAGATT
AGGGCTAATGCAAATCTCTTTTTCACGACACAGCTCGTGTATGTCAAAAATGTTATTAAAATAAAGGCTACAAGC
TACCCGCCAATAGAGCTGATTTTTATGCCTTTTATAAAAATAGTGGATAGCCTTTGTAAAATTATGTCGTAATGC
CAGGGCAAACCAAAACTTTGTTAATAGGAGGTGCGCCGTATCCCCCGTCAACGGAATGTTTGAACAGGTGTACGT
AACTGTGTCTAAAGTGGTTCTAGTTACGGTTTCCAAGAGTGGATTATGACAAAACATGTCATAACCCAGTAGAAC
TCCTGCACAGGATTTTAGGTTGGCCACTTCTTTTAAAATTTCCAGAAGACGGGGTTCGGATACAGGCGTCAAGCC
TCCCAGTTCCGCACACAGCCGCTTTAGATACACGGCAGGAACACGTATAAGCCCATATTCAGGATTTGCGCCCCA
ATCCACAAATAAACGTATAAGTTCAAGATTATCGCTCTTCACGGCCTTTACTAGCGCCGCTTCAAGACAAAGATC
ATCCTCAGAAAAACACTGTAAATGTTTATACGAAAAAACTTGCTTACAATTGTTACATAGGTGAATAGGACCTAA
ATCCCACCACAAACCAAAACGCTGCAACGTATAATCATAGTCACTTGAAAGATAATTGCATGCCACAACTTTTTT
GGCCAACGTTTGTAAAGACAACAT
SEQ ID No. 67-Malawi Lil-20/1 (1983) MGF 360 14L (AY261361.1:30193-31266)
TTAGTCTATAACTACAATTTCTGGATGGGCTGTAAAATACTCTTCGGCTTGTTTTAGATTTTTTGACGTATATGT
ATTTAGCATATCATATATTGCCTGGGGCTCAGTTAGATCTAATATCAAGCTCACATCACGGCTGAAAAGCTGTTT
TACTAAAAAAAGGTTGCTCAAGTTATACATATAAACTCTACGCGCAATGAGTCGCGCCCTATCAAAGTTAGTAGC
CCCCAAATCAATGCAGAAAAATAGGTTTAAAGTATTATTGTTATAGATAGATAGATTCATGCCATAATCAAGACT
AGCCCCTAACATATGACAGTAATAAATGGCTGCATAATTTTCTTCCCGCAAGCAAGCAAATTTCATCATCAGATT
AGGGCTAATGCAAATCTCTTTTTTACAACACAGCTCATGCAAGTCAAAAATGTTGTTAAAATAAAGGCTACAAGT
CAGTCGCCAATAGAGCTGATTTTTATGCTTTTCGTAGAAATAGTGAATAGCTTTTGTAAAATTATGTCGTAATGC
CAGGGCAAACCAAAACTTTGATAATAGGAGGTGCGCCGTATCCCCCGTCAACGGAATTTTTGAACGGGTGTACAT
AACTGTGTCTAAGGTGGTTCTAGTCACGGTTTCCAAGAGTGGATTATGACAAAACATGTCATAACCCAGTAGAAC
TCCTGCACAGGATTTCAGATCGGCCACTTCTTTTAAAATCTCCAGAAGACGAGATTCGGATACAGGTGTTAAGCC
TCCTAGTTCCATGCACAGCCGCTTTAGATGCACGGCAGGAACACGTATAAGTCCATATTCAGGATTTGCGCCCCA
ATCCACAAACAATCGTATAAGCTCAAGATTATCGCTCTTCACGGCCTTTACTAGCGCCGCTTCAAGACAAAGATC
ATCCTCAGAAAAACACTGTAAATGTTTATATGAAAAAATTTGCTTACATGTATTACATAAGTGAATAGGACCTAA
ATCCCACCACAAACCAAAACGCTGCAGCGTATAATCATAATCACTTGAAAGATAATTGCATGCCACAACTTTTTT
GGCCAGCGTTTGTAAAGACAACAT
SEQ ID No. 68-Mkuzi 1979 MGF 360 14L (AY261362.1:33547-34620)
TTAGTCTATAACTATAATTTCTGGATGGGCTGTAAGATACTCTTCGGCCCGTTTCATATTTTTTGAAGTATATGT
CTTTAGCATATCATATATTTCCTGGGGTTCGGTTACATCTAATACCAAGGTCACATCACGGCTGAAAAGCTGCTT
TACTAAGAAAATGTTGCTCAAGTTATACATATAAGCTTTGTGCGCAATGAGTTGTGCCCTATCAAAATCGGCAGC
CCCCAAATCAATACAGAAAAACATGTTTAAAGTATTATTGTTATAGATAGAAAGATTCATGCCATAATCGAGACT
AGCCCCCAACCTATGACAGTAATAAATGGCCGCGTAATTTTTTTCCCGCAAGCAAGCAAATTTCATCATCAGATT
AGGGCTAATGCAAATCTCTTTTTCACGACACAGCTCGTGTATGTCAAAAATGTTATTAAAATAAAGGCTACAAGC
TACCCGCCAATAGAGCTGATTTTTATGCCTTTTATAAAAATAGTGGATAGCCTTTGTAAAATTATGTCGTAATGC
CAGGGCAAACCAAAACTTTGTTAATAGGAGGTGCGCCGTATCCCCCGTCAACGGAATGTTTGAACAGGTGTACGT
AACTGTGTCTAAAGTGGTTCTAGTTACGGTTTCCAAGAGTGGATTATGACAAAACATGTCATAACCCAGTAGAAC
TCCTGCACAGGATTTTAGGTTGGCCACTTCTTTTAAAATTTCCAGAAGACGGGGTTCGGATACAGGCGTTAAGCC
TCCCAGTTCCGCACACAGCCGCTTTAGATACACGGCAGGAACACGTATAAGCCCATATTCAGGATTTGCGCCCCA
ATCCACAAATAAACGTATAAGTTCAAGATTATCGCTCTTCACGGCCTTTACTAGCGCCGCTTCAAGACAAAGATC
ATCCTCAGAAAAACACTGTAAATGTTTATACGAAAAAACTTGCTTACAATTGTTACATAGGTGAATAGGACCTAA
ATCCCACCACAAACCAAAACGCTGCAACGTATAATCATAGTCACTTGAAAGATAATTGCATGCCACAACTTTTTT
GGCCAACGTTTGTAAAGACAACAT
SEQ ID No. 69-Pretorisuskop/96/4 MGF 360 14L (AY261363.1:32525-33598)
TTAGTCTATAACTATAATTTCTGGATGGGCTGTAAGATACTCTTCGGCTCGTTTCAGATTTTTTGAAGTATATGA
CTTTAGCCTATCATATATTTCCTGGGGTTCGGTTACATCTAATGCCAAGGTCACATCACGGCTGAAAAGCTGCTT
TACTAAGAAAATGTTGCTCAAGTTATACATATAAGCTTTGTGCGCAATGTGTTGTGCCCTATCAAAATCAGTAGC
CCCCAAATCGATGCAGAAAAACATGTTTAAAGTATTATTGTTATAGATAGAAAGATTCATGCCATAATCGAGACT
AGCCCCTAACCTATAACAGTAATAAATGGCCGCATAATTTTTTTTCTGCAAGCAAGCAAATTTCATCATCAGATT
AGGGCTGATGCAAATCTCTTTTTCACGACACAGCTCGTGTATGTCAAAAATGTTATTAAAATAAAGGCTACAAGC
TACCCGCCAATAGAGGTGATTTTTATGCCTTTTATAGAAATAGTGAATAGCCTTTGTAAAATTATGTCGTAATGC
CAGGGCAAACCAAAACTTTGTTAATAGGAGGTGCGCCGTATCCCCCGTCAACGGAATGTTTGAACAGGTGTACGT
AACTGTGTCTAAAGTGGTTCTAGTTACGGTTTCCAAGAGTGGATTATGACAAAACATGTCATAACCCAGTAGAAC
TCCTGCACAGGATTTTAGCTTGGCCACTTCTTTTAAAATTTCCAGAAGACGGGGTTCGGTTACAGGCGTTAAGCC
TCCCAGTTCCGTGCACAGCCGCTTTAGATGCACGGCAGGAACACGTATAAGTCCATATTCAGGATTTGCGCCCCA
ATCCACAAATAAACGTATAAGCTCAAGATTATCGCTCTTCACGGCCTTTACTAGCGCCGCTTCAAGACAAAGATC
ATCCTCAGAAAAACACTGTAAATGTTTATACGAAAAAATTTGCTTACAATTGTTACACAGGTGAATAGGACCTAA
ATCCCACCACAAACCAAAACGCTGCAACGTATAATCATAGTCACTTGAAAGATAATTGCATGCCACAACCTTTTT
GGCCAACGTTTGTAAAGACAACAT
SEQ ID No. 70-Tengani 62 MGF 360 14L (AY261364.1:26983-28056)
TTAGTCTATAACTATAATTTCTGGATGGGCTGTAAGATACTCTTCAGCTCGTTTCAGATTTTTTGAAGTATATGA
CTTTAGCATATCATATATTTCCTGGGGTTCGGTTACATCTAATGCCAAGGTCACATCACGGCTGAAAAGCTGCTT
TACTAAGAAAATGTTGCTCAAGTTATACATATAAGCTTTGTGCGCAATGCGTTGTGCCCTATCAAAATCAGTAGC
CCCCAAATCGATGCAGAAAAACATGTTTAAAGTATTATTGTTATAGATAGAAAGATTCATGCCATAATCGAGACT
AGCCCCTAACCTATAACAGTAATAAATGGCCGCATAATTTTTTTTCTGCAAGCAAGCAAATTTCATCATCAGATT
AGGGCTGATGCAAATCTCTTTTTCACGACACAACTCGTGTATGTCAAAAATGTTATTAAAATAAAGGCTACAAGC
TACCCGCCAATAGAGGTGATTTTTATGCCTTTTATAGAAATAGTGAATAGCCTTTGTAAAATTATGTCGTAATGC
CAGGGCAAACCAAAACTTTGTTAATAGGTGGTGCGCCGTATCCCCCGTCAACGGAATGTTTGAACAGGTGTACGT
AACTGTGTCTAAAGTGGTTCTAGTTACGGTTTCCAAGAGTGGATTATGACAAAACATGTCATAACCCAGCAGAAC
TCCTGCACAGGATTTTAGCCTGGCCACTTCTTTTAAAATTTCCAGAAGACGGGATTCGGATACAGGCGTTAAGCC
ACCCAGTTCCGTGCACAGCCGCTTTAGATGCACGGCAGGAACACGTATAAGTCCATATTCAGGATTTGCGCCCCA
ATCCACAAATAAACGTATAAGCTCAAGATTATCGCTCTTCACGGCCTTTACTAGCGCCGCTTCAAGACAAAGATC
ATCCTCAGAAAAACACTGTACATGTTTATACGAAAAAATTTGCTTACAATTATTACATAGGTGAATAGGACCTAA
ATCCCACCACAAACCAAAACGCTGCAACGTATAATCATAGTCACTTGAAAGATAATTGCATGCCACAACCTTTTT
GGCCAATGTTTGTAAAGACAACAT
SEQ ID No. 71-Warmbaths MGF 360 14L (AY261365.1:31747-32820)
TTAGTCTATAACTATAATTTCTGGATGGGCTGTAAGATACTCTTCGGCTCGTTTCATATTTTTTGAAGTATATGC
CTTTAGCCTATCATATATTTCCTGGGGTTCGGTTACATCTAATGCCAAGGTCACATCACGGCTGAAAAGCTGCTT
TACTAAAAGAATGTTGCTCAAGTTATACATATAAGCTTTGTGCGCAATGCGTTGTGCCCTATCAAAATCAGTAGC
CCCCAAATCGATGCAGAAAAACAGGTTTAAAGAATTATTGTTATAGATAGAAAGATTCATGCCATAATCGAGACT
AGCTCCCAACCTATAACAGTAATAAATGGCCGCGTAATTTTTTTCCCGCAAGCAAGCAAATTTCATCATCAGATT
AGGGCTAATGCAAATCTCTTTTTCACGACACAGCTCGTGTATGTCAAAAATGTTATTAAAATAAAGGCTACAAGC
TACCCGCCAATAGAGGTGATTTTTATGCCTTTTATAGAAATAGTGAATAGCCTTTGTAAAATTATGTCGTAATGC
CAGCGCAAACCAAAACTTTGTTAATAGGTGGTGCGCCGTATCCCCCATCAACGGAATTTTTGAACAGCTGTACAT
AACTGTGTCTAAAGTGGTTCTAGTTATGGTTTCCAAGAGTGGGTTATAACAAAACATGTCATAACCCAGTAGAAC
TCCTGCACAGGATTTTAGGTTGGCCACTTCTTTTAAAATTTCCAGAAGACGGGGTTCGGATACAGGTGTTAAGCC
TCCCAGTTCTGTGCACAGCCGCTTTAGATGCACGGTAGGAACACATATAAGTCCATATTCAGGATTTGCACCCCA
ATCCACAAATAAACGTATAAGCTCAAGATTATCCCTCTTCACGGCCTTTACTAGCGCCGCTTCAAGACAGAGATC
ATCCTCAGAAAAACACTGTAAATGTTTATACGAAAAAATTTGCTTACAATTGTTACACAGGTGAATAGGACCTAA
ATCCCACCACAAACCAAAACGCTGCAACATATAATCATAGTCACTTGAAAGATAATTGCATGCCACAACCTTTTT
GGCCAACGTTTGTAAAGACAACAT
SEQ ID No. 72-Warthog MGF 360 14L (AY261366.1:28495-29568)
TTAGTCTATAACTATAATTTCTGGATGGGCTGTAAGATACTCTTCGGCTCGTTTCAGATTTTTTGAAGTATATGA
CTTTAGCATATCATATATTTCCTGGGGTTCGGTTACATCTAATGCCAAGGTCACATCACGGCTGAAAAGCTGCTT
TACTAAAAAAATGTTGCTCAAGTTATACATATAAGTTTTGTGCGCAATGCGTTGTGCCCTATCAAAATCAGTAGC
CCCCAAATCGATGCAGAAAAACATGTTTAAAGTATTATTGTTATAGATAGAAAGATTCATGCCATAATCGAGACT
AGCCCCTAACCTATAACAGTAATAAATGGCCGCATAATTTTTTTTCCGCAAGCAAGCAAATTTCATCATCAGATT
AGGGCTGATACAAATCTCTTTTTCACGACACAGCTCGTGTATGTCAAAAATGTTATTAAAATAAAGGCTACAAGC
TACCCGCCAATAGAGGTGATTTTTATGCCTTTTATAAAAATAGTGAATAGCCTTTGTAAAATTATGTCGTAATGC
CAGGGCAAACCAAAACTTTGTTAATAGGTGGTGCGCCGTATCCCCCGTCAACGGAATGTTTGAACAGGTGTACGT
AACTGTGTCTAAAGTGGTTCTAGTTACGGTTTCCAAGAGTGGATTATGACAAAACATGTCATAACCCAGTAGAAC
TCCTGCACAGGATTTTAGCTTGGCCACTTCTTTTAAAATTTCCAGAAGACGGGGTTCGGATACAGGCGTTAAGCC
TCCCAGTTCCGTGCACAGCCGCTTTAGATGCACGGCAGGAACACGTATAAGTCCATATTCAGGATTTGCGCCCCA
ATCCACAAATAAACGTATAAGCTCAAGATTATCGCTCTTCACGGCCTTTACTAGCGCCGCTTCAAGACAAAGATC
ATCCTCAGAAAAACACTGTAAATGTTTATACGAAAAAATTTGCTTACAATTGTTACATAGGTGAATAGGACCTAA
ATCCCACCACAAACCAAAACGCTGCAACATATAATCATAGTCACTTGAAAGATAATTGCATGCCACAACCTTTTT
GGCCAACGTTTGTAAAGACAACAT

In an embodiment of the attenuated ASFV of the invention, the expression and/or activity of the MGF 360 14L gene is disrupted. Suitably the MGF 360 14L gene comprises the 45 sequence of SEQ ID No. 61, 62, 63, 64, 65, 66, 67, 68, 69, 70, 71 or 72. Suitably the MGF 360 13L gene comprises a sequence having at least 70%, at least 80%, at least 90% or at least 95% identity with SEQ ID No. 61, 62, 63, 64, 65, 66, 67, 68, 69, 70, 71 or 72. Suitably the MGF 360 14L gene consists of the sequence of SEQ ID No. 61, 62, 63, 64, 65, 66, 67, 68, 69, 70, 71 or 72.

MGF 505 1R gene sequences
SEQ ID No. 73- Benin 97/1 MGF 505 1R (NC_044956.1:21971-23566)
ATGTTCTCTCTCCAGAACTTATGTCGAAAAACATTACCTGACTGTAAACTTCCTGAATTTTTTGACGATTATATA
TTACAACTGCTGGGATTATACTGGGAAAACCATGGAACTATTCAACGGGCAGGAAACAACTGTGTGCTTATACAA
CAACATACCCTCATTCCCGTAAATGAAGCCCTAAGAATAGCAGCATCTGAAGAAAATTATGAGATCGTGGGCCTT
TTATTAGCGTGGGAGGGGAACCTTTACTATGCTATTATAGGGGCTCTAGAGGGTAACCGCTACAACTTAATTCGT
AAATATGATGATCAAATCAAGGACCATCATGACATTCTGCCATTCATTGATGATCCAATCATATTTCACAAATGC
CATATCATGCGGCGATGCTTTTTTGATTGTATTTTATATCAAGCTGTAAAATATAGTAAGTTTCGTGTTCTTCTT
TATTTTAAATATACATTAGAGGATGATTTGCCCCTCGTTCATTTACTTATTGAAAAGGCATGTGAAGATCATAAT
TATGAAGTTATTAAATGGATATATGAAAACCTACATGTCTGCCATATAATAGATACCTTTGACTGTGCTATTGCC
CATAAAGATCTACGTTTATATTGTTTGGGGTATACATTTATATACAACAGGATTGTACCCTATAAGTATCATCAT
TTAGATATTCTCATACTTTCAAGCCTACAACTCCTACATAAGGTGGCGGCCAAAGGATACTTAGATTTTATCCTA
GAAACCTTAAAGTATGATCATAATATAGATAATTTAGATGTTATTCTAACACAAGCTGCAACATATAACCATAGA
AAAATTTTAACCTATTTTATTCCTCAATCAACCTACGCACAAATAGAACAATGTTTGTTCGTGGCGATAAAAACA
AAATCTTCCAAGAAAACCTTGAACTTACTACTGTCTCACCTAAACCTTTCCATCAAACTCATCCAAAAAATCAGC
CAATATGTTGCCACTTTCAATTCAACAAATATAATAGGCATTCTGAGTATGAAGCGGAAAAAGAAGATATATTTG
GATATCATATTGACAAAATTTGTAAAAAATGCTATTTTTAATAAATTTGTCGTTCGATGTATGGAGAGATTTTCT
ATAAACCCGGAAAGAATCGTCAAAATGGCTGCGCGTATAAATAAGATGATGTTAGTGAAAAAAATATCTGAACAT
GTTTGGAAAAATCATGCGGCTAGACTTAAACACCTTAAACATGCGGTACACACGATGAAGCATAAAGATGGGAAA
AATAGACTCATGAACTTTATCTATGAACACTGCTATTACCATATGCAAGGGGAAGAAATTTTTAGCCTCGCAAGA
TTTTATGCAATCCATCATGCACCAAAGTTGTTCGACGTTTTTTATAATTGTTGTATCCTAGATACGATACGATTC
AAAAGCCTTCTTTTAGATTGTTCACATATCATAGGTAAAAACGCTCATGATGCTACTAATATCAACATCGTGAAC
AAGTATATTGGCAACCTGTTTGCTATGGGAGTTCTTAGCAAAAAAGAAATCTTACAGGACTATCCATCCATCTAT
TCTAAACATTATATGCCTTAG
SEQ ID No. 74-China/2018/AnhuiXCGQ MGF 505 1R (MK128995.1:27736-29331)
ATGTTCTCTCTCCAGAACTTATGTCGAAAAACATTACCTAACCGTAAACTTCCTGAATTTTTTGACGAATATATA
TTACAACTGCTGGGATTATACTGGGAAAACCATGGAACTATTCAACGAGCAGGAAACAACTGTGTGCTTATACAG
CAACATACCCTCATTCCCGTAAATGAAGCCCTGAGAACAGCAGCATCTGAAGAAAATTATGAGATCGTGAGCCTT
TTATTAGCGTGGGAGGGGAACCTTTACTATGCTATTATAGGGGCTCTAGAGGGCAACCGCCACGACTTAATTCGT
AAATATGATGACCAAATCAAGGACCATCATGAAATTCTGCCATTCATTGACGATCCAGTCATATTTCACAAATGC
CATATCATGCGGCAATGCTTTTTTGATTGTATTTTATATCAAGCTGTAAAATATAGTAAGTTTCGCGTTCTTCTT
TACTTTAAACATAGATTAGAGGATGATTTGCCCTTCACTCATTTACTTATTGAAAAGGCATGTAAAGATCATAAT
TATGAAGTTATTAAATGGATATATGAAAACCTACATATCTACAATATGATAGATACCTTTGAATGTGCTATTGCC
CATAAGGATCTACATCTATATTGTTTGGGGTATAGATTTATATATAACAGAATCGTACCCGATAAGTATCATCAT
TTAGATATTCGCATGCTTTCAAGCCTACAACTCCTACATAAGGTGGCAGCCAAAGGATACTTAGATTTTATCCTA
GAAACCTTAAAGTATGATCATAATAAAGATAATATAAATATTATTCTAACACAAGCTGCAACCTATAACCATAGA
AAAATTTTAATCTATTTCATTCCTCAATCAACCCACGCACAGATAGAACAATGTTTACTAGTGGCGATAAAAGCA
AAATCTTCCAGGAAAACCTTGAACTTACTACTGTCTCACCTAAACCTTTCCATCAACCTCATCAAAAAAATAAGC
CATTATGTTGCCACTTACAATTCAACAAATATAATAGGCATTCTGAGTATGCGGCGGAAAAAGAAGATATATTTA
GATATCATATTGACAAAATTTGTAAAAAAAGCTATTTTTAATAAGTTTGTCGTTCGATGTATGGATACATTTTCT
ATAAACCCGGAAAGAATCCTTAAAATAGCCGCGCGAATAAATAGGATGATGTTAGTGAAAAAAATATCTGAACAT
GTTTGGAAAAATCATGCGGTTAGACTTAAATACCTTAAACATGCGGTACACACGATGAAGCATAAAGATGGGAAA
AATAGACTCATGAACTTTATCTATGATCGCTGTTATTACCATATGCAAGGGGAAGAAATCTTTAGCCTCGCAAGA
TTTTATGCAATCCATCATGCACCAAAGTTGTTTGACGTTTTTTATGATTGTTGTATCCTAGATACGATACGATTC
AAAAGCCTTCTTTTAGATTGTTCACATATCATAGGTAAAAACGCTCATGATGCTACCAATATCAACATCGTGAAC
AAGTATATCGGCAACCTGTTTGTTATGGGAGTTCTTAGCAAAAAAGAAATCTTACAGGACTATCCATCCATTTAT
TCTAAACAATACATGCCTTAG
SEQ ID No. 75-Georgia 2007/1 MGF 505 1R (LR743116.1:28707-30302)
ATGTTCTCTCTCCAGAACTTATGTCGAAAAACATTACCTAACCGTAAACTTCCTGAATTTTTTGACGAATATATA
TTACAACTGCTGGGATTATACTGGGAAAACCATGGAACTATTCAACGAGCAGGAAACAACTGTGTGCTTATACAG
CAACATACCCTCATTCCCGTAAATGAAGCCCTGAGAACAGCAGCATCTGAAGAAAATTATGAGATCGTGAGCCTT
TTATTAGCGTGGGAGGGGAACCTTTACTATGCTATTATAGGGGCTCTAGAGGGCAACCGCCACGACTTAATTCGT
AAATATGATGACCAAATCAAGGACCATCATGAAATTCTGCCATTCATTGACGATCCAGTCATATTTCACAAATGC
CATATCATGCGGCAATGCTTTTTTGATTGTATTTTATATCAAGCTGTAAAATATAGTAAGTTTCGCGTTCTTCTT
TACTTTAAACATAGATTAGAGGATGATTTGCCCTTCACTCATTTACTTATTGAAAAGGCATGTAAAGATCATAAT
TATGAAGTTATTAAATGGATATATGAAAACCTACATATCTACAATATGATAGATACCTTTGAATGTGCTATTGCC
CATAAGGATCTACATCTATATTGTTTGGGGTATAGATTTATATATAACAGAATCGTACCCGATAAGTATCATCAT
TTAGATATTCGCATGCTTTCAAGCCTACAACTCCTACATAAGGTGGCAGCCAAAGGATACTTAGATTTTATCCTA
GAAACCTTAAAGTATGATCATAATAAAGATAATATAAATATTATTCTAACACAAGCTGCAACCTATAACCATAGA
AAAATTTTAATCTATTTCATTCCTCAATCAACCCACGCACAGATAGAACAATGTTTACTAGTGGCGATAAAAGCA
AAATCTTCCAGGAAAACCTTGAACTTACTACTGTCTCACCTAAACCTTTCCATCAACCTCATCAAAAAAATAAGC
CATTATGTTGCCACTTACAATTCAACAAATATAATAGGCATTCTGAGTATGCGGCGGAAAAAGAAGATATATTTA
GATATCATATTGACAAAATTTGTAAAAAAAGCTATTTTTAATAAGTTTGTCGTTCGATGTATGGATACATTTTCT
ATAAACCCGGAAAGAATCCTTAAAATAGCCGCGCGAATAAATAGGATGATGTTAGTGAAAAAAATATCTGAACAT
GTTTGGAAAAATCATGCGGTTAGACTTAAATACCTTAAACATGCGGTACACACGATGAAGCATAAAGATGGGAAA
AATAGACTCATGAACTTTATCTATGATCGCTGTTATTACCATATGCAAGGGGAAGAAATCTTTAGCCTCGCAAGA
TTTTATGCAATCCATCATGCACCAAAGTTGTTTGACGTTTTTTATGATTGTTGTATCCTAGATACGATACGATTC
AAAAGCCTTCTTTTAGATTGTTCACATATCATAGGTAAAAACGCTCATGATGCTACCAATATCAACATCGTGAAC
AAGTATATCGGCAACCTGTTTGTTATGGGAGTTCTTAGCAAAAAAGAAATCTTACAGGACTATCCATCCATTTAT
TCTAAACAATACATGCCTTAG
SEQ ID No. 76-Ken05/Tk1 MGF 505 1R (NC_044945.1:29657-31249)
ATGTTCTCTCTCCAAAACTTATGTCGAAAAACCTTACCTGACTGTAAACTTCCTGAATTTTTTGACGATTATGTA
TTACAACTGCTGGGATTATACTGGGAAAACCATGGAACGATTCAACGAGCAGGAAACAACTGTGTACTTATCCAA
CAACATAACCTCATTCCCGTAAATGAAGCCCTAAGAATAGCAGCATCTGAAGAAAATTATGAGATCGTGAGCCTT
TTATTAGCGTGGGAGGGGAACCTTTACTATGCTATTATAGGGGCTCTAGAGGGTAACCGCCACAACTTAATTCGT
AAATATGATGACCAAATCAAGGACCATCATGAAATTCTGCCATTTATTGACGATCCAGTTATATTTCACAAATGT
CATATAATGCGGCGATGCTTTTTTAATTGTATGTTGTATCAAGCTGTAAAATATAGTAAGTTTAGCGTTCTTCTA
TATTTTAAATATATATTAAAGGAAAATTTGCCCCTCGTCCACTCACTCATTGAAAAAGCGTATAAATATCATAAT
TATGAGGTTATTAAATGGATCTATGAAAATCTACATATCTATGATATCATAAATACTTTTAAATATGCTATTGCC
CATAAAGATCTACGTTTATATTGTTTAGGGTATACATTTGTATATAATAGGATCGTACCCTATAAGTATTATCAT
TTAGATATTCGCATCCTTTTAAGGCTACAACTTCTACATAAGGTGACAGCCAAAGGATACTTGGATTTTATCCTA
GAAACCTTAAAGTATGATCATAATACAAACAATATAGATATTATCCTAACACAGGCTGCAACCTATAACCATAGA
AACATTTTAACCTATTTCATTCCTCAATCAACCTACGCACAAATAGAACAATGCTTATTCGTGGCAATAAAAACA
AACGCTTCCAAAAAAACCTTGAATTTACTATTATCTCACCTAAACCTTTCTATCAAACTCGTCAAAAAATTAAGC
CAATATGTTGTCGCTTACAAGTCAACAAATATCATAAGCATTCTGAGTATGCAGCAAAAAAAGAAGATATATTTA
GATATCATTTTGACAAAGGTTGTAAAAAACGCTGTTTTTATTAAATTTGTCATTGGATGTATGGTTACATTTTCC
ATAAACCCAGAAAGAATTGTCAAAATGGCCGCGCGAATAAAAAAGATGAAGTTAGTAAAAAATATATCTGAACAT
GTTTGGAAAAATCATGCGGCTAAACTTAAACATCTTAAGCATGCGGTACACACGATGAAGCATCAAGAAGGAAAA
AATAGACTCATGAACTTTATCTATGATCACTGCTATTACCACATGCAAGGAGAAGAGATCTTTAGTCTTGCAAGA
TTTTATGCAATCCATCATGCGCCAAAATTGTTCGACGTTTTTTATGATTGTTGTATCCTAGATACGATACGATTT
AAAAGCCTGCTTTTAGATTGTTCACATATTATAGCTAAAAACGCTCATGATGCTAGTATTAACATCGTGAATAAG
TATATTGGCAATCTATTTGCTATGGGAGTTCTTAGCAAAAAAGAAATCTTACAAGACTATCCATCCATCTATTCT
AAATATGACATACTTTAG
SEQ ID No. 77-Ken06.Bus MGF 505 1R (NC_044946.1:25784-27376)
ATGTTCTCTCTCCAGAACTTATGTCGAAAAACCTTACCTGACTGTAAACTTCCTGAGTTTTTTGACGATTATGTA
TTACAACTGCTGGGCTTATACTGGGAAAATCATGGTACGATTCAACGAGCAGGAAACAACTGTGTACTTATCCAA
CAACATAACCTCATTCCCGTAAATGAAGCCCTAAGAATAGCAGCATCTGAGGAAAATTATGAGATCGTGAGCCTT
TTATTAGCGTGGGAGGGGAACCTTTACTATGCTATTATAGGGGCTCTAGAGGGGAACCACCACAACTTAATTCGT
AAATACGATGACCAAATCAAGGACCATCATGAAATTCTGCCATTTATTGACGATCCAGTCATATTTCACAAATGT
CATACAATGCGGCGATGCTTTTTTAATTGTATTTTATATCAAGCTGTAAAATATAGTAAGTTCAGCGTTCTTCTA
TACTTTAAATATATATTAAAGGAGAATTTGCCCCTCGTCCACTCACTTATTGAAAAGGCGTATAAATATCATAAT
TATGAGGTTATTAAATGGATCTATGAAAACCTACATATCTATGATATCATAAATACTTTTAAATGTGCTATTGCC
CATAAAGATCTACGTTTATATTGTTTAGGGTATACATTTGTATATAATAGAATCGTACCCTATAAGTATTATCAT
TTAGATATTCGCATCCTTTTAAGGCTACAACTTCTACATAAGGTGACAGCCAAAGGATACTTGGATTTTATCCTA
GAAACCTTAAAGTATGATCATAATACAAACAATATAGATATTATCCTAACACAGGCTGCAACCTATAACCATAGA
AACATTTTAACCTATTTCATTCCTCAATCAACCTACGCACAAATAGAACAATGTTTATTCGTGGCGATAAAAACA
AATGCTTCCAAGAAAACCTTGAATTTACTATTATCTCACCTAAATCTTTCTATCAAACTCGTCAAAAAATTAAGC
CAATATGTTGTCGCTTACAAGTCAACAAATATCATAAGCATTCTGAGTAGGCAGCAAAAAAAGAAGATATATTTA
GATATCATTTTGACAAAGGTTGTAAAAAATGCTGTTTTTAATAAATTTGTCATTGGATGTATGGTTACATTTTCC
ATAAACCCAGAAAGAATTGTCAAAATGGCCGCGCGAATAAAAAAGATGAAGTTAGTAAAAAATATATCTGAACAT
GTTTGGAAAAATCATGCGGTTAAACTTAAATATCTTAAGCATGCGGTACACACGATGAAGCATCAAGAAGGAAAA
AATAGATTAATGAACTTTATCTATGATCACTGTTATTACCACATGCAAGAAGAAGAGATCTTTAGCCTCGCAAGA
TTTTATGCAATCCATCATGCGCCAAAATTGTTCGACGTTTTTTATGATTGTTGTATCCTAGATACGATACGATTT
AAAAGCCTGCTTTTAGATTGTTCACATATCATAGTTAAAAACGCTCATGATGCTAGTATTAACATCGTGAACAAG
TATATTGGCAATCTATTTGCTATGGGAGTTCTTAGCAAAAAAGAAATCTTACAAGACTATCCATCTATCTATTCT
AAGGATTACATGCTTTAG
SEQ ID No. 78-Kenya 1950 MGF 505 1R (AY261360.1:31904-33496)
ATGTTTTCTCTCCAGAACTTATGTCGAAAAACCTTACCTGACCGTAAACTTCCTGAATTTTTTGACGATTATGTA
TTACAACTGCTGGGATTATACTGGGAAAACCATGGAACGATTCAACGAGCAGGAAACAACTGTGTACTTATCCAA
CAACATAACCTCATTCCCGTAAATGAAGCCCTAAGAATAGCAGCATCTGAAGAAAATTATGAGATCGTGAGCCTT
TTATTAGCTTGGGAGGGGAACCTTTACTATGCTATTATAGGGGCTCTAGAGGGTAATCGCCACAACTTAATTCGT
AAATATGATGACCAAATCAAGGACCATCATGAAATTCTGCCATTTATTGACGATCCAGTCATATTTCACAAATGT
CATATGATGCGGCGATGCTTTTTTAATTGTATTTTGTATCAAGCTGTAAAATATAGTAAGTTTAGCGTTCTTCTA
TATTTTAAATATATATTAAAGGAGAATTTGCCCCTCGTTCATTCACTTATTGAAAAGGCGTGTGAAGATCATAAT
TATGAAGTTATTAAATGGATCTATGAAAACCTACATATCTATGAAATAATGGATACCTTTAAATGTGCTATTGCC
CATAAAGATCTAGATTTATATAGTTTAGGATATACATTTATATATAATAGGATCGTACCCTATAAGTATCATCAT
TTAGATATTCGCATACTTTCAAGGCTACAACTTCTACATAAGGTGACAGCCAAAGGATACTTGGACTTTATCCTA
GAAACCTTAAAGTATGATCATAATAAAGATAATATAAATATTATTCTGACACAAGCTGCAACCTATAACCATAGA
AACATTTTAACCTATTTCATTCCTCAATCAACCTACGCGCAGATAGAACAATGCTTATTCGTGGCAATAAAAACA
AACGCTTCCAAGAAAACCTTGAATTTACTATTATCTCACCTAAACCTTTCTATCAAACTCGTCAAAAAATTAAGC
CAATATGTTGTTGCTTACAAGTCAACAAATATCATAAGCATTCTGAGTATGCAGCAAAAAAAGAAGATATATTTA
GATATCATTTTGACAAAGGTTGTAAAAAACGCTATTTTTATTAAATTTGTCATTGGATGTATGGTTACATTTTCC
ATAAACCCAGAAAGAATTGTCAAAATGGCCGCGCGAATAAAAAAGATGAAGTTAGTGAAAAATATATCTGAACAT
GTTTGGAAAAATCATGCGGCTAAACTTAAACACCTTAAGCATGCGGTACACACGATGAAGCATCAAGAAGGAAAA
AATAGATTAATGAACTTTATCTATGATCACTGTTATTACCACATGCAAGGAGAAGAGATCTTTAGTCTTGCAAGA
TTTTATGCAATCCATCATGCGCCAAAATTGTTCGACGTTTTTTATGATTGTTGTATCCTAGATACGATACGATTT
AAAAGCCTGCTTTTAGATTGTTCATATATCATAGCTAAAAACGCTCATGATGCTAGTATTAACATCGTGAATAAG
TATATTGGCAATCTATTTGCTATGGGGGTTCTTAGCAAAAAAGAAATCTTACAAGACTATCCATCCATCTATTCT
AAGGATTACATGCTTTAG
SEQ ID No. 79-L60 MGF 505 1R (NC_044941.1:22320-23915)
ATGTTCTCTCTCCAGAACTTATGTCGAAAAACATTACCTGACTGTAAACTTCCTGAATTTTTTGACGATTATATA
TTACAACTGCTGGGATTATACTGGGAAAACCATGGAACTATTCAACGGGCAGGAAACAACTGTGTGCTTATACAA
CAACATACCCTCATTCCCGTAAATGAAGCCCTAAGAATAGCAGCATCTGAAGAAAATTATGAGATCGTGGGCCTT
TTATTAGCGTGGGAGGGGAACCTTTACTATGCTATTATAGGGGCTCTAGAGGGTAACCGCTACAACTTAATTCGT
AAATATGATGATCAAATCAAGGACCATCATGACATTCTGCCATTCATTGATGATCCAATCATATTTCACAAATGC
CATATCATGCGGCGATGCTTTTTTGATTGTATTTTATATCAAGCTGTAAAATATAGTAAGTTTCGTGTTCTTCTT
TATTTTAAATATACATTAGAGGATGATTTGCCCCTCGTTCATTTACTTATTGAAAAGGCATGTGAAGATCATAAT
TATGAAGTTATTAAATGGATATATGAAAACCTACATGTCTGCCATATAATAGATACCTTTGACTGTGCTATTGCC
CATAAAGATCTACGTTTATATTGTTTGGGGTATACATTTATATACAACAGGATTGTACCCTATAAGTATCATCAT
TTAGATATTCTCATACTTTCAAGCCTACAACTCCTACATAAGGTGGCGGCCAAAGGATACTTAGATTTTATCCTA
GAAACCTTAAAGTATGATCATAATATAGATAATTTAGATGTTATTCTAACACAAGCTGCAACATATAACCATAGA
AAAATTTTAACCTATTTTATTCCTCAATCAACCTACGCACAAATAGAACAATGTTTGTTCGTGGCGATAAAAACA
AAATCTTCCAAGAAAACCTTGAACTTACTACTGTCTCACCTAAACCTTTCCATCAAACTCATCCAAAAAATCAGC
CAATATGTTGCCACTTTCAATTCAACAAATATAATAGGCATTCTGAGTATGAAGCGGAAAAAGAAGATATATTTG
GATATCATATTGACAAAATTTGTAAAAAATGCTATTTTTAATAAATTTGTCGTTCGATGTATGGAGAGATTTTCT
ATAAACCCGGAAAGAATCGTCAAAATGGCTGCGCGTATAAATAAGATGATGTTAGTGAAAAAAATATCTGAACAT
GTTTGGAAAAATCATGCGGCTAGACTTAAACACCTTAAACATGCGGTACACACGATGAAGCATAAAGATGGGAAA
AATAGACTCATGAACTTTATCTATGAACACTGCTATTACCATATGCAAGGGGAAGAAATTTTTAGCCTCGCAAGA
TTTTATGCAATCCATCATGCACCAAAGTTGTTCGACGTTTTTTATAATTGTTGTATCCTAGATACGATACGATTC
AAAAGCCTTCTTTTAGATTGTTCACATATCATAGGTAAAAACGCTCATGATGCTACTAATATCAACATCGTGAAC
AAGTATATTGGCAACCTGTTTGCTATGGGAGTTCTTAGCAAAAAAGAAATCTTACAGGACTATCCATCCATCTAT
TCTAAACATTATATGCCTTAG
SEQ ID No. 80-Malawi Lil-20/1 (1983) MGF 505 1R (AY261361.1:26041-27633)
ATGTTCTCTCTCCAGAACTTATGTCGAAAAACATTACCTGACTGTAAACTTCCTGAATTTTTTGACGAATATATA
TTACAACTGCTGGGATTATACTGGGAAAACCATGGAACTATTCAACGAGCAGGAAACAACTGTGTGCTTATCCAG
CAACATAACCTTATTCCCGTAAATGAAGCCCTAAGAATAGCAGCATCTGAAGAAAATTATGAGATCGTGAGCCTT
TTATTAGCGTGGGAGGGAAACCTTTACTATGCTATTATAGGGGCTCTAGAGGGCAACCGCCCCGACTTAATTCGT
AAATATGATGACCAAATCAAGGACCATCATGAAATTTTGCCATTCATCGACGATCCAATCATATTTCACAAATGT
CACATAATGCGGCGATGCTTTTTTAATTGTATTTTATATCAAGCTGTAAAATATAGTAAGTTTCGTGTTCTTCTT
TATTTTAAACATAGGTTAGGGGATGATTTGCCCCTCACTCATTTACTTATTGAAAAGGCATGTGAAGATCATAAT
TATGAAGTTATTAAATGGATCTATGAAAACCTACATAGCTACAATATAATGGATACCTTTGAATGTGCTATTGCC
CATAAGGATCTACGTCTATATTGTTTGGGGTATACATTTATATATAATAGGATCGTACCCTATAAGTATCATCAT
TTAGATATTTGCATACTTTCAAGCCTACAACTCCTGCATAAGGTGGCAGCCAAAGGATACTTGGATTTTATCCTA
GAAACCTTAAAGTATGATCATAACATAAATAATATAGATATTATTCTAACACAAGCTGCAACCTATAACCATAGA
AAAATTTTAACCTATTTCATTCCTCAATTAACCTACGCACAGATAGAACAATGTTTACTAGTGGCGATAAAAACA
AAAGCTTCCAAGAAAACCTTGAACTTACTACTGTCTCACCTAAACCTTTCCATCAAACTCATCAAAAAAATAAGC
CAATATGTTGTCACTTACAATTCAACAAATATCATAAGCATTCTGAGTATGCGGCGGAAAAAGAAGATATATTTA
GATATCATTTTGACAGAGTTTGTAAAAAACGCTATTTTTAATAAATTTGTCGTTCGATGTATGGATACATTTTCC
ATAAACCCGGAAAGAATTGTCAAAATGGCCGCACGAATAAATAGGATGATGTTAGTGAAAAATATATCTGAACGT
GTTTGGAAAAATCATGCGGTTAAACTTAAACACCTTAAGCATGCGGTACATACGATGAAGCATCAAGAAGGAAAA
AATAGACTCATGAACTTTATCTATGATCACTGCTATTACCACATGCAAGGGGAAGAGATCTTTGGCCTCGCAAGA
TTTTATGCAATCCATCATGCACCCAAGTTGTTTGACGTTTTTTATGATTGCTGCATGCTAGATGCTACACGATTT
AAAAGCCTGCTTTTAGATTGTCCACATATCATAGGTAAAAACGCTTATGATGCTGGTATCAACCTCGTGAACAAA
TATATTGGCAACCTATTTGCTATGGGGGTTCTTAGCAAAAAAGAAATCTTACAGGACTATCCATCTATCTATTCT
AAACATGATATGTTTTAG
SEQ ID No. 81-Mkuzi 1979 MGF 505 1R (AY261362.1:29425-31020)
ATGTTCTCTCTCCAGAACTTATGTCGAAAAACATTACCTGATTGTAAACTTCCTGAATTTTTTGACGATTATATA
TTACAACTGCTGGGATTATACTGGGAAAACCATGGAACTATTCAACGGGCAGGAAACAACTGTGTGCTTATACAG
CAACATACCCTTATTCCCATAAATGAAGCCCTGAGAACAGCAGCATCTGAAGAAAATTATGAGATCGTGAGCCTT
TTATTAGCATGGGAGGGGAATCTTTACTATGCTATTATAGGGGCTCTAGAGGGCAACCGCCACGACTTAATTCGT
AAATATGATGACCAAATCAAGGACCATCATGAAATTCTGCCATTCATTGACGATCCAGTAATATTTCACAAATGC
CATATCATGCGGCGATGCTTTTTTGATTGTATTTTATATCAAGCTGTAAAATATAGTAAGTTTCGCGTTCTTCTT
TATTTTAAATATAGGTTAGAGGATGATTTACCCCTCACTCATTTACTTATTGAAAAGGCATGTGAAAATCATAAT
TATGAAGTTATTAGATGGATATATGAAAACCTACATATCTACAATATGATAGATACCTTTGAATGTGCTATTGCC
CATAAGGATCTACGTCTATATTGTTTGGGGTATACATTTATATATAACAGGATCGTACCCAATAAGTATCATCAT
ATAGATATTCTCATTCTTTCAAGCCTACAACTCCTGCATAAGGTGGCAGCCAAAGGATACTTAGACTTTATCCTA
GAAACCTTAAAGTATGATCATAACAATGATAATTTAGATATTATTCTAACACAAGCTGCAACATATAACCATAGA
AAAATTTTAACCTATTTTATTCCTCAATCAACCTACGCACAAATAGAACAATGTTTGATGGTGGCGATAAAAACA
AAATCTTCCAAGAAAACCTTGAATTTACTATTGTCCCACCTAAACCTTTCCATCAAACTCATCAAAAAAATAAGC
CAATATGTTGTCACTTACAATTCAACAAATATAATAGGCATTCTGAGTATGAAGCGAAAAAAGAAGATATATTTA
GATATCTTGTTGACAAAATTTGTAAAAAATGCTATTTTTAATAAATTTGTCGTTCGATATATGGATACGTTTTCT
ATAAACCCGGAAAAAATCGTCAAAATGGCCGCGCGAATAAATAAGATGATGTTAGTGAAAAAAATATCTGAACAT
ATTTGGAAAAATCATGCGGCTAGACTTGAACACCTTAAACATGCGGTACACACGATGAAGCATAAAGATGGGAAA
AATAGACTCATGAACTTTATCTATGAGTACTGCTATTACCATATGCAAGGGGAAGAAATTTTTAGCCTCGCAAGA
TTTTATGCAATCCATCATGCACCAAAGTTGTTCGACGTTTTTTATAATTGTTGTATCCTAGATACGATACGATTC
AAAAGCCTTCTTTTAGATTGTTCACATATCATAGGTAAAAACGCTCATGATGCTACTAATATCAACATCGTGAAC
AAGTATATTGACAACCTGTTTGCTATGGGAGTTCTTAGCAAAAAAGAAATCTTACAGGACTATCCATCCATTTAT
TCTAAACATTATATGCCTTAG
SEQ ID No. 82-Pretorisuskop/96/4 MGF 505 1R (AY261363.1:28449-30044)
ATGTTCTCTCTCCAGAACTTATGTCGAAAAACATTACCTGACTGTAAACTTCCTGAATTTTTTGACGAATATATA
TTACAACTGCTGGGATTATACTGGGAAAACCATGGAACTATTCAACGAGCAGGAAACAACTGTGTGCTTGTACAA
CAACATACCCTCATTCCCGTAAACGAAGCCCTGAGAATAGCAGCGTCTGAAGAAAATTATGAGATCGTGAGCCTT
TTATTAGCGTGGGAGGGAAACCTTTACTATGCTATTATAGGAGCTCTAGAGGGCAACCGCCACGACTTAATTCGT
AAATATGATGACCAAATCAAGGACCATCATGAAATTCTGCCATTCATTGACAATCCAGTCATATTTCACAAATGC
CATATAATGCGGCGATGCTTTTTTGATTGTATTTTATATCAAGCTGTAAAATATAGTAAGTTTCGCGTTCTTCTT
TATTTTAAATATAGGTTAGAGAATGATTTGCCCCTCGCTCATTTACTTGTTGAAAAGGCATGTGAAGATCATAAT
TATGAAGTTATTAAATGGTTATATGAAAACCTACATATCTACAATATAATGGAGACCTTTGAATGTGCTATTGCC
CATAAGGATCTACGTCTATATCGTTTGGGGTATACATTTATATATAACAGGATCGTACCCTATAAGTATCATTAT
TTAGATGTTCTCATTCTTTCAGGCCTACATCTCCTGTATAAGGTGGCAGCCAAAGGATACTTAGATTTTATCCTA
GAAACCTTAAAGTATGATCATAACAATGATAATTTAGATATTATTCTAACACAAGCTGTAACATATAACCATAGA
AAAATTTTAACCTATTACATTCCTCAATTAACCTATGCACAAATAGAACAATGTTTGTTCATGGCGATTAAAAAA
AAATCTTCCAAGAAAACCTTGAACTTACTACTGTCTCACCTAAAGCTTTCCATCAAACTCATCAAAAAAATAAGC
CAATATGTTGCCACTTACAATTCAACAAATATAATAGGCATTCTGAATATGAAGCGGAAAAAGAAGATATATTTA
GATATCATATTGACAAAATTTGTAAAATACGCTATTTTTAATAAATATGTCGTTCGTTGTATGGATACATTTTCC
ATAAACCCGGAAAGAATCATCAAAATGGCCGCGCGAATAAATAAGATGTTGTTAGTGAAAAAAATATCTCAACAT
GCTTGGAAAAATCATGCGGCTAGACTTAAACACCTTAAGCATGCGGTATACACGATGAAACATAAAGATGGGAAA
AATAGACTCATGAACTTGATCTATGATCACTACTATTACCATATGCAAGGGGAAGAAATCTTTAGCCTCGCAAGA
TTTTATGCAATCCATCATGCACCAAAGTTGTTTGACGTTTTTTATGATTGTTGTCTCCTAGATACTATACGATTT
AAAAGCCTTCTTTTAGATTGTTCACACATCATAGGTAAAAACGCTCATGATGCTACTAATATCACTATCGTGAAC
AAGTATATTGGCAACCTGTTTGCTATGGGAGTTCTTAGCAAAAAAGAAATCTTACAGGACTATCCATCCATCTAT
TCTAAACATTACATGCCTTAG
SEQ ID No. 83-Tengani 62 MGF 505 1R (AY261364.1:22891 -24486)
ATGTTCTCTCTTCAGAACCTATGTCGAAAAACATTACCTGACTGTAAACTTCCTGAATTTTTTGACGAATATATA
TTACAACTGCTGGGATTATACTGGGAAAACCATGGAACTATTCAACGAGCAGGAAACAACTGTGTGCTTATACAG
CAACATAACCTCATTCCCGTAAATGAAGCCCTAAGAATAGCAGCATCTGAAGAAAATTATGAGATCGTGAGTCTT
TTATTAGCATGGGAGGGGAACCTTTACTATGCTATTATAGGGGCTCTAGAGGGCAACCGCCACGACTTAATTCGT
AAGTATGATGACCAAATCAAGGACCATCATGAAATTCTGCCATTCATTGACGATCCAGTAATATTTCACAAATGC
CATATCATGCGGCAATGCTTTTTTGATTGTATTTTATATCAAGCTGTAAAATATAGTAAGTTTCGCGTTCTTCTT
TCTTTTAAACATAGATTAAGGGATGATTTGCCCTTCACTCATTTACTTATTGAAAAGGCATGTAAAGATCATAAT
TATGAAGTAATTAAATGGATATATGAAAACCTACATATCTACAATATGATAGATACCTTTGAATGTGCTATTGCC
CATAAGGATCTACGTCTATATTGTTTGGGGTATAGATTTATATATAACAGGATCGTACCCGATAAGTATCATCAT
TTAGATATTCGCATGCTTTCAAGCCTACAACTCCTGCATAAGGTGGCAGCCAAAGGATACTTAGATTTTATCCTA
GAAACCTTAAAGTATGATCATAATAAAGATAATATAAATATTATTCTAACACAAGCTGCAACCTATAACCATAGA
AAAATTTTAATCTATTTCATTCCTCAATCAACCCACGCACAGATAGAACAATGTTTACTAGTGGCGATAAAAACA
AAATCTTCCAAGAAAACCTTGAACTTACTACTGTCTCACCTAAACCTTTCCATCAATCTCATCAAAAAAATAAGC
CATTATGTTGCCACTTACAATTCAACAAATATAATAGGCATTCTGAGTATGCGGCGGAAAAAGAAGATATATTTA
GATATCATATTGACAAAATTTGTAAAAAAAGCTATTTTTAATAAATTTGTCGTTCGATGTATGGATACATTTTCT
ATAAACCCGGAAAGAATCCTTAAAATAGCCGCGCGAATAAATAGGACGATGTTAGTGAAAAAAATATCTGAACAT
GTTTGGAAAAATCATGCGGTTAGACTTAAATACCTTAAACATGCGGTACACACGATGAAGCATAAAGATGGGAAA
AATAGACTCATGAACTTTATCTATGATCGCTGTTATTACCATATGCAAGGGGAAGAAATCTTTAGCCTCGCAAGA
TTTTATGCAATCCATCATGCACCAAAGTTGTTTGACGTTTTTTATGATTGTTGTATCCTAGATACGATACGATTC
AAAAGCCTTCTTTTAGATTGTTCACATATCATAGGTAAAAACGCTCATGATGCTACCAATATCAACATCGTGAAC
AAGTATATCGGCAACCTGTTTGTTATGGGAGTTCTTAGCAAAAAAGAAATCTTACAGGACTATCCATCCATCTAT
TCTAAACATTACATGCCTTAG
SEQ ID No. 84-Warmbaths MGF 505 1R (AY261365.1:27651-29246)
ATGTTCTCTCTCCAGAACCTATGTCGAAAAACTTTACCTGACTGTAAACTTCCTGAATTTTTTGACGAATATATA
TTACAACTGCTGGGATTATACTGGGAAAACCATGGAACTATTCAACGAGCAGGAAACAACTGTGTGCTTATACAG
CAACGTACCCTCATTCCCGTAAATGAAGCCCTGAGAATAGCAGCATCTGAAGAAAATTATGAGATCGTGGGCCTT
TTATTAGCGTGGGAGGGGAACCTTTACTATGCTATTATAGGAGCTCTAGAGGGCAACCGCCACGACTTAATTCGT
AAATATGATGACCAAATCAAGGACCATCATGAAATTCTGCCATTCATTGACGATCCAGTCATATTTCACAAATGC
CATATAATCCGACGATGCTTTTTTAATTGTATTTTATATCAAGCTGTAAAATATAGTAAGTTTCGCGTTCTTCTT
TACTTTAAACATAGATTAGAGGATGATTTGCCCCTCACTCATTTACTTATTGAAAAGGCATGTGAAGATCATAAT
TATGAAGTTATTAAATGGATATATGAAAACCTACATACCTACGATATAATGGATACCTTTGAATGTGCTATTGCC
CATAAGGATCTACGTCTATATTGTTTGGGGTATACATTTATATATAACAGGATCGTACCCTATGAGTATCATCAT
TTAGATATTCTCATTCTTTCAAGCCTACAACTCCTGCATAAGGTGGCAGCCAAAGGATACTTAGATTTTATCCTA
GAAACCTTAAAGTATGATCATAACAATGATAATTTAGATATTATTCTAACACAAGCTGCAACATATAACCATAGA
AAAATTTTAACCTATTTCATTCCTCAATTAACCTACGCACAAATAGAACAATGTTTGTTCATGGCGATAAAAAGA
AAATCTTCCAAGAAAACCTTGAACTTACTACTGTCTCACCTAACCCTTTCCATCGAACTCATCAAAAAAATAAGC
CAATATGTTGTCACTTACAATTCAACAAATATAATAGGCATTCTGAGTATGAAGCGGAAAAAGAAGATATATTTA
GATATCATGTTGACAAAATATGTAAAATACGCTATTTTTAATAAATATGTCGTTCGATGTATGGATAGATTTTCC
ATAAACCCGGAAAGAATCATCAAAATGGCCGCGCGAATAAATAGGATGATGTTAGTGAAAAAAATATCTGAACAT
GTTTGGAAAAATCATGCGGCTAGACTTAAACACCTTAAGCATGCGGTACACACGATGAAGCATAAAGATGGGAAA
AATAGACTCATGAACTTTATCTATGATCGCTGCTATTACCATATGCAAGGGGAAGAAATCTTTAGCCTCGCAAGA
TTTTATGCAATCCATCATGCACCAAAGTTGTTTGACGTTTTTTATGATTGTTGTATCCTAGATACTATACAATTC
AAAAGCCTTCTTTTAGATTGTTCACATATCATAGGTAAAAACGCTCATGATGCTACCAATATCAACATCGTGAAC
AAGTATATTGGCGACCTGTTTGCTATGGGAGTTCTTAGCAAAAAAGAAATCTTACAGGACTATCCATCCATCTAT
TCTAAACATTACATGCTTTAG
SEQ ID No. 85-Warthog MGF 505 1R (AY261366.1:24387-25982)
ATGTTTTCTCTCCAGAACTTGTGTCGAAAAACATTACCTGACTGTAAACTTCCTGAATTTTTTGACGAATATATA
TTACAACTGCTGGGATTATACTGGGAAAACCATGGAACTATTCAACGAGCAGGAAACAACTGTGTGCTTATACAG
CAACATACCCTCATTCCCGTAAACGAAGCCCTGAGAATAGCAGCATCTGAAGAAAATTATGAGATCGTGAGCCTT
TTATTAGCGTGGGAGGGAAACCTTTACTATGCTATTATAGGAGCTCTAGAGGGCAACCGCCACGACTTAATTCGT
AAATATGATGACCAAATCAAGGACCATCATGAAATTCTGCCATTCATTGACGATCCAGTCATATTTCACAAATGC
CATATCATGCGGCGATGCTTTTTTGATTGTATTTTATATCAAGCTGTAAAATATAGTAAGTTTCGCGTTCTTCTT
TATTTTAAATATAGGTTAGAGAATGATTTGCCCCTCGCTCATTTACTTATTAAAAAGGCATGTGAAGATCATAAT
TATGAAGTTATTAAATGGATATATGAAAACCTACATATCTACAATATAATGGATACCTTTGGATGTGCTATTGCC
CATAAGGATCTACGTCTATATCGTTTGGGGTATACATTTATATATAACAGGATCGTACCCTATAAGTATCATTAT
TTAGATGTTCTCATTCTTTCAGGCCTACATCTCCTGTATAAGGTGGCAGCCAAAGGATACTTAGATTTTATCCTA
GAAACCTTAAAGTATGATCATAACAATGATAATTTAGATATTATTCTAACACAAGCTGCAACATATAACCATAGA
AAAATTTTAACCTATTACATTCCTCAATTAACCTATGCACAAATAGAACAATGTTTGTTCATGGCGATTAAAAAA
AAATCTTCCAAGAAAACCTTGAACTTACTACTGTCTCACCTAAAGCTTTCCATCAAACTCATCAAAAAAATAAGC
CAATATGTTGCCACTTACAATTCAACAAATATAATAGGCATTCTGAATATGCGGCGGAAAAAGAAGATATATTTA
GATATCATATTGACAAAATTTGTAAAAAAAGCTATTTTTAATAAATTTGTCGTTCGATGTATGGATACATTTTCC
ATAAACCCGGAAAGAATCATCAAAATGGCCGCGCGAATAAATAAGATGTTGTTAGTGAAAAAAATATCTGAACAT
GCTTGGAAAAATCATGCGGCTAGACTTAAACACCTTAAGCATGCGGTATACACGATGAAACATAAAGATGGGAAA
AATAGACTCATGAACTTGATCTATGATCACTACTATTACCATATGCAAGGGGAAGAAATCTTTAGCCTCGCAAGA
TTTTATGCAATCCATCATGCACCAAAGTTGTTTGACGTTTTTTATGATTGTTGTCTCCTAGATACTATACGATTT
AAAAACCTTCTTTTAGATTGTTCACACATCATAGGTAAAAACGCTCATGATGCTACTAATATCACTATCGTGAAC
AAGTATATTGGCAACCTGTTTGCTATGGGAGTTCTTAGCAAAAAAGAAATCTTACAGGACTATCCATCCATCTAT
TCTAAACATTACATGCCTTAG

In an embodiment of the attenuated ASFV of the invention, the expression and/or activity of 25 the MGF 505 1R gene is disrupted. Suitably the MGF 505 1R gene comprises the sequence of SEQ ID No. 73, 74, 75, 76, 77, 78, 79, 80, 81, 82, 83, 84 or 85. Suitably the MGF 505 1R gene comprises a sequence having at least 70%, at least 80%, at least 90% or at least 95% identity with SEQ ID No. 73, 74, 75, 76, 77, 78, 79, 80, 81, 82, 83, 84 or 85. Suitably the MGF 505 1R gene consists of the sequence of SEQ ID No. 73, 74, 75, 76, 77, 78, 79, 80, 30 81, 82, 83, 84 or 85.

MGF 505 2R gene sequences
SEQ ID No. 86-Benin 97/1 MGF 505 2R (NC_044956.1:27352-28932)
ATGTTTTCCCTTCAAGACCTTTGCCGAAAGCATCTTTTTATTCTTCCCGATGTTTTTGGCGAGCATGTACTACAA
CGATTAGGACTGTATTGGAGATGTCACGGCTCCCTTCAACGCATAGGAGACGACCACATACTCATACGACGGGAT
CTCATCCTTTCCACCAACGAGGCCTTAAGAATGGCGGGAGAGGAAGGAAACAATGAAGTAGTAAAGCTCTTGTTA
CTGTGGAAGGGAAATCTTCATTACGCCGTCATAGGAGCCCTGCAGGGTGATCAATATGACCTGATCCATAAGTAT
GAAAACCAAATCGGCGACTTTCATTTTATCTTACCATTGATTCAAGACGCGAATACGTTTGAAAAATGCCACGCT
TTAGAACGTTTTTGTGGTGTTTCATGTCTGCTAAAACATGCTACAAAATACAACATGCTCCCTATTCTCCAAAAA
TACCAAGAAGAGCTGTCTATGAGAGCGTATCTTCACGAAACCCTATTTGAACTAGCATGCCTATGGCAGAGGTAT
GATGTCCTTAAATGGATAGAGCAGACCATGCATGTTTACGACCTAAAGATTATGTTTAATATTGCCATCTCCAAG
AGGGATCTGACTATGTACTCCTTAGGATATATTTTCCTTTTTGATAGAGGGAACACCGAAGCTACGTTGCTAACG
CAACATCTCGAGAAGACAGCGGCCAAAGGGCTCCTCCACTTTGTGCTAGAAACGTTAAAATACGGCGGCAACATA
GATACCGTCCTGACCCAAGCCGTAAAGTACAACCATAGAAAACTTTTAGATTATTTTCTGCGTCAACTACCTCGT
AAACATATTGAAAAGCTTTTGTTGCTGGCTGTTCAGGAAAAGGCTTCTAAAAAAACATTGAACTTACTGTTGTCA
CATTTAAACTACTCCGTGAAACGCATCAAAAAACTGCTGCGCTATGTGATAGAGTATGAGTCCACCTTGGTGATA
AAGATTTTGTTAAAAAAAAGAGTAAACCTGATAGATGCCATGTTGGAAAAGATGGTAAGATATTTTTCTGCGACG
AAAGTGAGGACGATCATGGATGAGCTTTCGATTAGTCCGGAAAGAGTCATTAAGATGGCTATACAGAAAATGAGA
ACGGATATCGTAATCCATACTTCTTATGTTTGGGAGGATGATCTAGAACGTCTTACTCGTCTTAAAAATATGGTA
TACACCATAAAGTACGAACATGGGAAAAAAATGTTAATTAAAGTCATGCACGGCATATACAAAAACTTATTATAC
GGCGAAAGGGAAAAAGTCATGTTTCATTTAGCCAAGCTCTATGTTGCTCAAAATGCGGCCACCCAATTCAGAGAC
ATTTGTAAGGACTGTTACAAACTGGATGTGGCACGGTTTAAACCGCGGTTTAAGCAACTAATATTAGACTGTTTA
GAAATTGTTACTAAAAAATCTTGCTATAGTATCCTGGAAATCTTAGAAAAACATATTATTTCCCTATTTACTATG
AAAGTTATGACTGAAGAAGAAAAAAACCTATGTTTAGAAATATTATATAAAGTAATTCATTATAAAACAATACAA
TGTTAA
SEQ ID No. 87-China/2018/AnhuiXCGQ MGF 505 2R
ATGTTTTCCCTTCAAGACCTTTGCCGAAAGCATCTTTTTATTCTTCCCGATGTTTTTGGCGAGCATGTACTACAA
CGATTAGGACTGTATTGGAGATGTCACGGCTCCCTTCAACGCATAGGAGACGACCACATACTCATACGACGGGAT
CTCATCCTTTCCACCAACGAGGCCTTAAGAATGGCGGGAGAGGAAGGAAACAATGAAGTAGTAAAGCTCTTGTTA
CTGTGGAAGGGAAATCTTCATTACGCCGTCATAGGAGCCTTGCAGGGTGATCAATATGACCTGATCCATAAGTAT
GAAAACCAAATCGGCGACTTTCATTTTATCTTACCATTGATTCAAGACGCGAATACGTTTGAAAAATGCCACGCT
TTAGAACGTTTTTGTGGTGTTTCATGTCTGCTAAAACATGCTACAAAATACAACATGCTCCCTATTCTCCAAAAA
TACCAAGAAGAGCTGTCTATGAGAGCGTATCTTCACGAAACCCTATTTGAACTAGCATGCCTATGGCAGAGGTAT
GATGTCCTTAAATGGATAGAGCAAACCATACATGTTTACGACCTAAAGATTATGTTTAATATTGCCATCTCCAAG
AGGGATCTGACTATGTACTCCTTAGGATATATTTTCCTTTTTGATAGAGGGAACACCGAAGCTACGTTGCTAACG
CAACATCTCAAGAAGACAGCGGCCAAAGGGCTCCTCCACTTTGTGCTAGAAACGTTAAAATACGGCGGCAACATA
GATACCGTCCTGACCCAAGCCGTAAAGTACAATCATAGAAAACTTTTAGATTATTTTCTGCGTCAACTACCTCGT
AAACATATTGAAAAACTTTTGTTGCTGGCCGTGCAGGAAAAGGCTTCTAAAAAAACATTGAACTTACTGTTGTCA
CATTTAAACTACTCCGTGAAACGCATCAAAAAACTACCGCGCTATGTGATAGAGTACGAGTCCACCTTGGTGATA
AAGATTTTATTAAAAAAAAGAGTGAACCTGATAGATGCCATGTTGGAAAAGATGGTAAGATATTTTTCTGCGACG
AAAGTGAGGACGATCATGGATGAGCTTTCGATTAGTCCGGAAAGAGTCATTAAGATGGCTATACAGAAAATGAGA
ACGGATATCGTAATCCATACTTCTTATGTTTGGGAGGATGATCTAGAACGTCTTACTCGTCTTAAAAATATGGTA
TACACCATAAAGTACGAACATGGGAAAAAAATGTTAATTAAAGTCATGCACGGCATATACAAAAACTTATTATAC
GGCGAAAGGGAAAAAGTCATGTTTTATTTAGCCAAGCTCTATGTTGCTCAAAACGCGGCCACCCAATTCAGAGAC
ATTTGTAAGGACTGTTACAAACTGGATGTGGCACGGTTTAAACCGCGGTTTAAGCAACTAATATTAGACTGTTTA
GAAATTATTACTAAAAAATCTTGCTATAGTATCCTGGAAATCTTAGAAAAACATATTATTTCCCTGTTTACTATG
AAAGTTATGACTGAAGAAGAAAAAAACCTATGTTTAGAAATATTATATAAAGTAATTCATTATAAAACAATACAA
TGTTAA
SEQ ID No. 88-Georgia 2007/1 MGF 505 2R (NC_044959.1:33119-34699)
ATGTTTTCCCTTCAAGACCTTTGCCGAAAGCATCTTTTTATTCTTCCCGATGTTTTTGGCGAGCATGTACTACAA
CGATTAGGACTGTATTGGAGATGTCACGGCTCCCTTCAACGCATAGGAGACGACCACATACTCATACGACGGGAT
CTCATCCTTTCCACCAACGAGGCCTTAAGAATGGCGGGAGAGGAAGGAAACAATGAAGTAGTAAAGCTCTTGTTA
CTGTGGAAGGGAAATCTTCATTACGCCGTCATAGGAGCCTTGCAGGGTGATCAATATGACCTGATCCATAAGTAT
GAAAACCAAATCGGCGACTTTCATTTTATCTTACCATTGATTCAAGACGCGAATACGTTTGAAAAATGCCACGCT
TTAGAACGTTTTTGTGGTGTTTCATGTCTGCTAAAACATGCTACAAAATACAACATGCTCCCTATTCTCCAAAAA
TACCAAGAAGAGCTGTCTATGAGAGCGTATCTTCACGAAACCCTATTTGAACTAGCATGCCTATGGCAGAGGTAT
GATGTCCTTAAATGGATAGAGCAAACCATACATGTTTACGACCTAAAGATTATGTTTAATATTGCCATCTCCAAG
AGGGATCTGACTATGTACTCCTTAGGATATATTTTCCTTTTTGATAGAGGGAACACCGAAGCTACGTTGCTAACG
CAACATCTCAAGAAGACAGCGGCCAAAGGGCTCCTCCACTTTGTGCTAGAAACGTTAAAATACGGCGGCAACATA
GATACCGTCCTGACCCAAGCCGTAAAGTACAATCATAGAAAACTTTTAGATTATTTTCTGCGTCAACTACCTCGT
AAACATATTGAAAAACTTTTGTTGCTGGCCGTGCAGGAAAAGGCTTCTAAAAAAACATTGAACTTACTGTTGTCA
CATTTAAACTACTCCGTGAAACGCATCAAAAAACTACCGCGCTATGTGATAGAGTACGAGTCCACCTTGGTGATA
AAGATTTTATTAAAAAAAAGAGTGAACCTGATAGATGCCATGTTGGAAAAGATGGTAAGATATTTTTCTGCGACG
AAAGTGAGGACGATCATGGATGAGCTTTCGATTAGTCCGGAAAGAGTCATTAAGATGGCTATACAGAAAATGAGA
ACGGATATCGTAATCCATACTTCTTATGTTTGGGAGGATGATCTAGAACGTCTTACTCGTCTTAAAAATATGGTA
TACACCATAAAGTACGAACATGGGAAAAAAATGTTAATTAAAGTCATGCACGGCATATACAAAAACTTATTATAC
GGCGAAAGGGAAAAAGTCATGTTTTATTTAGCCAAGCTCTATGTTGCTCAAAACGCGGCCACCCAATTCAGAGAC
ATTTGTAAGGACTGTTACAAACTGGATGTGGCACGGTTTAAACCGCGGTTTAAGCAACTAATATTAGACTGTTTA
GAAATTATTACTAAAAAATCTTGCTATAGTATCCTGGAAATCTTAGAAAAACATATTATTTCCCTGTTTACTATG
AAAGTTATGACTGAAGAAGAAAAAAACCTATGTTTAGAAATATTATATAAAGTAATTCATTATAAAACAATACAA
TGTTAA
SEQ ID No. 89-Ken05/Tk1 MGF 505 2R (NC_044945.1:35094-36674)
ATGTTTTCCCTTCAAGACCATTGCCGAAAGCATCTTTTTATTCTTCCCGATGTTTTTGGCGAGCATGTACTACAA
CGGTTAGGACTGTACTGGGGACGTCACGGCTCTCTTCAACGAATCGGGGACGATCACATACTCATACGGCGGGAC
CTCATCCTTTCCACCAACGAGGCCTTAAAAATGGCGGGAGAAGAAGGAAACAATGAAGTAGTAAAGCTCTTGTTA
CTGTGGGAGGGAAATCTTCATTATGCTATCATAGGGGCTTTACAGGGTGATCAATATGACCTAATCCATAAGTAT
GAAAACCAAATCGAAGACTATCATCATATCTTACCATTGATTCAAGATGCGGAAACGTTTGAAAAATGCCACGCC
TTAGAACGTTTTTGTGATGTTCCATGTCTGCTAGAACATGCTACAAAACACAACATGCTCCCTATTCTCCAAAAA
TATCAAGAAGAGTTGTCTATAAGAGTGTATCTACGCGAAACCCTATTCGAACTAGCATGCCTATGGCAGAGGTAT
GATATTCTTAAATGGATAGAGCAAACCATGCATGTTTACGATCTAAAAATTATATTTAATATTGCCATCTCCAAG
AGGGATCTAAGCATGTACTCCTTAGGATATGTTCTCCTTTTTGATAGAGGGAACACCGAAGCTACCTTGTTAACG
CAACACCTCGAGAAGACAGCGGCCAAGGGGCTCCTCCACTTTGTGCTAGAAACGTTAAAATACGGCGGTAACTTA
AATATCGTCCTGTTCCAAGCCGTAAAATACAATCATAGAAAACTTTTAGATTATTTTCTGCGTCAACTACCCCGT
AAAAATATTGAAAAACTTTTGTTGCTGGCTGTGCAGGAAAAAGCTTCTAAGAAAACATTGAACTTACTGTTGTCA
CATTTAAACTACTCCGTGAAACGCATCAAAAAACTGCTGCGCTATGTGATAGAGTACGAGTCTACCTTGGTGATA
AAGATTTTATTAAAAAAAAGAGTAAACCTAATAGACGCCGTGTTGGAAAAGACTGTAAGATATTTTTCTGAGACG
AAAGTAAAGACTATTATGGATGAGCTTTCGATTAATCCGGAAAAAGTCATTAAGATGGCCATACAGAAAATGAGA
ACGGATATTGTGATCCAAACTTCTTATATTTGGGAGGATGATCTAGAAAGACTTATTCGTCTTAAAAATATGGTA
TACACCATAAAGTATGAACATGGGAAAAAAATGTTAATGAAAGTTATTCACGGCATATACAAAAACTTATTACAC
GATGAAAAAGAAAAAGTCATGTTTCATTTAGCCAAGTTCTATATTGCTCAAAACGCGGCCACCCAATTCAGAGAC
ATTTGTAAAGACTGTTGCAAACTGGATGTGGCGCGGTTTAAACCGCGGTTTAAACAACTAATTTTAGACTGTTTA
GAAATTGTTACTAAAAAATCTTGCTTTAGTATTATAGAAATTTTAGAAAACCATATTATTTCCCTATTTATGATG
AAAGTTATCACTGAAGATGAAAAAAACCTAGGTTTAGAATTATTATATAAAGTAATTAGTTACAAAATGATATCA
TATTAA
SEQ ID No. 90-KenO6.Bus MGF 505 2R (NC_044946.1:31225-32799)
ATGTTTTCCCTTCAAGACCTTTGCCGAAAGCATCTTTTTATTCTTCCCGATGTTTTTGGCGAGCATGTACTACAA
CAATTGGGGCTGTACTGGGAAAAACACGGCTCTCTTCAACGAATAGGGAACGATCACATACTCATACGGCGGGAT
CTCATCCTTTCTATCAACGAGGCCTTAAAAATAGCGGCAGAGGAAGGAAACAATGAAGTAGTAAAGCTCTTGTTA
CTGTGGAAGGGAAATCTTCATTATGCCATCATAGGAGCCTTGCAGGGTGACCAATATGACCTCATCCATACGTAC
GAAAACCAAATCGAAGACTATCATCATATCTTGCCATTGATTCAAGATGCGAAAACGTTTGAAAAATGCCACGCC
TTGGAACGTTTTTGTGATGTTCCATGCCTACTAGAACATGCTACAAAACACAACATGCTCCCTATTCTCCAAAAA
TACCAAGAAGAGCTGTTTATAAGAGTGTATCTCCGCGAAACCCTATTTGAACTAGCATGCCTATGGCAGAGGTAT
GATATCCTTAAATGGATAGAGCAAACCATGCATGTTTACGACCTAAAGATTATGTTTAATATTGCCATCTCCAAG
AGGGATCTAAGCATGTACTCTTTAGAATATATTCTCTTTTTTAATAGAGGGAACACCGATGTTGCGTTAGCAACG
TTGCTAACGCAACATCTCGAGAAGACAGCGGCCAAGGGGCTCCTCCACTTTGTGCTAGAAACGTTAAAATACGGC
GGTAACATAAACATCGTCCTCTCCCAAGCCGTAAAATACAATCATAGAAAACTTTTAGATTATTTTCTGCGTCAA
CTACCTCGTAAAAATATTGAAAAACTTTTGTTGCTGGCCGTACAGGAAAGGGCTTCTAAGAAAACATTGAACTTA
CTGTTGTCTTATTTAAACTACTCCGTGAAACGCATCAAAAAACTGCTGCGCTATGTGATAGAGCATGAGTCCACA
CTGGTGATAAGGATTTTATTAAAAAAAAGAATAAACCTAATAGACGCCGTGTTGGAAAAGACTGTAAGATATTTT
TCTATGACTAAAGTGAGGACGATCATGGATGAGCTTTCGATTAATCCGGAAAAAGTCATTAAAATGGCCGTGCAG
AAAATGAGAACGGATATCGTGATCCATACTTCTTATGTTTGGGAGGATGATCTAGAAAGACTTATTCGTCTTAAA
AATATGCTATACACTATAAAGTATGAGCATGGAAAAAAAATGCTAATTAAAGTCATTCACGGCATATACAAAAAC
TTATACGGCGAAAAAGAAAAAGTCATGTTTAATTTGGCCAAGTTCTATGCTGCTCAAAACGCGGCCACCCAATTC
AAAGACACTTGTAAAGACTGTTGCAAACTGGATGTGGCGCGGTTTAAACAACTAATTTTAGACTGTTTAGACATT
ATTACTAAAAAAACGTGCCTCAGTATCATGGAAATCTTAGAAAATCATATTATTTCGCTATTTGCTATGAAAATG
ATGACGGAGGATGAAAAAAACTTAGGTTTAGAAATATTATATAAAGTAATTAGTTACAAAATGATATCATATTAA
SEQ ID No. 91-Kenya 1950 MGF 505 2R (AY261360.1:37419-38999)
ATGTTTTCCCTTCAAGACCTTTGCCGAAAGCATCTTTTTATTCTTCCCGATGTTTTTGGCGAGCACGTACTACAA
CAATTGGGGCTGTACTGGGAAAAACACGGCTCTCTTCAACGAATAGGGAACGATCACATACTCATACGGCGGGAT
CTCATCCTTTCTATCAACGAGGCCTTAAAAATAGCGGCAGAGGAAGGAAACAATGAAGTAGTAAAGCTTTTGTTA
CTATGGAAGGGAAATCTTCATTATGCCATCATAGGAGCTTTGGAGGGTGACCAATATGACCTGATCTATACGTAT
GAAAACCAAATTGAAGACTATCATCATATCTTGCCATTGATTCAAGATGCGAAAACGTTTGAAAAATGCCACGCC
TTGGAACGTTTTTGTGATGTTCCATGCCTGCTAGAGCATGCTATAAAACACAACATGCTCCCTATTCTCCAAAAA
TATCAAGAAGAACTGTTTATAAGAGTGTATCTCCGCGAAACCCTATTTGAACTAGCATGCCTATGGCAGAGGTAT
GATATTCTTAAATGGATAGAGCAAACCATGCATGTTTACGATCTAAAAATTATATTTAATATTGCCATCTCCAAG
AGGGATCTAAGCATGTACTCCTTAGGATATGTTCTCCTTTTTGATAGAGGGAACACCGAAGCTACCTTGTTAACG
CAACACCTCGAGAAGACAGCGGCCAAGGGGCTCCTCCACTTTGTGCTAGAAACGTTAAAATACGGCGGTAACATA
AACATCGTCCTATCCCAAGCCGTAAAATACAATCATAGGAAACTTTTAGATTATTTTCTGCGTCAACTACCTCGT
AAAAATATTGAAAAACTTTTGTTGTTAGCCGTACAGGAAAAGGCTTCTAAGAAAACATTGAACTTACTGTTGTCT
TATTTAAACTACTCCGTGAAACGCATCAAAAAACTGCTGCGCTATGTGATAGAGTACGAGTCCACACTGGTGATA
AGGATTTTATTAAAAAAAAGAATAAACCTAATAGACGCCGTGTTGGAAAAGACTGTAAGATATTTTTCTGAGACG
AAAGTAAAGACTATTATGGATGAGCTTTCGATTAATCCGGAAAAAGTTATTAAGATGGCCATACAGAAAATGAGA
ACGGATATTGTGATCCAAACTTCTTATATTTGGGAGGATGATCTAGAAAGACTTATTCGTCTTAAAAATATGGTA
TACACCATAAAGTATGAACATGGGAAAAAAATGTTAATGAAAGTTATTCACGGCATATACAAAAACTTATTACAC
GATGAAAAAGAAAAAGTCATGTTTCATTTAGCCAAGTTCTATATTGCTCAAAACGCGGCCACCCAATTCAGAGAC
ATTTGTAAAGACTGTTGCAAACTGGATGTGGCGCGGTTTAAACCGCGGTTTAAACAACTAATTTTAGACTGTTTA
GATATTATTACTAAAAAAACGTGCCTCAATATTATGGAAATCTTAGAAAATCATATTATTTCGCTATTTGCTATG
AAAATGATGAGTGAAGATGAAAAAAACCTAGGTTTAGAATTATTATATAAAGTAATTAGTTACAAAATGATATCA
TATTAA
SEQ ID No. 92-L60 MGF 505 2R (NC_044941.1:27701-29281)
ATGTTTTCCCTTCAAGACCTTTGCCGAAAGCATCTTTTTATTCTTCCCGATGTTTTTGGCGAGCATGTACTACAA
CGATTAGGACTGTATTGGAGATGTCACGGCTCCCTTCAACGCATAGGAGACGACCACATACTCATACGACGGGAT
CTCATCCTTTCCACCAACGAGGCCTTAAGAATGGCGGGAGAGGAAGGAAACAATGAAGTAGTAAAGCTCTTGTTA
CTGTGGAAGGGAAATCTTCATTACGCCGTCATAGGAGCCCTGCAGGGTGATCAATATGACCTGATCCATAAGTAT
GAAAACCAAATCGGCGACTTTCATTTTATCTTACCATTGATTCAAGACGCGAATACGTTTGAAAAATGCCACGCT
TTAGAACGTTTTTGTGGTGTTTCATGTCTGCTAAAACATGCTACAAAATACAACATGCTCCCTATTCTCCAAAAA
TACCAAGAAGAGCTGTCTATGAGAGCGTATCTTCACGAAACCCTATTTGAACTAGCATGCCTATGGCAGAGGTAT
GATGTCCTTAAATGGATAGAGCAGACCATGCATGTTTACGACCTAAAGATTATGTTTAATATTGCCATCTCCAAG
AGGGATCTGACTATGTACTCCTTAGGATATATTTTCCTTTTTGATAGAGGGAACACCGAAGCTACGTTGCTAACG
CAACATCTCGAGAAGACAGCGGCCAAAGGGCTCCTCCACTTTGTGCTAGAAACGTTAAAATACGGCGGCAACATA
GATACCGTCCTGACCCAAGCCGTAAAGTACAACCATAGAAAACTTTTAGATTATTTTCTGCGTCAACTACCTCGT
AAACATATTGAAAAGCTTTTGTTGCTGGCTGTTCAGGAAAAGGCTTCTAAAAAAACATTGAACTTACTGTTGTCA
CATTTAAACTACTCCGTGAAACGCATCAAAAAACTGCTGCGCTATGTGATAGAGTATGAGTCCACCTTGGTGATA
AAGATTTTGTTAAAAAAAAGAGTAAACCTGATAGATGCCATGTTGGAAAAGATGGTAAGATATTTTTCTGCGACG
AAAGTGAGGACGATCATGGATGAGCTTTCGATTAGTCCGGAAAGAGTCATTAAGATGGCTATACAGAAAATGAGA
ACGGATATCGTAATCCATACTTCTTATGTTTGGGAGGATGATCTAGAACGTCTTACTCGTCTTAAAAATATGGTA
TACACCATAAAGTACGAACATGGGAAAAAAATGTTAATTAAAGTCATGCACGGCATATACAAAAACTTATTATAC
GGCGAAAGGGAAAAAGTCATGTTTCATTTAGCCAAGCTCTATGTTGCTCAAAATGCGGCCACCCAATTCAGAGAC
ATTTGTAAGGACTGTTACAAACTGGATGTGGCACGGTTTAAACCGCGGTTTAAGCAACTAATATTAGACTGTTTA
GAAATTGTTACTAAAAAATCTTGCTATAGTATCCTGGAAATCTTAGAAAAACATATTATTTCCCTATTTACTATG
AAAGTTATGACTGAAGAAGAAAAAAACCTATGTTTAGAAATATTATATAAAGTAATTCATTATAAAACAATACAA
TGTTAA
SEQ ID No. 93-Malawi Lil-20/1 (1983) MGF 505 2R (AY261361.1:31541-33121)
ATGTTTTCCCTTCAAGATCTTTGCCGAAAGCATCTTTTTATTCTTCCCGATGTTTTTGGCGAGCATGTACTACAA
CAGTTAGGACTGTACTGGAAACGTCACGGCTCTCTTCAACGCATAGGAGACGACCACATACTCATACGGCGGGAC
CTCATCCTTTCCACCAACGAGGCCTTAAAAATGGCGGGAGAGGAAGGAAACAATGAAGTAGTAAAGCTCTTGTTA
CTGTGGGAGGGAAACCTTCATTATGCCATCATAGGGGCCTTACAGGGTGATCAATATGATCTGATCCATAAGTAT
GAAAACCAAATCGAAGACTATCATCATATCTTGCCATTGATTCAAGATGCGAAAACGTTTGAAAAATGCCACGCC
TTAGAACGTTTTTGTGATGTTCCATGTCTGCTAGAACATGCTACAAAACACAACATGCTCCCTATTCTCCAAAAA
TACCAAGAAGAGCTGTCTATAAGAGTGTATCTTCGCGAAACCCTATTCGAACTAGCATGCCTATGGCAGAGGTAT
GATGTTCTTAAATGGATAGAGCAAACCATGCATGTTTACGACCTAAAGATTATGTTTAATATTGCCATCTCCAAG
AGGGATCTAAGCATGTACTCCTTAGGATATGTTCTCCTTTTTGATAGAGGAAACATCGAAGCTACGTTCCTAACG
CAACATCTCGAGAAGACAGCGGCCAAAGGGCTCCTCCACTTTGTGCTAGAAACGTTAAAATACGGCGGTAACTTA
AATATCGTCCTGTCCCAAGCCGTAAAATACAATCATAGAAAACTTTTGGATTATTTTCTGCGTCAACTACCTCGT
AAAAATATTGAAAAACTTTTGTTGCTGGCCGTGCAGGAAAAGGCTTCTAAAAAAACATTGAACCTACTGTTGTCA
CACTTAAACTACTCCGTGAAACACATCAAAAAACTGCTGCGCTATGTGATAGAGTACGAGTCTACCTTGGTGATA
AAACTTTTATTGAAAAAAAGAGTAAACCTGATAGACGCCGTGTTGGAAAAGAATGTAAGATATTTTTCTGCGATT
AAAGTGAGGACTATTATGGATGAGCTTTCGATTAGTCCGGAAAGAGTCATTAAGATGGCCATACAGAAAATGAGA
ACGGATATTGTGATTCAGACTTCTTATATTTGGGAGGATGATCTAGAAAGACTTATTCGTCTTAAAAATATGGTA
TACACCATAAAGTATGAACATGGGAAAAAAATGTTAATTAAAGTTATTCACGGCATATACAAAAACTTATTATAC
GGCGAAAAAGAAAAAGTCATGTTTCATTTAGCCAAACTCTATGTTGCTCAAAACGCGGCCACCCAATTCAGGGAC
ATTTGTAAGGACTGTTGCAAACTGGATGTGGCGCGGTTTAAACCGCGGTTTAAACAACTAATTTTAGACTGTTTA
GAAATGGTTACTAAAAAATCTTGCTTTAGTATTATAGAAATCTTAGAAAACTATATTATTTCCCTATTTGTGATG
AAAGTCATCACTGAAGAAGAAAAAAACCTATGTTTAGAACTATTATATAAAGTAATTAGTTACAAAACGATATAA
TGTTAA
SEQ ID No. 94-Mkuzi 1979 MGF 505 2R (AY261362.1:34826-36406)
ATGTTTTCCCTTCAAGACCTTTGCCGAAAGCATCTTTTTATTCTTCCCGATGTTTTTGGCGAGCATGTACTACAA
CGATTAGGACTGTATTGGAGATGTCACGGCTCCCTTCAACGCATAGGAGACGACCACATACTCATACGACGGGAT
CTCATCCTTTCCACCAACGAGGCCTTAAGAATGGCGGGAGAGGAAGGAAACAATGAAGTAGTAAAGCTCTTGTTA
CTGTGGAAGGGAAATCTTCATTACGCCGTCATAGGAGCCTTGCAGGGTGATCAATATGACCTGATCCATAAGTAT
GAAAACCAAATCGGCGACTTTCATTTTATCTTACCATTGATTCAAGACGCGAATACGTTTGAAAAATGCCACGCT
TTAGAACGTTTTTGTGGTGTTTCATGTCTGCTAAAACATGCTACAAAATACAACATGCTCCCTATTCTCCAAAAA
TACCAAGAAGAGCTGTCTATGAGAGCGTATCTTCACGAAACCCTATTTGAACTAGCATGCCTATGGCAGAGGTAT
GATGTCCTTAAATGGATAGAGCAGACCATGCATGTTTACGACCTAAAGATTATGTTTAATATTGCCATCTCCAAG
AGGGATCTGACTATGTACTCCTTAGGATATATTTTCCTTTTTGATAGAGGGAACACCGAAGCTACGTTGCTAACG
CAACATCTCGAGAAGACAGCGGCCAAAGGGCTCCTCCACTTTGTGCTAGAAACGTTAAAATACGGCGGCAACATA
GATACCGTCCTGACCCAAGCCGTAAAGTACAATCATAGAAAACTTTTAGATTATTTTCTGCGTCAACTACCTCGT
AAACATATTGAAAAACTTTTGTTGCTGGCTGTTCAGGAAAAGGCTTCTAAAAAAACATTGAACTTACTGTTGTCA
CATTTAAACTACTCCGTGAAACGCATCAAAAAACTGCTGCGCTATGTGATAGAGTATGAGTCCACCTTGGTGATA
AAGATTTTGTTAAAAAAAAGAGTAAACCTGATAGATGCCATGTTGGAAAAGATGGTAAGATATTTTTCTGCGACA
AAAGTGAGGACGATCATGGATGAGCTTTCGATTAGTCCGGAAAGAGTCATTAAGATGGCTATACAGAAAATGAGA
ACGGATATCGTAATCCATACTTCTTATGTTTGGGAGGATGATCTAGAACGTTTTACTCGTCTTAAAAATATGGTA
TACACCATAAAGTACGAACATGGGAAAAAAATGTTAATTAAAGTCATGCACGGCATATACAAAAACTTATTATAC
GGCGAAAGGGAAAAAGTCATGTTTCATTTAGCCAAGCTCTATGTTGCTCAAAATGCGGCCACCCAATTCAGAGAC
ATTTGTAAGGACTGTTACAAACTGGATGTGGCACGGTTTAAACCGCGGTTTAAGCAACTAATATTAGACTGTTTA
GAAATTGTTACTAAAAAATCTTGCTATAGTATCCTGGAAATCTTAGAAAAACATATTATTTCCCTATTTACTATG
AAAGTTATGACTGAAGAAGAAAAAAACCTATGTTTAGAAATATTATATAAAGTAATTCATTATAAAACAATACAA
TGTTAA
SEQ ID No. 95-Pretorisuskop/96/4 MGF 505 2R (AY261363.1:33795-35374)
ATGTTTTCCCTTCAAGACCTTTGCCGAAAGCATCTTTTTATTCTTCCCGATGTTTTTGGCGAGCATGTACTACAA
CGATTAGGACTGTATTGGAGATGTCACGGCTCCCTTCAACGCGTAGGAGACGACCACATCCTCATACGGCGGGAT
CTCATCCTTTCCACCAACGAGGCCTTAAGAATGGCGGGAGAAGAAGGAAACAATGAAGTAGTAAAGCTCTTGTTA
CTGTGGAAGGGAAATCTTCATTACGCCATCATAGGAGCCTTGCAGGGTGATCAATATGACCTGATCCATAAGTAT
GAAAACCAAATCGGCGACTTTCATCTTATCTTACCATTGATTCAAGATGCGAAAACGTTTGAAAAATGCCACGCT
TTAGAACGTTTTTGTGGTGTTTCATGTCTGCTAGAACATGCTACAAAATACAACATGCTCCCTATTCTCCAAACA
TACCAAGAAGAGCTGTCTATGAGAGCATATCTTCGCGAAACCCTATTTGAACTAGCGTGCCTGTGGCAGAGGTAT
GATGTCCTTAAATGGATAGAGCAAACCATGCATGTTTACGACCTAAAGATTATGTTTAATATTGCCATCTCCAAG
AGGGATCTGACGATGTATTCCTTAGGATATATTCTTCTTTTTGATAGAGAGAACACCGAAGCTACGTTGTTAACA
CAACATCTCGAGAAGACAGCGGCCAAAGGGCTCCTCTACTTTGTGCTAGAAACGTTAAAATACGGCGGTAACATA
GATATCGTCCTGACTCAAGCCGTAAAGTACAATCATAGAAAACTTTTAGATTATTTTCTGCGTCAACTACCTCGT
AAACATATTGAAAAGCTTTTGTTGCTGGCCGTGCAGGAAAAGGCTTCTAAAAAAACATTGAACTTACTGTTGTCC
CATCTAAACTACTCCGTGAAACGTATCAAAAAACTGCTGCGCTATGTGATAGAGTACGAGTCCACCTTGGTGATA
AAGATTTTATTAAAAAAAAGAGTAAACCTGATAGATGCCATGTTGGAAAAGATGGTAAGATATTTTTCTGCGACG
AAAGTGAGGACGATCATGGATGAGCTTTTGATTAGTCCGGAAAGAGTCATTAAGATGGCTATACAGAAAATGAGA
ACGGATATCGTAATCCATACTTCTTATGTTTGGGAGGATGATCTAGAACGACTTACTCGTCTTAAAAATATGGTA
TACACCATAAAGTACGAACATGGGAAAAAAATGTTAATTAAAGTCATGCACGGCATATACAAAAACTTATTATAC
GACGAAAGAGAAAAGGTCATGTTTCATTTAGCCAAGCTCTATGTTGCTCAAAACGCGGCCACCCAATTCAGAGAC
ATTTGTAAGGACTGTTACAAACTGGATGTGGCACGGTTTAAACCGCGGTTTAAGCAACTAATGTTAGACTGTTTA
GAAATTGTTACTAAAAAATCTTGCTATAGTATCCTGGAAATCTTAGAAAAACATATTATTTCCCTATTTACTATG
AAAGTTATGACTGAAGAGGAAAAAAACCTATGTTTAGAAATATTATATAAGTAA
SEQ ID No. 96-Tengani 62 MGF 505 2R (AY261364.1:28261-29830 )
ATGTTTTCCCTTCAAGACCTTTGCCGAAAGCATCTTTTTATTCTTCCCGATGTTTTTGGCGAGCATGTACTACAA
CGATTAGGACTGTATTGGAGATGTCACGGCTCCCTTCAACGCGTAGGAGACGACCACATACTCATACGGCGGGAT
CTCATCCTTTCCACCAACGAGGCCTTAAGAATGGCGGGAGAGGAAGGAAACAATGAAGTAGTAAAGCTCTTGTTA
CTGTGGAAGGGAAATCTTCATTACGCCGTCATAGGAGCCTTGCAGGGTGATCAATATGACCTGATCCATAAGTAT
GAAAACCAAATCGGCGACTTTCATCTTATCTTACCATTGATTCAAGACGCGAAAACGTTTGAAAAATGCCATGCT
TTAGAACGTTTTTGTGGTGTTTCATGTCTGCTAAAACATGCTACAAAATACAACATGCTCCCTGTTCTCCAAAAA
TACCAAGAAGAGCTGTCCATGAGAGCATATCTTTGCGAAACCCTATTTGAACTAGCATGCCTATGGCAGAGGTAT
GATGTCCTTAAATGGATAGAGCAAACCATGCATGTTTACGACCTAAAGATTATGTTTAATATTGCCATCTCCAAG
AGGGATCTGACTATGTACTCCTTAGGATATATTTTCCTTTTTGATAGAGGGAACACCGAAGCTACGTTGCTAACG
CAATATCTCGAGAAGACAGCGGCCAAAGGGCTCCTCCACTTTGTGCTAGAAACGTTAAAATACGGCGGTAACATA
GATATTGTCCTGACCCAAGCCGTAAAGTACAATCATAGAAAACTTTTAGATTATTTTCTGCGTCAACTACCTCGT
AAACATATTGAAAAGCTTTTGTTGCTGGCCGTGCAGGAAAAGGCTTCTAAAAAAACATTGAACTTACTGTTGTCA
CATTTAAACTACTCCGTGAAACGCATCAAAAAACTGCTGCGCTATGTGATAGAGTACGAGTCCACCTTGGTGATA
AAGATTTTATTAAAAAAAAGAGTAAACCTGATAGATGCCATGTTGGAAAAGATGGTAAGATATTTTTCTGCGACG
AAAGTGAGGACGATCATGGATGAGCTTTTGATTAGTCCGGAAAGAGTCATTAAGATGGCTATACAGAAAATGAGA
ACGGATATCGTAATCCATACTTCTTATGTATGGGAGGATGATCTAGAACGACTTACTCGTCTTAAAGATATGGTA
TACACCGTAAAGTACGAACATGGGAAAAAAATGTTAATTAAAGTCATACACGGCATATACAAAAACTTATTATAC
GGCGAAAAAGAAAAGGTCATGTTTCATTTAGCCAAGCTCTATGTTGCTCAAAACGCGGCCACCCAATTCAGAGAC
ATTTGTAAGGACTGTTACAAACTGGATGTGGCACGGTTTAAACCGCGGTTTAAGCAACTAATGTTAGACTGTTTA
GAAATTGTTACTAAAAAATCTTGCTATAGTATTCTGGAAATCTTAGAAAAACATATGATTTCCCTATTTACTATG
AAAGTTATGACTGAAGAAGAAAAAAACCTATGTTTAGAAATATTATATAAGTAA
SEQ ID No. 97-Warmbaths MGF 505 2R (AY261365.1:33029-34597)
ATGTTTTCCCTTCAAGACCTTTGCCGAAAGCATCTTTTTATTCTTCCCGATGTTTTTGGCGAGCATGTATTACAA
CGATTAGGACTGTATTGGAGATGTCACGGCTCCCTTCAACGCGTAGGAGACGACCACATACTCATACGGCGGGAT
CTCATCCTTTCCACCAACGAGGCCTTAAGAATGGCGGGAGAAGAAGGAAACAATGAAGTAGTAAAGCTCTTGTTA
CTGTGGAAGGGAAATCTTCATTACGCCATCATAGGAGCCTTGCAGGGTGATCAATATGACCTAATCCATAAGTAT
GAAAACCAAATCGGCGACTTTCATCTTATCTTACCATTGATTCAAGATGCGAAAACGTTTGAAAAATGCCACGCT
TTAGAACGTTTTTGTGGTGTTTCATGTCTGCTAAAACATGCTACAAAATACAACATGCTCCCTATTCTCCAAACA
TACCAAGAAGAGCTGTCTATGAGAGTATATCTTCGCGAAACCCTATTTGAACTAGCATGCCTATGGCAGAGGTAT
GATGTCCTTAAATGGATAGAGCAAACCATGCATGTTTACGACCTAAAGGTTATGTTTAATATTGCCATCTCCAAG
AGAGATCTGACTATGTATTCCTTAGGATATATTCTTCTTTTTGATAGAGAGAACACCGAAGCTACGTTGCTAACG
CAACATCTCGAGAAAACAGCGGCCAAGGGGCTCCTCCACTTTGTGCTAGAAACGTTAAAATACGGCGGTAACATA
GATATCGTCCTGTCCCAAGCCGTAAAGTACAATCATAGAAAACTTTTAGATTATTTTCTGCGTCAACTACCTCGT
AAAAATATTGAAAAACTTTTGTTGCTGGCTGTTCAGGAAAAGGCTTCTAAAAAAACATTGAACTTACTGTTGTCC
CATTTAAACTACTCCGTGAAACGCATCAAAAAACTGCTGCGCTATGTGATAGAGTACGAGTCCACCTTGGTGATA
AAGATTTTATTAAAAAAAAGAGTAAACCTGATAGATGCCATGTTGGAAAAGATGGTAAGATATTTTTCTGCGACG
AAAGTGAGGACGATCATGGATGAGCTTTCGATTAGTCCGGAAAGAGTCATTAAGATGGCTATACAGAAAATGAGA
ACAGATATCGTAATCCATACTTCTTATGTTTGGGAGGATGATCTAGAACGACTTACTCGTCTTAAAAATATGGTA
TACGCCATAAAGTATGAACATGGGAAAAAAATGTTAATGAAAGTCATGCACGGCATATACAAAAACTTATTATAC
GGCGAAAGAGAAAAGGTCATGTTTCATTTAGCCAAGCTCTATGTTGCTCAAAACGCGGCCACCCAATTCAGAGAC
ATTTGTAAGGACTGTTACAAACTGGATGTGGCACGGTTTAAGCAACTAACGTTAAACTGTTTAGAAATTATTACT
AAAAAATCTTGCTATAGTATCCTGGAAATCCTAGAAAAACATATTATTTCCCTATTTACTATGAAAGTTATGACT
GAAGAAGAAAAAAACCTATGTTTAGAAATATTATATAAAGTAATTCATTATAATACAATACAATGTTAA

In an embodiment the attenuated ASFV of the invention comprises a functional version of MGF 505 2R. Suitably the functional version of MGF 505 2R comprises the sequence of SEQ ID No. 86, 87, 88, 89, 90, 91, 92, 93, 94, 95, 96 or 97. Suitably the functional version of MGF 505 2R comprises a sequence having at least 70%, at least 80%, at least 90% or at least 95% identity with SEQ ID No. 86, 87, 88, 89, 90, 91, 92, 93, 94, 95, 96 or 97. Suitably 40 the functional version of MGF 505 2R consists of the sequence of SEQ ID No. 86, 87, 88, 89, 90, 91, 92, 93, 94, 95, 96 or 97.

MGF 505 3R gene sequences

SEQ ID No. 98-Benin 97/1 MGF 505 3R
ATGTCCTCTTCCCTTCAGGAACTTTGTCGAAAAAAGCTGCCTGACTGCATACTTCCAGAGTTTTTTGACGACTAT
GTATTGCAACTGTTAGGACTGCACTGGCAAGATCATGGTTCCCTTCAGCGTATCGAGAAGAACCAGATACTTGTT
CAACAGGAACCCATCCATATCAATGAAGCACTCAAAGTAGCAGCATCGGAAGGGAACTATGAAATCGTAGAGCTG
TTGTTGTCATGGGAGGCAGATCCCCGCTACGCCGTCGTAGGAGCTCTAGAAAGCAAATACTATGACCTGGTTTAC
AAATACTATGACCTGGTTAAAGACTGCCATGATATCTTGCCGCTGATTCAAAATCCGGAAACATTTGAAAAATGT
CATGAGTTAAACAACCCCTGTTCTCTTAAATGCTTATTCAAGCATGCTGTGATACATGACATGCTGCCGATTCTT
CAAAAATATACATACTTTCTGGATGGGTGGGAGTATTGCAACCAGATGCTGTTCGAACTGGCATGTAGTAAAAAA
AAATATGAGATGGTTGTGTGGATAGAGGGAGTTCTAGGCATCGGCAAAGTTACATCTCTTTTCACTATTGCGATT
AGCAACAGAGACCTGCACCTGTATTCCCTGGGCCACTTAATCATTCTTGAGAGAATGCAGTCCTGTGGACAAGAC
CCCACGTTTTTACTAAATCATTTCCTGCGAGACGTTTCAATAAAAGGGCTTCTCCCTTTTGTACTGAAAACCATA
GAATATGGTGGAAGCAAGGAGATAGCCATAACTCTGGCTAAAAAATATCAGCATAAACATATTTTGAAATACTTC
GAAACCGGGAAATGCTAA
SEQ ID No. 99-China/2018/AnhuiXCGQ MGF 505 3R
ATGTCCTCTTCTCTTCAGGAACTTTGTCGAAAAAAGCTGCCTGACTGCATACTTCCAGAGTTTTTTGACGACTAT
GTATTGCAACTGTTAGGACTGCACTGGCAAGATCATGGTTCCCTTCAGCGTATCGAGAAGAACCAGATACTTGTT
CAACAGGAACCCATCCATATCAATGAAGCACTCAAAGTAGCAGCATCGGAAGGGAACTATGAAATCGTAGAGCTG
TTGTTGTCATGGGAGGCAGATCCCCGCTACGCCGTCGTAGGAGCCCTAGAAAGCAAATACTATGACCTGGTTTAC
AAATACTATGACCAAGTTAAAGACTGCCATGATATCTTGCCGCTGATTCAAAATCCGGAAACATTCGAAAGATGT
CATGAGTTAAACAGCACCTGTTCACTGAAATGCTTATTCAAGCATGCTGTGATAAATGACATGCTGCCGATTCTT
CAAAAATATACAGACTATCTGGATAGGTGGGAGTATTGCAGCCAGATGCTGTTCGAACTGGCATGTAGTAAAAAA
AAATATGAGATGGTTGTGTGGATAGAGGGAGTTCTAGGCGTCGGCAAAGTTACATCTCTTTTCACCATTGCGATT
AGCAACAGAGACCTACAGCTGTATTCTCTGGGCTACTCAATTATCCTTGAGAATTTGTACTCCTGTGGACAGGAC
CCCAAGTTTTTACTAAATCATTTCCTGCGAGACGTTTCAATAAAAGGGCTTCTACCCTTTGTAATCAAAACCATA
GAATATGGTGGAAGCAAGGAGATAGCCATAACTCTGGCTAAAAAATATCAGCATAAACATATTTTGAAATACTTC
GAAACCTGGGAAAGCTAG
SEQ ID No. 100-Georgia 2007/1 MGF 505 3R
ATGTCCTCTTCTCTTCAGGAACTTTGTCGAAAAAAGCTGCCTGACTGCATACTTCCAGAGTTTTTTGACGACTAT
GTATTGCAACTGTTAGGACTGCACTGGCAAGATCATGGTTCCCTTCAGCGTATCGAGAAGAACCAGATACTTGTT
CAACAGGAACCCATCCATATCAATGAAGCACTCAAAGTAGCAGCATCGGAAGGGAACTATGAAATCGTAGAGCTG
TTGTTGTCATGGGAGGCAGATCCCCGCTACGCCGTCGTAGGAGCCCTAGAAAGCAAATACTATGACCTGGTTTAC
AAATACTATGACCAAGTTAAAGACTGCCATGATATCTTGCCGCTGATTCAAAATCCGGAAACATTCGAAAGATGT
CATGAGTTAAACAGCACCTGTTCACTGAAATGCTTATTCAAGCATGCTGTGATAAATGACATGCTGCCGATTCTT
CAAAAATATACAGACTATCTGGATAGGTGGGAGTATTGCAGCCAGATGCTGTTCGAACTGGCATGTAGTAAAAAA
AAATATGAGATGGTTGTGTGGATAGAGGGAGTTCTAGGCGTCGGCAAAGTTACATCTCTTTTCACCATTGCGATT
AGCAACAGAGACCTACAGCTGTATTCTCTGGGCTACTCAATTATCCTTGAGAATTTGTACTCCTGTGGACAGGAC
CCCAAGTTTTTACTAAATCATTTCCTGCGAGACGTTTCAATAAAAGGGCTTCTACCCTTTGTAATCAAAACCATA
GAATATGGTGGAAGCAAGGAGATAGCCATAACTCTGGCTAAAAAATATCAGCATAAACATATTTTGAAATACTTC
GAAACCTGGGAAAGCTAG
SEQ ID No. 101-Ken05/Tk1 MGF 505 3R
ATGTCCTCTTCCCTTCAGGAACTTTGCCGAAAAAACTTACCCGACTACATACTTCCAGAGTTCTTTGACGACTAT
GTGTTGCAACTATTAGGACTACACTGGCAAGATCATGGTTCTCTTCAACGTACCGGAAAGAATCAGGTACTTGTT
CAACAGGAACCCATTCATATCAATGAAGCACTAAAAGTGGCAGCATCAGAGGGAAACTTTGAAATCGTAGAACTG
TTGTTGTCATGGAAGGCAGACCCCCGCTACGCTGTCGTAGGAGCGCTAGAAAGCAAATACTATGACCTCGTTTAC
AAGTATTACAACCTAGTTGAAGACCGCCATGATATGTTGCCGCTGATCCAAAATTCAGAAACGTTCGAAAGATGT
CATGAGTTAAACAACTGTTCTCTTAAATGCTTATTCAAGCATGCTGTAATATATGACAAGCTGCCGATTCTACAA
AAATATGCAGACTATTTGGACGGGTGGCCGTATTGCAACCAGATGCTGTTCGAGTTGGCATGTAAAAAACAAAAA
TATAACATGGTTGTGTGGATAGAGGGAGTCCTGGGCGTCGGCAACTTCACAATTCTTTTCACAATTGCGATTATC
AAAAGGGACCTACAGCTGTATTCCCTGGGCTACTCCATCATCCTTGAGAGAATGTATTCCTGTGGATACGACCCC
ACGTTTTTACTAAATCACTATCTGCGAGTGGTTTCAACAAAAGGGCTTCTGCCTTTTGTACTGAAAACCATAGAA
TATGGTGGAAGCAAAGAGATAGCCTTAACTTTGGCTAAAAAATATCAGCATGAAACTATTTTGAGATACTTCGAA
ACCAGGAAATCCCAGGAGTGCTAA
SEQ ID No. 102-Ken06.Bus MGF 505 3R
ATGTCCTCTTCCCTTCAGGAACTTTGCCGAAAAAACTTACCCGACCACATACTTCCAGAGTTCTTTGACGACTAT
ATATTGCAACTATTAGGACTGCACTGGCAAGATCATGGTTCTCTTCAGCGTACCGAGAAGAATCAGGTACTTGTT
CAACAGGAACCCATCCATATCAATGAAGCACTAAAAGTGGCAGCATCAGAAGGAAACTACGAAATAGTAGAGCTG
TTGTTGTCATGGAAGGCAGACCCCCGATACGCTGTCGTAGGAGCGCTAGAAAGCAAATACTATGACCTCGTTTAC
AAATATTACGACCAAGTTAAAGACTGCCATGATATCTTGCCGCTGATCCAAAATTCGGAAACATTCGAAAGATGT
CATGAGTTGAACAATTGTTCTCTTAAATGCTTATTCAAGCATGCTGTGATATATGACAAGCTGCCGATTCTTCAA
AAATATGCAAGCTATCTGGATGGGTGGCCGTATTGCAACCAGATGCTGTTCGAGTTGGCATGTAAAAAACAAAAA
TATAACATGGCTGTATGGATAGAGGGAGTCCTGGGCGTCGGCAACTTCACAATTCTTTTCACGATTGCGATTATC
AAAAGAGACCTACAGCTGTATTCCCTGGGCTACTCAATGATTCTTGAGAAAATGTACTCCTGTGGATACGACCCT
ACGTTTTTACTAAATCATTATCTGCGAATCGTTTCAACAAAGGGGCTTCTGCCCTTTGTGCTGAAAACCATAGAA
TATGGTGGAAGCAAAGAGATAGCCATCACTTTGGCTAAAAAGTATCAGCATGAAAATATTTTGAGATACTTCGAA
ACCAGGAAAACCCAAGAGTGCTAA
SEQ ID No. 103-Kenya1950 MGF 505 3R
ATGTCCTCTTCCCTTCAGGAACTTTGCCGAAAAAACTTACCCGACGACATACTTCCAGAGTTCTTTGACGACTAT
GTGTTGCAACTATTAGGACTACACTGGCAAGATCATGGTTCTCTTCAACGTACCGGAAAGAATCAGGTACTTGTT
CAACAGGAACCCATTCATATTAATGAAGCACTAAAAGTGGCAGCATCAGAGGGAAACTTTGAAATCGTAGAACTG
TTGTTGTCATGGAAGGCAGATCCCCGCTACGCTGTCGTAGGAGCGCTAGAAAGCAAATACTATGACCTCGTTTAC
AAGTATTACAACCTAATTGAAGACCGCCATGATATGTTGCCGCTGATCCAAAATTCAGAAACGTTCGAAAGATGT
CATGAGTTAAACAACTGTTCTCTTAAATGCTTATTCAAGCATGCTGTGATATATGACAAGCTGCCGATTCTACAA
AAATATGCAGACTATTTGGACGGGTGGCCGTATTGCAACCAGATGCTGTTCGAGTTGGCATGTAAAAAACAAAAA
TATAACATGGTTGTGTGGATAGAGGGAGTCCTGGGCGTCGGCAACTTCACAATTCTTTTCACGATTGCGATTATC
AAAAGGGACCTACAGCTGTATTCCCTGGGCTACTCCATCATCCTTGAGAGAATGTACTCCTGTGGATACGACCCC
ACGTTTTTACTAAATCACTATCTGCGAGTGGTTTCAACAAAAGGGCTTCTGCCTTTTGTACTGAAAACCATAGAA
TATGGTGGAAGCAAAGAGATAGCCATCACTTTGGCTAAAAAGTATCAGCATGAAACTATTTTGAGATACTTCGAA
ACCAGGAAATCCCAGGAGTGCTAA
SEQ ID No. 104-L60 MGF 505 3R
ATGTCCTCTTCCCTTCAGGAACTTTGTCGAAAAAAGCTGCCTGACTGCATACTTCCAGAGTTTTTTGACGACTAT
GTATTGCAACTGTTAGGACTGCACTGGCAAGATCATGGTTCCCTTCAGCGTATCGAGAAGAACCAGATACTTGTT
CAACAGGAACCCATCCATATCAATGAAGCACTCAAAGTAGCAGCATCGGAAGGGAACTATGAAATCGTAGAGCTG
TTGTTGTCATGGGAGGCAGATCCCCGCTACGCCGTCGTAGGAGCTCTAGAAAGCAAATACTATGACCTGGTTTAC
AAATACTATGACCTGGTTAAAGACTGCCATGATATCTTGCCGCTGATTCAAAATCCGGAAACATTTGAAAAATGT
CATGAGTTAAACAACCCCTGTTCTCTTAAATGCTTATTCAAGCATGCTGTGATACATGACATGCTGCCGATTCTT
CAAAAATATACATACTTTCTGGATGGGTGGGAGTATTGCAACCAGATGCTGTTCGAACTGGCATGTAGTAAAAAA
AAATATGAGATGGTTGTGTGGATAGAGGGAGTTCTAGGCATCGGCAAAGTTACATCTCTTTTCACTATTGCGATT
AGCAACAGAGACCTGCACCTGTATTCCCTGGGCCACTTAATCATTCTTGAGAGAATGCAGTCCTGTGGACAAGAC
CCCACGTTTTTACTAAATCATTTCCTGCGAGACGTTTCAATAAAAGGGCTTCTCCCTTTTGTACTGAAAACCATA
GAATATGGTGGAAGCAAGGAGATAGCCATAACTCTGGCTAAAAAATATCAGCATAAACATATTTTGAAATACTTC
GAAACCGGGAAATGCTAA
SEQ ID No. 105-Malawi Lil-20/1/1983 MGF 505 3R
ATGTCCCTTCAGGAACTTTGCCGAAAAAACCTGCCCGACTGCGAACTTCCAGAGTTCTTTGACGACTATGTATTG
CAACTGTTAGGATTGCACTGGCAAGATCATGGTTCTCTTCAGCGTACCGGGAAGAATCAGGTACTTGTTCAACAG
GAACCCATCCATATCAATGAAGCACTAAAAAGTGCGGCATCAGAAGGGAACTATGAAATCGTAGAGCTGCTGTTG
TCATGGGAGGCAGATCCCCGCTACGCTGTCGTAGGAGCCCTAGAAAGCAATTACTATGACCTGGTTCACAAATAT
TATGACCAAGTTAAAGACTGCCATGATATGCTACCACTGATCCAAAATCCGGAAATGTTCGAAAAATGTCATGAG
TTAAACAACACCTGTTCTCTTAAATGCTTATTCAAACATGCTGTGATACATGACATGCTGCCGATTCTTCAAAAA
TATTCAGACTATTTGGATGGGTGGCAGTATTGCAATCAGATACTGTTCGAATTGGCATGTAAAAGACAAAAATAT
AATATGGTTGTGTGGATAGAGGGAGTTCTAGGCGTCGGCAACTTTAAAATTCTTTTCACCATTGCCATTAACAAC
AGAGATCTACAGCTGTATTCTCTGGGGTACTTAATCATTCTTGAAAGATTGTACTCCTGTGGACAAGACCCCACG
TTTTTACTAAACCATTTCCTACGAGACGTTTCAATGAAGGGGCTTCTGCCCTTTGTACTGAAAACCATAGAATTT
GGTGGAAGTAAGGAGATAGCCATAACTCTGGCTAAAAAATATCAGCATAAACATATTTTGAAGTATTTCGAAACC
GAAGAATGCTAA
SEQ ID No. 106-Mkuzi 1979 MGF 505 3R
ATGTCCTCTTCCCTTCAGGAACTTTGTCGAAAAAAGCTGCCTGACTGCATACTTCCAGAGTTTTTTGACGACTAT
GTATTGCAACTGTTAGGACTGCACTGGCAAGATCATGGTTCCCTTCAGCGTATCGAGAAGAACCAGATACTTGTT
CAACAGGAACCCATCCATATCAATGAAGCACTCAAAGTAGCAGCATCGGAAGGGAACTATGAAATCGTAGAGCTG
TTGTTGTCATGGGAGGCAGATCCCCGCTACGCCGTCGTAGGAGCTCTAGAAAGCAAATACTATGACCTGGTTTAC
AAATACTATGACCAAGTTAAAGACTGCCATGATATCTTGCCGCTGATTCAAAATCCGGAAACATTTGAAAAATGT
CATGAGTTAAACAACCCCTGTTCTCTTAAATGCTTATTCAAGCATGCTGTGATACATGACATGCTGCCGATTCTT
CAAAAATATACATACTTTCTGGATGGGTGGGAGTATTGCAGCCAGATGCTGTTCGAATTGGCATGTAGTAAAAAA
AAATACGAGATGGTTGTGTGGATAGAGGGAGTTCTAGGCATCGGCAAAGTTACATCTCTTTTCACTATTGCGATT
AGCAACAGAGACCTGCACCTGTATTCCCTGGGCCACTTAATCATTCTTGAGAGAATGCAGTCCTGTGGACAAGAC
CCCAAGTTTTTACTAAATCATTTCTTGCGAGACGTTTCAATAAAAGGGCTTCTCCCCTTTGTACTGAAAACCATA
GAATATGGTGGAAGCAAGGAGATAGCCATAACTCTGGCTAAAAAATATCAGCATAAACATATTTTGAAATACTTC
GAAACCGGGAAATGCTAA
SEQ ID No. 107-NHV MGF 505 3R
ATGGTTGTGTGGATAGAGGGAGTTCTAGGCATCGGCAAAGTTACATCTCTTTTCACTATTGCGATTAGCAACAGA
GACCTGCACCTGTATTCCCTGGGCCACTTAATCATTCTTGAGAGAATGCAGTCCTGTGGACAAGACCCCACGTTT
TTACTAAATCATTTCCTGCGAGACGTTTCAATAAAAGGGCTTCTCCCTTTTGTACTGAAAACCATAGAATATGGT
GGAAGCAAGGAGATAGCCATAACTCTGGCTAAAAAATATCAGCATAAACATATTTTGAAATACTTCGAAACCGGG
AAATGCTAA
SEQ ID No. 108-OURT 88/3 MGF 505 3R
ATGGTTGTGTGGATAGAGGGAGTTCTAGGCATCGGCAAAGTTACATCTCTTTTCACTATTGCGATTAGCAACAGA
GACCTGCACCTGTATTCCCTGGGCCACTTAATCATTCTTGAGAGAATGCAGTCCTGTGGACAAGACCCCACGTTT
TTACTAAATCATTTCCTGCGAGACGTTTCAATAAAAGGGCTTCTCCCTTTTGTACTGAAAACCATAGAATATGGT
GGAAGCAAGGAGATAGCCATAACTCTGGCTAAAAAATATCAGCATAAACATATTTTGAAATACTTCGAAACCGGG
AAATGCTAA
SEQ ID No. 109-Pretorisuskop/96/4 MGF 505 3R
ATGTCTTCTTCCCTTCAGGAACTTTGTCGAAAAAAGCTGCCTGACTGCATACTTCCAGAGTTTTTTGACGACTAT
GTATTGCAACTGTTAGGACTGCACTGGCAAGATCATGGTTCTCTTCAGCGTATCGAGAAGAACCAGATACTTGTT
CAACAGGAACCCATCCATATCAATGAAGCACTCAAAGTAGCAGCATCGGAAGGGAACTATGAAATCGTAGAGCTG
TTGTTGTCATGGGAGGCAGATCCCCGCTACGCCGTCGTAGGAGCTCTCGAAAGCAAATACTATGACCTGGTTCAC
AAATACTATGGCCAAGTTAAAGACTGCCATGATATCTTGCCGCTGATTCAAAATCCGGAAACATTCGAAAAATGT
CATGAGTTAAACAGCACCTGTTCACTGAAATGCTTATTCAAGCATGCTGTGATGAATGACATGCTGCCGATTCTT
GAAAAATATACAGACTATCTGGATAGGTGGGAGTATTGCAGCCAGATGCTGTTCGAATTGGCATGCAGAAAAAAA
AAATATGAGATGGTTGTGTGGATAGAGGGAGTTCTATGCGTCGGCAAAGTTACGTCTCTTTTCACCATTGCGATT
AGCAACAGAGACCTACAGCTGTATTCTCTGGGCTACTCAATTATTCTTGAGAAAATGTACTCCTGTCGACAGGAC
CCCACGTTTTTACTAAATCATTTCCTGCGAGACGTTTCGATAAAGGGGCTTCTGCCCTTTGTACTGAAAACCATA
GAATATGGTGGAAGCAAGGAGATAGCCATAACTCTGGCTAAAAAATATCAGCATAAACATATTTTGAAATACTTC
GAAACCTGGGAAAGCTAG
SEQ ID No. 110-Tengani 62 MGF 505 3R
ATGTCCTCTTCCTCCCTTCAGGAACTTTGCCGAAAAAAGCTGCCTGACTGCATCCTTCCAGAGTTTTTCGACGAC
TATGTATTGCAACTGTTAGGACTGCACTGGCAAGATCATGGTTCTCTTCAGCGTATCGAGAAGAACCAGATACTT
GTTCAACAGGAACCCATCCATATCAATGAAGCACTCAAAGTAGCAGCATCGGAAGGGAACTATGAAATCGTAGAG
CTGTTGTTGTCATGGGAGGCAGATCCCCGCTACGCCGTCGTAGGAGCTCTCGAAAGCAAATACTATGACCTGGTT
CACAAATACTATGACCAAGTTAAAGACTGCCATGATATCTTGCCGCTGATTCAAAATCCGGAAACATTCGAAAAG
TGTCATGAGTTAAACAGCACCTGTTCACTGAAATGCTTATTCAAGCATGCTGTGATAAATGACATGCTGCCGATT
CTTCAAAAATATACAGACTATCTGGATAGGTGGGAGTATTGTAGCCAGATGCTGTTCGAATTGGCATGTAGAAAA
AAAAAGTATGAGATGGTTGTGTGGATAGAGGGAGCTCTGGGCGTCGGCAAAGTTACATCTCTTTTCACCATTGCG
ATTAGCAACAGAGACCTGCAGCTGTATTCTCTGGGCTACTCAATGATTCTTGAGAAATTGTACTCCTGTGGACAG
GACCCCACGTTTTTACTAAATCATTTCCTGCGAGACGTTTCGATAAAGGGGCTTCTGCCCTTTGTACTCAAAACC
ATAGAATATGGTGGAAGCAAGGAGATAGCCATAACTCTGGCTAAAAAATATCAGCATAAACATATTTTGAAATAC
TTCGAAACTTGGGAAAGCTAG
SEQ ID No. 111-Warmbaths MGF 505 3R
ATGTCCTCTTCCCTTCAGGAACTTTGTCGAAAAAAGCTGCCTGACTGCATACTTCCAGAGTTTTTCGACGACTAT
GTATTGCAACTGTTAGGACTGCACTGGCAAGATCATGGTTCTCTTCAGCGTATCGAGAAGAACCAGATACTTGTT
CAACAGGAACCCATCCATATCAATGAAGCACTCAAAGTAGCAGCATCGGAAGGGAACTATGAAATCGTAGAGCTG
TTGTTGTCATGGGAGGCAGATCCCCGCTACGCCGTCGTAGGAGCTCTCGAAAGCAAATACTATGACCTGGTTCAC
AAATACTATGGCCAAGTTAAAGACTGCCATGATATCTTGCCGCTGATTCAAAATCCGGAAACATTCGAAAAATGT
CATGAGTTAAACAGCACCTGTTCACTGAAATGCTTATTCAAGCATGCTGTGATGAATGACATGCTGCCGATTCTT
GAAAAATATACAGACTATCTGGATAGGTGGGAGTATTGCAGCCAGATGCTGTTCGAATTGGCATGTAGAAAAAAA
AAATATGAGATGGTTGTGTGGATAGAGGGAGTTCTAGGCATCGGCAAAGTTACGCCTCTTTTCACCATTGCGATT
AGCAACAGAGACCTACAGCTGTATTCTCTGGGCTACTCAATTATTCTTGAGAGAATGTATTCCTGTGAACAGGAT
CCCACGTTTTTACTAAATCATTTCCTGCGAGACGTTTCGATAAAGGGGCTTCTGCCCTTTGTACTGAAAACCATA
GAATATGGTGGAAGCAAAGAGATAGCCATAACTCTGGCTAAAAAATATCAGCATAAACATATTTTGAAATACTTC
GAAACATGGGAAAGGTAA
SEQ ID No. 112-Warthog MGF 505 3R
ATGTCCTCTTCCCTTCAGGAACTTTGCCGAAAAAAGCTGCCTGACTGCATACTTCCAGAGTTTTTCGACGACTAT
GTATTGCAACTGTTAGGATTGCATTGGCAAGATCATGGTTCTCTTCAGCGTATCGAGAAGAACCAGATACTTGTT
CAACAGGAACCCATCCATATCAATGAAGCACTCAAAGTAGCAGCATCGGAAGGGAACTATGAAATCGTAGAGCTG
TTGTTGTCATGGGAGGCAGATCCCCGCTACGCCGTCGTAGGAGCTCTCGAAAGCAAATACTATGACCTGGTTCAC
AAATACTATGGCCAAGTTAAAGACTGCCATGATATCTTGCCGCTGATTCAAAATCCGGAAACATTTGAAAAGTGT
CATGAGTTAAACAGCACCTGTTCACTGAAATGCTTATTCAAGCATGCTGTGATGAATGACATGCTGCCGATTCTT
GAAAAATATACAGACTATCTGGATAGGTGGGAGTATTGCAGCCAGATGCTGTTCGAATTGGCATGTAGAAAAAAA
AAATATGAGATGGTTGTGTGGATAGAGGGAGTTCTAGGCGTCGGCAAAGTTACGTCTCTTTTCACCATTGCGATT
AGCAACAGAGACCTACAGCTGTATTCTCTGGGCTTCTCAATTATTCTTGAGAAATTGTACTCCTGTGGACAGGAC
CCCACGTTTTTACTAAATCATTTCCTGCGAGACGTTTCGATAAAGGGGCTTCTGCCCTTTGTACTCAAAACCATA
GAATATGGTGGAAGCAAGGAGATAGCCATAACTCTGGCTAAAAAATATCAGCATAAACATATTTTGAAATACTTC
GAAACCTGGGAAAGCTAG

In an embodiment the attenuated ASFV of the invention comprises a functional version of MGF 505 3R. Suitably the functional version of MGF 505 3R comprises the sequence of SEQ ID No. 98, 99, 100, 101, 102, 103, 104, 105, 106, 107, 108, 109, 110, 111 or 112. Suitably the functional version of MGF 505 3R comprises a sequence having at least 70%, at least 80%, at least 90% or at least 95% identity with SEQ ID No. 98, 99, 100, 101, 102, 103, 104, 105, 106, 107, 108, 109, 110, 111 or 112. Suitably the functional version of MGF 505 3R consists of the sequence of SEQ ID No. 98, 99, 100, 101, 102, 103, 104, 105, 106, 107, 108, 109, 110, 111 or 112. In an embodiment the invention provides an attenuated ASFV which comprises the following sequences from the Benin 97/1 strain:
(i) SEQ ID No. 11 (MGF 360 10L) and/or SEQ ID No. 24 (MGF 360 11L), and
(ii) SEQ ID No. 86 (MGF 505 2R) and/or SEQ ID No. 98 (MGF 505 3R).

In an embodiment the invention provides an attenuated ASFV which comprises the following sequences from the China/2018/AnhuiXCGQ strain:
(i) SEQ ID No. 12 (MGF 360 10L) and/or SEQ ID No. 25 (MGF 360 11L), and
(ii) SEQ ID No. 87 (MGF 505 2R) and/or SEQ ID No. 99 (MGF 505 3R).

In an embodiment the invention provides an attenuated ASFV which comprises the following sequences from the Georgia 2007/1 strain:
(i) SEQ ID No. 13 (MGF 360 10L) and/or SEQ ID No. 26 (MGF 360 11L), and
(ii) SEQ ID No. 88 (MGF 505 2R) and/or SEQ ID No. 100 (MGF 505 3R).

In an embodiment the invention provides an attenuated ASFV which comprises the following sequences from the Ken05/Tk1 strain:
(i) SEQ ID No. 14 (MGF 360 10L) and/or SEQ ID No. 27 (MGF 360 11L), and
(ii) SEQ ID No. 89 (MGF 505 2R) and/or SEQ ID No. 101 (MGF 505 3R).

In an embodiment the invention provides an attenuated ASFV which comprises the following sequences from the Ken06.Bus strain:
(i) SEQ ID No. 15 (MGF 360 10L) and/or SEQ ID No. 28 (MGF 360 11L), and
(ii) SEQ ID No. 90 (MGF 505 2R) and/or SEQ ID No. 102 (MGF 505 3R).

In an embodiment the invention provides an attenuated ASFV which comprises the following sequences from the Kenya 1950 strain:
(i) SEQ ID No. 16 (MGF 360 10L) and/or SEQ ID No. 29 (MGF 360 11L), and
(ii) SEQ ID No. 91 (MGF 505 2R) and/or SEQ ID No. 103 (MGF 505 3R).

In an embodiment the invention provides an attenuated ASFV which comprises the following sequences from the L60 strain:
(i) SEQ ID No. 17 (MGF 360 10L) and/or SEQ ID No. 30 (MGF 360 11L), and
(ii) SEQ ID No. 92 (MGF 505 2R) and/or SEQ ID No. 104 (MGF 505 3R).

In an embodiment the invention provides an attenuated ASFV which comprises the following sequences from the Malawi Lil-20/1 strain:
(i) SEQ ID No. 18 (MGF 360 10L) and/or SEQ ID No. 31 (MGF 360 11L), and
(ii) SEQ ID No. 93 (MGF 505 2R) and/or SEQ ID No. 105 (MGF 505 3R).

In an embodiment the invention provides an attenuated ASFV which comprises the following sequences from the Mkuzi 1979 strain:
(i) SEQ ID No. 19 (MGF 360 10L) and/or SEQ ID No. 32 (MGF 360 11L), and
(ii) SEQ ID No. 94 (MGF 505 2R) and/or SEQ ID No. 106 (MGF 505 3R).

In an embodiment the invention provides an attenuated ASFV which comprises the following sequences from the Pretorisuskop/96/4 strain:
(i) SEQ ID No. 20 (MGF 360 10L) and/or SEQ ID No. 33 (MGF 360 11L), and
(ii) SEQ ID No. 95 (MGF 505 2R) and/or SEQ ID No. 109 (MGF 505 3R).

In an embodiment the invention provides an attenuated ASFV which comprises the following sequences from the Tengani 62 strain:
(i) SEQ ID No. 21 (MGF 360 10L) and/or SEQ ID No. 34 (MGF 360 11L), and
(ii) SEQ ID No. 96 (MGF 505 2R) and/or SEQ ID No. 110 (MGF 505 3R).

In an embodiment the invention provides an attenuated ASFV which comprises the following sequences from the Warmbaths strain:
(i) SEQ ID No. 22 (MGF 360 10L) and/or SEQ ID No. 35 (MGF 360 11L), and
(ii) SEQ ID No. 97 (MGF 505 2R) and/or SEQ ID No. 111 (MGF 505 3R).

In an embodiment the invention provides an attenuated ASFV which comprises the following sequences from the Warthog strain:
(i) SEQ ID No. 23 (MGF 360 10L) and/or SEQ ID No. 36 (MGF 360 11L), and
(ii) SEQ ID No. 112 (MGF 505 3R).

The translation products (i.e. protein sequences) of these genes from different strains are given below:

SEQ ID No. 1-Georgia 2007/1 MGF 360 10L protein
MFPSLQSFAKKVLARQQVSIEYHVILERCGLWWYKAPISLDCKHMLIKLPSFADGLDLNTALMLATKENN
YQLIKLFTEWGADINYGLICANTPPVREFCWELGAKYRVDKKKIMHMFFKLIHPGTTSSNIILCLKLFND
NPFPAYVIIREIRSSIYWKLKRLVEDTDILSNMSDGDMLTIYCFIVALQDNLREAISYVYQHFKYLNTWW
LICALCFNKLFDLHNLYEKEKIRMDVDEMMRMACTKDNNFLTIYYCFLLGANINSAMLACVRFYNMDNLF
FCIDLGADAFEEAKALAEQRNYYLISHHLSLDIYSADSSLLTLKEADPNKIYHLLKNYKLKSMLA
SEQ ID No. 2-Georgia 2007/1 MGF 360 11L protein
MLPSLQSLTKKVLAGQCIPVDQYHVLKCCGLWWHNGPIMLHIRRNKLFIRSTCFSQGIELNIGLMKAVKE
NNHDLIKLFTEWGADINYGMICALTENTRDLCKELGAKEYLEREYILKIFFDTTRDKTSSNIIFCHEVFS
NNPNLRIIDNLDLRGEIMWELRGLMEITFMLDHDDSFSTVLTKYWYAIAVDYDLKDAIRYFYQKYPRLHR
WRLMCALFYNNVFDLHELYEIERVRMDIDEMMHIACIQDYSYSAIYYCFIMGANINQAMLVSIQNYNLGN
LFFCIDLGADAFEEGKALAEQKENYLIAHALSLKHYNPVISLLSIVTDPEKINCMLKNYHSINMGIFLDY
EQR
SEQ ID No. 7-Georgia 2007/1 MGF 505 2R protein
MFSLQDLCRKHLFILPDVFGEHVLQRLGLYWRCHGSLQRIGDDHILIRRDLILSTNEALRMAGEEGNNEV
VKLLLLWKGNLHYAVIGALQGDQYDLIHKYENQIGDFHFILPLIQDANTFEKCHALERFCGVSCLLKHAT
KYNMLPILQKYQEELSMRAYLHETLFELACLWQRYDVLKWIEQTIHVYDLKIMFNIAISKRDLTMYSLGY
IFLFDRGNTEATLLTQHLKKTAAKGLLHFVLETLKYGGNIDTVLTQAVKYNHRKLLDYFLRQLPRKHIEK
LLLLAVQEKASKKTLNLLLSHLNYSVKRIKKLPRYVIEYESTLVIKILLKKRVNLIDAMLEKMVRYFSAT
KVRTIMDELSISPERVIKMAIQKMRTDIVIHTSYVWEDDLERLTRLKNMVYTIKYEHGKKMLIKVMHGIY
KNLLYGEREKVMFYLAKLYVAQNAATQFRDICKDCYKLDVARFKPRFKQLILDCLEIITKKSCYSILEIL
EKHIISLFTMKVMTEEEKNLCLEILYKVIHYKTIQC
SEQ ID No. 8-Georgia 2007/1 MGF 505 3R protein
MSSSLQELCRKKLPDCILPEFFDDYVLQLLGLHWQDHGSLQRIEKNQILVQQEPIHINEALKVAASEGNY
EIVELLLSWEADPRYAvvGALESKYYDLVYKYYDQVKDCHDILPLIQNPETFERCHELNSTCSLKCLFKH
AVINDMLPILQKYTDYLDRWEYCSQMLFELACSKKKYEMVVWIEGVLGVGKVTSLFTIAISNRDLQLYSL
GYSIILENLYSCGQDPKFLLNHFLRDVSIKGLLPFVIKTIEYGGSKEIAITLAKKYQHKHILKYFETWES
MGF 360 12L protein sequences
SEQ ID No. 113-Benin 97/1 MGF 360 12L protein
MLPSLQSLTKKVLAGQCVPTNQHYLLKYYDLWWYNAPITFDHNLRLIKSSGIKEGLDLNTALVKAVRENNYSLIK
LFTEWGADINYGLVSVNTEHTRDLCQELGAKEILNEEEILQIFIDLKFHKTSSNIILCHEVFSNNPILQKVNNLK
LRIEIFWELRELIEKTDLLNNEFLLSTLLLKYWYAIAVRYSLKEAIQYFYQKYTHMNTWRLTCALCFNNVFDLHE
AYEKDKIHMDIEEMMRIACIKDHNLSTMYYCYMLGANINQAMLTSIQYYNIENMFFCMDLGADVFEEGTTALGEG
YELIKNILSLKIYSPTTIPLPKSTDPEIIDHALKNYFSKNMMIFLSYDLR*
SEQ ID No. 114-China/2018/AnhuiXCGQ MGF 360 12L protein
MLPSLQSLTKKVLAGQCVPTNQHYLLKCYDLWWHDAPITFDHNLRLIKSAGIKEGLNLNTALVKAVRENNYNLIK
LFAEWGADINYGLVSVNTEHTWDLCRELGAKETLNEEEILQIFIDLKFHKTSSNIILCHEVFSNNPILQKVNNIK
MRIEIFWELRELIVKTDLLNNEFSLSTLLLKYWYAIAIRYNLKEAIQYFYQKYTHLNTWRLTCALCFNNVFDLHE
AYEKDKIHMDIEEMMRIACIKDHNLSTMYYCYVLGANINQAMLSSIQYYNIENMFFCIDLGADVFEEGTTALGEG
YELIKNILSLKIYSPATTPLPKSTDPEIIDHALKNYVSKNMMIFLTYDLR*
SEQ ID No. 3-Georgia 2007/1 MGF 360 12L protein
MLPSLQSLTKKVLAGQCVPTNQHYLLKCYDLWWHDAPITFDHNLRLIKSAGIKEGLNLNTALVKAVRENN
YNLIKLFAEWGADINYGLVSVNTEHTWDLCRELGAKETLNEEEILQIFIDLKFHKTSSNIILCHEVFSNN
PILQKVNNIKMRIEIFWELRELIVKTDLLNNEFSLSTLLLKYWYAIAIRYNLKEAIQYFYQKYTHLNTWR
LTCALCFNNVFDLHEAYEKDKIHMDIEEMMRIACIKDHNLSTMYYCYVLGANINQAMLSSIQYYNIENMF
FCIDLGADVFEEGTTALGEGYELIKNILSLKIYSPATTPLPKSTDPEIIDHALKNYVSKNMMIFLTYDLR
SEQ ID No. 115-Ken06.Bus MGF 360 12L protein
MLPSLQSLTKKVLARQCLPEDQHYLLKCYDLWWNNAPITFDHNLRLIKLAGIQEGLDLNMALVKAVKENNYSLIK
LFTEWGANINYGLISVNTEHTWDLCRELGAKKTLNEGDILQIFIDLKFYKTSSNIILCHEVFSDNLLLKRVNNLK
MRIEIFWELREIIEKTDLLNNEFSLNTLLLKYWYAIAVRYNLKEAIQYFYQKYTHLNTWRLTCALCFNNVFDLHE
AYEKDKICMDLEEMMRIACIKDHNLSTIYYCYMLGANINQAMLTSIQYYNIENIFFCMDLGADAFEEGMALVGQE
GYEPIRNILSLKIYSPATTPLPKSTDPEIIDHALKNYFSKNMMVFLTYDLR*
SEQ ID No. 116-Kenya 1950 MGF 360 12L protein
MLPSLQSLTKKVLAGQCLPEDQHYLLKCYDLWWNNAPITFDHNLRLIKSAGLQEGLDLNMALVKAVKENNYSLIK
LFTEWGANINYGLISVNTEHTWDLCRELGAKKTLNEGDILQIFIDLKFHKTSSNIILCHEVFSDNLLLKKVNNLK
MRIEIFWELREIIEKTDLLNNEFSLNTLLLKYWYAIAVRYNLKEAIQYFYQKYTHLNTWRLTCALCFNNVFDLHE
AYEKDKICMDLEEMMRIACIKDHSLSTIYYCYMLGANINQAMLTSIQYYNIENIFFCMDLGADAFEEGMALVGQE
GYEPIRNILSLKIYSPATTPLPKSTDPEIIDHELKNYFSKNMMVFLTYDLR*
SEQ ID No. 117-L60 MGF 360 12L protein
MLPSLQSLTKKVLAGQCVPTNQHYLLKYYDLWWYNAPITFDHNLRLIKSSGIKEGLDLNTALVKAVRENNYSLIK
LFTEWGADINYGLVSVNTEHTRDLCQELGAKEILNEEEILQIFIDLKFHKTSSNIILCHEVFSNNPILQKVNNLK
LRIEIFWELRELIEKTDLLNNEFLLSTLLLKYWYAIAVRYSLKEAIQYFYQKYTHLNTWRLTCALCFNNVFDLHE
AYEKDKIHMDIEEMMRIACIKDHNLSTMYYCYMLGANINQAMLTSIQYYNIENMFFCMDLGADVFEEGTTALGEG
YELIKNILSLKIYSPTTIPLPKSTDPEIIDHALKNYFSKNMMIFLSYDLR*
SEQ ID No. 118-Malawi Lil-20/1 MGF 360 12L protein
MLPSLQSLTKKVLAGQCLPTDQYYLLKCYDLWWYDSPITFDHNLGLIKSAGIKDGLDLNTALVKAVRENNYNLIK
LFTEWGADINYGLVSVNTEHTRDLCRELGAKETLNEEEILRIFIDLKFYKTSSNIILCHEVFSNNPLLQKVNNLK
MRIEIFWELRELIKKTDLLNNEFSLNTLLLKYWYAIAVRYNLKEAIQYFYQKYTHLNTWRLTCALCFNNVFDLHE
AYEKDKIYMDLEEMMRVACIKDHNLSTIYYCYVLGANINQAMLASIQYYNIENMFFCMDLGADVFEENMPVGEGY
ELIRNILSLKIYSPSTAPLPKNTDPEIIDHVLKNYKSKNMMTFLSYDLR*
SEQ ID No. 119-Mkuzi 1979 MGF 360 12L protein
MLPSLQSLTKKVLAGQCVPTNQHYLLKYYDLWWHDAPITFDHNLRLIKSAGIKEGLDLNTALVKAVKENNYNLIK
LFAEWGADINYGLVSVSSEHTWDLCRELGAKETLNEKEILQIFIDLKFHKTSSNIILCHEVFSNNPILQKVNNIK
MRIEIFWELRELIEKTDLLNNEFSLSTLLLKYWYAIAIRYNLKEAIQYFYQKYTHLNTWRLTCALCFNNVFDLHE
AYEKDKIYMDIEEMMRIACIKDHNLSTMYYCYMLGANINQAMLTSIQYYNIENMFFCMDLGADVFEEGTTALGEG
YELIKNILSLKIYSPTTIPLPKSTDPEIIDHALKNYFSKNMMIFLSYDLR*
SEQ ID No. 120-Pretorisuskop/96/4 MGF 360 12L protein
MLPSLQSLTKKVLAGQCVPTNQHYLLKCYDLWWHDGPITFDHNLKLIKSAGIKEGLDLNTALVKAVRENNYNLIK
LFAEWGANINYGLVSVNTEHTWDLCRELGAKETLNEEEILQIFIDLKFHKTSSNIILCHEVFSNNPILQKVNNIK
MRIEIFWELRELIEKTDLLNNEFSLSTLLLKYWYAIAIRYNLKEAIQYFYQKYTHLNTWRLTCALCFNNVFDLHE
AYEKDKIHMDIEEMMRIACIKDHNLSTMYYCYVLGGNINQAMLTSIQYYNIENMFFCMDLGADAFEEGTIALGEG
YKLIKNILSLKIYSPATTPLPKSTDPEIIDHALKNYVSKNMMIFLTYDLR*
SEQ ID No. 121-Tengani 62 MGF 360 12L protein
MLPSLQSLTKKVLAGQCVPTNQHYLLKCYDLWWHDAPITFDHNLRLIKSAGIKEGLNLNTALVKAVRENNYNLIK
LFAEWGADINYGLVSVNTEHTWDLCRELGAKETLNEEEILQIFIDLKFHKTSSNIILCHEVFSNNPILQKVNNIK
MRIEIFWELRELIEKTDLLNNEFSLSTLLLKYWYAIAIRYNLKEAIQYFYQKYTHLNTWRLTCALCFNNVFDLHE
AYEKDKIHMDIEEMMRIACIKDHNLSTMYYCYVLGANINQAMLSSIQYYNIENMFFCIDLGADVFEEGTTALGEG
YELIKNILSLKIYSPATTPLPKSMDPEIIDHALKNYVSKNMMIFLTYDLR*
SEQ ID No. 122-Warmbaths MGF 360 12L protein
MLPSLQSLTKKVLAGQCVPTNQHYLLKCYDLWWHDGPITFDHNLKLIKSAGIKEGLDLNTALVKAVRENNYNLIK
LFAEWGADINYGLVSVNTEHTRDLCRELGAKETLNEEEILQIFIDLKFHKTSSNIILCHEVFSNNPILQKVNNIK
MRIEIFWELRELIEKTDLLNNEFSLSTLLLKYWYAIAIRYNLKEAIQYFYQKYTHLNTWRLTCALCFNNVFDLHE
AYEKDKIYMDIEEMMRIACIKDHNLSTMYYCYVLGANINQAMLTSIQYYNVENMFFCMDLGADAFEEGTIALGEG
YKLIKNILSLKIYSPATTPLPKSTDPEIIDHAVKNYVSKNMMIFLTYDLR*
SEQ ID No. 123-Warthog MGF 360 12L protein
MLPSLQSLTKKVLAGQCVPTNQHYLLKCYDLWWHDGPITFDHNLKLIKSAGIKEGLDLNTALVKAVRENNYNLIK
LFAEWGANINYGLVSVNTEHTRDLCRELGAKETLNEEEILQIFIDLKFHKTSSNIILCHEVFSNNPILQKVNNIK
MRIEIFWELRELIEKTDLLNNEFSLSALLLKYWYAIAIRYNLKEAIQYFYQKYTHLNTWRLTCALCFNNVFDLHE
AYEKDKIHMDIEEMMRIACIKDHNLSTMYYCYVLGANINQAMLTSIQYYNIENMFFCMDLGADAFEEGTIALGEG
YKLIKNILSLKIYSPATTPLPKSTDPEIIDHALKNYVSKNMMIFLTYDLR*
MGF 360 13L protein sequences
SEQ ID No. 124-Benin 97/1 MGF 360 13L protein
MSLPLSLQTLVKKTVASQCLSIDEHCILKYCGLWWHDAPLKLCMDRGRIQIKSGFLGEDIDLRVALIIAVKENNY
SLIKLFTEWGANINYSLLSINTKHIRELCRQLGAKETLEDNDIFRIFTRIMHNKTSGSIILCHEIFMNNPMLENK
FVIQLRGLIYKRLWGLIEIKETDELNDLLVKYWYAKAVQYVCKNAICFLDEKYTDLNEWRLKCLLYYNKIYELHE
MYHKKKVQIDVHDMICLACAKDNNLLTIYYCYALGGNINQAMLTSVQYYNVGNIFFCIDLGGNAFEEGRAIAEQK
GYNFLSHSLTLDIYSSDASLPLNLKDPEKISSLLKDYKSKNLSIIWEYSHNIL*
SEQ ID No. 125-China/2018/AnhuiXCGQ MGF 360 13L protein
MSLPLSLQTLVKKTIASQCLSIDEHCILKYCGLWWHDAPLKLCMDRGRIQIKSGFLGEDIDLRVALIIAVKENNY
SLIKLFTEWGANXNYGLLSINTKHIRELCRQLGAKETLEDNDIFRIFTRIMHNKTSGSIILCHEIEWNNPILENK
FVIQLRGLIYKRLWGLIEIKETDELNGLLVKYWYAKAVQYDCKDAICFLDEKYTDLNEWRLKCLLYYNKIYELHE
MYHKENIQIDVHDMICLASTKDNNPLTIYYCYALGGNINQAMLTSVQYYNIGNIFFCIDLGGNAFEEGRAIAEQK
GYNFLSHSLALDIYSSDASLPLNLKDPEEISSLLKDYKSKNLSIIWEYSHNIL*
SEQ ID No. 4-Georgia 2007/1 MGF 360 13L protein
MSLPLSLQTLVKKTIASQCLSIDEHCILKYCGLWWHDAPLKLCMDRGRIQIKSGFLGEDIDLRVALIIAV
KENNYSLIKLFTEWGANINYGLLSINTKHIRELCRQLGAKETLEDNDIFRIFTRIMHNKTSGSIILCHEI
FMNNPILENKFVIQLRGLIYKRLWGLIEIKETDELNGLLVKYWYAKAVQYDCKDAICFLDEKYTDLNEWR
LKCLLYYNKIYELHEMYHKENIQIDVHDMICLASTKDNNPLTIYYCYALGGNINQAMLTSVQYYNIGNIE
FCIDLGGNAFEEGRAIAEQKGYNFLSHSLALDIYSSDASLPLNLKDPEEISSLLKDYKSKNLSIIWEYSH
NIL
SEQ ID No. 126-Ken06.Bus MGF 360 13L protein
MSSPLSLQTLVKKTVAGTSCLSIDEHILKYCGLWWHDAPLKLYIDRGRIYIKSGFLGEDIDLCVALIIAVKENNY
SMIKLFTEWGAYINYSLLSINTKHARDLCRQLGAKETLDDYDIFCIFNKIMHNKTSGSIILCHEIFINNPNLENN
FAAQLRRLIYKRLCGLIEIKETDELSELLVKYWYANAVQYDHKDAICFLDEKYTDLDEW*LKCYLCYNKIYELHD
IYHKEKIQIDVNEMLSLACIRDNNLLTIYYCYALGGNINQAMLTSVQYYNIGNIYFCIDLGGNAFEEGSAIARQN
GYNFLSHSLVLNIYSSDASLPLNLKDPEEISSLLKNYKSKNLSIILDYSHKIL*
SEQ ID No. 127-Kenya 1950 MGF 360 13L protein
MSSPLSLQTLVKKTVASTSCLSIDEHILKYCGLWWHDAPLKLYIDRGRIYIKSGFLGEDIDLCVALIIAVKENNY
SLIKLFTEWGAYINYSLLSINTKHARDLCRQLGAKETLDDYDIFCIFNKIMHNKTSGSIILCHEIFINNPKLENN
FAAQLRRLIYKRLCGLIEIKETDELSELLVKYWYANAVQYDHKDAICFLDEKYTDLDEWRLKCYLCYNKIYELHD
IYHKKKIQIDVNEMLSLACIRDNNLLTIYYCYALGGNINQAMLTSVQYYNIGNIYFCIDLGGNAFEEGSAIARQN
GYNFLCHSLILNIYSSDASLPLNLKVPEEISSLLKNYKSKNLSIILDYSHKIL*
SEQ ID No. 128-L60 MGF 360 13L protein
MSLPLSLQTLVKKTVASQCLSIDEHCILKYCGLWWHDAPLKLCMDRGRIQIKSGFLGEDIDLRVALIIAVKENNY
SLIKLFTEWGANINYSLLSINTKHIRELCRQLGAKETLEDNDIFRIFTRIMHNKTSGSIILCHEIFMNNPMLENK
FVIQLRGLIYKRLWGLIEIKETDELNDLLVKYWYAKAVQYVCKNAICFLDEKYTDLNEWRLKCLLYYNKIYELHE
MYHKKKVQIDVHDMICLACAKDNNLLTIYYCYALGGNINQAMLTSVQYYNVGNIFFCIDLGGNAFEEGRAIAEQK
GYNFLSHSLTLDIYSSDASLPLNLKDPEKISSLLKDYKSKNLSIIWEYSHNIL*
SEQ ID No. 129-Malawi Lil-20/1 (1983) MGF 360 13L protein
MSAPLSLQTLVKKTVAST5CLSIDEHILKYCDLWWHDAPLKLYMDRGRIQIKSGFLGEDIDLCVALIIAVKENNY
NLIKLFTELGANINYSLLSINTKHVRDLCRQLGAKETLEDYDIFCIFNKIMHNKTSGSVILCHEIFINNPNLENK
FAAQLRRLIYKRLCGLIEIKETDELSELLVKYWYAKAVQYDYKDAICFLDEKYTDLNEWRLKCYLYYNKIYELHD
IYHKEKIQIDVNEMLSLACIRDNNPLTIYYCYALGGNINQAMLTSVQYYNIGNIYFCIDLGGNAFEEGSAIARQK
GYNFLSHSLVLNIYSSDASLPLNLKDPEEISSLLKNYKSKNLSIILDYSHNIL*
SEQ ID No. 130-Mkuzi 1979 MGF 360 13L protein
MSLPLSLQTLVKKTVASQCLSIDEHCILKYCGLWWHDAPLKLCMDRGRIQIKSGFLGEDIDLRVALIIAVKENNY
SLIKLFTEWGANINYSLLSINTKHIRELCRQLGAKETLEDNDIFRIFTRIMHNKTSGSIILCHEIFMNNPILENK
FVIQLRGLIYKRLWGLIEIKETDKLNDLLVKYWYAKAVQYVCKNAICFLDEKYTDLNEWRLKCLLYYNKIYELHE
MYHKKKVQIDVHDMICLACAKDNNLLTIYYCYALGSNINQAMLTSVQYYNIGNIFFCIDLGGNAFEEGRAIAEQK
GYNFLSHSLTLDIYSSDASLPLNLKDPEEISSLLKDYKSKNLSIIWEYSHNIL*
SEQ ID No. 131-Pretorisuskop/96/4 MGF 360 13L protein
MSLPLSLQTLVKKTVASQCLSIDEHCILKHCGLWWHDVPLKLCMDRGQIQIKSGFLGEDIDLHIALIIAVKENNY
SLIKLFTEWGAHINYSLLSINTEHIRELCRQLGAKETLEDDDVFRIFTKIMHNKTSGRIILCHDIFMNNPNIENK
FTIQLRGLICKRLWGLIEIKETDELNDLLVKYWYAKAVQYECKDAICFLEEKYTDLNEWRLKCLLYFNKIYELHE
MYHKEKVQIDVHDMICLASTKDNNPLTIYYCYALGGNINQAMLTSIQYYNIGNIFFCIDLGGNAFEEGRAIAEQK
GYNFLSHSLALDIFSSDASLPLNLKDPEEISSFLKDYKSKNLSIIWEYSHNIL*
SEQ ID No. 132-Tengani 62 MGF 360 13L protein
MSLPLSLQTLVKKTVANQSLSIDEHCILKHCGLWWHDVPLKLCMDRGQIQIKSGFLGEDIDLHIALIIAVKENNY
SLIKLFTEWGANINYSLLSINTKHIRELCRQLGAKETLEDDDIFRIFTKIMHNKTSGSIILCHDIFMNNPNIEDK
FTTQLRGLIYKRLWGLIEIKETDELNGLLVKYWYAKAVQYECKDAICFLDEKYTDLNEWRLKCLLYYNKIYELHE
MYHKENIQIDVHDMICLASTKDNNPLTIYYCYALGGNINQAMLTSVQYYNIGNIFFCIDLGGNAFEEGRAIAEQK
GYNFLSHSLALDIYSSDASLPLNLKDPEEISSLLKDYKSKNLSIIWEYSHNIL*
SEQ ID No. 133-Warmbaths MGF 360 13L protein
MSLPLSLQTLVKKTVASQCLSTDEHCILKHCGLWWHDVPLKLCMDRGQIQIKSGFLGEDIDLHVALIIAVKENNY
SLIKLFTEWGAHINYSLLSINTEHIRELCRQLGAKKTLEDNDIFRIFTKIMHNKTSGRIILCHEIFMNNPNIENR
FTIOLRGLICKRLWRLIEIKETDELNDLLVKYWYAKAVQYECKDAICFLDEKYTGLNEWRLKCLLYYNKIYELHE
MYHKDKVQIDVHDMICLASTKDNNPLTIYYCYALGGNINQAMLTSVQYYNIGNIFFCIDLGGNAFEEGRAIAEQR
GYHFLSHSLALDIYSSDASLPLNLKDPEEISSLLKGYKSKNLSIIWEYSHNIL*
SEQ ID No. 134-Warthog MGF 360 13L protein
MSLPLSLQTLVKKTVASQCLSTDEHCILKHCGLWWHDVPLKLCMDRGQIQIKSGFLGEDIDLHIALIIAVKENNY
SLIKLFTEWGAHINYSLLSINTEHIRELCRQLGAKETLEDNDIFRIETKIMHNKTSGRIILCHEIFMNNPNIENK
FTTQLRGLICKRLWGLIEIKETDELNDLLVKYWYAKAVQYECKDAICFLDEKYTDLNEWRLKCLLYYNKIYELHE
MYHKEKVQIDVHDMICLASTKDNNPLTIYYCYALGGNINQAMLTSVQYYNIGNIFFCIDLGGNAFEEGRAIAEQK
GYHFLSHSLTLDIYSSDASLPLNLKDPEEISSLLKDYKSKNLSIIWEYSHNIL*
MGF 360 14L protein sequences
SEQ ID No. 135-Benin 97/1 MGF 360 14L protein
MLSLQTLAKKWACNYLSSDYDYTLQRFGLWWDLGPIHLCNNCKQVFSYKHLQCFSEDDLCLEAA.LVKAVKSDNL
ELIRLFVDWGANPEYGLIRVPAVYLKRLCAELGGLTPVSEPRLLEILKEVANLKSCAGVLLGYDMFCHNPLLETV
TRTTLDTVTYTCSNIPLTGDTAHLLLTKFWFALALRHNFTKAIHYFYKRHKNQLYWRVACSLYFNNIFDIHELCR
EKEICISPNLMMKFACLREKNYAAIYYCHRLGASLDYGMNLSIYNNNTLNMFFCIDLGAADFDRAQLIAHKAYMY
NLSNIFLVKQLFSRDVTLVLDVTEPQEIYDMLKTYTSKNMKRAEEYLTAHPEIIVID*
SEQ ID No. 136-China/2018/AnhuiXCGQ MGF 360 14L protein
MLSLQTLAKKVVACNYLSSDYDYTLQRFGLWWDLGPIHLCNNCKQVFSYKHLQCFSEDDLCLEAALVKAVKSDNL
ELIRLFVDWGANPEYGLIRVPAVYLKRLCAELGGLTPVSEPRLLEILKEVARLKSCAGVLLGYDMFCHNPLLETV
TRTTLDTVTYTCSNIPLTGDTAHHLLTKFWFALALRHNFTKAIHYFYKRHKNHLYWRVACSLYFNNIFDIHELCR
EKEICISPNLMMKFACLREKNYAAIYYCHRLGASLDYGMNLSIYNNNTLNMFFCIDLGAADFDRAQLIAHKAYMY
NLSNIFLVKQLFSRDVTLVLDVTEPQEIYDMLKTYTSKNLKRAEEYLTAHPEIIVID*
SEQ ID No. 5-Georgia 2007/1 MGF 360 14L protein
MLSLQTLAKKVVACNYLSSDYDYTLQRFGLWWDLGPIHLCNNCKQVFSYKHLQCFSEDDLCLEAALVKAV
KSDNLELIRLFVDWGANPEYGLIRVPAVYLKRLCAELGGLTPVSEPRLLEILKEVARLKSCAGVLLGYDM
FCHNPLLETVTRTTLDTVTYTCSNIPLTGDTAHHLLTKFWFALALRHNFTKAIHYFYKRHKNHLYWRVAC
SLYFNNIFDIHELCREKEICISPNLMMKFACLREKNYAAIYYCHRLGASLDYGMNLSIYNNNTLNMFFCI
DLGAADFDRAQLIAHKAYMYNLSNIFLVKQLFSRDVTLVLDVTEPQEIYDMLKTYTSKNLKRAEEYLTAH
PEIIVID
SEQ ID No. 137-KenO6.Bus MGF 360 14L protein
MLSLQTLAKKVVACNYLSSDYDYTLQRFGLWWDLGPIHLCNTCKQIFSYQHLQCFSVDDLCLDAALVKAVKSDNL
ELIRLFVDWGANPEYGLIRVPAVHLKRLCTELGGLTPVSESRLLEILKEVADLKSCAGVLLGYDMFCHNPLMETV
TRTTLNTVMYTRSKIPLTGDTAHLLLTKFWFALALRHNFTKAIHYFYEKHKNQLYWRLTCSLYFNNIFDLHELCS
KKEICISPNLMMKFACLREENYAAIYYCHMLGASPDYGMNLSIYNNNTLNLFFCIDLGATNFDRARLIAHRVYMY
NLSNIFLVKQLFSRDASLVLNITEPQAIYDMLKTYTSKNLKRAEEYFTAHPEIVVID*
SEQ ID No. 138-Kenya 1950 MGF 360 14L protein
MLSLQTLAKKVVACNYLSSDYDYTLQRFGLWWDLGPIHLCNTCKQIFSYQHLQCFSEDDLCLDAALVKAVKSDNL
ELIRLFVDWGANPEYGLIRVPAVDLKRLCTELGGLTPVSESRLLEILKEVADLKSCAGVLLGYDMFCHNPLLETV
TRTTLNTVMYTRSKIPLTGDTAHLLLTKFWFALALRHNFTKAIHYFYEKHKNQLYWRLACSLYFNNIFDLHELCS
KKEICISPNLMMKFACLREENYAAIYYCHMLGASPDYGMNLSIYNNNTLNLFFCIDLGATNFDRARLIAHRVYMY
NLSNIFLVKQLFSRDASLVLNITEPQAIYDMLKTYTSKNLKRAEEYFTAHPEIVVID*
SEQ ID No. 139-L60 MGF 360 14L protein
MLSLQTLAKKVVACNYLSSDYDYTLQRFGLWWDLGPIHLCNNCKQVFSYKHLQCFSEDDLCLEAALVKAVKSDNL
ELIRLFVDWGANPEYGLIRVPAVYLKRLCAELGGLTPVSEPRLLEILKEVANLKSCAGVLLGYDMFCHNPLLETV
TRTTLDTVTYTCSNIPLTGDTAHLLLTKFWFALALRHNFTKAIHYFYKRHKNQLYWRVACSLYFNNIFDIHELCR
EKEICISPNLMMKFACLREKNYAAIYYCHRLGASLDYGMNLSIYNNNTLNMFFCIDLGAADFDRAQLIAHKAYMY
NLSNIFLVKQLFSRDVTLVLDVTEPQEIYDMLKTYTSKNMKRAEEYLTAHPEIIVID*
SEQ ID No. 140-Malawi Lil-20/1 MGF 360 14L protein
MLSLQTLAKKVVACNYLSSDYDYTLQRFGLWWDLGPIHLCNTCKQIFSYKHLQCFSEDDLCLEAALVKAVKSDNL
ELIRLFVDWGANPEYGLIRVPAVHLKRLCMELGGLTPVSESRLLEILKEVADLKSCAGVLLGYDMFCHNPLLETV
TRTTLDTVMYTRSKIPLTGDTAHLLLSKFWFALALRHNFTKAIHYFYEKHKNQLYWRLTCSLYFNNIFDLHELCC
KKEICISPNLMMKFACLREENYAAIYYCHMLGASLDYGMNLSIYNNNTLNLFFCIDLGATNFDRARLIARRVYMY
NLSNLFLVKQLFSRDVSLILDLTEPQAIYDMLNTYTSKNLKQAEEYFTAHPEIVVID*
SEQ ID No. 141-Mkuzi 1979 MGF 360 14L protein
MLSLQTLAKKVVACNYLSSDYDYTLQRFGLWWDLGPIHLCNNCKQVFSYKHLQCFSEDDLCLEAALVKAVKSDNL
ELIRLFVDWGANPEYGLIRVPAVYLKRLCAELGGLTPVSEPRLLEILKEVANLKSCAGVLLGYDMFCHNPLLETV
TRTTLDTVTYTCSNIPLTGDTAHLLLTKFWFALALRHNFTKAIHYFYKRHKNQLYWRVACSLYFNNIFDIHELCR
EKEICISPNLMMKFACLREKNYAAIYYCHRLGASLDYGMNLSIYNNNTLNMFFCIDLGAADFDRAQLIAHKAYMY
NLSNIFLVKQLFSRDVTLVLDVTEPQEIYDMLKTYTSKNMKRAEEYLTAHPEIIVID*
SEQ ID No. 142-Pretorisuskop/96/4 MGF 360 14L protein
MLSLQTLAKKVVACNYLSSDYDYTLQRFGLWWDLGPIHLCNNCKQIFSYKHLQCFSEDDLCLEAALVKAVKSDNL
ELIRLFVDWGANPEYGLIRVPAVHLKRLCTELGGLTPVTEPRLLEILKEVAKLKSCAGVLLGYDMFCHNPLLETV
TRTTLDTVTYTCSNIPLTGDTAHLLLTKFWFALALRHNFTKAIHYFYKRHKNHLYWRVACSLYFNNIFDIHELCR
EKEICISPNLMMKFACLQKKNYAAIYYCYRLGASLDYGMNLSIYNNNTLNMFFCIDLGATDFDRAQHIAHKAYMY
NLSNIFLVKQLFSRDVTLALDVTEPQEIYDRLKSYTSKNLKRAEEYLTAHPEIIVID*
SEQ ID No. 143-Tengani 62 MGF 360 14L protein
MLSLQTLAKKVVACNYLSSDYDYTLQRFGLWWDLGPIHLCNNCKQIFSYKHVQCFSEDDLCLEAALVKAVKSDNL
ELIRLFVDWGANPEYGLIRVPAVHLKRLCTELGGLTPVSESRLLEILKEVARLKSCAGVLLGYDMFCHNPLLETV
TRTTLDTVTYTCSNIPLTGDTAHHLLTKFWFALALRHNFTKAIHYFYKRHKNHLYWRVACSLYFNNIFDIHELCR
EKEICISPNLMMKFACLQKKNYAAIYYCYRLGASLDYGMNLSIYNNNTLNMFFCIDLGATDFDRAQRIAHKAYMY
NLSNIFLVKQLFSRDVTLALDVTEPQEIYDMLKSYTSKNLKRAEEYLTAHPEIIVID*
SEQ ID No. 144-Warmbaths MGF 360 14L protein
MLSLQTLAKKvvACNYLSSDYDYMLQRFGLWWDLGPIHLCNNCKQIFSYKHLQCFSEDDLCLEAALVKAVKRDNL
ELIRLFVDWGANPEYGLICVPTVHLKRLCTELGGLTPVSEPRLLEILKEVANLKSCAGVLLGYDMFCYNPLLETI
TRTTLDTVMYSCSKIPLMGDTAHHLLTKFWFALALRHNFTKAIHYFYKRHKNHLYWRVACSLYFNNIFDIHELCR
EKEICISPNLMMKFACLREKNYAAIYYCYRLGASLDYGMNLSIYNNNSLNLFFCIDLGATDFDRAQRIAHKAYMY
NLSNILLVKQLFSRDVTLALDVTEPQEIYDRLKAYTSKNMKRAEEYLTAHPEIIVID*
SEQ ID No. 145-Warthog MGF 360 14L protein
MLSLQTLAKKVVACNYLSSDYDYMLQRFGLWWDLGPIHLCNNCKQIFSYKHLQCFSEDDLCLEAALVKAVKSDNL
ELIRLFVDWGANPEYGLIRVPAVHLKRLCTELGGLTPVSEPRLLEILKEVAKLKSCAGVLLGYDMFCHNPLLETV
TRTTLDTVTYTCSNIPLTGDTAHHLLTKFWFALALRHNFTKAIHYFYKRHKNHLYWRVACSLYFNNIFDIHELCR
EKEICISPNLMMKFACLRKKNYAAIYYCYRLGASLDYGMNLSIYNNNTLNMFFCIDLGATDFDRAQRIAHKTYMY
NLSNIFLVKQLFSRDVTLALDVTEPQEIYDMLKSYTSKNLKRAEEYLTAHPEIIVID*
MGF 505 1R protein sequences
SEQ ID No. 146-Benin 97/1 MGF 505 1R protein
MFSLQNLCRKTLPDCKLPEFFDDYILQLLGLYWENHGTIQRAGNNCVLIQQHTLIPVNEALRIAASEENYEIVGL
LLAWEGNLYYAIIGALEGNRYNLIRKYDDQIKDHHDILPFIDDPIIFHKCHIMRRCFFDCILYQAVKYSKFRVLL
YFKYTLEDDLPLVHLLIEKACEDHNYEVIKWIYENLHVCHIIDTFDCAIAHKDLRLYCLGYTFIYNRIVPYKYHH
LDILILSSLQLLHKVAAKGYLDFILETLKYDHNIDNLDVILTQAATYNHRKILTYFIPQSTYAQIEQCLFVAIKT
KSSKKTLNLLLSHLNLSIKLIQKISQYVATFNSTNIIGILSMKRKKKIYLDIILTKFVKNAIFNKFVVRCMERFS
INPERIVKMAARINKMMLVKKISEHVWKNHAARLKHLKHAVHTMKHKDGKNRLMNFIYEHCYYHMQGEEIFSLAR
FYAIHHAPKLFDVFYNCCILDTIRFKSLLLDCSHIIGKNAHDATNINIVNKYIGNLFAMGVLSKKEILQDYPSIY
SKHYMP*
SEQ ID No. 147-China/2018/AnhuiXCGQ MGF 505 1R protein
MFSLQNLCRKTLPNRKLPEFFDEYILQLLGLYWENHGTIQRAGNNCVLIQQHTLIPVNEALRTAASEENYEIVSL
LLAWEGNLYYAIIGALEGNRHDLIRKYDDQIKDHHEILPFIDDPVIFHKCHIMRQCFFDCILYQAVKYSKFRVLL
YFKHRLEDDLPFTHLLIEKACKDHNYEVIKWIYENLHIYNMIDTFECAIAHKDLHLYCLGYRFIYNRIVPDKYHH
LDIRMLSSLQLLHKVAAKGYLDFILETLKYDHNKDNINIILTQAATYNHRKILIYFIPQSTHAQIEQCLLVAIKA
KSSRKTLNLLLSHLNLSINLIKKISHYVATYNSTNIIGILSMRRKKKIYLDIILTKFVKKAIFNKFvvRCMDTFS
INPERILKIAARINRMMLVKKISEHVWKNHAVRLKYLKHAVHTMKHKDGKNRLMNFIYDRCYYHMQGEEIFSLAR
FYAIHHAPKLFDVFYDCCILDTIRFKSLLLDCSHIIGKNAHDATNINIVNKYIGNLFVMGVLSKKEILQDYPSIY
SKQYMP*
SEQ ID No. 6-Georgia 2007/1 MGF 505 1R protein
MFSLQNLCRKTLPNRKLPEFFDEYILQLLGLYWENHGTIQRAGNNCVLIQQHTLIPVNEALRTAASEENY
EIVSLLLAWEGNLYYAIIGALEGNRHDLIRKYDDQIKDHHEILPFIDDPVIFHKCHIMRQCFFDCILYQA
VKYSKFRVLLYFKHRLEDDLPFTHLLIEKACKDHNYEVIKWIYENLHIYNMIDTFECAIAHKDLHLYCLG
YRFIYNRIVPDKYHHLDIRMLSSLQLLHKVAAKGYLDFILETLKYDHNKDNINIILTQAATYNHRKILIY
FIPQSTHAQIEQCLLVAIKAKSSRKTLNLLLSHLNLSINLIKKISHYVATYNSTNIIGILSMRRKKKIYL
DIILTKFVKKAIFNKFvvRCMDTFSINPERILKIAARINRMMLVKKISEHVWKNHAVRLKYLKHAVHTMK
HKDGKNRLMNFIYDRCYYHMQGEEIFSLARFYAIHHAPKLFDVFYDCCILDTIRFKSLLLDCSHIIGKNA
HDATNINIVNKYIGNLFVMGVLSKKEILQDYPSIYSKQYMP
SEQ ID No. 148-Ken05/Tk1 MGF 505 1R protein
MFSLQNLCRKTLPDCKLPEFFDDYVLQLLGLYWENHGTIQRAGNNCVLIQQHNLIPVNEALRIAASEENYEIVSL
LLAWEGNLYYAIIGALEGNRHNLIRKYDDQIKDHHEILPFIDDPVIFHKCHIMRRCFFNCMLYQAVKYSKFSVLL
YFKYILKENLPLVHSLIEKAYKYHNYEVIKWIYENLHIYDIINTFKYAIAHKDLRLYCLGYTFVYNRIVPYKYYH
LDIRILLRLQLLHKVTAKGYLDFILETLKYDHNTNNIDIILTQAATYNHRNILTYFIPQSTYAQIEQCLFVAIKT
NASKKTLNLLLSHLNLSIKLVKKLSQYVVAYKSTNIISILSMQQKKKIYLDIILTKVVKNAVFIKFVIGCMVTFS
INPERIVKMAARIKKMKLVKNISEHVWKNHAAKLKHLKHAVHTMKHQEGKNRLMNFIYDHCYYHMQGEEIFSLAR
FYAIHHAPKLFDVFYDCCILDTIRFKSLLLDCSHIIAKNAHDASINIVNKYIGNLFAMGVLSKKEILQDYPSIYS
KYDIL*
SEQ ID No. 149-Ken06.Bus MGF 505 1R protein
MFSLQNLCRKTLPDCKLPEFFDDYVLQLLGLYWENHGTIQRAGNNCVLIQQHNLIPVNEALRIAASEENYEIVSL
LLAWEGNLYYAIIGALEGNHHNLIRKYDDQIKDHHEILPFIDDPVIFHKCHTMRRCFFNCILYQAVKYSKFSVLL
YFKYILKENLPLVHSLIEKAYKYHNYEVIKWIYENLHIYDIINTFKCAIAHKDLRLYCLGYTFVYNRIVPYKYYH
LDIRILLRLQLLHKVTAKGYLDFILETLKYDHNTNNIDIILTQAATYNHRNILTYFIPQSTYAQIEQCLFVAIKT
NASKKTLNLLLSHLNLSIKLVKKLSQYVVAYKSTNIISILSRQQKKKIYLDIILTKVVKNAVFNKFVIGCMVTFS
INPERIVKMAARIKKMKLVKNISEHVWKNHAVKLKYLKHAVHTMKHQEGKNRLMNFIYDHCYYHMQEEEIFSLAR
FYAIHHAPKLFDVFYDCCILDTIRFKSLLLDCSHIIVKNAHDASINIVNKYIGNLFAMGVLSKKEILQDYPSIYS
KDYML*
SEQ ID No. 150-Kenya 1950 MGF 505 1R protein
MFSLQNLCRKTLPDRKLPEFFDDYVLQLLGLYWENHGTIQRAGNNCVLIQQHNLIPVNEALRIAASEENYEIVSL
LLAWEGNLYYAIIGALEGNRHNLIRKYDDQIKDHHEILPFIDDPVIFHKCHMMRRCFFNCILYQAVKYSKFSVLL
YFKYILKENLPLVHSLIEKACEDHNYEVIKWIYENLHIYEIMDTFKCAIAHKDLHLYSLGYTFIYNRIVPYKYHH
LDIRILSRLQLLHKVTAKGYLDFILETLKYDHNKDNINIILTQAATYNHRNILTYFIPQSTYAQIEQCLFVAIKT
NASKKTLNLLLSHLNLSIKLVKKLSQYVVAYKSTNIISILSMQQKKKIYLDIILTKVVKNAIFIKFVIGCMVTFS
INPERIVKMAARIKKMKLVKNISEHVWKNHAAKLKHLKHAVHTMKHQEGKNRLMNFIYDHCYYHMQGEEIFSLAR
FYAIHHAPKLFDVFYDCCILDTIRFKSLLLDCSYIIAKNAHDASINIVNKYIGNLFAMGVLSKKEILQDYPSIYS
KDYML*
SEQ ID No. 151-L60 MGF 505 1R protein
MFSLQNLCRKTLPDCKLPEFFDDYILQLLGLYWENHGTIQRAGNNCVLIQQHTLIPVNEALRIAASEENYEIVGL
LLAWEGNLYYAIIGALEGNRYNLIRKYDDQIKDHHDILPFIDDPIIFHKCHIMRRCFFDCILYQAVKYSKFRVLL
YFKYTLEDDLPLVHLLIEKACEDHNYEVIKWIYENLHVCHIIDTFDCAIAHKDLRLYCLGYTFIYNRIVPYKYHH
LDILILSSLQLLHKVAAKGYLDFILETLKYDHNIDNLDVILTQAATYNHRKILTYFIPQSTYAQIEQCLFVAIKT
KSSKKTLNLLLSHLNLSIKLIQKISQYVATFNSTNIIGILSMKRKKKIYLDIILTKFVKNAIFNKFVVRCMERFS
INPERIVKMAARINKMMLVKKISEHVWKNHAARLKHLKHAVHTMKHKDGKNRLMNFIYEHCYYHMQGEEIFSLAR
FYAIHHAPKLFDVFYNCCILDTIRFKSLLLDCSHIIGKNAHDATNINIVNKYIGNLFAMGVLSKKEILQDYPSIY
SKHYMP*
SEQ ID No. 152-Malawi Lil-20/1 (1983) MGF 505 1R protein
MFSLQNLCRKTLPDCKLPEFFDEYILQLLGLYWENHGTIQRAGNNCVLIQQHNLIPVNEALRIAASEENYEIVSL
LLAWEGNLYYAIIGALEGNRPDLIRKYDDQIKDHHEILPFIDDPIIFHKCHIMRRCFFNCILYQAVKYSKFRVLL
YFKHRLGDDLPLTHLLIEKACEDHNYEVIKWIYENLHSYNIMDTFECAIAHKDLRLYCLGYTFIYNRIVPYKYHH
LDICILSSLQLLHKVAAKGYLDFILETLKYDHNINNIDIILTQAATYNHRKILTYFIPQLTYAQIEQCLLVAIKT
KASKKTLNLLLSHLNLSIKLIKKISQYVVTYNSTNIISILSMRRKKKIYLDIILTEFVKNAIFNKFvvRCMDTFS
INPERIVKMAARINRMMLVKNISERVWKNHAVKLKHLKHAVHTMKHQEGKNRLMNFIYDHCYYHMQGEEIFGLAR
FYAIHHAPKLFDVFYDCCMLDATRFKSLLLDCPHIIGKNAYDAGINLVNKYIGNLFAMGVLSKKEILQDYPSIYS
KHDMF*
SEQ ID No. 153-Mkuzi1979 MGF 505 1R protein
MFSLQNLCRKTLPDCKLPEFFDDYILQLLGLYWENHGTIQRAGNNCVLIQQHTLIPINEALRTAASEENYEIVSL
LLAWEGNLYYAIIGALEGNRHDLIRKYDDQIKDHHEILPFIDDPVIFHKCHIMRRCFFDCILYQAVKYSKFRVLL
YFKYRLEDDLPLTHLLIEKACENHNYEVIRWIYENLHIYNMIDTFECAIAHKDLRLYCLGYTFIYNRIVPNKYHH
IDILILSSLQLLHKVAAKGYLDFILETLKYDHNNDNLDIILTQAATYNHRKILTYFIPQSTYAQIEQCLMVAIKT
KSSKKTLNLLLSHLNLSIKLIKKISQYVVTYNSTNIIGILSMKRKKKIYLDILLTKFVKNAIFNKFVVRYMDTFS
INPEKIVKMAARINKMMLVKKISEHIWKNHAARLEHLKHAVHTMKHKDGKNRLMNFIYEYCYYHMQGEEIFSLAR
FYAIHHAPKLFDVFYNCCILDTIRFKSLLLDCSHIIGKNAHDATNINIVNKYIDNLFAMGVLSKKEILQDYPSIY
SKHYMP*
SEQ ID No. 154-Pretorisuskop/96/4 MGF 505 1R protein
MFSLQNLCRKTLPDCKLPEFFDEYILQLLGLYWENHGTIQRAGNNCVLVQQHTLIPVNEALRIAASEENYEIVSL
LLAWEGNLYYAIIGALEGNRHDLIRKYDDQIKDHHEILPFIDNPVIFHKCHIMRRCFFDCILYQAVKYSKFRVLL
YFKYRLENDLPLAHLLVEKACEDHNYEVIKWLYENLHIYNIMETFECAIAHKDLRLYRLGYTFIYNRIVPYKYHY
LDVLILSGLHLLYKVAAKGYLDFILETLKYDHNNDNLDIILTQAVTYNHRKILTYYIPQLTYAQIEQCLFMAIKK
KSSKKTLNLLLSHLKLSIKLIKKISQYVATYNSTNIIGILNMKRKKKIYLDIILTKFVKYAIFNKYVVRCMDTFS
INPERIIKMAARINKMLLVKKISQHAWKNHAARLKHLKHAVYTMKHKDGKNRLMNLIYDHYYYHMQGEEIFSLAR
FYAIHHAPKLFDVFYDCCLLDTIRFKSLLLDCSHIIGKNAHDATNITIVNKYIGNLFAMGVLSKKEILQDYPSIY
SKHYMP*
SEQIDNo. 155- T engani 62 MGF 505 1R protein
MFSLQNLCRKTLPDCKLPEFFDEYILQLLGLYWENHGTIQRAGNNCVLIQQHNLIPVNEALRIAASEENYEIVSL
LLAWEGNLYYAIIGALEGNRHDLIRKYDDQIKDHHEILPFIDDPVIFHKCHIMRQCFFDCILYQAVKYSKFRVLL
SFKHRLRDDLPFTHLLIEKACKDHNYEVIKWIYENLHIYNMIDTFECAIAHKDLRLYCLGYRFIYNRIVPDKYHH
LDIRMLSSLQLLHKVAAKGYLDFILETLKYDHNKDNINIILTQAATYNHRKILIYFIPQSTHAQIEQCLLVAIKT
KSSKKTLNLLLSHLNLSINLIKKISHYVATYNSTNIIGILSMRRKKKIYLDIILTKFVKKAIFNKFvvRCMDTFS
INPERILKIAARINRTMLVKKISEHVWKNHAVRLKYLKHAVHTMKHKDGKNRLMNFIYDRCYYHMQGEEIFSLAR
FYAIHHAPKLFDVFYDCCILDTIRFKSLLLDCSHIIGKNAHDATNINIVNKYIGNLFVMGVLSKKEILQDYPSIY
SKHYMP*
SEQ ID No. 156-Warmbaths MGF 505 1R protein
MFSLQNLCRKTLPDCKLPEFFDEYILQLLGLYWENHGTIQRAGNNCVLIQQRTLIPVNEALRIAASEENYEIVGL
LLAWEGNLYYAIIGALEGNRHDLIRKYDDQIKDHHEILPFIDDPVIFHKCHIIRRCFFNCILYQAVKYSKFRVLL
YFKHRLEDDLPLTHLLIEKACEDHNYEVIKWIYENLHTYDIMDTFECAIAHKDLRLYCLGYTFIYNRIVPYEYHH
LDILILSSLQLLHKVAAKGYLDFILETLKYDHNNDNLDIILTQAATYNHRKILTYFIPQLTYAQIEQCLFMAIKR
KSSKKTLNLLLSHLTLSIELIKKISQYVVTYNSTNIIGILSMKRKKKIYLDIMLTKYVKYAI FNKYWRCMDRFS
INPERIIKMAARINRMMLVKKISEHVWKNHAARLKHLKHAVHTMKHKDGKNRLMNFIYDRCYYHMQGEEIFSLAR
FYAIHHAPKLFDVFYDCCILDTIQFKSLLLDCSHIIGKNAHDATNINIVNKYIGDLFAMGVLSKKEILQDYPSIY
SKHYML*
SEQ ID No. 157-Warthog MGF 505 1R protein
MFSLQNLCRKTLPDCKLPEFFDEYILQLLGLYWENHGTIQRAGNNCVLIQQHTLIPVNEALRIAASEENYEIVSL
LLAWEGNLYYAIIGALEGNRHDLIRKYDDQIKDHHEILPFIDDPVIFHKCHIMRRCFFDCILYQAVKYSKFRVLL
YFKYRLENDLPLAHLLIKKACEDHNYEVIKWIYENLHIYNIMDTFGCAIAHKDLRLYRLGYTFIYNRIVPYKYHY
LDVLILSGLHLLYKVAAKGYLDFILETLKYDHNNDNLDIILTQAATYNHRKILTYYIPQLTYAQIEQCLFMAIKK
KSSKKTLNLLLSHLKLSIKLIKKISQYVATYNSTNIIGILNMRRKKKIYLDIILTKFVKKAIFNKFvvRCMDTFS
INPERIIKMAARINKMLLVKKISEHAWKNHAARLKHLKHAVYTMKHKDGKNRLMNLIYDHYYYHMQGEEIFSLAR
FYAIHHAPKLFDVFYDCCLLDTIRFKNLLLDCSHIIGKNAHDATNITIVNKYIGNLFAMGVLSKKEILQDYPSIY
SKHYMP*

The complete genome for the African swine fever virus Georgia 2007/1 pathogenic isolate is given in Genbank Locus: FR682468.1. The complete genome for the African swine fever virus Benin 97/1 pathogenic isolate is given in Genbank Locus: AM712239.1. The complete BA71 isolate genome encodes 151 open reading frames (ORFs), the Benin 97/1 isolate encodes 157 ORFs and the OURT88/3 isolate encodes 151 ORFs.

### Inhibition of IRF3 and NF-κB Activity

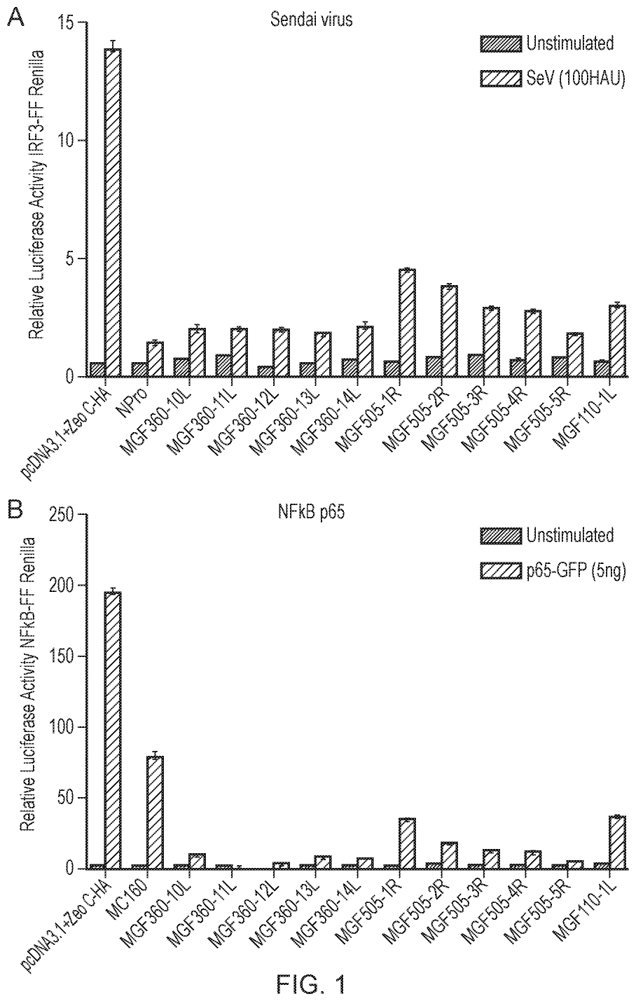

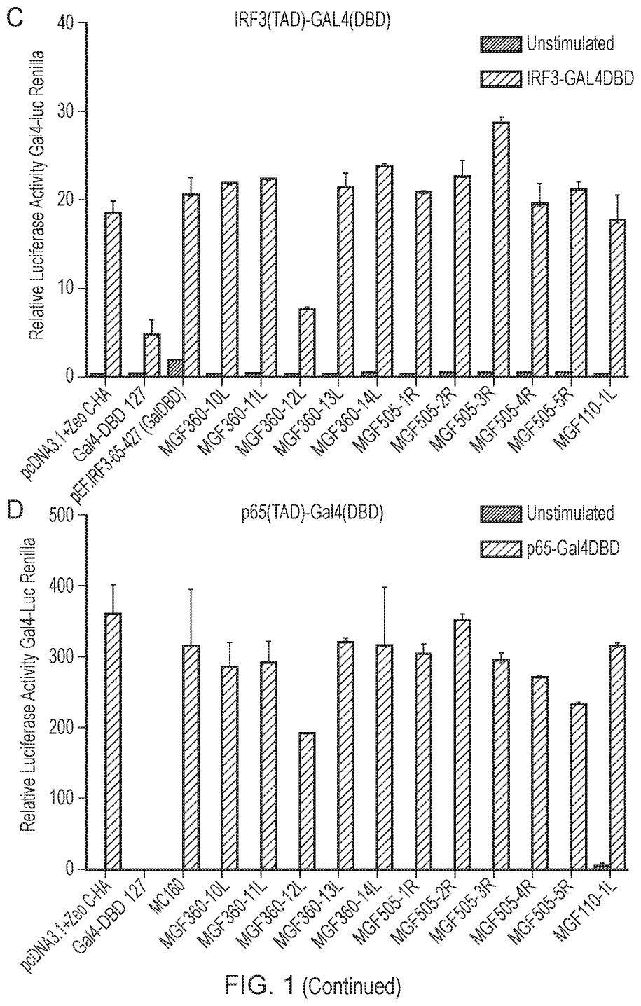

MGF genes MGF360-10L, 11L, 12L, 13L and 14L, MGF 505-1R, 2R, 3R, 4R and 5R, and MGF110-1L inhibit activity of the transcription factors interferon regulatory factor 3 (IRF3) and NF-κB, as demonstrated in Example 2 herein (see FIGS. 1A and 1B). MGF360-12L inhibits activity of IRF3 and NF-κB specifically by inhibiting their ability to activate transcription of target genes (see FIGS. 1C and 1D).

IRF3 and NF-κB are transcription factors that control expression of interferon and pro-inflammatory cytokines as part of the host's innate immune system response to viral infection. The inhibition of IRF3 and NF-κB by MGF genes results in decreased amounts of type I interferon (IFN) and pro-inflammatory cytokines produced by cells infected with ASFV. Expression of MGF genes allows ASFV to circumvent the host innate immune response, favouring virus replication and disrupting the development of adaptive responses.

The ability of MGF genes to inhibit IRF3 and/or NF-κB activity may be measured using luciferase reporter assays, such as described in Example 2 herein. For example, to assess whether a given MGF gene can inhibit IRF3 or NF-κB activity, the MGF gene can be expressed in cells comprising a luciferase reporter under control of a promoter that is activated by IRF3 or NF-κB, stimulating the cells in a manner that activates IRF3 or NF-κB respectively then measuring luciferase activity. The ability of modified or mutated versions of MGF genes to inhibit IRF3 and/or NF-κB activity may be assessed using such luciferase reporter assays.

### K145R Gene

The K145R gene is a late gene.

The gene (i.e. nucleotide) sequences of K145R genes from different ASFV strains are given below.

SEQ ID No. 158-Benin 97/1 K145R (NC_044956.1:58961-59398)
ATGGATCATTATCTTAAAAAATTACAAGATATTTATACGAAGCTCGAGGGCCATCCCTTTCTTTTTAGCCCGTCG
AAAACCAATGAAAAAGAGTTTATTACTCTGCTAAACCAGGCCTTGGCCTCAACGCAGCTTTACCGCAGCATACAA
CAGCTGTTTTTAACGATGTATAAGCTAGATCCCATTGGGTTTATTAACTATATTAAAACGAGTAAACAAGAGTAT
TTATGCCTGTTGATTAATCCTAAACTCGTTACTAAGTTTTTAAAAATAACGAGCTTTAAAATTTACATTAATTTC
AGACTGAAAACTTTTTATATAAGTCCTAATAAGTATAATAATTTTTACACTGCTCCCTCTGAAGAAAAGACTAAC
CATCTCCTAAAAGAAGAAAAAACTTGGGCAAAGATTGTTGAAGAAGGAGGAGAAGAATCCTAA
SEQ ID No. 159-China/2018/AnhuiXCGQ K145R (MK128995.1:64748-65185)
ATGGATCATTATCTTAAAAAATTACAAGATATTTATACGAAGCTCGAGGGTCATCCCTTTCTTTTTAGCCCGTCG
AAAACCAATGAAAAAGAGTTTATTACTCTGCTAAACCAGGCCTTGGCCTCAACGCAGCTTTACCGCAGCATACAA
CAGCTGTTTTTAACGATGTATAAGCTAGATCCCATTGGGTTTATTAACTATATTAAAACGAGTAAACAAGAGTAT
TTATGCCTGTTAATTAATCCTAAACTCGTTACTAAGTTTTTAAAAATAACGAGCTTTAAAATTTACATTAATTTC
AGGCTGAAAACTTTTTATATAAGTCCTAATAAGTATAATAATTTTTACACCGCTCCCTCTGAAGAAAAGACTAAC
CATCTTCTAAAAGAAGAAAAAACTTGGGCAAAGATTGTTGAAGAAGGAGGAGAAGAATCCTAA
SEQ ID No. 160-Georgia 2007/1 K145R (NC_044959.1:64734-65171)
ATGGATCATTATCTTAAAAAATTACAAGATATTTATACGAAGCTCGAGGGTCATCCCTTTCTTTTTAGCCCGTCG
AAAACCAATGAAAAAGAGTTTATTACTCTGCTAAACCAGGCCTTGGCCTCAACGCAGCTTTACCGCAGCATACAA
CAGCTGTTTTTAACGATGTATAAGCTAGATCCCATTGGGTTTATTAACTATATTAAAACGAGTAAACAAGAGTAT
TTATGCCTGTTAATTAATCCTAAACTCGTTACTAAGTTTTTAAAAATAACGAGCTTTAAAATTTACATTAATTTC
AGGCTGAAAACTTTTTATATAAGTCCTAATAAGTATAATAATTTTTACACCGCTCCCTCTGAAGAAAAGACTAAC
CATCTTCTAAAAGAAGAAAAAACTTGGGCAAAGATTGTTGAAGAAGGAGGAGAAGAATCCTAA
SEQ ID No. 161-Ken05/Tk1 K145R (NC_044945.1:65442-65879)
ATGGATCATTATCTTAAAAAATTACAGGATATTTATAAGAAGCTTGAGGGTCACCCCTTTCTTTTTAGTCCGTCG
AAAACCAATGAAAAAGAGTTTATTACTCTGCTAAACCAAGCCTTGGCCTCGACGCAGCTTTACCGCAGCATACAA
CAGCTGTTTTTAACGATGTATAAGCTAGATCCCATTGGGTTTGTTAACTATATTAAAGCGAGTAAACAAGAGTAT
TTATGTCTGTTGATTAATCCTAAACTAGTCACTAAGTTTTTAAAAATAACGAGCTTTAAAATTTACATTAATTTT
AGGCTGAAAACTTTTTATATAAGTCCTAATAAGTATAATAATTTTTACATCGCTCCCTCTGAAGAAAAGGCCAAT
CATCTCCTAAAAGAAGAAAAAACCTGGGCAAAGATTGTTGAAGAAGGAGGAGAAGAATCCTAA
SEQ ID No. 162-Ken06.Bus K145R (NC_044946.1:61226-61663)
ATGGATCATTATCTTAAAAAATTACAGGATATTTATAAGAAGCTTGAGGGTCACCCCTTTCTTTTTAGTCCGTCG
AAAACCAATGAAAAAGAGTTTATTACTCTGCTAAACCAAGCCTTGGCCTCGACGCAGCTTTACCGCAGCATACAA
CAGCTGTTTTTAACGATGTATAAGCTAGATCCCATTGGGTTTGTTAACTATATTAAAGCGAGTAAACAAGAGTAT
TTATGTCTGTTGATTAATCCTAAACTAGTCACTAAGTTTTTAAAAATAACGAGCTTTAAAATTTACATTAATTTC
AGGCTAAAAACTTTCTATATAAGTCCTAATAAGTATAATAATTTTTACATCGCTCCCTCTGAAGAAAAGGCCAAT
CATCTCCTAAAAGAAGAAAAAACCTGGGCAAAGATTGTTGAAGAAGGAGGAGAAGAATCCTAA
SEQ ID No. 163-Kenya 1950 K145R (AY261360.1:68066-68503)
ATGGATCATTATCTTAAAAAATTACAGGATATTTATAAGAAGCTTGAGGGTCACCCCTTTCTTTTTAGTCCGTCG
AAAACCAATGAAAAAGAGTTTATTACTCTGCTAAACCAAGCCTTGGCCTCGACGCAGCTTTACCGCAGCATACAA
CAGCTGTTTTTAACGATGTATAAGCTAGATCCCATTGGGTTTGTTAACTATATTAAAGCGAGTAAACAAGAGTAT
TTATGTCTGTTGATTAATCCTAAACTAGTCACTAAGTTTTTAAAAATAACGAGCTTTAAAATTTACATTAATTTT
AGGCTGAAAACTTTTTATATAAGTCCTAATAAGTATAATAATTTTTACATCGCTCCCTCTGAAGAAAAGGCCAAT
CATCTCCTAAAAGAAGAAAAAACCTGGGCAAAGATTGTTGAAGAAGGAGGAGAAGAATCCTAA
SEQ ID No. 164-L60 K145R (NC_044941.1:59310-59747)
ATGGATCATTATCTTAAAAAATTACAAGATATTTATACGAAGCTCGAGGGCCATCCCTTTCTTTTTAGCCCGTCG
AAAACCAATGAAAAAGAGTTTATTACTCTGCTAAACCAGGCCTTGGCCTCAACGCAGCTTTACCGCAGCATACAA
CAGCTGTTTTTAACGATGTATAAGCTAGATCCCATTGGGTTTATTAACTATATTAAAACGAGTAAACAAGAGTAT
TTATGCCTGTTGATTAATCCTAAACTCGTTACTAAGTTTTTAAAAATAACGAGCTTTAAAATTTACATTAATTTC
AGACTGAAAACTTTTTATATAAGTCCTAATAAGTATAATAATTTTTACACTGCTCCCTCTGAAGAAAAGACTAAC
CATCTCCTAAAAGAAGAAAAAACTTGGGCAAAGATTGTTGAAGAAGGAGGAGAAGAATCCTAA
SEQ ID No. 165-Malawi Lil-20/1 (1983) K145R (AY261361.1:62661-63098)
ATGGATCATTATCTTAAAAAATTAGAGGATATTTATAAAAAGCTTGAGGGTCATCCCTTTCTTTTTAGTCCGTCG
AAAACCAATGAAAAAGAGTTTATTACTCTGTTAAACCAAGCCTTGGCCTCAACGCAGCTTTATCGCAGCATACAA
CAGCTGTTTTTAACGATGTATAAGCTAGATCCCATTGGGTTTATTAACTATATTAAAACGAGTAAACAAGAGTAT
TTATGCCTGTTGATCAATCCTAAACTAGTTACGAAGTTTTTAAAAATAACGAGTTTTAAAATTTACATTAATTTC
AGGCTGAAAACTTTTTATATAAGTCCTAATAAATATAATAATTTTTACACCGCTCCCTCTGAAGAAAAGGCCAAT
CATCTCCTAAAAGAAGAAAAAACCTGGGCAAAGATTGTTGAAGAAGGAGGAGAAGAATCCTAA
SEQ ID No. 166-Mkuzi 1979 K145R (AY261362.1:66481-66921)
ATGGATCATTATCTTAAAAAATTACAAGATATTTATACGAAGCTCGAGGGCCATCCCTTTCTTTTTAGCCCGTCG
AAAACCAATGAAAAAGAGTTTATTACTCTGCTAAACCAGGCCTTGGCCTCAACGCAGCTTTACCGCAGCATACAA
CAGCTGTTTTTAACGATGTATAAGCTAGATCCTATAGGGTTTATTAACTATATTAAAACGAGTAAACAAGAGTAT
TTATGCCTGTTGATTAATCCTAAACTCGTTACTAAGTTTTTAAAAATAACGAGCTTTAAAATTTACATTAATTTC
AGACTGAAAACTTTTTATATAAGTCCTAATAAGTATAATAATTTTTACACCGCTCCCTCTGAAGAAAAGACCAAC
CATCTCCTAAAAGAAGAAAAAACTTGGGCAAAGATTGTTGAAGAAGGAGGAGGAGAAGAATCCTAA
SEQ ID No. 167-Pretorisuskop/96/4 K145R (AY261363.1:65440-65877)
ATGGATCATTATCTTAAAAAATTACAAGAAATTTATACGAAGCTCGAGGGCCATCCCTTTCTTTTTAGCCCGTCG
AAAACCAATGAAAAAGAGTTTATTACTCTGCTAAACCAGGCCTTGGCCTCAACGCAGCTTTATCGCAGCATACAA
CAGCTGTTTTTAACGATGTATAAGCTAGATCCCATTGGGTTTATTAACTATATTAAAACGAGTAAACAAGAGTAT
TTATGCCTGTTGATTAATCCTAAACTCGTCACTAAGTTTTTAAAAATAACGAGCTTTAAAATTTACATTAATTTC
AGACTGAAAACTTTTTATATAAGTCCTAATAAGTATAATAATTTTTACACCGCTCCCTCTGAAGAAAAGACCAAC
CATCTCCTAAAAGAAGAAAAAACTTGGGCAAAGATTGTTGAAGAAGGAGGAGAAGAATCCTAA
SEQ ID No. 168 -Tengani 62 K145R (AY261364.1:60111-60548)
ATGGATCATTATCTTAAAAAATTACAAGATATTTATACGAAGCTCGAGGGTCATCCCTTTCTTTTTAGCCCGTCG
AAAACCAATGAAAAAGAGTTTATTACTCTGCTAAACCAGGCCTTGGCCTCAACGCAGCTTTACCGCAGCATACAA
CAGCTGTTTTTAACGATGTATAAGCTAGATCCCATTGGGTTTATTAACTATATTAAAACGAGTAAACAAGAGTAT
TTATGCCTGTTGATTAATCCTAAACTCGTTACTAAGTTTTTAAAAATAACGAGCTTTAAAATTTACATTAATTTC
AGACTGAAAACTTTTTATATAAGTCCTAATAAGTATAATAATTTTTACACCGCTCCCTCTGAAGAAAAGACTAAC
CATCTCCTAAAAGAAGAAAAAACTTGGGCAAAGATTGTTGAAGAAGGAGGAGAAGAATCCTAA
SEQ ID No. 169-Warmbaths K145R (AY261365.1:64642-65082)
ATGGATCATTATCTTAAAAAATTACAAGATATTTATACGAAGCTCGAGGGCCATCCCTTTCTTTTTAGCCCGTCG
AAAACCAATGAAAAAGAGTTTATTACTCTGCTAAACCAGGCCTTGGCCTCAACGCAGCTTTACCGCAGCATACAA
CAGCTGTTTTTAACGATGTATAAGCTAGATCCTATAGGGTTTATTAACTATATTAAAACGAGTAAACAAGAGTAT
TTATGCCTGTTGATTAATCCTAAACTCGTTACTAAGTTTTTAAAAATAACGAGCTTTAAAATTTACATTAATTTC
AGACTGAAAACTTTTTATATAAGTCCTAATAAGTATAATAATTTTTACACCGCTCCCTCTGAAGAAAAGACCAAC
CATCTCCTAAAAGAAGAAAAAACTTGGGCAAAGATTGTTGAAGAAGGAGGAGGAGAAGAATCCTAA
SEQ ID No. 170-Warthog K145R (AY261366.1:61399-61836)
ATGGATCATTATCTTAAAAAATTACAAGATATTTATACGAAGCTCGAGGGCCATCCCTTTCTTTTTAGCCCGTCG
AAAACCAATGAAAAAGAGTTTATTACTCTGCTAAACCAGGCCTTGGCCTCAACGCAGCTTTACCGCAGCATACAA
CAGCTGTTTTTAACGATGTATAAGCTAGATCCCATTGGGTTTATTAACTATATTAAAACGAGTAAACAAGAGTAT
TTATGCCTGTTGATTAATCCTAAACTCGTCACTAAGTTTTTAAAAATAACGAGCTTTAAAATTTACATTAATTTC
AGACTGAAAACTTTTTATATAAGTCCTAATAAGTATAATAATTTTTACACCGCTCCCTCTGAAGAAAAGACCAAC
CATCTCCTAAAAGAAGAAAAAACTTGGGCAAAGATTGTTGAAGAAGGAGGAGAAGAATCCTAA

In an embodiment the attenuated ASFV of the invention lacks a functional version of the K145R gene. Suitably the K145R gene comprises the sequence of SEQ ID No. 158, 159, 160, 161, 162, 163, 164, 165, 166, 167, 168, 169 or 170. Suitably the K145R gene comprises a sequence having at least 70%, at least 80%, at least 90% or at least 95% identity with SEQ ID No. 158, 159, 160, 161, 162, 163, 164, 165, 166, 167, 168, 169 or 170. Suitably the K145R gene consists of the sequence of SEQ ID No. 158, 159, 160, 161, 162, 163, 164, 165, 166, 167, 168, 169 or 170.

The accessions numbers of K145R proteins from different strains of ASFV are listed below in Table 3.

TABLE 3

| | K145R protein |
|---|---|
| Strain | accession number |
| BA71V | Q07385 |
| PretoriuskopPr4/1996 | P0CA50 |
| Warthog | P0CA51 |
| Malawi-Lil/20 | P0CA49 |
| Kenya 1950 | P0CA48 |
| Georgia 2007/1 | E0WM19 |
| Portugal/OURT88/1988 | A9JLR1 |
| E75 | D4I5N9 |
| Benin 97/1 | A9JKZ8 |

The amino acid sequence of K145R proteins from different ASFV strain are depicted below.

SEQ ID No. 171-Benin 97/1 K145R protein
MDHYLKKLQDIYTKLEGHPFLFSPSKTNEKEFITLLNQALASTQLYRSIQQLFLTMYKLD
PIGFINYIKTSKQEYLCLLINPKLVTKFLKITSFKIYINFRLKTFYISPNKYNNFYTAPS
EEKTNHLLKEEKTWAKIVEEGGEES*
SEQ ID No. 172-China/2018/AnhuiXCGQ protein
MDHYLKKLQDIYTKLEGHPFLFSPSKTNEKEFITLLNQALASTQLYRSIQQLFLTMYKLD
PIGFINYIKTSKQEYLCLLINPKLVTKFLKITSFKIYINFRLKTFYISPNKYNNFYTAPS
EEKTNHLLKEEKTWAKIVEEGGEES*
SEQ ID No. 9-Georgia 2007/1 K145R protein
MDHYLKKLQDIYTKLEGHPFLFSPSKTNEKEFITLLNQALASTQLYRSIQQLFLTMYKLDPIGFINYIKTSKQEY
LCLLINPKLVTKFLKITSFKIYINFRLKTFYISPNKYNNFYTAPSEEKTNHLLKEEKTWAKIVEEGGEES
SEQ ID No. 173-Ken05/Tk1 K145R protein
MDHYLKKLQDIYKKLEGHPFLFSPSKTNEKEFITLLNQALASTQLYRSIQQLFLTMYKLD
PIGFVNYIKASKQEYLCLLINPKLVTKFLKITSFKIYINFRLKTFYISPNKYNNFYIAPS
EEKANHLLKEEKTWAKIVEEGGEES*
SEQ ID No. 174-Ken06.Bus K145R protein
MDHYLKKLQDIYKKLEGHPFLFSPSKTNEKEFITLLNQALASTQLYRSIQQLFLTMYKLD
PIGFVNYIKASKQEYLCLLINPKLVTKFLKITSFKIYINFRLKTFYISPNKYNNFYIAPS
EEKANHLLKEEKTWAKIVEEGGEES*
SEQ ID No. 175-Kenya 1950 K145R protein
MDHYLKKLQDIYKKLEGHPFLFSPSKTNEKEFITLLNQALASTQLYRSIQQLFLTMYKLD
PIGFVNYIKASKQEYLCLLINPKLVTKFLKITSFKIYINFRLKTFYISPNKYNNFYIAPS
EEKANHLLKEEKTWAKIVEEGGEES*
SEQ ID No. 176-L60 K145R protein
MDHYLKKLQDIYTKLEGHPFLFSPSKTNEKEFITLLNQALASTQLYRSIQQLFLTMYKLD
PIGFINYIKTSKQEYLCLLINPKLVTKFLKITSFKIYINFRLKTFYISPNKYNNFYTAPS
EEKTNHLLKEEKTWAKIVEEGGEES*
SEQ ID No. 177-Malawi Lil-20/1 (1983) K145R protein
MDHYLKKLEDIYKKLEGHPFLFSPSKTNEKEFITLLNQALASTQLYRSIQQLFLTMYKLD
PIGFINYIKTSKQEYLCLLINPKLVTKFLKITSFKIYINFRLKTFYISPNKYNNFYTAPS
EEKANHLLKEEKTWAKIVEEGGEES*
SEQ ID No. 178-Mkuzi 1979 K145R protein
MDHYLKKLQDIYTKLEGHPFLFSPSKTNEKEFITLLNQALASTQLYRSIQQLFLTMYKLD
PIGFINYIKTSKQEYLCLLINPKLVTKFLKITSFKIYINFRLKTFYISPNKYNNFYTAPS
EEKTNHLLKEEKTWAKIVEEGGGEES*
SEQ ID No. 179-Pretorisuskop/96/4 K145R protein
MDHYLKKLQEIYTKLEGHPFLFSPSKTNEKEFITLLNQALASTQLYRSIQQLFLTMYKLD
PIGFINYIKTSKQEYLCLLINPKLVTKFLKITSFKIYINFRLKTFYISPNKYNNFYTAPS
EEKTNHLLKEEKTWAKIVEEGGEES*
SEQ ID No. 180-Tengani 62 K145R protein
MDHYLKKLQDIYTKLEGHPFLFSPSKTNEKEFITLLNQALASTQLYRSIQQLFLTMYKLD
PIGFINYIKTSKQEYLCLLINPKLVTKFLKITSFKIYINFRLKTFYISPNKYNNFYTAPS
EEKTNHLLKEEKTWAKIVEEGGEES*
SEQ ID No. 181-Warmbaths K145R protein
MDHYLKKLQDIYTKLEGHPFLFSPSKTNEKEFITLLNQALASTQLYRSIQQLFLTMYKLD
PIGFINYIKTSKQEYLCLLINPKLVTKFLKITSFKIYINFRLKTFYISPNKYNNFYTAPS
EEKTNHLLKEEKTWAKIVEEGGGEES*
SEQ ID No. 182-Warthog K145R protein
MDHYLKKLQDIYTKLEGHPFLFSPSKTNEKEFITLLNQALASTQLYRSIQQLFLTMYKLD
PIGFINYIKTSKQEYLCLLINPKLVTKFLKITSFKIYINFRLKTFYISPNKYNNFYTAPS
EEKTNHLLKEEKTWAKIVEEGGEES*

K145R inhibits the host endoplasmic reticulum (ER) stress response (Barber 2015 Stress modulators encoded by African swine fever virus; PhD thesis, St Georges, University of London, 2016). This response is caused by the accumulation of unfolded proteins and may be activated during viral infections due to the substantial amounts of viral proteins being produced. ER stress leads to the increase in expression of the transcription factor CCAAT-enhancer-binding protein homologous protein (CHOP) and its accumulation in the nucleus of the cells. CHOP activity ultimately results in cell apoptosis, thus limiting viral replication.

K145R function may be tested by methods including immunofluorescence using an antibody against CHOP and assessment of its presence in the nucleus of cells following induction of ER stress, and luciferase reporter assay, where the luciferase gene is under control of the CHOP promoter.

### B125R Gene

The gene (i.e. nucleotide) sequences of B125R genes from different ASFV strains are given below.

SEQ ID No. 183-Benin 97/1 B125R (NC_044956.1:100206-100583)
ATGGCGGTTTATGCGAAGGATCTTGATAATAACAAAGAGTTAAACCAAAAATTAATTAACGATCAGCTTAAAATT
ATTGACACGCTCTTGCTAGCAGAAAAAAAAAACTTTTTGGTGTATGAATTGCCTGCCCCTTTTGACTTTTCCTCC
GGCGACCCTTTGGCCAGTCAGCGCGACATATACTATGCCATCATAAAAAGCCTCGAGGAGCGCGGGTTTACTGTC
AAAATATGTATGAAAGGGGATCGTGCCCTCCTTTTCATCACTTGGAAAAAAATACAATCCATTGAGATAAACAAA
AAAGAAGAATATCTGCGCATGCACTTCATACAAGACGAAGAGAAAGCATTTTATTGTAAATTTTTAGAGTCTAGA
TGA
SEQ ID No. 184-China/2018/AnhuiXCGQ B125R (MK128995.1:105587-105964)
ATGGCGGTTTATGCGAAGGATCTTGATAATAACAAAGAGTTAAACCAAAAATTAATTAACGATCAGCTTAAAATT
ATTGACACGCTCTTGCTGGCAGAAAAAAAAAACTTTTTGGTGTATGAACTACCTGCCCCTTTTGACTTTTCCTCC
GGCGACCCTTTGGCCAGTCAGCGCGACATATACTATGCCATCATAAAAAGCCTCGAGGAGCGCGGGTTTACTGTC
AAAATATGTATGAAAGGGGATCGTGCCCTCCTTTTCATCACCTGGAAAAAAATACAATCCATTGAGATAAACAAA
AAAGAAGAATATCTGCGCATGCACTTCATACAAGACGAAGAGAAAGCATTTTATTGTAAATTTTTAGAGTCTAGA
TGA
SEQ ID No. 185-Georgia 2007/1 B125R (NC_044959.1:105570-105947)
ATGGCGGTTTATGCGAAGGATCTTGATAATAACAAAGAGTTAAACCAAAAATTAATTAACGATCAGCTTAAAATT
ATTGACACGCTCTTGCTGGCAGAAAAAAAAAACTTTTTGGTGTATGAACTACCTGCCCCTTTTGACTTTTCCTCC
GGCGACCCTTTGGCCAGTCAGCGCGACATATACTATGCCATCATAAAAAGCCTCGAGGAGCGCGGGTTTACTGTC
AAAATATGTATGAAAGGGGATCGTGCCCTCCTTTTCATCACCTGGAAAAAAATACAATCCATTGAGATAAACAAA
AAAGAAGAATATCTGCGCATGCACTTCATACAAGACGAAGAGAAAGCATTTTATTGTAAATTTTTAGAGTCTAGA
TGA
SEQ ID No. 186-Ken05/Tk1 B125R (NC_044945.1:106516-106893)
ATGGCGGTTTATGCGAAGGACCTTGATAATAACAAAGAGTTAAACCAAAAATTGATCAACGATCAGCTTAAAATC
ATTGACACGCTTTTGCTAGCAGAAAAAAAAAACTTTTTGGTGTACGAACTACCTGCCCATTTTGACTTTTCCTCC
GGCGACCCTTTGGGCAGTCAGCGCGACATTTACTATGCCATCATAAAAAGTCTCGAGGAGCGCGGATTTACTGTC
AAAATATGTATGAAGGGAGACCGCGCCCTTCTTTTCATCACCTGGAAAAAAATACAATCCATTGAGATCAACAAA
AAAGAAGAATATCTGCGCATGCACTTCATACAAGACGAAGAGAAAGCGTTTTATTGTAAATTTTTAGAGTCTAGA
TGA
SEQ ID No. 187-Ken06.Bus B125R (NC_044946.1:102432-102809)
ATGGCGGTTTATGCGAAGGACCTTGATAATAACAAAGAGTTAAACCAAAAATTGATCAACGATCAGCTTAAAATT
ATTGACACGCTTTTGCTGGCAGAAAAAAAAAACTTTTTGGTGTACGAACTACCTGCCCATTTTGACTTTTCCTCC
GGCGACCCTTTGGGCAGTCAGCGCGACATTTACTATGCCATCATAAAAAGTCTCGAGGAGCGCGGATTTACTGTC
AAAATATGTATGAAGGGAGACCGCGCCCTTCTTTTCATCACCTGGAAAAAAATACAATCCATTGAGATCAACAAA
AAAGAAGAATATCTGCGCATGCACTTCATACAAGACGAAGAGAAAGCGTTTTATTGTAAATTTTTAGAGTCTAGA
TGA
SEQ ID No. 188-Kenya 1950 B125R (AY261360.1:109401-109778)
ATGGCGGTTTATGCGAAGGACCTTGATAATAACAAAGAGTTAAACCAAAAATTGATCAACGATCAGCTTAAAATC
ATTGACACGCTTTTGCTAGCAGAAAAAAAAAACTTTTTGGTGTACGAACTACCTGCCCATTTTGACTTTTCCTCC
GGCGACCCTTTGGGCAGTCAGCGCGACATTTACTATGCCATCATAAAAAGTCTCGAGGAGCGCGGATTTACTGTC
AAAATATGTATGAAGGGAGACCGCGCCCTTCTTTTCATCACCTGGAAAAAAATACAATCCATTGAGATCAACAAA
AAAGAAGAATATCTGCGCATGCACTTCATACAAGACGAAGAGAAAGCGTTTTATTGTAAATTTTTAGAGTCTAGA
TGA
SEQ ID No. 189-L60 B125R (NC_044941.1:100308-100685)
ATGGCGGTTTATGCGAAGGATCTTGATAATAACAAAGAGTTAAACCAAAAATTAATTAACGATCAGCTTAAAATT
ATTGACACGCTCTTGCTAGCAGAAAAAAAAAACTTTTTGGTGTATGAATTGCCTGCCCCTTTTGACTTTTCCTCC
GGCGACCCTTTGGCCAGTCAGCGCGACATATACTATGCCATCATAAAAAGCCTCGAGGAGCGCGGGTTTACTGTC
AAAATATGTATGAAAGGGGATCGTGCCCTCCTTTTCATCACTTGGAAAAAAATACAATCCATTGAGATAAACAAA
AAAGAAGAATATCTGCGCATGCACTTCATACAAGACGAAGAGAAAGCATTTTATTGTAAATTTTTAGAGTCTAGA
TGA
SEQ ID No. 190-Malawi Lil-20/1 (1983) B125R (AY261361.1:103937-104314)
ATGGCAGTTTATGCGAAAGATCTTGATAATAACAAAGAGTTAAACCAAAAATTAATTAACGATCAACTTAAAATT
ATTGACACGCTGTTGCTGGCAGAAAAAAAAAACTTTTTGGTGCATGAACTACCTGCCCACTATGACTTTTCCTCC
GGCGACCCTTTGGCCAGTCAGCGCGACATATACTATGCCATCATAAAAAGTCTCGAGGAGCGCGGATTTACTGTC
AAAATTTGTATGAAAGGAGATCGCGCCCTTCTTTTCATCACCTGGAAAAAAATACAATCCATTGAGATAAACAAA
AAAGAAGAATATCTGCGCATGCACTTCATACAAGATGAAGAGAAAGCATTTTATTGTAAATTTTTAGAGTCTAGA
TGA
SEQ ID No. 191 -Mkuzi 1979 B125R (AY261362.1:107379-107756)
ATGGCGGTTTATGCGAAGGATCTTGATAATAACAAAGAGTTAAACCAAAAATTAATTAACGATCAGCTTAAAATT
ATTGACACGCTCTTGCTAGCAGAAAAAAAAAACTTTTTGGTGTATGAATTGCCTGCCCCTTTTGACTTTTCCTCC
GGCGACCCTTTGGCCAGTCAGCGCGACATATACTATGCCATCATAAAAAGCCTCGAGGAGCGCGGGTTTACTGTC
AAAATATGTATGAAAGGGGATCGTGCCCTCCTTTTCATCACTTGGAAAAAAATACAATCCATTGAGATAAACAAA
AAAGAAGAATATCTGCGCATGCACTTCATACAAGACGAAGAGAAAGCATTTTATTGTAAATTTTTAGAGTCTAGA
TGA
SEQ ID No. 192-Pretorisuskop/96/4 B125R (AY261363.1:106504-106881)
ATGGCGGTTTATGCGAAGGATCTTGATAATAACAAAGAGTTAAACCAAAAATTAATTAACGATCAGCTTAAAATT
ATTGACACGCTCTTGCTGGCAGAAAAAAAAAACTTTTTGGTGTATGAACTACCTGCCCCTTTTGACTTTTCCTCC
GGCGACCCTTTGGCCAGTCAGCGCGACATATACTATGCCATCATAAAAAGTCTCGAGGAGCGCGGGTTTACTGTC
AAAATATGTATGAAAGGGGATCGCGCCCTCCTTTTCATCACCTGGAAAAAAATACAATCCATTGAGATAAACAAA
AAAGAAGAATATCTGCGCATGCACTTCATACAAGACGAAGAGAAAGCATTTTATTGTAAATTTTTAGAGTCTAGA
TGA
SEQ ID No. 193-Tengani 62 B125R (AY261364.1:100837-101214)
ATGGCGGTGTATGCGAAGGACCTTGATAATAACAAAGAGTTAAACCAAAAATTAATTAACGATCAGCTTAAAATT
ATTGACACGCTCTTGCTGGCAGAAAAAAAAAACTTTTTGGTGTATGAACTACCTGCCCCTTTTGACTTTTCCTCC
GGCGACCCTTTGGCCAGTCAGCGTGACATATACTATGCCATCATAAAAAGTCTTGAGGAGCGCGGGTTTACTGTC
AAAATATGTATGAAAGGGGATCGTGCCCTCCTTTTCATCACCTGGAAAAAAATACAATCCATTGAAATAAACAAA
AAAGAAGAATATCTGCGCATGCACTTCATACAAGACGAAGAGAAATCATTTTATTGTAAATTTTTAGAGTCTAGA
TGA
SEQ ID No. 194-Warmbaths B125R (AY261365.1:105532-105909)
ATGGCGGTTTATGCGAAGGATCTTGATAATAACAAAGAGTTAAACCAAAAATTAATTAACGATCAGCTTAAAATT
ATTGACACGCTCTTGCTGGCAGAAAAAAAAAACTTTTTGGTGTATGAACTACCTGCCCCTTTTGACTTTTCCTCC
GGCGACCCTTTGGCCAGTCAGCGCGACATATACTATGCCATCATAAAAAGTCTCGAGGAGCGCGGGTTTACTGTC
AAAATATGTATGAAAGGGGATCGCGCCCTCCTTTTCATCACCTGGAAAAAAATACAATCCATTGAGATAAACAAA
AAAGAAGAATATCTGCGCATGCACTTCATACAAGACGAAGAGAAAGCATTTTATTGTAAATTTTTAGAGTCTAGA
TGA
SEQ ID No. 195-Warthog B125R (AY261366.1:102258-102635)
ATGGCGGTTTATGCGAAGGACCTTGATAATAACAAAGAGTTAAACCAAAAATTAATTAACGATCAGCTTAAAATT
ATTGACACGCTCTTGCTGGCAGAAAAAAAAAACTTTTTGGTGTATGAACTACCTGCCCCTTTTGACTTTTCCTCC
GGCGACCCTTTGGCCAGTCAGCGCGACATATACTATGCCATCATAAAAAGTCTCGAGGAGCGCGGGTTTACTGTC
AAAATATGTATGAAAGGGGATCGCGCCCTCCTTTTCATCACCTGGAAAAAAATACAATCCATTGAGATAAACAAA
AAAGAAGAATATCTGCGCATGCACTTCATACAAGACGAAGAGAAAGCATTTTATTGTAAATTTTTAGAGTCTAGA
TGA

In an embodiment the attenuated ASFV of the invention lacks a functional version of the B125R gene. Suitably the B125R gene comprises the sequence of SEQ ID No. 183, 184, 185, 186, 187, 188, 189, 190, 191, 192, 193, 194 or 195. Suitably the B125R gene comprises a sequence having at least 70%, at least 80%, at least 90% or at least 95% identity with SEQ ID No. 183, 184, 185, 186, 187, 188, 189, 190, 191, 192, 193, 194 or 195. Suitably the B125R gene consists of the sequence of SEQ ID No. 183, 184, 185, 186, 187, 188, 189, 190, 191, 192, 193, 194 or 195.

The accessions numbers of B125R proteins from different strains of ASFV are listed below in Table 4.

TABLE 4

| | B125R protein |
|---|---|
| Strain | accession number |
| BA71V | Q65171 |
| PretoriuskopPr4/1996 | P0CA34 |
| Warthog | P0CA35 |
| Georgia 2007/1 | E0WMM1 |
| Portugal/OURT88/1988 | A9JLX0 |
| E75 | D4I5S1 |
| Benin 97/1 | A9JL41 |

The amino acid sequences of B125R proteins from different ASFV strains are depicted below.

SEQ ID No. 196-Benin 97/1 B125R protein
MAVYAKDLDNNKELNQKLINDQLKIIDTLLLAEKKNFLVYELPAPFDFSSGDPLASQRDIYYAIIKSLEERGFTV
KICMKGDRALLFITWKKIQSIEINKKEEYLRMHFIQDEEKAFYCKFLESR*
SEQ ID No. 197-China/2018/AnhuiXCGQ B125R protein
MAVYAKDLDNNKELNQKLINDQLKIIDTLLLAEKKNFLVYELPAPFDFSSGDPLASQRDIYYAIIKSLEERGFTV
KICMKGDRALLFITWKKIQSIEINKKEEYLRMHFIQDEEKAFYCKFLESR*
SEQ ID No. 10-Georgia 2007/1 B125R protein
MAVYAKDLDNNKELNQKLINDQLKIIDTLLLAEKKNFLVYELPAPFDFSSGDPLASQRDIYY
AIIKSLEERGFTVKICMKGDRALLFITWKKIQSIEINKKEEYLRMHFIQDEEKAFYCKFLES
R
SEQ ID No. 198-Ken05/Tk1 B125R protein
MAVYAKDLDNNKELNQKLINDQLKIIDTLLLAEKKNFLVYELPAHFDFSSGDPLGSQRDIYYAIIKSLEERGFTV
KICMKGDRALLFITWKKIQSIEINKKEEYLRMHFIQDEEKAFYCKFLESR*
SEQ ID No. 199-Ken06.Bus B125R protein
MAVYAKDLDNNKELNQKLINDQLKIIDTLLLAEKKNFLVYELPAHFDFSSGDPLGSQRDIYYAIIKSLEERGETV
KICMKGDRALLFITWKKIQSIEINKKEEYLRMHFIQDEEKAFYCKFLESR*
SEQ ID No. 200-Kenya 1950 B125R protein
MAVYAKDLDNNKELNQKLINDQLKIIDTLLLAEKKNFLVYELPAHFDFSSGDPLGSQRDIYYAIIKSLEERGFTV
KICMKGDRALLFITWKKIQSIEINKKEEYLRMHFIQDEEKAFYCKFLESR*
SEQ ID No. 201-L60 B125R protein
MAVYAKDLDNNKELNQKLINDQLKIIDTLLLAEKKNFLVYELPAPFDFSSGDPLASQRDIYYAIIKSLEERGFTV
KICMKGDRALLFITWKKIQSIEINKKEEYLRMHFIQDEEKAFYCKFLESR*
SEQ ID No. 202-Malawi Lil-20/1 B125R protein
MAVYAKDLDNNKELNQKLINDQLKIIDTLLLAEKKNFLVHELPAHYDFSSGDPLASQRDIYYAIIKSLEERGFTV
KICMKGDRALLFITWKKIQSIEINKKEEYLRMHFIQDEEKAFYCKFLESR*
SEQ ID No. 203-Mkuzi 1979 B125R protein
MAVYAKDLDNNKELNQKLINDQLKIIDTLLLAEKKNFLVYELPAPFDFSSGDPLASQRDIYYAIIKSLEERGFTV
KICMKGDRALLFITWKKIQSIEINKKEEYLRMHFIQDEEKAFYCKFLESR*
SEQ ID No. 204-Pretorisuskop/96/4 B125R protein
MAVYAKDLDNNKELNQKLINDQLKIIDTLLLAEKKNFLVYELPAPFDFSSGDPLASQRDIYYAIIKSLEERGFTV
KICMKGDRALLFITWKKIQSIEINKKEEYLRMHFIQDEEKAFYCKFLESR*
SEQ ID No. 205-Tengani 62 B125R protein
MAVYAKDLDNNKELNQKLINDQLKIIDTLLLAEKKNFLVYELPAPFDFSSGDPLASQRDIYYAIIKSLEERGFTV
KICMKGDRALLFITWKKIQSIEINKKEEYLRMHFIQDEEKSFYCKFLESR*
SEQ ID No. 206-Warmbaths B125R protein
MAVYAKDLDNNKELNQKLINDQLKIIDTLLLAEKKNFLVYELPAPFDFSSGDPLASQRDIYYAIIKSLEERGFTV
KICMKGDRALLFITWKKIQSIEINKKEEYLRMHFIQDEEKAFYCKFLESR*
SEQ ID No. 207 -Warthog B125R protein
MAVYAKDLDNNKELNQKLINDQLKIIDTLLLAEKKNFLVYELPAPFDFSSGDPLASQRDIYYAIIKSLEERGFTV
KICMKGDRALLFITWKKIQSIEINKKEEYLRMHFIQDEEKAFYCKFLESR*

B125R was identified as one of the most abundant viral proteins expressed in infected wild boar cells (WSL-R) (Kapler et al. 2018 Sci. Rep. 8: 1471).

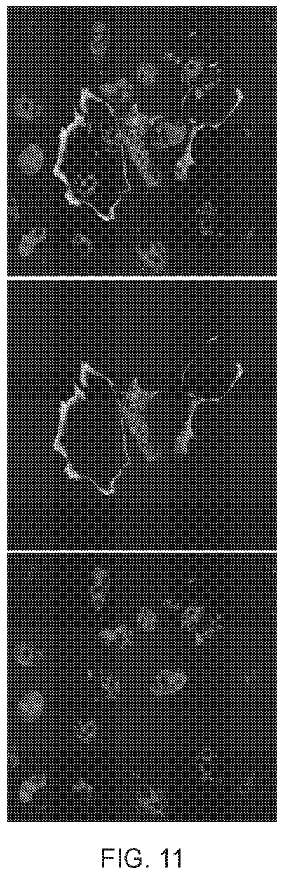

As shown in FIG. 11, B125R expression can be detected at the cell surface indicating that B125R is likely to be exposed to antibodies and likely to induce a strong antibody response.

### Expression and Activity

The attenuated African Swine Fever (ASF) virus of one embodiment of the present invention has disrupted expression and/or activity of the following genes:
MGF 360 genes 12L, 13L and 14L; and
MGF 505 gene 1R.

These genes may be referred to herein as the “disrupted genes”.

### Expression

In an embodiment the invention provides an attenuated African Swine Fever (ASF) virus in which the expression of the following genes is disrupted:
multigene-family (MGF) 360 12L, 13L and 14L, and
MGF 505 1R.

The term “expression” with respect to a gene refers to the ability of the ASF virus to produce the product of the gene, such as RNA and/or protein. Disruption of expression of a gene means that production of the gene product is decreased. Expression of the gene may be decreased by at least 50%, at least 60%, at least 70%, at least 80%, at least 90% and/or at least 95%. Expression of the gene may be decreased to the extent that production of the gene product, such as RNA and/or protein, is entirely abolished (i.e. the gene product is not produced at all). Disruption of gene expression decreases expression of the gene relative to the expression of the gene when it is not disrupted. For example, a mutated gene may have decreased expression in comparison to a wild type version of the gene.

A gene the expression of which is disrupted may not be fully transcribed and translated. Transcription of the gene may be decreased by at least 50%, at least 60%, at least 70%, at least 80%, at least 90% and/or at least 95%. Transcription of the gene may be abolished (i.e. the gene may not be transcribed). Translation of the gene may be decreased by at least 50%, at least 60%, at least 70%, at least 80%, at least 90% and/or at least 95%. Translation of the gene may be abolished. The gene may be transcribed but not translated. The gene may be transcribed and translated but the protein too rapidly degraded to carry out its function. The gene may be transcribed and translated but the protein may be non-functional.

Gene expression may be measured by techniques known in the art. For example, the amount of mRNA transcribed from a gene may be quantified, such as by using quantitative polymerase chain reaction (qPCR). Alternatively or additionally, the amount of protein may be quantified, such as by using Western blotting or mass spectrometry.

### Activity

In an embodiment the invention provides an attenuated African Swine Fever (ASF) virus in which the activity of the following genes is disrupted:
multigene-family (MGF) 360 12L, 13L and 14L, and
MGF 505 1R.

The term “activity” with respect to a gene refers to the ability of the gene to carry out its functions. Different genes have different activities i.e. different functions they fulfil. A given gene may have multiple activities; disruption of gene activity means disruption of one or more of those activities. One or more activity of the gene may be disrupted whilst one or more other activities are not disrupted. Disruption of gene activity decreases the activity of the gene relative to the activity of the gene when it is not disrupted. For example, a mutated gene may have decreased activity in comparison to a wild type version of the gene. Gene activity may be decreased to the extent that gene activity is entirely abolished.

The attenuated ASFV according to the present invention may comprise a non-functional version of the disrupted genes.

Disruption of expression of a gene may also disrupt activity of that gene as the decreased amount of gene product means the gene cannot as effectively carry out one or more of its activities.

In an embodiment, the activity of the gene that is disrupted is the ability of the gene to inhibit IRF3 and/or NFκB activity. In an embodiment, the invention provides an attenuated African Swine Fever (ASF) virus in which the ability of the following genes to inhibit IRF3 and/or NFκB activity is disrupted:
multigene-family (MGF) 360 12L, 13L and 14L, and
MGF 505 1R.

In an embodiment, the invention provides an attenuated ASF virus in which the ability of the MGF 360 12L, 13L and 14L, and MGF 505 1R genes to inhibit IRF3 activity is disrupted. In an embodiment, the invention provides an attenuated ASF virus in which the ability of the MGF 360 12L, 13L and 14L, and MGF 505 1R genes to inhibit NFκB activity is disrupted. The ability of MGF 360 12L, 13L and 14L, and/or MGF 505 1R to inhibit IRF3 and/or NFκB activity may be measured using a luciferase reporter assay as described herein.

Disruption of the ability to inhibit IRF3 and/or NFκB activity means that the ability to inhibit IRF3 and/or NFκB activity is decreased. In other words, the version of the MGF gene in the attenuated ASF virus of the invention inhibits IRF3 and/or NFκB activity less than would a wild type version of the same MGF gene. The ability to inhibit IRF3 and/or NFκB activity may be decreased by at least 50%, at least 60%, at least 70%, at least 80%, or at least 90%. In an embodiment the ability of the MGF gene to inhibit IRF3 and/or NFκB activity may be abolished i.e. the MGF gene does not inhibit IRF3 and/or NFκB activity. In that case, the activity of IRF3 and/or NFκB would be as high as if no MGF gene were present. A decrease in inhibition means IRF3 and/or NFκB activity is higher than it would be with the wild type version of the MGF gene. IRF3 and/or NFκB activity may be twice as high as it would be with the wild type version of the MGF gene, such as three times as high, four times as high, five times as high or six times as high.

IRF3 and NFκB are transcription factors that activate transcription of various genes, controlling expression of interferon and pro-inflammatory cytokines as part of the host's innate immune system response to viral infection. Inhibition of IRF3 and/or NFκB activity means decreasing activation of expression of their target genes. The degree of activation of IRF3 and/or NFκB target gene expression may be measured using a luciferase reporter assay in which an IRF3-activatable or NFκB-activatable promoter is linked to a luciferase reporter.

Suitably the ability of MGF 360 12L to inhibit IRF3 activity is disrupted. Suitably the ability of MGF 360 13L to inhibit IRF3 activity is disrupted. Suitably the ability of MGF 360 14L to inhibit IRF3 activity is disrupted. Suitably the ability of MGF 505 1R to inhibit IRF3 activity is disrupted. Suitably the ability of MGF 360 12L to inhibit NFκB activity is disrupted. Suitably the ability of MGF 360 13L to inhibit NFκB activity is disrupted. Suitably the ability of MGF 360 14L to inhibit NFκB activity is disrupted. Suitably the ability of MGF 505 1R to inhibit NFκB activity is disrupted.

IRF3 and NFκB function by binding to target DNA and activating transcription. An MGF gene may inhibit IRF3 and/or NFκB activity by inhibiting the ability of IRF3 and/or NFκB to activate transcription. In an embodiment the ability of MGF 360 12L to inhibit transcriptional activation by IRF3 and/or NFκB is disrupted. Suitably the ability of MGF 360 12L to inhibit transcriptional activation by IRF3 is disrupted. Suitably the ability of MGF 360 12L to inhibit transcriptional activation by NFκB is disrupted.

Mutation Disrupting Expression and/or Activity

Gene expression and/or activity are disrupted by mutating the ASFV genome i.e. by mutating the ASFV genome. A “mutation” means a change in the nucleotide sequence of the ASFV genome relative to a known ASFV genotype. Mutations include changing one or more nucleotides to different nucleotides (i.e. substitution), adding nucleotides, deleting nucleotides and/or a combination of these.

In an embodiment the attenuated ASF virus of the invention comprises mutations that disrupt the expression and/or activity of the following genes:
MGF 360 genes 12L, 13L and 14L; and
MGF 505 gene 1R.

Gene expression and/or activity may be disrupted by disrupting transcription of the gene into mRNA i.e. by decreasing gene transcription, such as completely abolishing gene transcription. Gene expression and/or activity may be disrupted by disrupting translation of mRNA into protein. In an embodiment the attenuated ASF virus comprises mutations that decrease transcription and/or translation of the genes. In an embodiment the attenuated ASF virus comprises mutations that cause the genes to not be transcribed and/or translated (i.e. complete abolition of transcription and/or translation).

Gene expression and/or activity may be disrupted by mutating a non-coding sequence associated with the gene, such as a promoter. In an embodiment the attenuated ASF virus comprises mutations in promoters of one or more of the disrupted genes.

Gene expression and/or activity may be disrupted by mutating a coding sequence of one or more of the disrupted genes.

### Functional Version of a Gene

The attenuated ASFV of the invention comprises a functional version of the following genes:
(i) MGF 360 10L and/or 11L, and
(ii) MGF 505 2R and/or 3R.

Suitably the attenuated ASFV of the invention comprises functional versions of MGF 360 10L and 11L. Suitably the attenuated ASFV of the invention comprises functional versions of MGF 505 2R and 3R. Suitably the attenuated ASFV of the invention comprises functional versions of MGF 360 10L and MGF 505 2R. Suitably the attenuated ASFV of the invention comprises functional versions of MGF 360 10L and MGF 505 3R. Suitably the attenuated ASFV of the invention comprises functional versions of MGF 360 11L and MGF 505 2R. Suitably the attenuated ASFV of the invention comprises functional versions of MGF 360 11L and MGF 505 3R. Suitably the attenuated ASFV of the invention comprises functional versions of MGF 360 10L and MGF 505 2R and 3R. Suitably the attenuated ASFV of the invention comprises functional versions of MGF 360 11L and MGF 505 2R and 3R. Suitably the attenuated ASFV of the invention comprises functional versions of MGF 360 10L and 11L and MGF 505 2R. Suitably the attenuated ASFV of the invention comprises functional versions of MGF 360 10L and 11L and MGF 505 3R. Suitably the attenuated ASFV of the invention comprises functional versions of MGF 360 10L and 11L and MGF 505 2R and 3R.

In an embodiment, the attenuated ASFV of the invention comprises a DIVA mutation that is lack of a functional version of the K145R gene and/or lack of a functional version of the B125R gene.

In another aspect the invention provides an attenuated ASFV which lacks a functional version of the K145R gene and/or the B125R gene.

The expression “functional version” of a gene refers to a gene the expression and activity of which have not been disrupted. In other words, a functional version of a gene is not mutated in a manner that disrupts gene expression or gene activity. A functional version of a gene may not comprise any mutations. The coding sequence of a functional version of a gene may be complete and uninterrupted. A functional version of a gene may be fully transcribed and translated.

The function of an MGF gene may refer its ability to inhibit IRF3 and/or NFκB activity. A functional version of an MGF gene may inhibit IRF3 and/or NFκB activity. In an embodiment the attenuated ASFV of the invention comprises a version of the following genes that inhibits IRF3 and/or NFκB activity:
(i) MGF 360 10L and/or 11L, and
(ii) MGF 505 2R and/or 3R.

The ability of MGF 360 10L and/or 11L, and MGF 505 2R and/or 3R to inhibit IRF3 and/or NFκB activity may be measured using a luciferase reporter assay as described herein.

A functional version of a gene may correspond to the gene in a wild-type ASFV isolate. A functional version of a gene may correspond to the gene in a virulent ASFV strain. The sequence of a functional version of a gene may be identical to the sequence of the gene in a wild-type ASFV isolate or virulent ASFV strain. The sequence of a functional version of a gene may be identical to the sequence of the gene in the wild-type ASFV isolate from which the attenuated ASFV of the invention is derived. A functional version of a gene may be a natural variant of the gene in a wild-type ASFV isolate.

A functional version of a gene may comprise mutations. However, the mutations should not disrupt the expression or activity of the gene. In other words, the mutations should not affect the function of the gene. A functional version of a gene may comprise one or more synonymous mutations (i.e. mutations which do not alter the amino acid sequence of the protein the gene encodes). A functional version of a gene may comprise one or more silent mutations, which may be synonymous or non-synonymous. A functional version of a gene may comprise deletions that do not disrupt the expression or activity of the gene. A functional version of a gene may comprise one or more single nucleotide polymorphisms (SNPs) that do not disrupt the expression or activity of the gene.

### Lack of Functional Version of a Gene

An attenuated ASFV that lacks a functional version of a gene may not express the gene i.e. the gene may not be correctly transcribed and translated. The gene may not be transcribed. The gene may be transcribed but not translated. The gene may be transcribed and translated but the protein too rapidly degraded to carry out its function. The gene may be transcribed and translated but the protein may be non-functional.

Gene expression may be measured by techniques known in the art. For example, the amount of mRNA transcribed from a gene may be quantified, such as by using quantitative polymerase chain reaction (qPCR). Alternatively or additionally, the amount of protein may be quantified, such as by using Western blotting or mass spectrometry.

An attenuated ASFV that lacks a functional version of a gene may comprise a mutation that causes the ASFV to not express the gene. The mutation may be a deletion as described herein. The mutation may be an interruption as described herein.

Lack of a functional version of the K145R gene may mean that the ability of the K145R gene to inhibit the host endoplasmic reticulum (ER) stress response is decreased. K145R function may be tested by methods including immunofluorescence using an antibody against CHOP and assessment of its presence in the nucleus of cells following induction of ER stress, and luciferase reporter assay, where the luciferase gene is under control of the CHOP promoter.

### DIVA Mutations

In an embodiment the attenuated ASFV of the invention comprises a Differentiating Infected from Vaccinated Animals (DIVA) mutation. Suitably the DIVA mutation is lack of a functional version of the K145R gene and/or lack of a functional version of the B125R gene.

In an embodiment, the invention provides an attenuated ASFV in which the expression and/or activity of the following genes is disrupted:
multigene-family (MGF) 360 12L, 13L and 14L, and
MGF 505 1R;

which comprises a functional version of the following genes:
(i) MGF 360 10L and/or 11L, and
(ii) MGF 505 2R and/or 3R;

and which comprises a DIVA mutation.

In an embodiment, the invention provides an attenuated ASFV in which the expression and/or activity of the following genes is disrupted:
multigene-family (MGF) 360 12L, 13L and 14L, and
MGF 505 1R;

which comprises a functional version of the following genes:
(i) MGF 360 10L and/or 11L, and
(ii) MGF 505 2R and/or 3R;

and which lacks a functional version of the K145R gene and/or the B125R gene.

In another aspect the invention provides an attenuated ASFV which lacks a functional version of the K145R gene and/or the B125R gene. Suitably the attenuated ASFV of the invention lacks a functional version of the K145R gene. Suitably the attenuated ASFV of the invention lacks a functional version of the B125R gene.

In another aspect the invention provides an attenuated ASFV that lacks a functional version of the DP148R gene and comprises a DIVA mutation. In an embodiment the attenuated ASFV of the invention lacks a functional version of the following genes:
(i) DP148R; and
(ii) K145R and/or B125R.

In an embodiment the attenuated ASFV of the invention may lack a functional version of the DP148R gene and the K145R gene. In an embodiment the attenuated ASFV of the invention may lack a functional version of the DP148R gene and the B125R gene. Suitably the DP148R gene is partially or completely deleted. Suitably the K145R gene is partially or completely deleted. Suitably the B125R gene is partially or completely deleted. In an embodiment of the attenuated ASFV the following genes are completely deleted: DP148R and K145R. In an embodiment of the attenuated ASFV the following genes are completely deleted: DP148R and B125R.

### Deletions

In the attenuated ASFV of the invention, expression and/or activity of genes may be disrupted by deletion. In other words, expression and/or activity of a gene may be disrupted by a mutation that is a deletion.

An attenuated ASFV of the invention may be made to lack a functional version of a gene by deletion. In other words, the mutation that causes the ASFV to lack a functional version of a gene may be a deletion.

“Deletion” means removal of part of the ASFV genome nucleotide sequence. The deletion may be continuous, or may comprise deletion of a plurality of sections of sequence. Deletion may disrupt gene expression and/or activity in any of the ways described herein. Deletion may cause the ASFV to lack a functional version of the gene in any of the ways described herein.

Deletion may disrupt transcription of the gene into mRNA. For example, deleting a promoter of a gene would disrupt transcription. Deletion may disrupt translation of mRNA into protein. For example, deleting a start codon would disrupt translation. Gene expression and/or activity may be disrupted by deleting non-coding sequence associated with the gene, such as a promoter.

Gene expression and/or activity may be disrupted by deleting coding sequence of the gene. The ASFV may be made to lack a functional version of the gene by deleting coding sequence of the gene. Deletion of coding sequence may be partial (i.e. part of the coding sequence is deleted). The deletion may, for example, remove at least 50, 60, 70, 80 or 90% of the coding sequence of the gene. The deletion may be complete, in which case 100% of the coding sequence of the gene is absent when compared to the corresponding genome of the wild-type isolate.

Deletion of coding sequence may be continuous, or may comprise deletion of a plurality of sections of coding sequence. The deletion should remove a sufficient amount of coding sequence such that deletion disrupts the expression and/or activity of the gene i.e. a functional gene product, such as a protein, is no longer produced from the gene. The expression “deletion of a gene” (such as “partially deleted”) refers to deletion of a sufficient amount of coding sequence such that expression and/or activity of the gene is disrupted. The amount of coding sequence required to be deleted to disrupt gene expression and/or activity may be very small. For example, deletion of just the start codon (ATG) may be sufficient to disrupt expression and/or activity of the gene to attenuate the virus.

Partial and full deletions of a gene can be made using known techniques in the art, such as conditional targeting via Cre-LoxP and Flp-FRT systems, or by inducing a double strand break (DSB) and repair using engineered nucleases such as meganucleases, zinc finger nucleases (ZFNs), transcription-activator like effector nucleases (TALENs) and Cas in CRISPR-Cas systems. The DSB repair can be exploited to introduce a desired mutation by providing a vector comprising the desired mutated nucleotide sequence within a sequence that is homologous to the sequences flanking either side of the DSB. This results in the desired mutation being inserted at the site of the DSB. Nucleases such as those above can be engineered to induce DSB at a specific site within the genome. For example, chimeric meganucleases can be readily generated by combining known protein units to recognise a target recognition sequence within a gene or genomic region of interest. ZFNs can also be designed to target specific sequences, for example combining zinc-finger units with known specificities to bind specific regions of DNA. TALENs are artificial restriction enzymes designed by fusing a nuclease domain to DNA-binding TALE (transcription activator-like effector) domains. TALE domains are tandem arrays of amino acid repeats that recognise a single nucleotide and can be designed to target a specific region of DNA. CRISPR-Cas systems consist of a Cas (CRISPR-associated protein) nuclease and a CRISPR (clustered regularly interspaced short palindromic repeat) RNA sequence that guides the Cas protein to recognise and cleave a specific strand of DNA complementary to the CRISPR sequence. Single-stranded guide RNA (sgRNA) can therefore be designed to bind to a specific region of DNA and guide the Cas to introduce a DSB. Accordingly, provided the nucleotide (e.g. DNA or cDNA) sequence of a gene is known, a known nuclease system can be utilised to introduce a partial or full deletion to the gene.

In an embodiment of the attenuated ASF virus of the present invention the following genes may be at least partially deleted:
MGF 360 genes 12L, 13L and 14L; and
MGF 505 gene 1R.

One or more (such as two or more, three or more or all four) of the disrupted genes may be at least partially deleted. The coding sequences of one or more (such as two or more, three or more or all four) of the disrupted genes may be at least partially deleted.

In an embodiment of the attenuated ASF virus of the present invention the following genes may be completely deleted:
MGF 360 genes 12L, 13L and 14L; and
MGF 505 gene 1R.

One or more (such as two or more, three or more or all four) of the disrupted genes may be completely deleted. The coding sequences of one or more (such as two or more, three or more or all four) of the disrupted genes may be completely deleted.

In an embodiment, an attenuated ASFV of the invention lacks a functional version of the K145R gene and/or the B125R gene. The gene may be wholly or partially deleted. The coding sequence of the gene may be wholly or partially deleted. Suitably the K145R gene coding sequence is wholly or partially deleted. Suitably the B125R gene coding sequence is wholly or partially deleted.

In an embodiment, an attenuated ASFV of the invention lacks a functional version of the K145R gene and/or the B125R gene. The K145R gene and/or the B125R gene may be wholly or partially deleted. The coding sequence of the gene may be partially or completely deleted. Suitably the K145R gene coding sequence is partially or completely deleted. Suitably the B125R gene coding sequence is partially or completely deleted.

In an embodiment of the attenuated ASFV of the present invention the following genes may be at least partially deleted:
MGF 360 genes 12L, 13L and 14L;
MGF 505 gene 1R; and
K145R gene and/or B125R gene.

One or more (such as two or more, three or more, four or more, five or more or all six) of the genes may be at least partially deleted. The coding sequences of one or more (such as two or more, three or more, four or more, five or more or all six) of the genes may be at least partially deleted.

In an embodiment of the attenuated ASFV of the present invention the following genes may be completely deleted:
MGF 360 genes 12L, 13L and 14L;
MGF 505 gene 1R; and
K145R gene and/or B125R gene.

One or more (such as two or more, three or more, four or more, five or more or all six) of the genes may be completely deleted. The coding sequences of one or more (such as two or more, three or more, four or more, five or more or all six) of the genes may be completely deleted.

### Interruptions

The expression and/or activity of the genes may be disrupted by interruption of the gene. In other words, the mutation that disrupts expression and/or activity of a gene may be one that interrupts the gene.

An attenuated ASFV of the invention may be made to lack a functional version of a gene by interruption of the gene. In other words, the mutation that causes the ASFV to lack a functional version of a gene may be one that interrupts the gene.

In an embodiment of the attenuated ASFV of the present invention the following genes may be interrupted:
MGF 360 genes 12L, 13L and 14L; and
MGF 505 gene 1R.

One or more (such as two or more, three or more or all four) of the genes may be interrupted.

In another embodiment of the attenuated ASF virus of the invention the K145R gene and/or the B125R gene may be interrupted. Suitably the K145R gene may be interrupted. Suitably the B125R gene may be interrupted.

In an embodiment of the attenuated ASFV of the present invention the following genes may be interrupted:
MGF 360 genes 12L, 13L and 14L;
MGF 505 gene 1R; and
K145R gene and/or B125R gene.

One or more (such as two or more, three or more, four or more, five or more or all six) of the genes may be interrupted.

“Interruption” means the mutation alters the coding sequence of the gene such that a functional gene product, such as a protein, is no longer produced. The term “interruption” may be used herein to refer to a mutation that interrupts a gene. The mutation(s) should interrupt the coding sequence in a manner such that expression and/or activity of the gene is disrupted i.e. a functional gene product, such as a protein, is no longer produced from the gene.

The interruption may entirely abolish gene product production. For example, where the gene product is a protein, the interruption may render the mRNA nonsensical, causing the mRNA to be degraded and the protein to not be translated, thereby abolishing protein production.

The interruption may alter the gene product that is produced. The interruption may cause the gene to not be transcribed and/or translated.

The interruption may be a point mutation (i.e. substitution, insertion or deletion of a single nucleotide). An interruption may be a deletion. A gene may comprise multiple mutations that lead to interruption of the gene.

The interruption may be a frame shift mutation. A frame shift causes the codons downstream of the frame shift to be read as different amino acids. The protein produced may be non-functional.

The interruption may be mutation of a start codon. A start codon is typically ATG. Mutation of a start codon (e.g. point mutation of one, two or three of the nucleotides) means that translation will not start at that codon. Translation may begin at a subsequent start codon further downstream. If the subsequent start codon is in frame a version of the protein is produced that is N-terminally truncated and so may be non-functional. If the subsequent start codon is not in frame an entirely different or nonsense protein is produced, which would be non-functional. If there is no subsequent start codon, translation is entirely abolished and no protein is produced.

The interruption may be mutation of a stop codon (TAG, TAA or TGA). Mutation of a stop codon (also referred to as a nonstop mutation) causes continued translation of mRNA into a sequence that should not be translated. The resulting protein may be non-functional due to its excessive length.

### Mutation Combinations

The mutations that disrupt gene expression and/or activity described herein may be combined in an attenuated ASFV of the invention. In other words, each of the disrupted genes (MGF 360 genes 12L, 13L and 14L, MGF 505 gene 1R) in an attenuated ASFV of the invention may be disrupted by the same type of mutation as any of the other genes or by a different type of mutation as any of the other genes.

In an embodiment of the attenuated ASFV of the invention, one or more of the disrupted genes may be disrupted by mutation of a promoter sequence, whilst one or more other disrupted genes may be disrupted by partial deletion of coding sequence, whilst one or more other genes may be disrupted by complete deletion of coding sequence, whilst one or more other genes may be disrupted by interruption of the gene.

### Vaccine/Pharmaceutical Composition

The present invention also provides a vaccine comprising an attenuated ASF virus of the invention.

The term “vaccine” as used herein refers to a preparation which, when administered to a subject, induces or stimulates a protective immune response. A vaccine can render an organism immune to a particular disease, in the present case ASF. The vaccine of the present invention thus induces an immune response in a subject which is protective against subsequent ASF virus challenge. A vaccine comprising an attenuated ASFV of the invention may be capable of inducing a cross-protective immune response against a plurality of ASF virus genotypes. In an embodiment a vaccine comprising an attenuated ASFV of the invention of a single genotype may be capable of inducing a cross-protective immune response against a plurality of ASF virus genotypes.

The vaccine may comprise a plurality of attenuated ASF viruses. The plurality of attenuated ASF viruses may correspond to a plurality of different isolates, for example, different isolates of high or unknown virulence. Such a vaccine may be capable of inducing a cross-protective immune response against a plurality of ASF virus genotypes.

The vaccine may be useful in preventing African Swine Fever. Accordingly the invention provides a vaccine of the invention for use in treating and/or preventing African Swine Fever in a subject.

The present invention also provides a pharmaceutical composition which comprises one or more attenuated ASF virus(es) of the invention. The pharmaceutical composition may be used for treating African Swine Fever.

The vaccine or pharmaceutical composition may comprise one or more attenuated ASF virus(es) of the invention and optionally one or more adjuvants, excipients, carriers and diluents. The choice of pharmaceutical excipient, carrier or diluent can be selected with regard to the intended route of administration and standard pharmaceutical practice. The pharmaceutical compositions may comprise as (or in addition to) the carrier, excipient or diluent, any suitable binder(s), lubricant(s), suspending agent(s), coating agent(s), solubilising agent(s) and other carrier agents. The pharmaceutical compositions typically should be sterile and stable under the conditions of manufacture and storage. Formulations for parenteral administration include, but are not limited to, suspensions, solutions, emulsions in oily or aqueous vehicles, pastes, and implantable sustained-release or biodegradable formulations. Sterile injectable formulations may be prepared using a nontoxic parenterally acceptable diluent or solvent. A pharmaceutical composition of the present invention may include pharmaceutically acceptable dispersing agents, wetting agents, suspending agents, isotonic agents, coatings, antibacterial and antifungal agents, carriers, excipients, salts, or stabilizers which are nontoxic to the subjects at the dosages and concentrations employed. Preferably, such a composition can further comprise a pharmaceutically acceptable carrier or excipient for use in the treatment of disease that that is compatible with a given method and/or site of administration, for instance for parenteral (e.g. sub-cutaneous, intradermal, or intravenous injection) or intrathecal administration.

The vaccine or pharmaceutical composition may comprise one or more attenuated ASF virus(es) of the invention in an effective amount.

In an embodiment the invention provides an attenuated ASF virus of the invention which when administered to a subject induces an immune response which is protective against subsequent challenge with virulent ASF virus. In an embodiment the invention provides an attenuated ASF virus of the invention which when administered to a subject induces an immune response which is protective against subsequent challenge with virulent ASF virus of a different genotype to the attenuated ASF virus of the vaccine.

### Methods of Prevention/Treatment

The present invention also provides a method of preventing and/or treating ASF in a subject by administration to the subject of an effective amount of an attenuated virus, vaccine, or pharmaceutical composition of the invention.

The term “preventing” is intended to refer to averting, delaying, impeding or hindering the contraction of ASF. The vaccine may, for example, prevent or reduce the likelihood of an infectious ASFV entering a cell.

The term “treating” is intended to refer to reducing or alleviating at least one symptom of an existing ASF infection.

The subject may be any animal which is susceptible to ASF infection. ASF susceptible animals include domestic pigs, warthogs, bush pigs and ticks.

The subject vaccinated according to the present invention may be a domestic pig.

### Administration

The vaccine of the invention may be administered by any convenient route, such as by intramuscular injection. Other suitable routes of administration include intranasal, oral, subcutaneous, transdermal and vaginal (e.g. during artificial insemination). In one embodiment, oral administration comprises adding the vaccine to animal feed or drinking water. In another embodiment, the vaccine may be added to bait for a wild animal, for example bait suitable for wild boar, wild pigs, bushpigs or warthogs.

The dose for pig immunisation may be less than 10⁴ HAD₅₀ or TCID₅₀ per pig. For example the dose may be between 10²-10³ HAD₅₀ or TCI D₅₀. The dose may be about 10² HAD₅₀ or TCID₅₀ per pig. The dose may be determined by a veterinary practitioner within the scope of sound veterinary judgment.

The vaccine may be administered following a prime-boost regime. For example, after the first inoculation, the subjects may receive a second boosting administration some time (such as about 7, 14, 21 or 28 days) later. Typically the boosting administration is at a higher dose than the priming administration. The boosting dose may be about 10², 10³ or 10⁴ HAD₅₀ or TCID₅₀ of the recombinant attenuated virus per pig.

### Method for Preparing an Attenuated Virus

The present invention also provides a method of attenuating an ASF virus, which comprises the step of disrupting the expression and/or activity of the following genes:
MGF 360 genes 12L, 13L and 14L; and
MGF 505 genes 1R,

whilst retaining the function of the following genes:
(i) MGF 360 10L and/or 11L, and
(ii) MGF 505 2R and/or 3R.

Disruption of gene expression and/or activity may be achieved by mutating the ASFV genome in any of the ways described herein.

The following genes may be partially or completely deleted:
MGF 360 genes 12L, 13L and 14L; and
MGF 505 genes 1R.

One or more (such as two or more, three or more or all four) of the disrupted genes may be at least partially or completely deleted. The coding sequences of one or more (such as two or more, three or more or all four) of the disrupted genes may be at least partially or completely deleted.

The following genes may be interrupted:
MGF 360 genes 12L, 13L and 14L; and
MGF 505 genes 1R.

One or more (such as two or more, three or more or all four) of the genes may be interrupted.

Methods for mutation of viral genes are known in the art. In particular, methods for deletion of viral genes are known in the art. For example, homologous recombination may be used, in which a transfer vector is created in which the relevant gene(s) are missing and used to transfect virus-infected cells. Recombinant viruses expressing the new portion of sequence may then be selected. Similar procedures may be used in order to interrupt gene expression, for example by deletion of the ATG start codon.

“Retaining the function” of a gene means that expression and activity of the gene is not affected during the attenuation process. The resultant attenuated virus should express a functional version of the gene. Suitably, the genes the function of which is to be retained are unaltered by the method of attenuation. Suitably, the sequences of the genes the function of which is to be retained are unaltered by the method of attenuation.

In some embodiments of the present invention, the method of attenuating an ASF virus may comprise retaining the function of MGF 360 10L and MGF 505 2R. In some embodiments, the method may comprise retaining the function of MGF 360 11L and MGF 505 2R. In some embodiments, the method may comprise retaining the function of MGF 360 10L and MGF 505 3R. In some embodiments, the method may comprise retaining the function of MGF 360 11L and MGF 505 3R. In some embodiments, the method may comprise retaining the function of MGF 360 10L and 11L and MGF 505 2R. In some embodiments, the method may comprise retaining the function of MGF 360 10L and 11L and MGF 505 3R. In some embodiments, the method may comprise retaining the function of MGF 360 10L and MGF 505 2R and 3R. In some embodiments, the method may comprise retaining the function of MGF 360 11L and MGF 505 2R and 3R. In some embodiments, the method may comprise retaining the function of MGF 360 10L and 11L and MGF 505 2R and 3R.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1—MGF Genes Inhibit Activity of Transcription Factors IRF3 and NF-κB

The effect of MGF genes on activity of interferon regulatory factor 3 (IRF3) and NF-κB was tested using a luciferase reporter assay. IRF-3 and NF-κB are transcription factors that control expression of interferon and pro-inflammatory cytokines as part of the host's innate immune system response to viral infection.

HEK293T cells were transfected with a luciferase reporter under control of a promoter specifically activated by IRF3. The cells were also transfected with a plasmid expressing an MGF gene (MGF360-10L, 11L, 12L, 13L or 14L, MGF 505-1R, 2R, 3R, 4R or 5R, or MGF110-1L), an empty vector (as negative control) or a plasmid expressing CSFV Npro (a known IRF3 inhibitor protein, as positive control). The cells were then stimulated with Sendai Virus (SeV) to activate IRF3. The levels of luciferase following stimulation provide a measure of the ability of MGFs to modulate the signalling cascades controlled by IRF-3 and NF-kB. As shown in FIG. 1A, the level of luciferase expression upon stimulation with SeV was high in cells transfected with empty vector, but low in cells transfected with IRF3 inhibitor or any of the MGF genes. These data thus indicate that all MGF genes tested suppress IRF3 activity.

An equivalent experiment was performed to test the effect of MGF genes on NF-κB expression. For this experiment, the cells were instead transfected with a luciferase reporter under control of a promoter specifically activated by NF-κB, a poxvirus MC160 protein that inhibits NF-κB was used for the positive control and NF-κB was activated by transfection of the cells with NF-κB sub-unit p65. The results, shown in FIG. 1B, indicate that all MGF genes tested suppress NF-κB activity.

### MGF360-12L Inhibits Transcriptional Activity of IRF3 and NF-κB

A further experiment was performed to further investigate how MGF genes inhibit IRF3 and NF-κB activity.

Transcription factors (TF) generally possess two major domains: a transcriptional activation domain (TAD) and a DNA binding domain (DBD). These domains are interchangeable and therefore chimeric proteins containing TAD and DBD from different TFs can be used to further explore cell signalling pathways.

Chimeras containing the NF-κB p65 or the IRF3 TADs fused to the GAL4 DBD were used to evaluate the ability of the MGFs to directly inhibit NF-κB p65 or IRF3 transcriptional activity. In this case, the reporter plasmid contained the luciferase gene under the control of a responsive element that is recognised by the GAL4 DBD. Therefore, the luciferase activity was not dependent on the ability of IRF3 or NF-κB to bind their promoters but rather solely on their activation status.

FIG. 1C and FIG. 1D respectively show that MGF360-12L inhibits luciferase activity induced by overexpression of IRF-3(TAD)-GAL4(DBD) and p65(TAD)-GAL4(DBD). In contrast the other members of the MGF families did not inhibit the luciferase activity in this assay.

Taken together, these results indicate that all the MGF genes tested are able to inhibit IRF-3 and NF-kB function by binding to DNA and/or recruitment of co-activators. MGF360-12 L also inhibits the activity of these transcription factors directly, therefore providing a possible explanation for the stronger inhibition of IRF3 and NF-kB observed with MGF 360-12L compared to the other MGF genes. The NF-kB promoter binding element is present in promoters for additional genes to type I interferon and inhibition of this pathway is likely to affect a broader range of genes including those involved in activating a proinflammatory response.

### Example 2—Deletion of MGFA with K145R or B125R Attenuates ASFV and Induces Protection Against Challenge

### MGFA and MGFB Deletions

Two sets of genes of the multigene families 360 and 505 were deleted in ASFV Georgia 2007/1 strain.

In MGFA the complete coding regions of genes MGF360-12L, MGF360-13L, MGF360-14L and MGF505-1R were deleted.

In MGFB the complete coding regions of genes MGF360-13L, MGF360-14L, MGF505-2R and MGF505-3R were deleted.

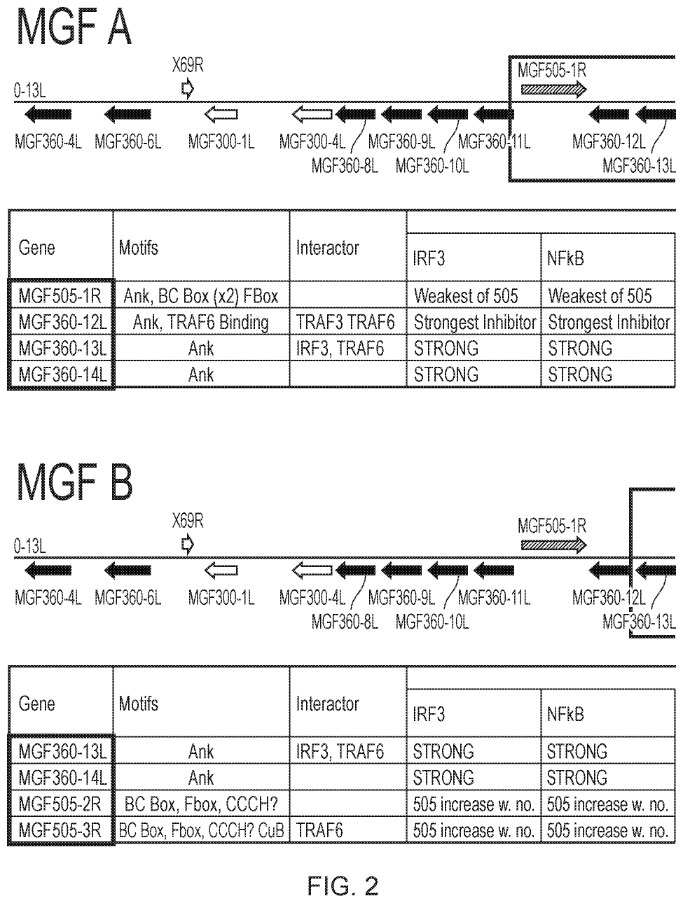

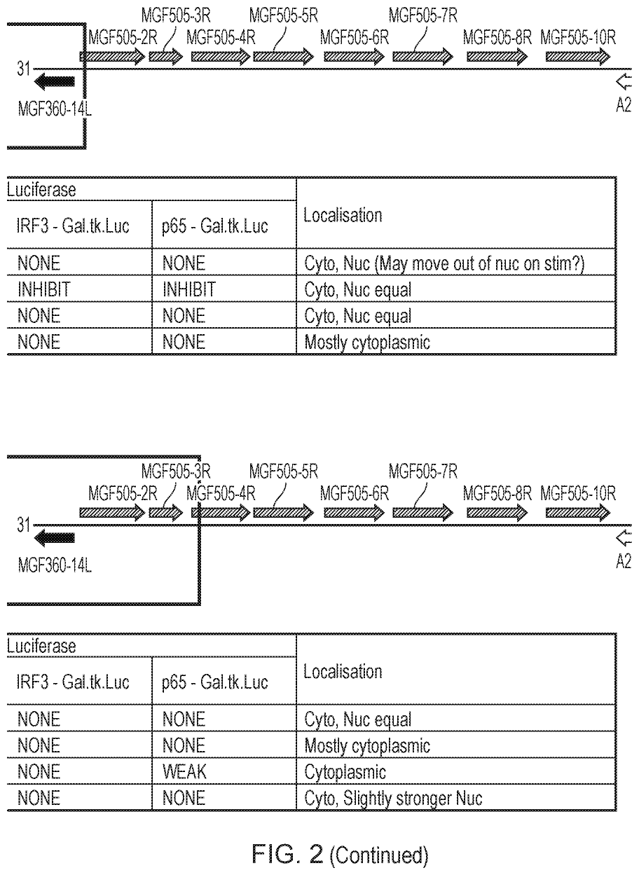

The MGFA and MGFB deletions are shown schematically in FIG. 2. Below each gene name, information on the inhibition of IRF3- and NF-κB-dependent luciferase reporters is summarised and any identified host targets listed.

The MGFA deletion was made in combination with deletion of the K145R gene (GeorgiaΔK145RΔMGFA) or the B125R gene (GeorgiaΔB125RΔMGFA).

The MGFB deletion was made in combination with deletion of the K145R gene (GeorgiaΔK145RΔMGFB).

### Vaccination Experimental Protocol

Groups of 6 large white landrace pigs were immunised intramuscularly with 10⁴ TCID50/m1 GeorgiaΔK145RΔMGFA (Group C), GeorgiaΔK145RΔMGFB (Group D) or GeorgiaΔB125RΔMGFA (Group BA) viruses.

Pigs from group D were culled at the humane endpoint after immunisation.

Pigs in Groups C and BA were boosted and then challenged with parental virulent Georgia strain.

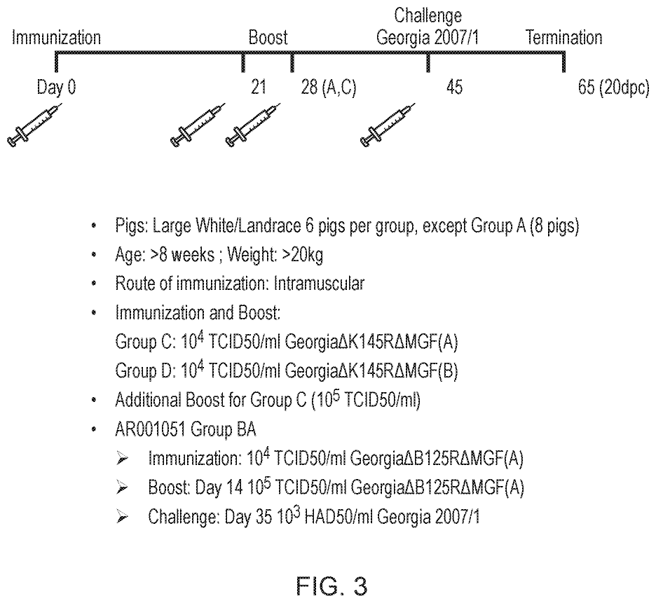

The experimental protocol is depicted in FIG. 3.

### Survival

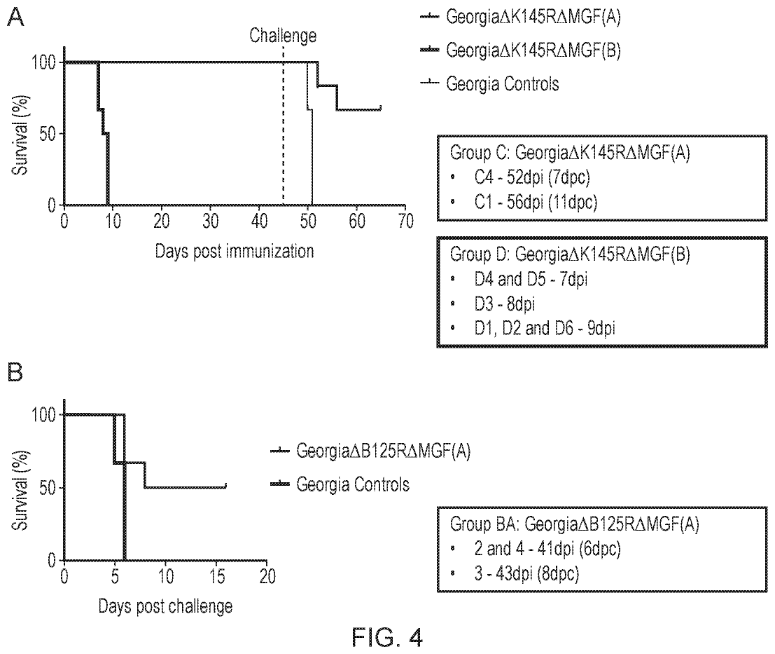

Pigs from Group D (GeorgiaΔK152RΔMGFB) were all culled between day 5 and 9 post-immunisation at moderate severity endpoint (FIG. 4A).

Pigs from Group C (GeorgiaΔK145RΔMGFA) and Group BA (GeorgiaΔB125RΔMGFA) survived immunisation and boost.

After challenge, 2 pigs from Group C were culled at days 7 or 11 (FIG. 4A) post-challenge and 3 pigs from Group BA were culled at 6 to 8 days post-challenge at the moderate severity endpoint (FIG. 4B). The remaining pigs survived until the end of the experiment. As expected, control non-immune pigs (Group F) were culled by day 6 post-challenge (FIG. 4A and FIG. 4B). Thus survival was 66%.

### Temperature and Clinical Scores

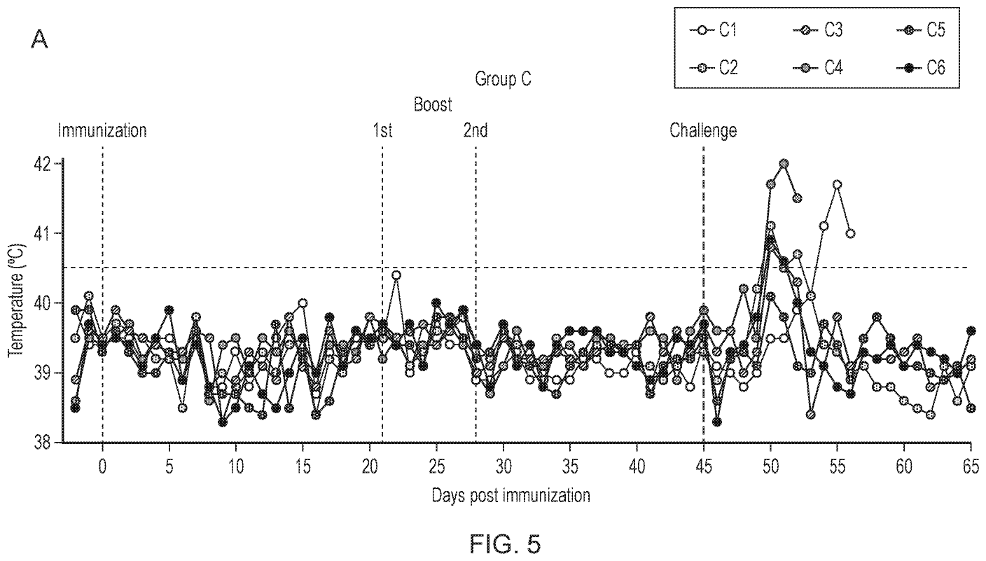

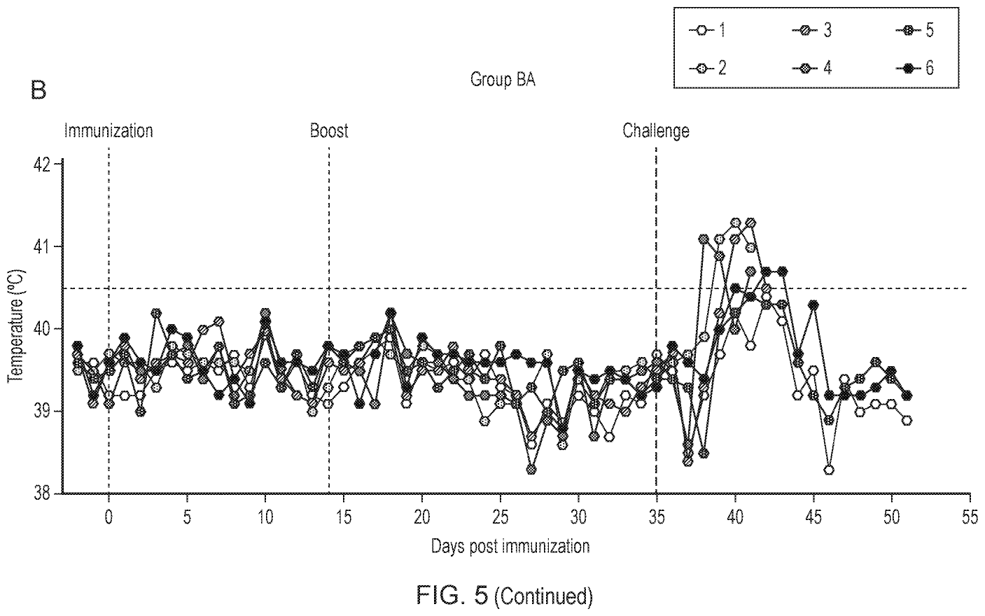

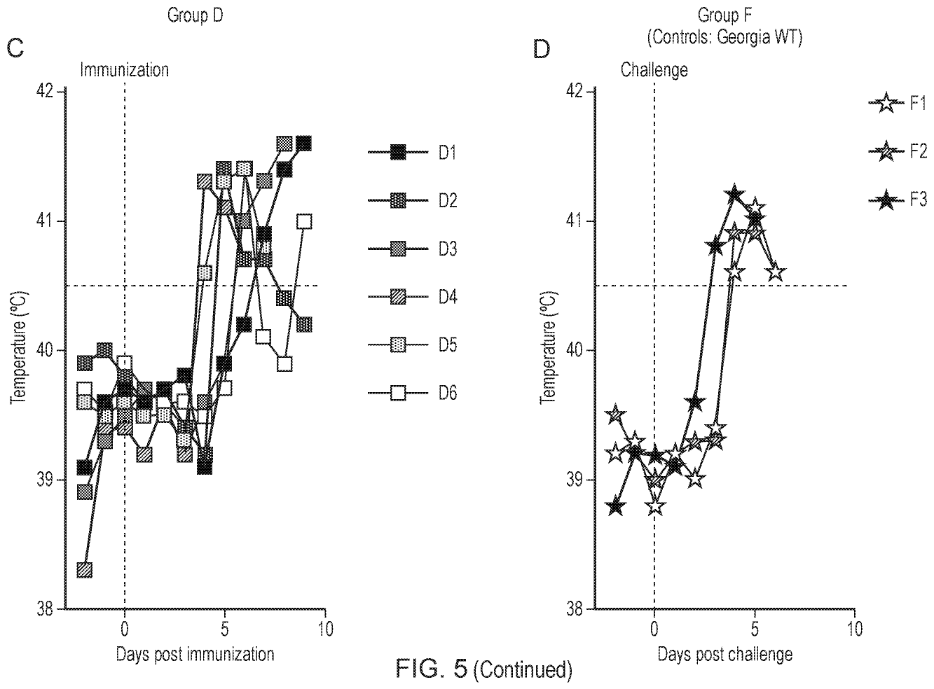

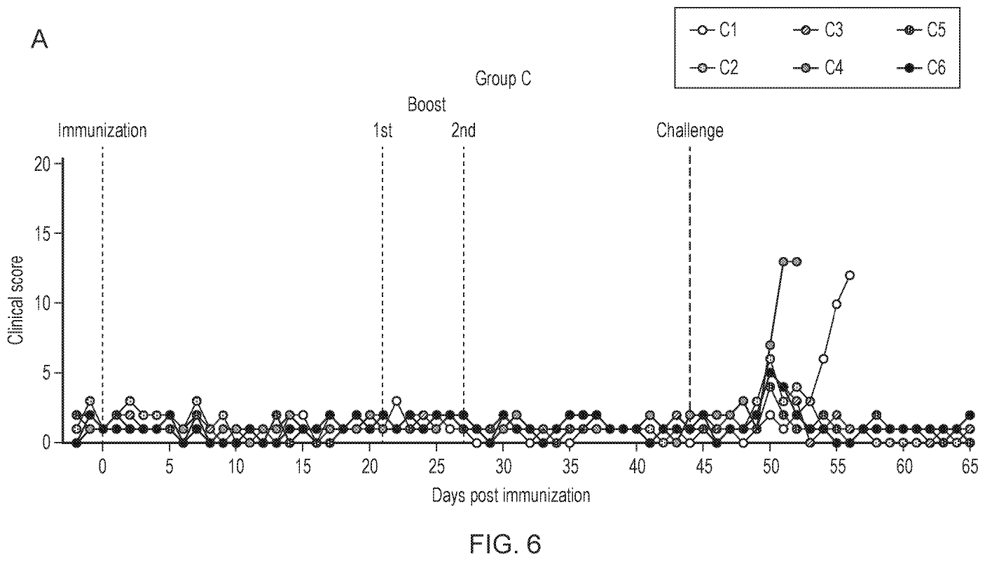

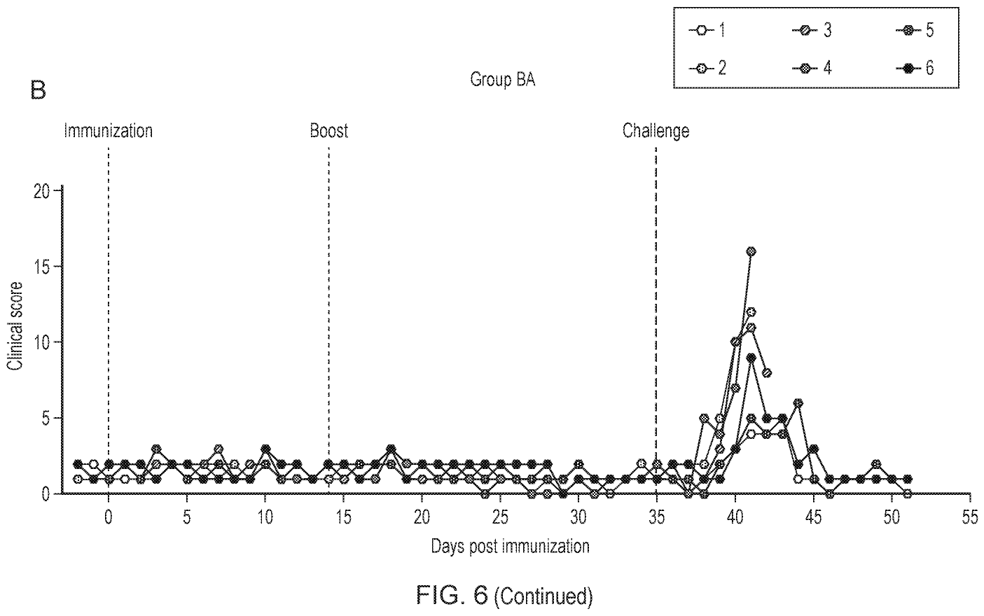

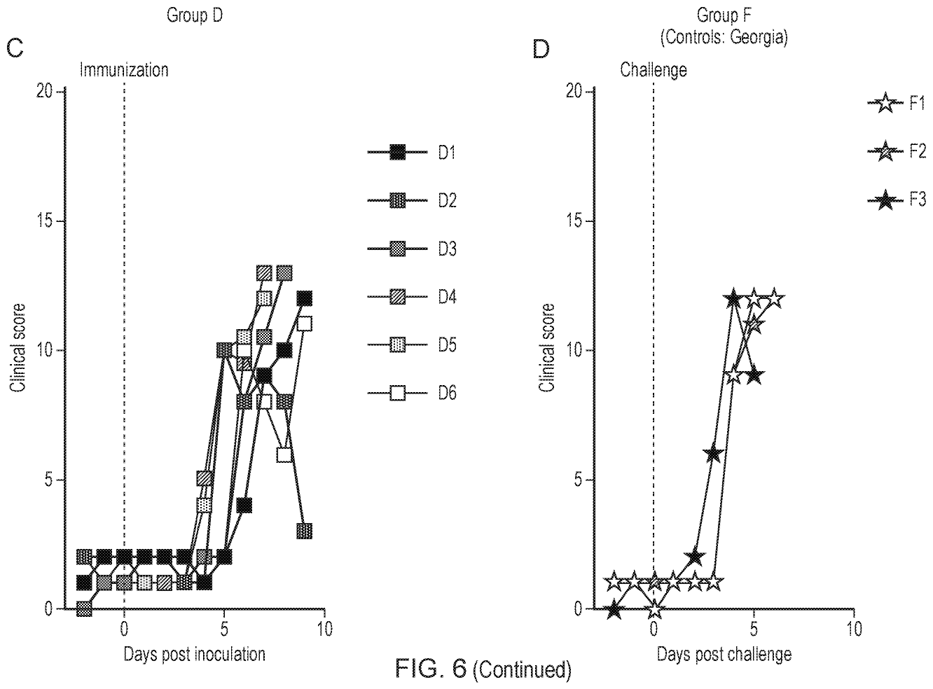

Pigs in Groups C and BA showed no rise in temperature (FIG. 5A and FIG. 5B) or increased clinical signs (FIG. 6A and FIG. 6B) following immunisation and boost.

At days 5 or 6 post-challenge all pigs (except one pig from Group C) developed temperatures above 40.5° C. and other signs which lasted between 1 and 3 days (except for pigs which were culled at the moderate severity end-point). Remaining pigs showed no further signs until the experiment was terminated.

### Viremia

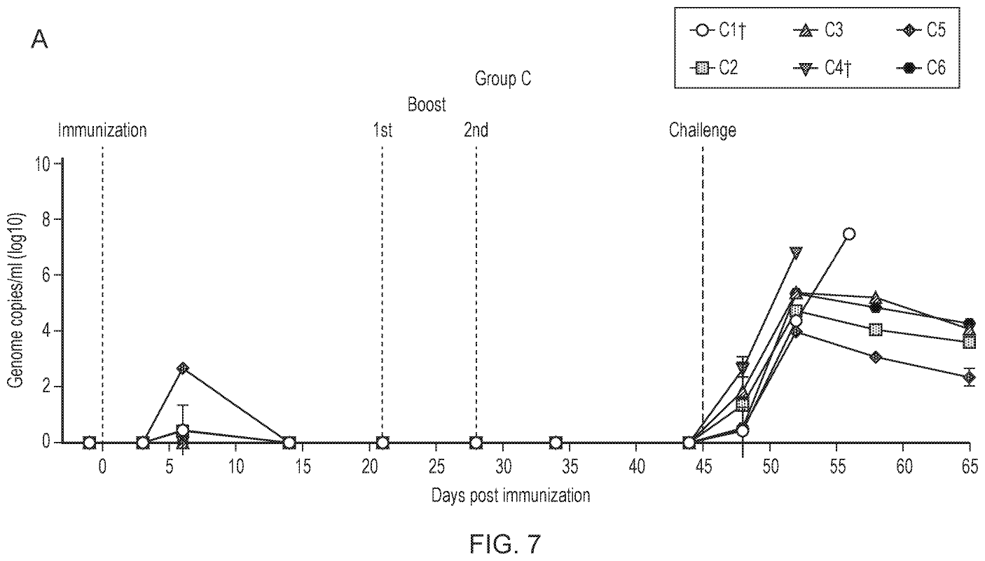

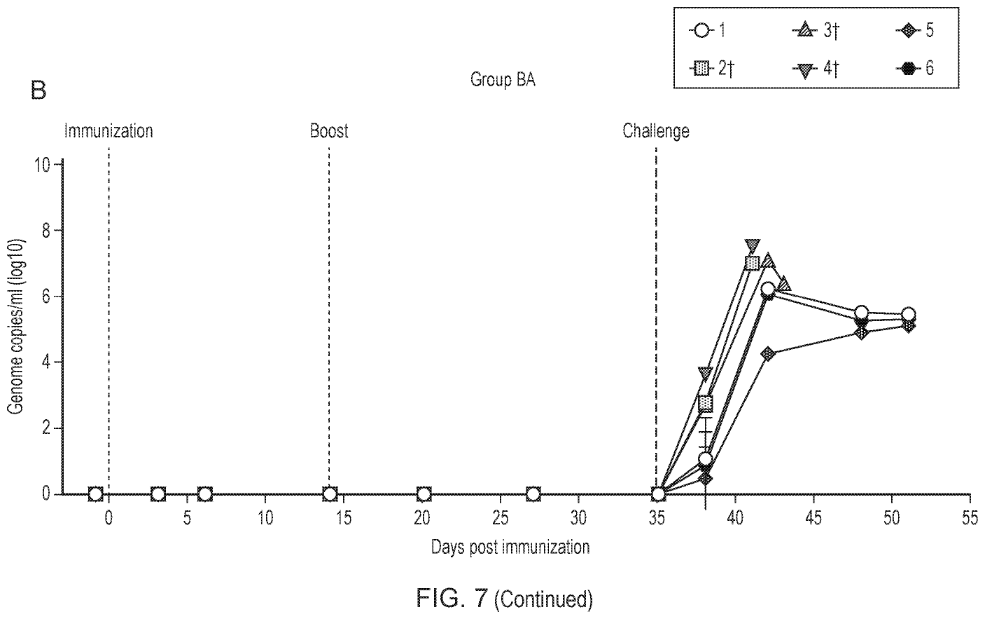

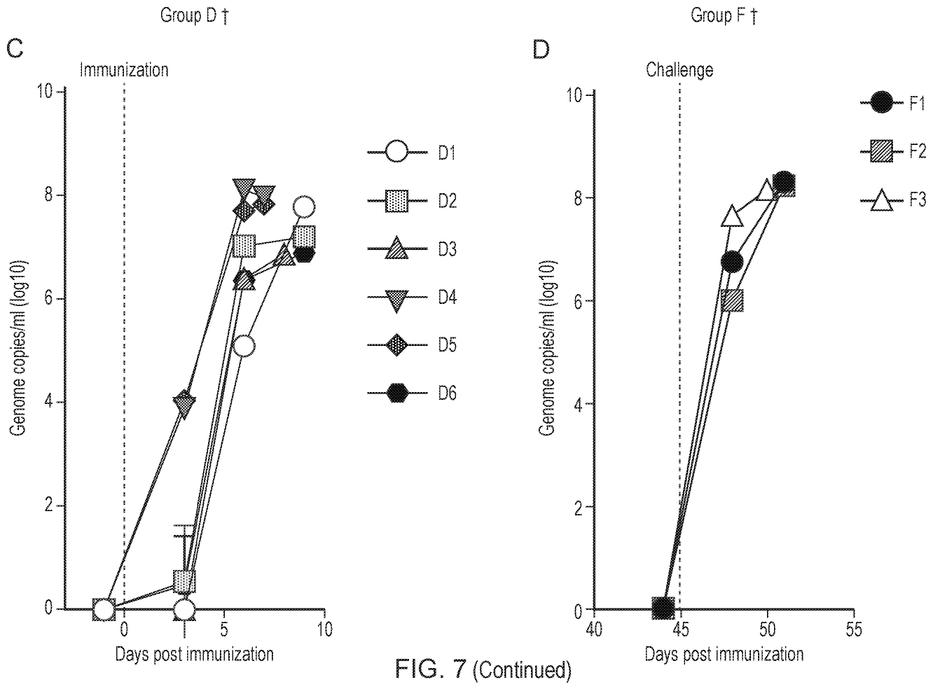

The levels of virus genome in whole blood were measured by quantitative PCR throughout the experiment. One pig immunised in Group C had very low genome level at day 6 post-immunisation but no other pigs in Group C or Group AB had detectable virus genome following immunisation or boost (FIG. 7A and FIG. 7B). In contrast all pigs in Group D had high levels of virus genome by day 6 post-immunisation as expected from the clinical signs (FIG. 7C).

After challenge virus genome was detected in blood from all pigs. In Group C a peak varying between 10e4 and 10e6 genome copies per ml was detected at day 7 post-challenge in all pigs that survived decreasing slowly after this. As expected in the two pigs that didn't survive higher levels of virus genome were detected. In Group BA, virus genome was detected at similar levels in pigs that survived and at higher levels in those that didn't survive.

### Infectious Virus in Blood

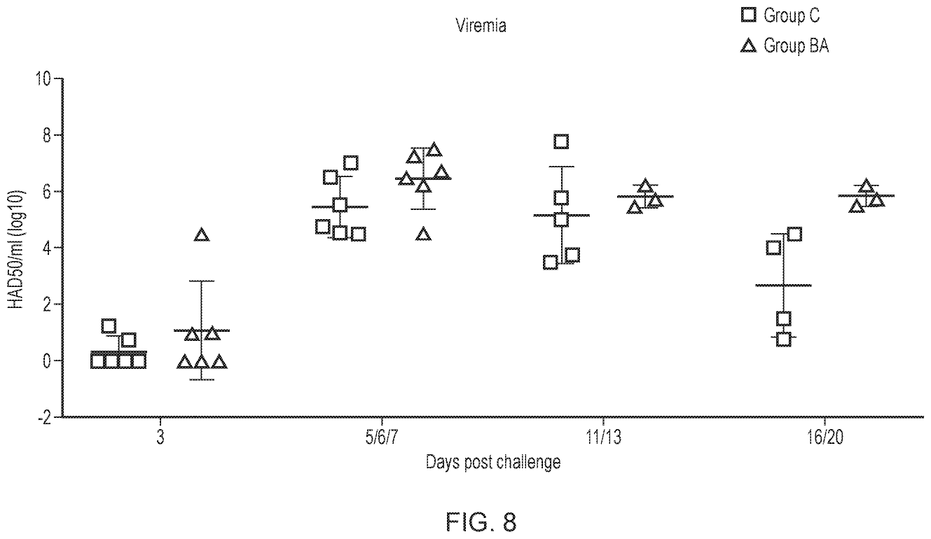

The levels of infectious virus in whole blood was measured after challenge (FIG. 8).

### T Cell Responses

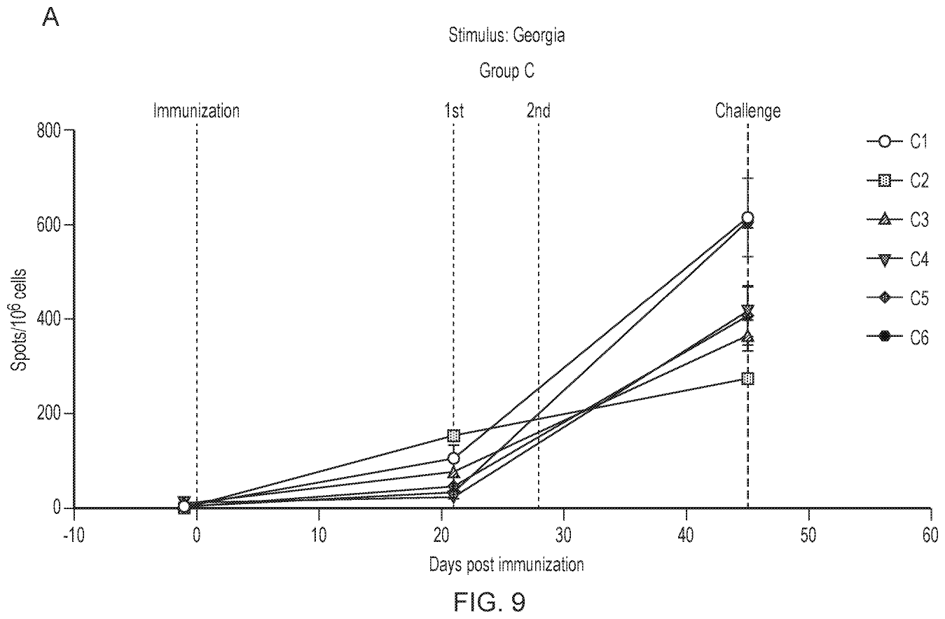

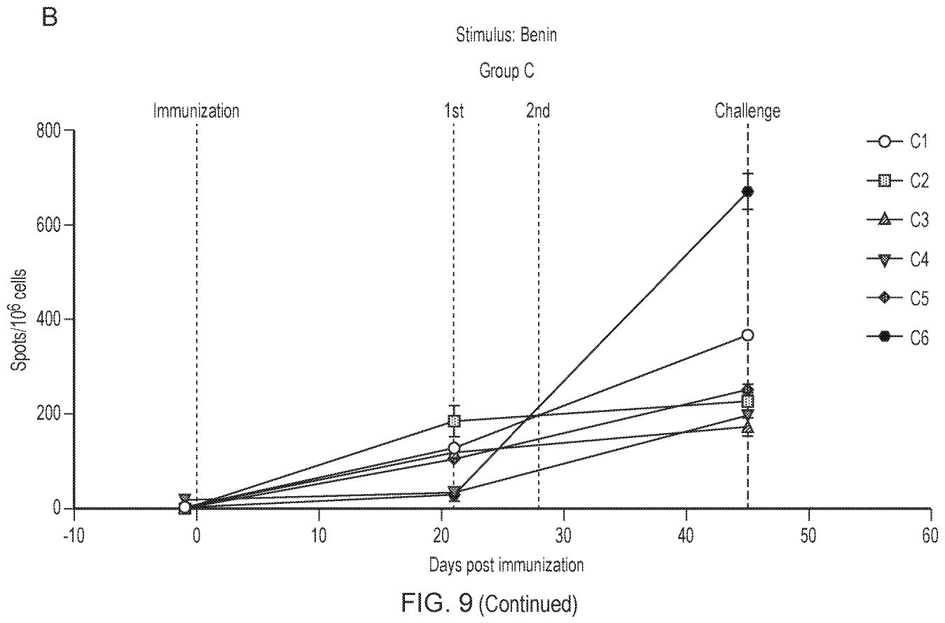

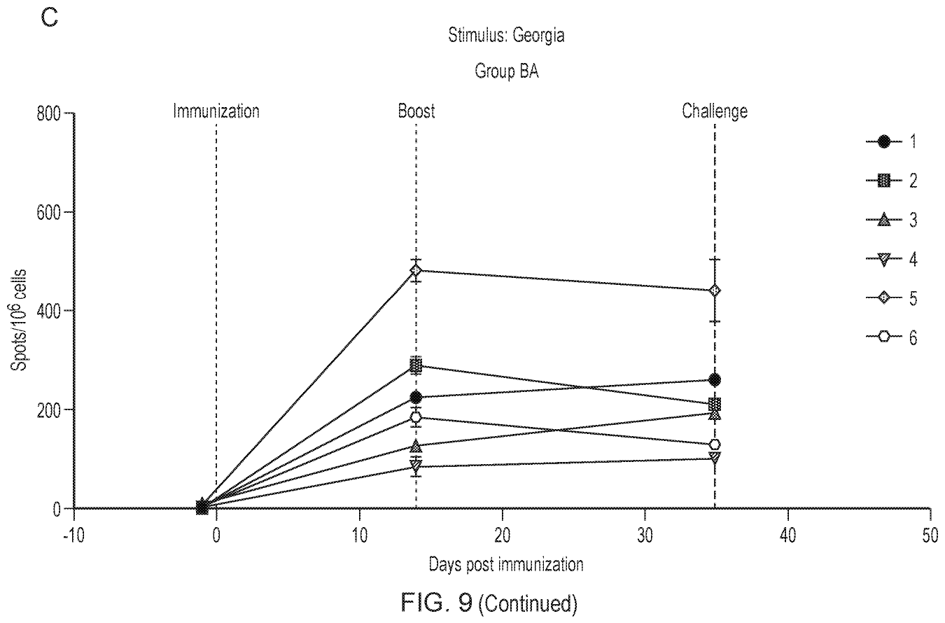

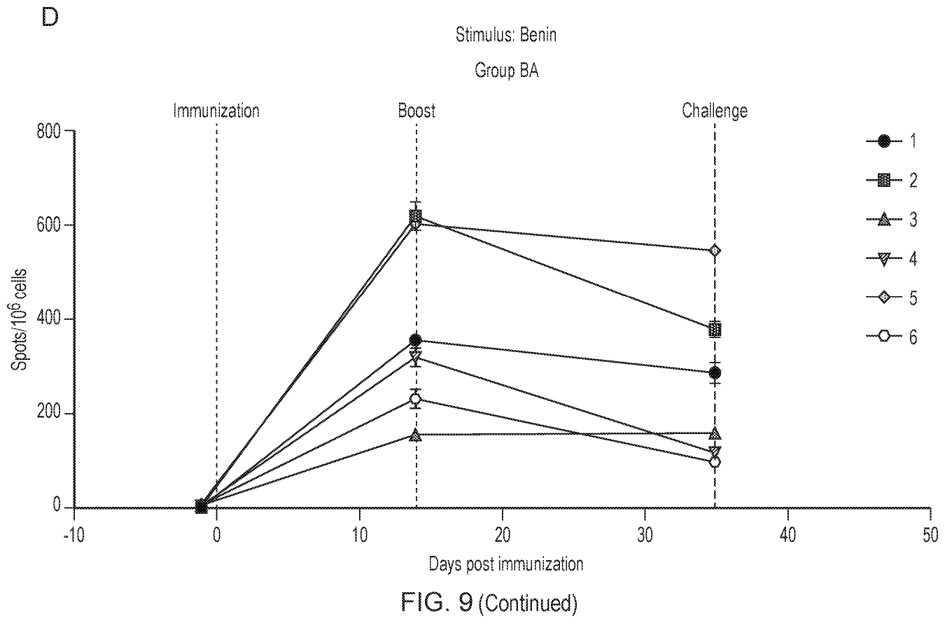

Peripheral blood mononuclear cells (PBMCs) were collected pre-immunisation, pre-boost and pre-challenge and stimulated with live ASFV (either the homologous virulent Georgia 2007/1 strain or heterologous genotype I virulent strain Benin 97/1). Numbers of interferon gamma producing cells were measured by Elispot assays (FIG. 9).

As expected no responses were detected pre-immunisation. In Group C low responses to Georgia virus stimulation were detected pre-boost but these increased several fold before challenge (FIG. 9A). Responses to virulent Benin were lower as expected except in one pig (FIG. 9B). In Group AB higher levels of IFNγ producing cells were observed before boost but this did not increase before challenge (FIG. 9C and FIG. 9D).

### Antibody Responses

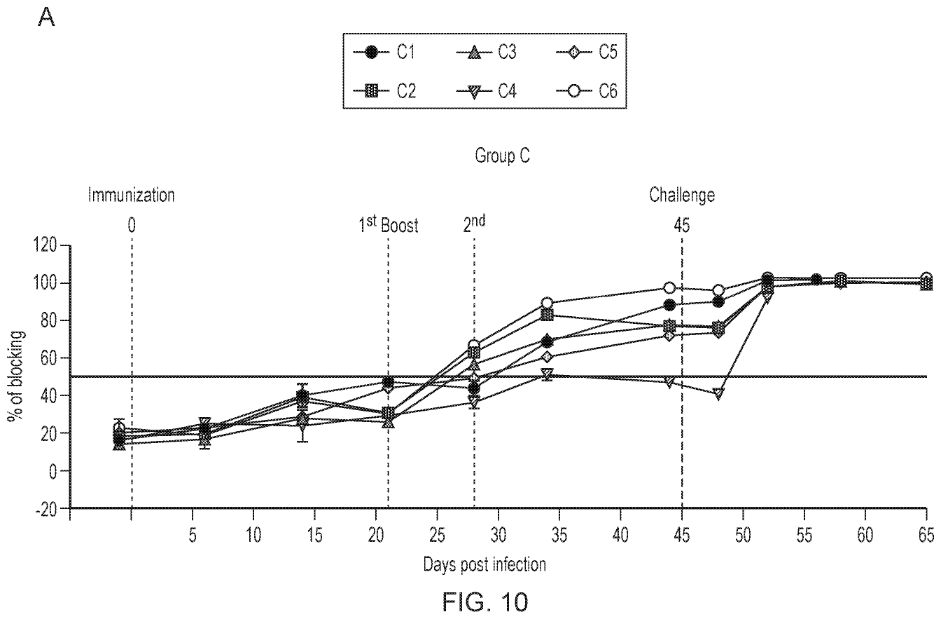

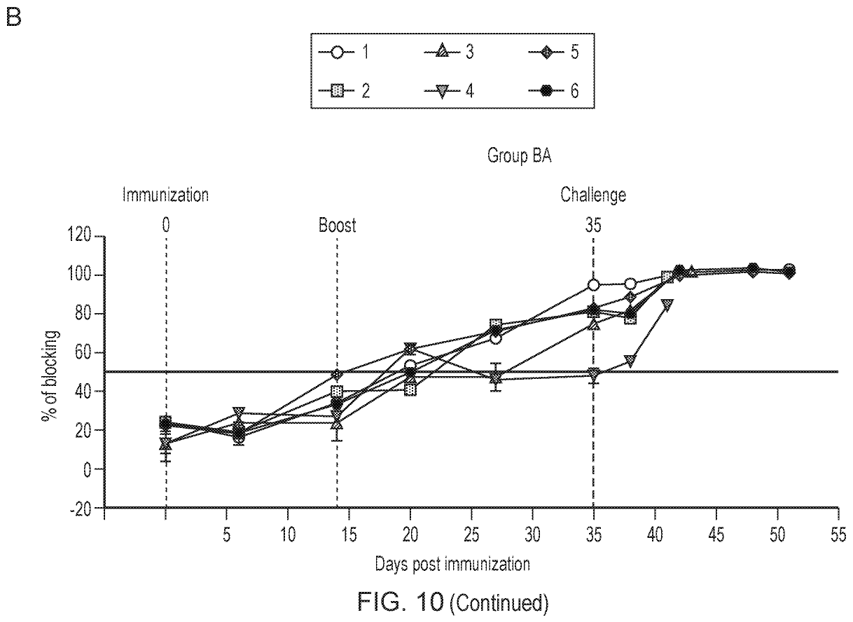

The antibody response to the major ASFV capsid protein p72/B646L was measured post-immunisation using a commercial competitive ELISA (Ingenasa). Antibody responses above the cut-off were detected by day 30 post-immunisation (after the boost) in pigs from Group C, increasing thereafter but not reaching 100% until after challenge (FIG. 10A). Similar results were observed in pigs from Group BA although antibody responses were first detected by day 20 post-immunisation (FIG. 10B).

### Example 4—Screening for DIVA Targets

To screen for genes that could serve as Differentiating Infected from Vaccinated Animals (DIVA) markers in ASFV, 71 plasmids coding for individual ASFV genes (excluding known essential genes) fused to an HA or V5 epitope tag were transfected into Vero cells. The cells were fixed and permeabilised and stained with antisera from different immunised pigs followed by a fluorescently labelled secondary antibody. Confocal microscopy was used to assess whether the expressed gene could be detected.

In parallel the cells were stained with an antibody against the HA or V5 tag fused to the ASFV gene and a different fluorescently labelled secondary antibody to confirm expression of the protein.

The pig sera used for staining the cells were from immunisation studies using the following viruses: BeninΔDP148R (5 pigs), BeninΔMGF (6 pigs), OURT88/3 (5 pigs) and GeorgiaΔMGF (4 pigs). For each pig, a pre-immunisation serum sample (as a control) and a post-immunisation, pre-challenge serum sample were used.

An initial screen of the genes was conducted using sera from BeninΔDP148R immunised pigs (pre-immunisation sera from day 0, post-immunisation sera from day 38 post-immunisation). ASFV genes CP204L, B646L and E183L were used as positive controls and were detected using post-immunisation sera. Pre-immunisation sera did not detect any genes.

Six ASFV genes were detected using BeninΔDP148R post-immunisation sera, as shown below in Table 5 (++ indicates strong detection, + indicates weak detection, − indicates no detection).

TABLE 5

| | BeninΔDP148R post-immunisation serum (day 38) | | | | |
|---|---|---|---|---|---|
| ASFV gene | Pig 1 | Pig 2 | Pig 3 | Pig 4 | Pig 5 |
| B125R | ++ | ++ | − | − | + |
| B175L | ++ | ++ | − | + | − |
| E184L | ++ | ++ | − | − | + |
| H339R | − | ++ | − | ++ | ++ |
| K145R | ++ | ++ | − | − | − |
| M448R | ++ | − | + | ++ | + |

The six ASFV genes detected in the initial screen were then tested with pig serum from other immunisation studies.

Table 6 below shows detection of ASFV genes using post-immunisation sera from 6 pigs immunised with BeninΔMGF virus (boosted on day 15, post-immunisation serum taken on day 38 post-immunisation; pre-immunisation sera were negative).

TABLE 6

| | BeninΔMGF post-immunisation serum (day 38) | | | | | |
|---|---|---|---|---|---|---|
| ASFV gene | Pig 1 | Pig 2 | Pig 3 | Pig 4 | Pig 5 | Pig 6 |
| B125R | ++ | ++ | + | ++ | ++ | ++ |
| B175L | ++ | ++ | ++ | ++ | ++ | ++ |
| E184L | ++ | ++ | ++ | ++ | ++ | + |
| H339R | − | − | + | + | ++ | ++ |
| K145R | + | − | − | ++ | ++ | ++ |
| M448R | ++ | − | ++ | − | + | ++ |

Table 7 below shows detection of ASFV genes using post-immunisation sera from 5 pigs immunised with OURT88/3 virus (post-immunisation serum taken on day 20 post-immunisation; pre-immunisation sera were negative except for pig 2).

TABLE 7

| | OURT88/3 post-immunisation serum (day 20) | | | | |
|---|---|---|---|---|---|
| ASFV gene | Pig 1 | Pig 2 | Pig 3 | Pig 4 | Pig 5 |
| B125R | ++ | ++ | + | + | − |
| B175L | ++ | ++ | + | ++ | + |
| E184L | ++ | ++ | + | ++ | ++ |
| H339R | ++ | ++ | + | ++ | + |
| K145R | ++ | ++ | + | + | + |
| M448R | ++ | ++ | + | ++ | + |

Table 8 below shows detection of ASFV genes using post-immunisation sera from 4 pigs immunised with GeorgiaΔMGF virus (post-immunisation serum taken on day 34 post-immunisation; pre-immunisation sera taken on day −3 were negative). 2 pigs (A) were immunised with 10³ GeorgiaΔMGF; 2 pigs (B) were immunised with 10⁴ GeorgiaΔMGF.

TABLE 8

| | GeorgiaΔMGF post-immunisation serum (day 34) | | | |
|---|---|---|---|---|
| ASFV gene | Pig 1 (A) | Pig 2 (A) | Pig 3 (B) | Pig 4 (B) |
| B125R | − | ++ | + | − |
| B175L | + | − | − | + |
| E184L | − | − | ++ | + |
| H339R | − | + | − | − |
| K145R | − | ++ | + | − |
| M448R | − | − | ++ | − |

### Example 5—Localisation of B125R at the Cell Surface

Vero cells were transfected with a plasmid expressing the B125R gene fused to an HA-epitope tag. Cells were fixed and permeabilised then stained with an anti-HA antibody followed by appropriate secondary antibody. FIG. 11 shows that the B125R protein (green) localises to the cell surface.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in virology, molecular biology or related fields are intended to be within the scope of the following claims.

1. An attenuated African Swine Fever (ASF) virus in which the expression and/or activity of the following genes is disrupted:
multigene-family (MGF) 360 12L, 13L and 14L, and
MGF 505 1R,
and which comprises a functional version of the following genes:
(i) MGF 360 10L and/or 11L, and
(ii) MGF 505 2R and/or 3R.

2. An attenuated ASF virus according to claim 1 comprising functional versions of MGF 360 10L and 11L.

3. An attenuated ASF virus according to claim 1 or 2 comprising functional versions of MGF 505 2R and 3R.

4. An attenuated ASF virus according to any of claims 1 to 3, wherein one or more of the following genes is at least partially deleted:
MGF 360 12L, 13L and 14L, and
MGF 505 1R.

5. An attenuated ASF virus according to any of claims 1 to 4, wherein one or more of the following genes is completely deleted:
MGF 360 12L, 13L and 14L, and
MGF 505 1R.

6. An attenuated virus according to any of claims 1 to 5, wherein one or more of the following genes is interrupted:
MGF 360 12L, 13L and 14L, and
MGF 505 1R.

7. An attenuated ASF virus according to any of claims 1 to 6 which comprises a Differentiating Infected from Vaccinated Animals (DIVA) mutation.

8. An attenuated ASF virus according to claim 7, wherein the DIVA mutation is lack of a functional version of the K145R gene and/or lack of a functional version of the B125R gene.

9. An attenuated ASF virus which lacks a functional version of the K145R gene and/or the B125R gene.

10. An attenuated ASF virus according to claim 8 or 9, wherein the K145R gene and/or the B125R gene is at least partially deleted.

11. An attenuated ASF virus according to any of claims 8 to 10, wherein the K145R gene and/or the B125R gene is completely deleted.

12. An attenuated ASF virus according to any of claims 8 to 11, wherein the K145R gene and/or the B125R gene is interrupted.

13. A vaccine comprising an attenuated ASF virus according to any of claims 1 to 12.

14. A vaccine according to claim 13 for use in treating and/or preventing African Swine Fever in a subject.

15. A method for treating and/or preventing African Swine Fever in a subject which comprises the step of administering to the subject an effective amount of a vaccine according to claim 13.

16. A vaccine for use according to claim 14, or a method according to claim 15, wherein the subject is a domestic pig.

17. A vaccine for use according to claim 14 or 16, or a method according to claim 15 or 16, in which the vaccine is administered following a prime-boost regime.

18. A method of attenuating an ASF virus which comprises the step of disrupting the expression and/or activity of the following genes:
MGF 360 12L, 13L and 14L, and
MGF 505 1R,
whilst retaining the function of the following genes:
(i) MGF 360 10L and/or 11L, and
(ii) MGF 505 2R and/or 3R.

19. A method according to claim 18 wherein the function of the MGF 360 10L and 11L genes is retained.

20. A method according to claim 18 or 19 wherein the function of the MGF 505 2R and 3R genes is retained.

21. A method according to any of claims 18 to 20 wherein one or more of the following genes is at least partially deleted:
MGF 360 12L, 13L and 14L, and
MGF 505 1R.

22. A method according to any of claims 18 to 21 wherein one or more of the following genes is completely deleted:
MGF 360 12L, 13L and 14L, and
MGF 505 1R.

23. A method according to any of claims 18 to 22 wherein one or more of the following genes is interrupted:
MGF 360 12L, 13L and 14L, and
MGF 505 1R.
